(19) United States
(12) Patent Application Publication
(10) Pub. No.: US 2011/0104109 A1
(43) Pub. Date: May 5, 2011

(54) TETRACYCLIC INDOLE DERIVATIVES AND THEIR USE FOR TREATING OR PREVENTING VIRAL INFECTIONS

(76) Inventors: Frank Bennett, Cranford, NJ (US); Qingbei Zeng, Edison, NJ (US); Srikanth Venkatraman, Edison, NJ (US); Mousumi Sannigrahi, Summit, NJ (US); Kevin X. Chen, Edison, NJ (US); Gopinadhan N. Anilkumar, Edison, NJ (US); Stuart B. Rosenblum, West Orange, NJ (US); Joseph A. Kozlowski, Princeton, NJ (US); F. George Njoroge, Warren, NJ (US)

(21) Appl. No.: 12/675,277

(22) PCT Filed: Aug. 27, 2008
(86) PCT No.: PCT/US08/10147

§ 371 Date: Dec. 20, 2010

Related U.S. Application Data
(60) Provisional application No. 60/698,684, filed on Jul. 13, 2005.

(51) Int. Cl. A61K 31/437; C07D 498/22; C07D 491/147; C07D 491/22; C07D 513/22; C07D 495/04; A61K 31/506; A61K 31/517; A61K 31/519; A61K 31/536; A61K 38/21; A61K 39/12; A61K 39/42; A61K 31/7088; A61P 31/12; A61P 37/04

(52) U.S. Cl. 424/85.4; 546/48; 546/64; 544/310; 544/284; 544/278; 544/92; 514/280; 514/287; 514/274; 514/266.21; 514/260.1; 514/230.5; 424/204.1; 424/159.1; 424/147.1; 514/44 A

(57) ABSTRACT

The present invention relates to Tetracyclic Indole Derivatives, compositions comprising at least one Tetracyclic Indole Derivative, and methods of using the Tetracyclic Indole Derivatives for treating or preventing a viral infection or a virus-related disorder in a patient.

### FIELD OF THE INVENTION

The present invention relates to Tetracyclic Indole Derivatives, compositions comprising at least one Tetracyclic Indole Derivative, and methods of using the Tetracyclic Indole Derivatives for treating or preventing a viral infection or a virus-related disorder in a patient.

### BACKGROUND OF THE INVENTION

HCV is a (+)-sense single-stranded RNA virus that has been implicated as the major causative agent in non-A, non-B hepatitis (NANBH). NANBH is distinguished from other types of viral-induced liver disease, such as hepatitis A virus (HAV), hepatitis B virus (HBV), hepatitis delta virus (HDV), as well as from other forms of liver disease such as alcoholism and primary biliary cirrhosis.

Hepatitis C virus is a member of the hepacivirus genus in the family Flaviviridae. It is the major causative agent of non-A, non-B viral hepatitis and is the major cause of transfusion-associated hepatitis and accounts for a significant proportion of hepatitis cases worldwide. Although acute HCV infection is often asymptomatic, nearly 80% of cases resolve to chronic hepatitis. About 60% of patients develop liver disease with various clinical outcomes ranging from an asymptomatic carrier state to chronic active hepatitis and liver cirrhosis (occurring in about 20% of patients), which is strongly associated with the development of hepatocellular carcinoma (occurring in about 1-5% of patients). The World Health Organization estimates that 170 million people are chronically infected with HCV, with an estimated 4 million living in the United States.

HCV has been implicated in cirrhosis of the liver and in induction of hepatocellular carcinoma. The prognosis for patients suffering from HCV infection remains poor as HCV infection is more difficult to treat than other forms of hepatitis. Current data indicates a four-year survival rate of below 50% for patients suffering from cirrhosis and a five-year survival rate of below 30% for patients diagnosed with localized resectable hepatocellular carcinoma. Patients diagnosed with localized unresectable hepatocellular carcinoma fare even worse, having a five-year survival rate of less than 1%.

HCV is an enveloped RNA virus containing a single-stranded positive-sense RNA genome approximately 9.5 kb in length. The RNA genome contains a 5′-nontranslated region (5′ NTR) of 341 nucleotides, a large open reading frame (ORF) encoding a single polypeptide of 3,010 to 3,040 amino acids, and a 3′-nontranslated region (3′-NTR) of variable length of about 230 nucleotides. HCV is similar in amino acid sequence and genome organization to flaviviruses and pestiviruses, and therefore HCV has been classified as a third genus of the family Flaviviridae.

The 5′ NTR, one of the most conserved regions of the viral genome, contains an internal ribosome entry site (IRES) which plays a pivotal role in the initiation of translation of the viral polyprotein. A single long open reading frame encodes a polyprotein, which is co- or post-translationally processed into structural (core, E1, E2 and p7) and nonstructural (NS2, NS3, NS4A, NS4B, NS5A, and NS5B) viral proteins by either cellular or viral proteinases. The 3′ NTR consists of three distinct regions: a variable region of about 38 nucleotides following the stop codon of the polyprotein, a polyuridine tract of variable length with interspersed substitutions of cytidines, and 98 nucleotides (nt) at the very 3′ end which are highly conserved among various HCV isolates. By analogy to other plus-strand RNA viruses, the 3′-NTR is thought to play an important role in viral RNA synthesis. The order of the genes within the genome is: NH₂-C-E1-E2-p7-NS2-NS3-NS4A-NS4B-NS5A-NS5B-COOH.

Processing of the structural proteins core (C), envelope protein 1 and (E1, E2), and the p7 region is mediated by host signal peptidases. In contrast, maturation of the nonstructural (NS) region is accomplished by two viral enzymes. The HCV polyprotein is first cleaved by a host signal peptidase generating the structural proteins C/E1, E1/E2, E2/p7, and p7/NS2. The NS2-3 proteinase, which is a metalloprotease, then cleaves at the NS2/NS3 junction. The NS3/4A proteinase complex (NS3 being a serine protease and NS4A acting as a cofactor of the NS3 protease), is then responsible for processing all the remaining cleavage junctions. RNA helicase and NTPase activities have also been identified in the NS3 protein. One-third of the NS3 protein functions as a protease, and the remaining two-thirds of the molecule acts as the helicase/ATPase that is thought to be involved in HCV replication. NS5A may be phosphorylated and acts as a putative cofactor of NS5B. The fourth viral enzyme, NS5B, is a membrane-associated RNA-dependent RNA polymerase (RdRp) and a key component responsible for replication of the viral RNA genome. NS5B contains the “GDD” sequence motif, which is highly conserved among all RdRps characterized to date.

Replication of HCV is thought to occur in membrane-associated replication complexes. Within these, the genomic plus-strand RNA is transcribed into minus-strand RNA, which in turn can be used as a template for synthesis of progeny genomic plus-strands. At least two viral enzymes appear to be involved in this reaction: the NS3 helicase/NTPase, and the NS5B RNA-dependent RNA polymerase. While the role of NS3 in RNA replication is less clear, NS5B is the key enzyme responsible for synthesis of progeny RNA strands. Using recombinant baculoviruses to express NS5B in insect cells and a synthetic nonviral RNA as a substrate, two enzymatic activities have been identified as being associated with it: a primer-dependent RdRp and a terminal transferase (TNTase) activity. It was subsequently confirmed and further characterized through the use of the HCV RNA genome as a substrate. Other studies have shown that NS5B with a C-terminal 21 amino-acid truncation expressed in Escherichia coli is also active for in vitro RNA synthesis. On certain RNA templates, NS5B has been shown to catalyze RNA synthesis via a de novo initiation mechanism, which has been postulated to be the mode of viral replication in vivo. Templates with single-stranded 3′ termini, especially those containing a 3′-terminal cytidylate moiety, have been found to direct de novo synthesis efficiently. There has also been evidence for NS5B to utilize di- or tri-nucleotides as short primers to initiate replication.

It is well-established that persistent infection of HCV is related to chronic hepatitis, and as such, inhibition of HCV replication is a viable strategy for the prevention of hepatocellular carcinoma. Present treatment approaches for HCV infection suffer from poor efficacy and unfavorable side-effects and there is currently a strong effort directed to the discovery of HCV replication inhibitors that are useful for the treatment and prevention of HCV related disorders. New approaches currently under investigation include the development of prophylactic and therapeutic vaccines, the identification of interferons with improved pharmacokinetic characteristics, and the discovery of agents designed to inhibit the function of three major viral proteins: protease, helicase and polymerase. In addition, the HCV RNA genome itself, particularly the IBES element, is being actively exploited as an antiviral target using antisense molecules and catalytic ribozymes.

Particular therapies for HCV infection include α-interferon monotherapy and combination therapy comprising α-interferon and ribavirin. These therapies have been shown to be effective in some patients with chronic HCV infection. The use of antisense oligonucleotides for treatment of HCV infection has also been proposed as has the use of free bile acids, such as ursodeoxycholic acid and chenodeoxycholic acid, and conjugated bile acids, such as tauroursodeoxycholic acid. Phosphonoformic acid esters have also been proposed as potentially for the treatment of various viral infections including HCV. Vaccine development, however, has been hampered by the high degree of viral strain heterogeneity and immune evasion and the lack of protection against reinfection, even with the same inoculum.

The development of small-molecule inhibitors directed against specific viral targets has become a major focus of anti-HCV research. The determination of crystal structures for NS3 protease, NS3 RNA helicase, and NS5B polymerase has provided important structural insights that should assist in the rational design of specific inhibitors.

NS5B, the RNA-dependent RNA polymerase, is an important and attractive target for small-molecule inhibitors. Studies with pestiviruses have shown that the small molecule compound VP32947 (3-R(2-dipropylamino)ethyl)thio]-5H-1,2,4-triazino[5,6-b]indole) is a potent inhibitor of pestivirus replication and most likely inhibits the NS5B enzyme since resistant strains are mutated in this gene. Inhibition of RdRp activity by (−)β-L-2′,3′-dideoxy-3′-thiacytidine 5′-triphosphate (3TC; lamivudine triphosphate) and phosphonoacetic acid also has been observed.

Despite the intensive effort directed at the treatment and prevention of HCV and related viral infections, there exists a need in the art for non-peptide, small-molecule compounds having desirable or improved physicochemical properties that are useful for inhibiting viruses and treating viral infections and virus-related disorders.

### SUMMARY OF THE INVENTION

In one aspect, the present invention provides compounds of formula (I):

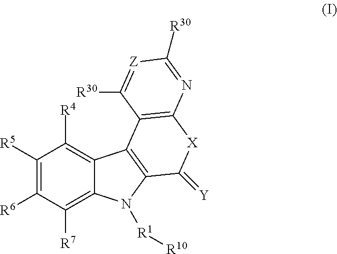

and pharmaceutically acceptable salts, solvates, esters and prodrugs thereof.
wherein:

X is —O—, —S—, —NH—, —N(R⁹)—, —OC(R⁸)₂O— or —OC(R⁸)₂N(R⁹)—;

Y is ═O, ═NH, ═NR⁹, ═NSOR¹¹, ═NSO₂R¹¹ or ═NSO₂N(R¹¹)₂;

Z is —N— or —C(R³¹)—;

R¹ is a bond, —[C(R¹²)₂]ᵣ—O—[C(R¹²)₂]_{q}—, —[C(R¹²)₂]ᵣ—N(R⁹)—[C(R¹²)₂]_{q}—, —[C(R¹²)₂]_{q}—CH═CH—[C(R¹²)₂]_{q}—C≡C—[C(R¹²)₂]_{q}—or —[C(R¹²)₂]_{q}—SO₂—[C(R¹²)₂]_{q}—;

R⁴, R⁵, R⁶ and R⁷ are each, independently, H, alkyl, alkenyl, alkynyl, aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹, —CN, —[C(R¹²)₂]_{q}—C(O)R⁸, —C(O)OR⁹, —[C(R¹²)₂]_{q}—C(O)N(R⁹)₂, —[C(R¹²)₂]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂R¹¹, —[C(R¹²)₂]_{q}—S(O)ₚR¹¹, —[C(R¹²)₂]_{q}—SO₂N(R⁹)₂—SO₂N(R⁹)C(O)N(R⁹)₂, or R⁴ and R⁵, together with the carbon atoms to which they are attached, join to form a 3- to 7-membered cyclic group, selected from cycloalkyl, heterocycloalkyl, aryl or heteroaryl, or R⁵ and R⁶, together with the carbon atoms to which they are attached, join to form a 3- to 7-membered cyclic group, selected from cycloalkyl, heterocycloalkyl, aryl or heteroaryl, or R⁶ and R⁷, together with the carbon atoms to which they are attached, join to form a 3- to 7-membered cyclic group, selected from cycloalkyl, heterocycloalkyl, aryl or heteroaryl;

each occurrence of R⁸ is independently H, alkyl, alkenyl, alkynyl, —[C(R¹²)₂]_{q}-aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, haloalkyl or hydroxyalkyl;

each occurrence of R⁹ is independently H, alkyl, alkenyl, alkynyl, —[C(R¹²)₂]_{q}-aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, haloalkyl or hydroxyalkyl;

R¹⁰ is H, halo, cycloalkyl, cycloalkenyl, heterocycloalkyl, heterocycloalkenyl, aryl, heteroaryl, wherein a cycloalkyl, cycloalkenyl, heterocycloalkyl, heterocycloalkenyl, aryl or heteroaryl group can be optionally and independently substituted with up to 4 substituents, which are each independently selected from H, alkyl, alkenyl, alkynyl, aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹, —CN, —[C(R¹²)₂]_{q}—C(O)R⁸, —[C(R¹²)₂]_{q}—C(O)OR⁹, —[C(R¹²)₂]_{q}—C(O)N(R⁹)₂, —[C(R¹²)2]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂R¹¹, —[C(R¹²)₂]_{q}—S(O)ₚR¹¹, —[C(R¹²)₂]_{q}—SO₂N(R⁹)₂ and —S(O)₂N(R⁹)C(O)N(R⁹)₂, such that when R¹ is a bond, R¹⁰ is other than H;

each occurrence of R¹¹ is independently alkyl, aryl, cycloalkyl, cycloalkenyl, heterocycloalkyl, heterocycloalkenyl, heteroaryl, haloalkyl, hydroxy or hydroxyalkyl, wherein a cycloalkyl, cycloalkenyl, heterocycloalkyl, heterocycloalkenyl, aryl or heteroaryl group can be optionally and independently substituted with up to 4 substituents, which are each independently selected from —H, alkyl, alkenyl, alkynyl, aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹, —CN, —[C(R¹²)₂]_{q}—C(O)R⁸, —[C(R¹²)₂]_{q}—C(O)OR⁹, —[C(R¹²)₂]_{q}—C(O)N(R⁹)₂, —[C(R¹²)₂]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂alkyl, —[C(R¹²)₂]_{q}—NHSO₂cycloalkyl, —[C(R¹²)₂]_{q}—NHSO₂aryl, —[C(R¹²)₂]_{q}—SO₂N(R⁹)₂ and —SO₂N(R⁹)C(O)N(R⁹)₂;

each occurrence of R¹² is independently H, halo, —N(R⁹)₂, —OR⁹, alkyl, cycloalkyl, cycloalkenyl, heterocycloalkyl or heterocycloalkenyl, wherein a cycloalkyl, cycloalkenyl, heterocycloalkyl or heterocycloalkenyl group can be optionally and independently substituted with up to 4 substituents, which are each independently selected from alkyl, halo, haloalkyl, hydroxyalkyl, hydroxy, —CN, —C(O)alkyl, —C(O)Oalkyl, —C(O)NHalkyl, —C(O)N(alkyl)₂, —O-alkyl, —NH₂, —NH(alkyl), —N(alkyl)₂, —NHC(O)alkyl, —NHSO₂alkyl, —SO₂alkyl or —SO₂NH-alkyl, or two geminal R¹² groups, together with the common carbon atom to which they are attached, join to form a 3- to 7-membered cycloalkyl, 3- to 7-membered heterocycloalkyl or C═O group;

each occurrence of R³⁰ is independently, H, alkyl, alkenyl, alkynyl, aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹, —CN, —[C(R¹²)₂]_{q}—C(O)R⁸, —[C(R¹²)₂]_{q}—C(O)OR⁹, —[C(R¹²)₂]_{q}—C(O)N(R⁹)₂, —[C(R¹²)₂]_{q}—OR⁹, —]C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂R¹¹, —[C(R¹²)₂]_{q}—S(O)ₚR¹¹, —[C(R¹²)₂]_{q}—SO₂N(R⁹)₂—SO₂N(R⁹)C(O)N(R⁹)₂, or any R³⁰ and R³¹, together with the carbon atoms to which they are attached, join to form a 3- to 7-membered cyclic group, selected from cycloalkyl, heterocycloalkyl, aryl and heteroaryl;

R³¹ is H, alkyl, alkenyl, alkynyl, aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹ or —CN;

each occurrence of p is independently 0, 1 or 2;

each occurrence of q is independently an integer ranging from 0 to 4; and

each occurrence of r is independently an integer ranging from 1 to 4.

The compounds of formula (I) (the “Tetracyclic Indole Derivatives”) and pharmaceutically acceptable salts, solvates, esters and prodrugs thereof can be useful for treating or preventing a viral infection or a virus-related disorder in a patient.

Also provided by the invention are methods for treating or preventing a viral infection or a virus-related disorder in a patient, comprising administering to the patient an effective amount of at least one Tetracyclic Indole Derivative.

The present invention further provides pharmaceutical compositions comprising an effective amount of at least one Tetracyclic Indole Derivative or a pharmaceutically acceptable salt, solvate thereof, and a pharmaceutically acceptable carrier. The compositions can be useful for treating or preventing a viral infection or a virus-related disorder in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

In an embodiment, the present invention provides Tetracyclic Indole Derivatives, pharmaceutical compositions comprising at least one Tetracyclic Indole Derivative, and methods of using the Tetracyclic Indole Derivatives for treating or preventing a viral infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “alkoxy,” etc.

As used herein, and throughout this disclosure, the following terms, unless otherwise indicated, shall be understood to have the following meanings:

A “patient” is a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “alkyl” as used herein, refers to an aliphatic hydrocarbon group, wherein one of the aliphatic hydrocarbon group's hydrogen atoms is replaced with a single bond. An alkyl group can be straight or branched and can contain from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or optionally substituted by one or more substituents which may be the same or different, each substituent being independently selected from the group consisting of halo, alkenyl, alkynyl, —O-aryl, aryl, heteroaryl, cycloalkyl, cycloalkenyl, cyano, hydroxy, —O-alkyl, —O-haloalkyl, -alkylene-O-alkyl, alkylthio, —NH₂, —NH(alkyl), —N(alkyl)₂, —NH-aryl, —NH-heteroaryl, —NHC(O)-alkyl, —NHC(O)NH-alkyl, —NHSO₂-alkyl, —NHSO₂-aryl, —NHSO₂-heteroaryl, —NH(cycloalkyl), —OC(O)-alkyl, —OC(O)-aryl, —OC(O)-cycloalkyl, —C(O)alkyl, —C(O)NH₂, —C(O)NH-alkyl, —C(O)OH and —C(O)O-alkyl. In one embodiment, an alkyl group is unsubstituted. In another embodiment, an alkyl group is a straight chain alkyl group. In another embodiment, an alkyl group is a branched alkyl group.

The term “alkenyl” as used herein, refers to an aliphatic hydrocarbon group having at least one carbon-carbon double bond, wherein one of the aliphatic hydrocarbon group's hydrogen atoms is replaced with a single bond. An alkenyl group can be straight or branched and can contain from about 2 to about 15 carbon atoms. In one embodiment, an alkenyl group contains from about 2 to about 10 carbon atoms. In another embodiment, an alkenyl group contains from about 2 to about 6 carbon atoms. Non-limiting examples of illustrative alkenyl groups include ethenyl, propenyl, n-butenyl, 3-methylbut-2-enyl, n-pentenyl, octenyl and decenyl. An alkenyl group may be unsubstituted or optionally substituted by one or more substituents which may be the same or different, each substituent being independently selected from the group consisting of halo, alkyl, alkynyl, —O-aryl, aryl, cycloalkyl, cycloalkenyl, cyano, hydroxy, —O-alkyl, —O-haloalkyl, -alkylene-O-alkyl, alkylthio, —NH₂, —NH(alkyl), —N(alkyl)₂, —NH-aryl, —NH-heteroaryl, —NHC(O)-alkyl, —NHC(O)NH-alkyl, —NHSO₂-alkyl, —NHSO₂-aryl, —NHSO₂-heteroaryl, —NH(cycloalkyl), —OC(O)-alkyl, —OC(O)-aryl, —OC(O)-cycloalkyl, —C(O)alkyl, —C(O)NH₂, —C(O)NH-alkyl, —C(O)OH and —C(O)O-alkyl. In one embodiment, an alkenyl group is unsubstituted. In another embodiment, an alkenyl group is a straight chain alkenyl group. In another embodiment, an alkyl group is a branched alkenyl group.

The term “alkynyl” as used herein, refers to an aliphatic hydrocarbon group having at least one carbon-carbon triple bond, wherein one of the aliphatic hydrocarbon group's hydrogen atoms is replaced with a single bond. An alkynyl group can be straight or branched and can contain from about 2 to about 15 carbon atoms. In one embodiment, an alkynyl group contains from about 2 to about 10 carbon atoms. In another embodiment, an alkynyl group contains from about 2 to about 6 carbon atoms. Non-limiting examples of illustrative alkynyl groups include ethynyl, propynyl, 2-butynyl and 3-methylbutynyl. An alkynyl group may be unsubstituted or optionally substituted by one or more substituents which may be the same or different, each substituent being independently selected from the group consisting of halo, alkyl, alkenyl, —O-aryl, aryl, cycloalkyl, cycloalkenyl, cyano, hydroxy, —O-alkyl, —O-haloalkyl, -alkylene-O-alkyl, alkylthio, —NH₂, —NH(alkyl), —N(alkyl)₂, —NH-aryl, —NH-heteroaryl, —NHC(O)-alkyl, —NHC(O)NH-alkyl, —NHSO₂-alkyl, —NHSO₂-aryl, —NHSO₂-heteroaryl, —NH(cycloalkyl), —OC(O)-alkyl, —OC(O)-aryl, —OC(O)-cycloalkyl, —C(O)alkyl, —C(O)NH₂, —C(O)NH-alkyl, —C(O)OH and —C(O)O-alkyl. In one embodiment, an alkynyl group is unsubstituted. In another embodiment, an alkynyl group is a straight chain alkynyl group. In another embodiment, an alkynyl group is a branched alkynyl group.

The term “alkylene” as used herein, refers to an alkyl group, as defined above, wherein one of the alkyl group's hydrogen atoms is replaced with a bond. Illustrative examples of alkylene include, but are not limited to, —CH₂—, —CH₂CH₂—, —CH₂CH₂CH₂—, —CH₂CH₂CH₂CH₂—, —CH(CH₃)CH₂CH₂—, —CH₂CH(CH₃)CH₂— and —CH₂CH₂CH(CH₃)—. In one embodiment, an alkylene group is a straight chain alkylene group. In another embodiment, an alkylene group is a branched alkylene group.

“Aryl” means an aromatic monocyclic or multicyclic ring system having from about 6 to about 14 ring carbon atoms. In one embodiment, an aryl group has from about 6 to about 10 ring carbon atoms. An aryl group can be optionally substituted with one or more “ring system substituents” which may be the same or different, and are as defined herein below. Non-limiting examples of illustrative aryl groups include phenyl and naphthyl. In one embodiment, an aryl group is unsubstituted. In another embodiment, an aryl group is a phenyl group.

The term “cycloalkyl” as used herein, refers to a non-aromatic mono- or multicyclic ring system having from about 3 to about 10 ring carbon atoms. In one embodiment, a cycloalkyl has from about 5 to about 10 ring carbon atoms. In another embodiment, a cycloalkyl has from about 5 to about 7 ring carbon atoms. Non-limiting examples of illustrative monocyclic cycloalkyls include cyclopropyl, cyclopentyl, cyclohexyl, cycloheptyl and the like. Non-limiting examples of illustrative multicyclic cycloalkyls include 1-decalinyl, norbomyl, adamantyl and the like. A cycloalkyl group can be optionally substituted with one or more “ring system substituents” which may be the same or different, and are as defined herein below. In one embodiment, a cycloalkyl group is unsubstituted.

The term “cycloalkenyl” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring carbon atoms. Non-limiting examples of illustrative monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted.

The term “halo” as used herein, means —F, —Cl, —Br or —I. In one embodiment, halo refers to —Cl or —F.

The term “haloalkyl” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of illustrative haloalkyl groups include —CH₂F, —CF₃, —CH₂Cl and —CCl₃.

The term “hydroxyalkyl” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of illustrative hydroxyalkyl groups include hydroxymethyl, 2-hydroxyethyl, 3-hydroxypropyl, 4-hydroxybutyl and —CH(OH)CH₂CH₃.

The term “heteroaryl” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is monocyclic and has 5 or 6 ring atoms and at least one nitrogen ring atom. A heteroaryl group can be optionally substituted by one or more “ring system substituents” which may be the same or different, and are as defined herein below. A heteroaryl group is joined via a ring carbon atom and any nitrogen atom of a heteroaryl can be optionally oxidized to the corresponding N-oxide. The term “heteroaryl” also encompasses a heteroaryl group, as defined above, which has been fused to a benzene ring. Non-limiting examples of illustrative heteroaryls include pyridyl, pyrazinyl, (uranyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. In one embodiment, a heteroaryl group is a 6-membered heteroaryl group. In another embodiment, a heteroaryl group is a 5-membered heteroaryl group.

The term “heterocycloalkyl” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(Boc), —N(CBz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocyclyl can be optionally oxidized to the corresponding N-oxide, S-oxide or S,S-dioxide. Non-limiting examples of illustrative monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is is pyrrolidonvl:

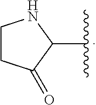

In one embodiment, a heterocycloalkyl group is a 6-membered heterocycloalkyl group. In another embodiment, a heterocycloalkyl group is a 5-membered heterocycloalkyl group.

The term “heterocycloalkenyl” as used herein, refers to a heterocycloalkyl group, as defined above, wherein the heterocycloalkyl group contains from 3 to 10 ring atoms, and at least one endocyclic carbon-carbon or carbon-nitrogen double bond. In one embodiment, a heterocycloalkenyl group has from 5 to 10 ring atoms. In another embodiment, a heterocycloalkenyl group is monocyclic and has 5 or 6 ring atoms. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of illustrative heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, pyridonyl (including N-substituted pyridone), dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. A ring carbon atom of a heterocycloalkenyl group may be functionalized as a carbonyl group. An illustrative example of such a heterocycloalkenyl group is:

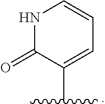

In one embodiment, a heterocycloalkenyl group is a 6-membered heterocycloalkenyl group. In another embodiment, a heterocycloalkenyl group is a 5-membered heterocycloalkenyl group.

The term “ring system substituent” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, haloalkyl, —O-alkyl, —O-haloalkyl, -alkylene-O-alkyl, —O-aryl, aralkoxy, acyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —OC(O)-alkyl, —OC(O)-aryl, —OC(O)-cycloalkyl, —C(═N—CN)—NH₂, —C(═NH)—NH₂, —C(═NH)—NH(alkyl), —NY₁Y₂, -alkylene-NY₁Y₂, —C(O)NY₁Y₂ and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on the same carbon atom (such as to to form a carbonyl group) or replaces two available hydrogen atome on two adjacent carbon atoms (one H on each carbon) on a ring system. Examples of such moiety are ═O, methylene dioxy, ethylenedioxy, —C(CH₃)₂— and the like which form moieties such as, for example:

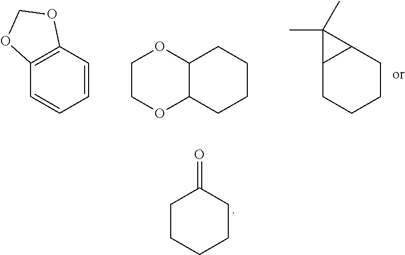

The term “substituted,” as used herein, means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “optionally substituted” as used herein, means optional substitution with the specified groups, radicals or moieties.

The terms “purified”, “in purified form” or “in isolated and purified form” as used herein, for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) , in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R¹¹, etc.) occurs more than one time in any constituent or in Formula (I), its definition on each occurrence is independent of its definition at every other occurrence, unless otherwise noted.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” as used herein, refers to a compound (e.g, a drug precursor) that is transformed in vivo to yield a Tetracyclic Indole Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Tetracyclic Indole Derivative or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Tetracyclic Indole Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a Tetracyclic Indole Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “effective amount” or “therapeutically effective amount” is meant to describe an amount of compound or a composition of the present invention that is effective to treat or prevent a viral infection or a virus-related disorder.

Metabolic conjugates, such as glucuronides and sulfates which can undergo reversible conversion to the Tetracyclic Indole Derivatives are contemplated in the present invention.

The Tetracyclic Indole Derivatives may form salts, and all such salts are contemplated within the scope of this invention. Reference to a Tetracyclic Indole Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Tetracyclic Indole Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a Tetracyclic Indole Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, choline, and salts with amino acids such as arginine, lysine and the like. Basic nitrogen-containing groups may be quartemized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

The Tetracyclic Indole Derivatives may contain asymmetric or chiral centers, and, therefore, exist in different stereoisomeric forms. It is intended that all stereoisomeric forms of the Tetracyclic Indole Derivatives as well as mixtures thereof, including racemic mixtures, form part of the present invention. In addition, the present invention embraces all geometric and positional isomers. For example, if a Tetracyclic Indole Derivative incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the Tetracyclic Indole Derivatives may be atropisomers (e.g., substituted biaryls) and are considered as part of this invention. Enantiomers can also be separated by use of chiral HPLC column.

The straight line [symbol] as a bond generally indicates a mixture of, or either of, the possible isomers, non-limiting example(s) include, containing (R)- and (S)-stereochemistry. For example,

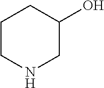

means containing both

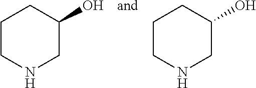

A dashed line [symbol] represents an optional bond.

Lines drawn into the ring systems, such as, for example:

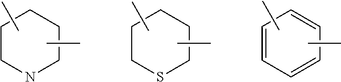

indicate that the indicated line (bond) may be attached to any of the substitutable ring atoms, non limiting examples include carbon, nitrogen and sulfur ring atoms.

As well known in the art, a bond drawn from a particular atom wherein no moiety is depicted at the terminal end of the bond indicates a methyl group bound through that bond to the atom, unless stated otherwise. For example:

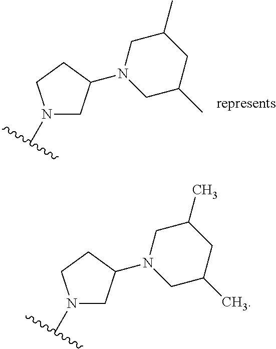

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). For example, if a Tetracyclic Indole Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Such compounds are useful as therapeutic, diagnostic or research reagents. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Tetracyclic Indole Derivatives (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled Tetracyclic Indole Derivatives can generally be prepared by following procedures analogous to those disclosed in the Schemes and/or in the Examples herein below, by substituting an appropriate isotopically labelled reagent for a non-isotopically labelled reagent.

Polymorphic forms of the Tetracyclic Indole Derivatives, and of the salts, solvates, hydrates, esters and prodrugs of the Tetracyclic Indole Derivatives, are intended to be included in the present invention.

The following abbreviations are used below and have the following meanings: ATP is adenosine-5′-triphosphate; BSA is bovine serum albumin; CDCl₃ is deuterated chloroform; CTP is cytidine-5′-triphosphate; DABCO is 1,4-diazabicyclo[2.2.2]octane; dba is dibenzylideneacetone; DME is dimethoxyethane; DMF is N,N-dimethylformamide; DMSO is dimethylsulfoxide; dppf is 1,1′-bis(diphenylphosphino)ferrocene; DTT is 1,4-dithio-threitol; EDCI is 1-(3-dimethylaminopropyl)-3-ethylcarbodiimide; EDTA is ethylenediaminetetraacetic acid; Et₃N is triethylamine; EtOAc is ethyl acetate; GTP is guanosine-5′-triphosphate; HPLC is high performance liquid chromatography; MeOH is methanol; TBAF is tetrabutylammonium fluoride; THF is tetrahydrofuran; TLC is thin-layer chromatography; TMS is trimethylsilyl; and UTP is uridine-5′-triphosphate.

### The Tetracyclic Indole Derivatives of Formula (I)

The present invention provides Tetracyclic Indole Derivatives having the formula:

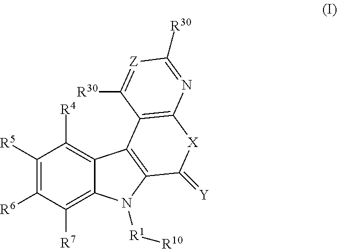

and pharmaceutically acceptable salts, solvates, esters and prodrugs thereof, wherein X, Y, Z, R¹, R⁴, R⁵, R⁶, R⁷, R¹⁰ and R³⁰ are defined above for the compounds of formula (I).

In one embodiment, X is —O—, —OCH₂O—, —NH— or —OCH₂NH—.

In another embodiment, X is —O—.

In another embodiment, X is —S—.

In another embodiment, X is —NH—.

In another embodiment, X is —N(R⁹)—.

In still another embodiment, X is —OC(R⁸)₂O—.

In yet another embodiment, X is —OC(R⁸)₂N(R⁹)—.

In one embodiment,Y is ═O.

In another embodiment, Y is ═NH.

In another embodiment, Y is ═NR⁹.

In still another embodiment, Y is ═NSOR¹¹.

In yet another embodiment, Y is ═NSO₂R¹¹.

In a further embodiment, Y is ═NSO₂N(R¹¹)₂.

In one embodiment, Z is —N—.

In another embodiment, Z is —C(R³¹)—.

In another embodiment, Z is —CH—.

In still another embodiment, Z is —C(R³¹) and R³¹ is halo.

In another embodiment, Z is —CF—.

In one embodiment, X is —O—, —OCH₂O—, —NH— or —OCH₂NH—; and Y is ═O, ═NH, ═N(R⁹)SOR¹¹, ═N(R⁹)SO₂R¹¹ or ═N(R9)SO₂N(R¹¹)₂.

In another embodiment, X is —O—, —OCH₂O—, —NH— or —OCH₂NH—; Y is ═O, ═NH, ═N(R⁹)SOR¹¹, ═N(R⁹)SO₂R¹¹ or ═N(R⁹)SO₂N(R¹¹)₂; and R¹¹ is alkyl, cycloalkyl, haloalkyl or heterocycloalkyl.

In another embodiment, X is —O—, —OCH₂O—, —NH— or —OCH₂NH—; Y is ═O, ═NH, ═N(R⁹)SOR¹¹, ═N(R⁹)SO₂R¹¹ or ═N(R⁹)SO₂N(R¹¹)₂; and R¹¹ is methyl, ethyl, isopropyl, cyclopropyl or phenyl.

In still another embodiment, X is —O—, —OCH₂O—, —NH— or —OCH₂NH—; Y is —O— or ═N(R⁹)SOR¹¹; and Z is —C(R³¹)—.

In another embodiment, X is —O—, —OCH₂O—, —NH— or —OCH₂NH—; Y is —O— or ═N(R⁹)SOR¹¹; Z is —C(R³¹)—; and R⁹ is H, methyl, ethyl or cyclopropyl.

In another embodiment, X is —O—, —OCH₂O—, —NH— or —OCH₂NH—; Y is —O— or ═N(R⁹)SOR¹¹; Z is —C(R³¹)—; and R⁹ is H or methyl.

In a further embodiment, X is —O—, —OCH₂O—, —NH— or —OCH₂NH—; and Z is —C(R³¹)—.

In one embodiment, X is —O—, —OCH₂O—, —NH— or —OCH₂NH—; Z is —C(R³¹)—; and R¹ is —[C(R¹²)₂]ᵣ—.

In another embodiment, X is —O—, —OCH₂O—, —NH— or —OCH₂NH—; Z is —C(R³¹)—; and R¹ is —CH₂—, —CH₂CH₂—, —CH(CH₃)— or

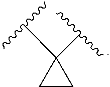

In another embodiment, X is —O—, —OCH₂O—, —NH— or —OCH₂NH—; Z is —C(R³¹)—; R¹ is —[C(R¹²)₂]ᵣ—; and R⁴ and R⁷ are each independently H, halo or hydroxy.

In another embodiment, X is —O—, —OCH₂O—, —NH— or —OCH₂NH—; Z is —C(R³¹)—; R¹ is —[C(R¹²)₂]ᵣ—; and R⁵ is H, alkyl, —O-alkyl, cycloalkyl, halo, haloalkyl, hydroxy, hydroxyalkyl, —NH₂ or —CN.

In still another embodiment, X is —O—, —OCH₂O—, —NH— or —OCH₂NH—; Z is —C(R³¹)—; R¹ is —[C(R¹²)₂]ᵣ—; and R⁴ and R⁵ groups, together with the common carbon atom to which they are attached, join to form a 3- to 7-membered cyclic group, selected from cycloalkyl, heterocycloalkyl, aryl or heteroaryl.

In another embodiment, X is —O—, —OCH₂O—, —NH— or —OCH₂NH—; Z is —C(R³¹)—; R¹ is —[C(R¹²)₂]ᵣ—; and R⁵ and R⁶ groups, together with the common carbon atom to which they are attached, join to form a 3- to 7-membered cyclic group, selected from cycloalkyl, heterocycloalkyl, aryl or heteroaryl.

In another embodiment, X is —O—, —OCH₂O—, —NH— or —OCH₂NH—; Z is —C(R³¹)—; R¹ is —[C(R¹²)₂]ᵣ—; and R⁶ and R⁷ groups, together with the common carbon atom to which they are attached, join to form a 3- to 7-membered cyclic group, selected from cycloalkyl, heterocycloalkyl, aryl or heteroaryl.

In another embodiment, X is —O—, —OCH₂O—, —NH— or —OCH₂NH—; Z is —C(R³¹)—; R¹ is —[C(R¹²)₂]ᵣ—; and R⁶ is H, alkyl, —O-alkyl, cycloalkyl, halo, haloalkyl, hydroxy, hydroxyalkyl, —NH₂ or —CN.

In yet another embodiment, X is —O—, —OCH₂O—, —NH— or —OCH₂NH—; Z is —C(R³¹)—; R¹ is —CH₂—, —CH₂CH₂—, —CH(CH₃)— or

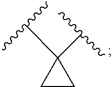

and R¹⁰ is aryl or heteroaryl.

In another embodiment, X is —O—, —OCH₂O—, —NH— or —OCH₂NH—; Z is —C(R³¹)—; R¹ is —CH₂—, —CH₂CH₂—, —CH(CH₃)— or

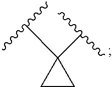

and R¹⁰ is phenyl, naphthyl, pyridyl, quinolinyl or quinoxalinyl.

In one embodiment, X is —O—, —OCH₂O—, —NH— or —OCH₂NH—; Z is —C(R³¹)—; R¹ is —CH₂—, —CH₂CH₂—, —CH(CH₃)— or

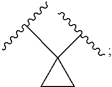

### and R¹⁰ is:

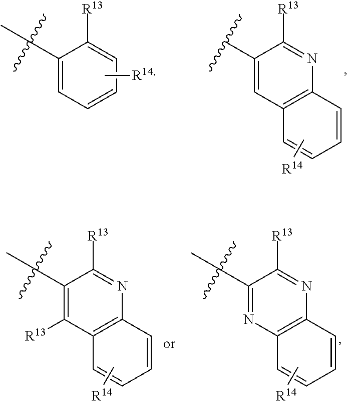

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is —O—, —OCH₂O—, —NH— or —OCH₂NH—; Z is —C(R³¹)—; R¹ is —CH₂—, —CH₂CH₂—, —CH(CH₃)— or

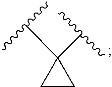

R⁵ is H, alkyl, —O-alkyl, cycloalkyl, halo, haloalkyl, hydroxy, hydroxyalkyl, —NH₂ or —CN; R⁶ is H, alkyl, —O-alkyl, cycloalkyl, halo, haloalkyl, hvdroxy , hydroxylakyl, —NH₂ or —CN; and R¹⁰ is:

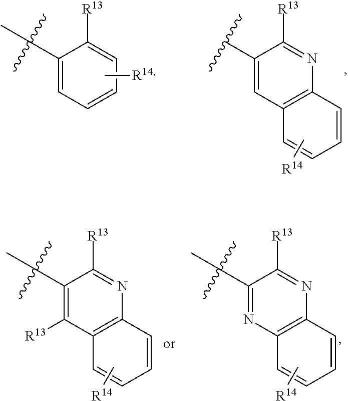

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is —O—, —OCH₂O—, —NH— or —OCH₂NH—; Z is —C(R³¹)—; R¹ is —CH₂—, —CH₂CH₂—, —CH(CH₃)— or

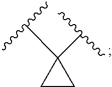

R⁵ is methyl, ethyl or cyclopropyl; R⁶ is H, Cl, F or hydroxy; and R¹⁰ is:

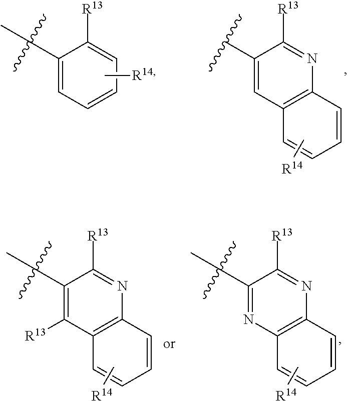

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In one embodiment, X and Y are each O; R¹ is —CH₂—; and R¹⁰ is:

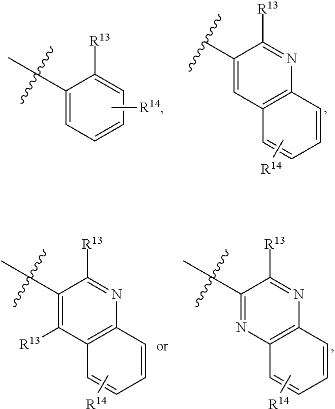

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X and Y are each O; Z is —CH—; R¹ is —CH₂—; and R¹⁰ is:

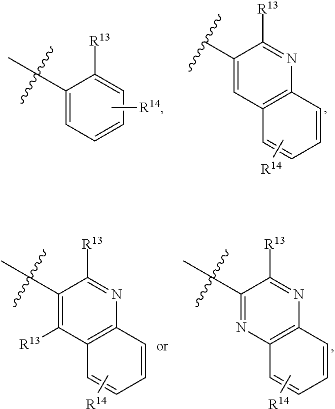

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O and Y is O.

In another embodiment, X is O, Y is O and Z is —C(R³¹)—.

In another embodiment, X is O, Y is O, Z is —C(R³¹)— and R³¹ is H or halo.

In still another embodiment, X is O, Y is O, Z is —C(R³¹)— and R³¹ is H or F.

In another embodiment, X is O, Y is O and Z is —CH—.

In another embodiment, X is O, Y is O and Z is —C(F)—.

In yet another embodiment, X is O, Y is O and Z is —C(Cl)—.

In another embodiment, X is O, Y is O, Z is —C(R³¹)—, each occurrence of R³⁰ is H, and R³¹ is H or halo.

In another embodiment, X is O, Y is O, Z is —C(F)—, and each occurrence of R³⁰ is H.

In a further embodiment, X is O, Y is O, Z is —CH—, and each occurrence of R³⁰ is H.

In one embodiment, X is O, Y is O, Z is —CH—, one occurrence of R³⁰ is alkyl and the other occurrence of R³⁰ is H.

In another embodiment, X is O, Y is O, Z is —CH—, one occurrence of R³⁰ is methyl and the other occurrence of R³⁰ is H.

In another embodiment, X is O, Y is O, Z is —CH—, one occurrence of R³⁰ is —O-alkylene-C(O)O—H, and the other occurrence of R³⁰ is H.

In still another embodiment, X is O, Y is O, Z is —CH—, one occurrence of R³⁰ is —O-alkylene-C(O)O-alkyl, and the other occurrence of R³⁰ is H.

In another embodiment, X is O, Y is O, Z is —C(R³¹)—, R³¹ is halo, and each occurrence of R³⁰ is H.

In one embodiment, R¹ is a bond or —[C(R¹²)₂]ᵣ—.

In another embodiment, R¹ is a bond

In another embodiment, R¹ is —[C(R¹²)^{2]}ᵣ—.

In another embodiment, R¹ is —[C(R¹²)₂]ᵣ—O—[C(R¹²)₂]_{q}—.

In still another embodiment, R¹ is —[C(R¹²)₂]ᵣ—N(R⁹)—[C(R¹²)₂]_{q}—.

In yet another embodiment, R¹ is —[C(R¹²)₂]_{q}—CH═CH—[C(R¹²)₂]_{q}—.

In another embodiment, R¹ is —[C(R¹²)₂]_{q}—C≡C—[C(R¹²)₂]_{q}—.

In a further embodiment, R¹ is —[C(R¹²)₂]_{q}—SO₂—[C(R¹²)₂]_{q}—.

In one embodiment, R¹ is —CH₂—, —CH₂CH₂—, —CH(CH₃)— or

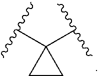

In another embodiment, R¹ is —CH₂—.

In another embodiment, R¹ is —CH₂CH₂—.

In still another embodiment, R¹ is —CH(CH₃)—.

In another embodiment, R¹ is:

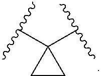

In one embodiment, R¹⁰ is H.

In another embodiment, R¹⁰ is aryl.

In another embodiment, R¹⁰ is cycloalkyl.

In yet another embodiment, R¹⁰ is cycloalkenyl.

In another embodiment, R¹⁰ is heterocycloalkenyl.

In another embodiment, R¹⁰ is heteroaryl.

In still another embodiment, R¹⁰ is heterocycloalkyl.

In another embodiment, R¹⁰ is phenyl.

In another embodiment, R¹⁰ is pyridyl.

In another embodiment, R¹⁰ is quinolinyl.

In a further embodiment, R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In one embodiment, R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, R¹⁰ is heteroaryl, which is substituted with from 1-4 groups independently selected from: halo, alkyl, —N(R⁹)₂ or —O-alkyl.

In another embodiment, R¹⁰ is

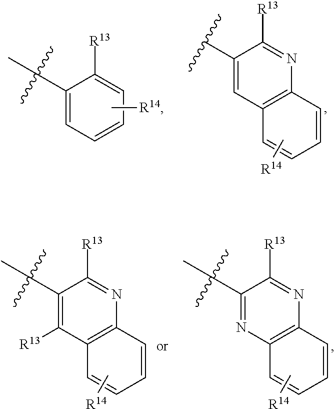

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In still another embodiment, R¹⁰ is

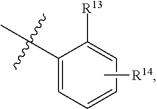

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl an heteroaryl.

In another embodiment, R¹⁰ is

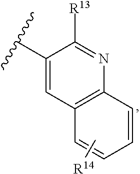

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl an heteroaryl.

In yet another embodiment, R¹⁰ is pyridyl or quinolinyl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, R¹⁰ is pyridyl or quinolinyl, which is substituted with from 1-4 groups independently selected from: halo, alkyl, —N(R⁹)₂ or —O-alkyl.

In another embodiment, R¹⁰ is pyridyl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In an further embodiment, R¹⁰ is pyridyl, which is substituted with from 1-4 groups independently selected from: halo, alkyl, —N(R⁹)₂ or —O-alkyl.

In one embodiment, R¹⁰ is pyridyl, which is substituted with an —N(R⁹)₂ group.

In another embodiment, R¹⁰ is pyridyl, which is substituted with an —NH₂ group.

In another embodiment, R¹⁰ is:

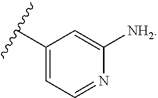

In still another embodiment, R¹⁰ is quinolinyl, which is substituted with from 1-3 groups independently selected from Cl and F.

In another embodiment, R¹⁰ is:

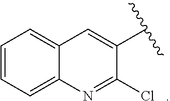

In one embodiment, R¹⁰ is phenyl, which can be optionally substituted with from 1-4 groups independently selected from: halo, alkyl, —CN, —NO₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In yet another embodiment, R¹⁰ is phenyl, which is substituted with one F atom and can be further and optionally substituted with from 1-3 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In one embodiment, R¹⁰ is phenyl, which is substituted with two F atoms and can be further and optionally substituted with from 1-2 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, R¹⁰ is phenyl, which is substituted with from 1-2 groups independently selected from halo and —NO₂.

In another embodiment, R¹⁰ is phenyl, which is substituted with from 1-2 groups independently selected from F and —NO₂.

In a further embodiment, —R¹⁰ is:

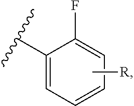

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, R¹⁰ is

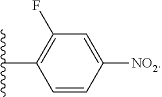

In one embodiment, R¹ is —CH₂—, —CH₂CH₂—, —CH(CH₃)— or

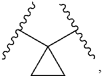

and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, R¹ is —CH₂—, —CH₂CH₂—, —CH(CH₃)— or

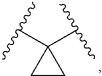

and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, R¹ is —CH₂—, —CH₂CH₂—, —CH(CH₃)— or

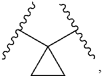

and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In still another embodiment, R¹ is —CH₂—, —CH₂CH₂—, —CH(CH₃)— or

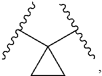

and R¹⁰ is heteroaryl, which is substituted with from 1-4 groups independently selected from: halo, alkyl, —N(R⁹)₂ or —O-alkyl.

In another embodiment, R¹ is —CH₂—, —CH₂CH₂—, —CH(CH₃)— or

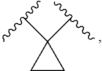

### and R¹⁰ is

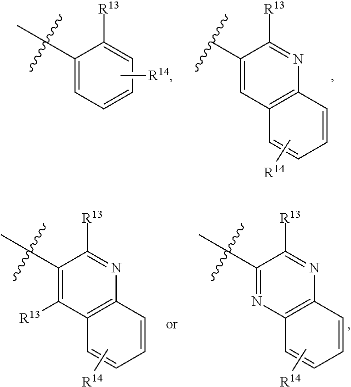

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In a further embodiment, R¹ is —CH₂—, —CH₂CH₂—, —CH(CH₃)— or

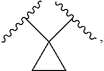

and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In one embodiment, R¹ is —CH₂— and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, R¹ is —CH₂— and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, R¹ is —CH₂— and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, R¹ is —CH₂— and R¹⁰ is heteroaryl, which is substituted with from 1-4 groups independently selected from: halo, alkyl, —N(R⁹)₂ or —O-alkyl.

In another embodiment, R¹ is —CH₂— and R¹⁰ is

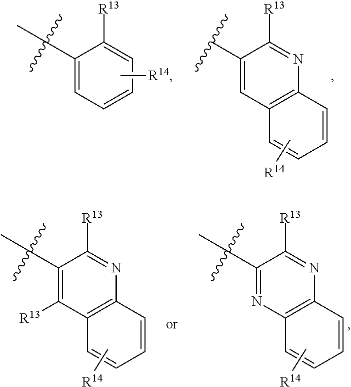

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In still another embodiment, R¹ is —CH₂— and R¹⁰ is

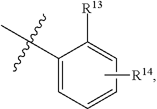

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, R¹ is —CH₂— and R¹⁰ is

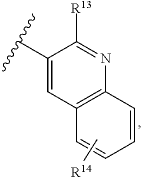

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In yet another embodiment, R¹ is —CH₂— and R¹⁰ is pyridyl or quinolinyl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, R¹ is —CH₂— and R¹⁰ is pyridyl or quinolinyl, which is substituted with from 1-4 groups independently selected from: halo, alkyl, —N(R⁹)₂ or —O-alkyl.

In another embodiment, R¹ is —CH₂— and R¹⁰ is pyridyl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In an further embodiment, R¹ is —CH₂— and R¹⁰ is pyridyl, which is substituted with from 1-4 groups independently selected from: halo, alkyl, —N(R⁹)₂ or —O-alkyl.

In one embodiment, R¹ is —CH₂— and R¹⁰ is pyridyl, which is substituted with an —N(R⁹)2 group.

In another embodiment, R¹ is —CH₂— and R¹⁰ is pyridyl, which is substituted with an —NH₂ group.

In another embodiment, R¹ is —CH₂— and R¹⁰ is:

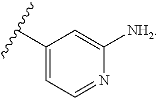

In still another embodiment, R¹ is —CH₂— and R¹⁰ is quinolinyl, which is substituted with from 1-3 groups independently selected from Cl and F.

In another embodiment, R¹ is —CH₂— and R¹⁰ is:

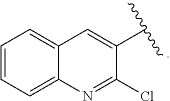

In one embodiment, R¹ is —CH₂— and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In yet another embodiment, R¹ is —CH₂— and R¹⁰ is phenyl, which is substituted with one F atom and can be further and optionally substituted with from 1-3 groups independently selected from: halo, alkyl, —CN, —NO₂, —N(R⁹)₂, —S(O)₂NH₂, —C(O)NH₂, —S(O)₂-C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In one embodiment, R¹ is —CH₂— and R¹⁰ is phenyl, which is substituted with two F atoms and can be further and optionally substituted with from 1-2 groups independently selected from: halo, alkyl, —CN, —NO₂, —N(R⁹)₂, —S(O)₂NH₂, —C(O)NH₂, —S(O)₂-C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, R¹ is —CH₂— and R¹⁰ is phenyl, which is substituted with from 1-2 groups independently selected from halo and —NO₂.

In another embodiment, R¹ is —CH₂— and R¹⁰ is phenyl, which is substituted with from 1-2 groups independently selected from F and —NO₂.

In a further embodiment, R¹ is —CH₂— and R¹⁰ is:

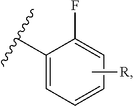

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, R¹ is —CH₂— and R¹⁰ is

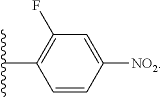

In one embodiment, —R¹-R¹⁰ is alkyl.

In another embodiment, —R¹-R¹⁰ is haloalkyl.

In yet another embodiment, —R¹-R¹⁰ is —R¹-R¹⁰ is benzyl, wherein the phenyl moiety of the benzyl group is substituted with 1 or 2 fluorine atoms.

In another embodiment, —R¹-R¹⁰ is —R¹-R¹⁰ is benzyl, wherein the phenyl moiety of the benzyl group is substituted with one fluorine atom and one nitro group.

In another embodiment, —R¹-R¹⁰ is —CH₂-cycloalkyl.

In one embodiment, R⁴, R⁵, R⁶ and R⁷ are each independently selected from H, halo, —O-alkyl, alkyl or haloalkyl.

In another embodiment, R⁴, R⁵, R⁶ and R⁷ are each independently selected from H, F, Cl, Br, —O-methyl, methyl, ethyl or —CF₃.

In another embodiment, R⁴ is H.

In another embodiment, R⁴ is F.

In one embodiment, R⁵ is halo, alkyl or haloalkyl.

In one embodiment, R⁵ is F, Cl, Br, —O-methyl, methyl, ethyl or —CF₃

In another embodiment, R⁵ is H.

In another embodiment, R⁵ is alkyl.

In another embodiment, R⁵ is methyl.

In another embodiment, R⁵ is ethyl.

In another embodiment, R⁵ is halo.

In another embodiment, R⁵ is F.

In another embodiment, R⁵ is haloalkyl.

In another embodiment, R⁵ is —CF₃.

In another embodiment, R⁶ is H.

In another embodiment, R⁶ is H, halo or —O-alkyl.

In another embodiment, R⁶ is F.

In another embodiment, R⁶ is methoxy.

In still another embodiment, R⁷ is H.

In another embodiment, R⁴ and R⁷ are each H.

In yet another embodiment, R⁴, R⁶ and R⁷ are each H.

In another embodiment, R⁴, R⁵, R⁶ and R⁷ are each H.

In a further embodiment, R⁴, R⁶ and R⁷ are each H and R⁵ is other than H.

In one embodiment, R⁵ is selected from H, halo, —O-alkyl, alkyl or haloalkyl and R⁴, R⁶ and R⁷ are each H.

In one embodiment, R⁵ is selected from F, Cl, Br, —O-methyl, methyl, ethyl or —CF₃ and R⁴, R⁶ and R⁷ are each H.

In one embodiment, R⁵ is selected from F, methyl, ethyl or —CF₃, and R⁴, R⁶ and R⁷ are each H.

In another embodiment, R⁵ is alkyl and R⁴, R⁶ and R⁷ are each H.

In another embodiment, R⁵ is methyl and R⁴, R⁶ and R⁷ are each H.

In another embodiment, R⁵ is ethyl and R⁴, R⁶ and R⁷ are each H.

In another embodiment, R⁵ is —CF₃ and R⁴, R⁶ and R⁷ are each H.

In another embodiment, R⁵ is halo and R⁴, R⁶ and R⁷ are each H.

In another embodiment, R⁵ is F and R⁴, R⁶ and R⁷ are each H.

In another embodiment, R⁵ is alkyl, R⁶ is H, halo or —O-alkyl, and R⁶ and R⁷ are each H.

In another embodiment, R⁵ is ethyl, R⁶ is H, halo or —O-alkyl, and R⁶ and R⁷ are each H.

In another embodiment, R⁵ is ethyl, R⁶ is H, F or methoxy, and R⁶ and R⁷ are each H.

### X, Y, R1, R10

In one embodiment, X is O; Y is O; R¹ is —CH₂—, —CH₂CH₂—, —CH(CH₃)— or

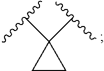

and R¹⁰ is aryl or heteroaryl, either of which can be optionally substituted with from 1-4 groups independently selected from: halo, alkyl, —N(R⁹)₂, —CN, —NO₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; R¹ is —CH₂—, —CH₂CH₂—, —CH(CH₃)— or

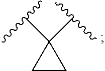

and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R¹ is —CH₂—, —CH₂CH₂—, —CH(CH₃)— or

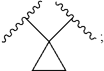

and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In still another embodiment, X is O; Y is O; R¹ is —CH₂—, —CH₂CH₂—, —CH(CH₃)— or

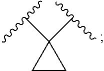

and R¹⁰ is heteroaryl, which is substituted with from 1-4 groups independently selected from: halo, alkyl, —N(R⁹)₂ or —O-alkyl.

In another embodiment, X is O; Y is O; R¹ is —CH₂—, —CH₂CH₂—, —CH(CH₃)— or

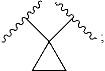

### and R¹⁰ is

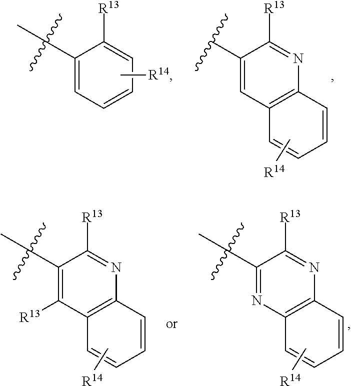

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In a further embodiment, X is O; Y is O; R¹ is —CH₂—, —CH₂CH₂—, —CH(CH₃)— or

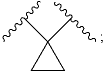

and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In one embodiment, X is O; Y is O; R¹ is —CH2-; and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; R¹ is —CH₂—; and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R¹ is —CH₂—; and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; R¹ is —CH₂—; and R¹⁰ is heteroaryl, which is substituted with from 1-4 groups independently selected from: halo, alkyl, —N(R⁹)₂ or —O-alkyl.

In another embodiment, X is O; Y is O; R¹ is —CH₂—; and R¹⁰ is

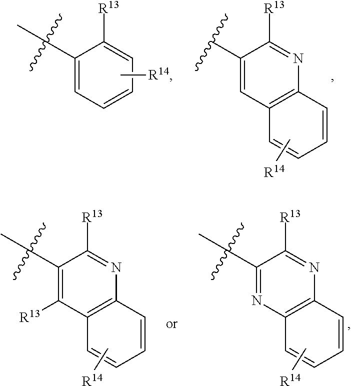

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In still another embodiment, X is O; Y is O; R¹ is —CH₂—; and R¹⁰ is

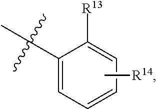

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O; Y is O; R¹ is —CH₂—; and R¹⁰ is

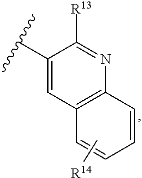

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl. In yet another embodiment, X is O; Y is O; R¹ is —CH₂—; and R¹⁰ is pyridyl or quinolinyl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; R¹ is —CH₂—; and R¹⁰ is pyridyl or quinolinyl, which is substituted with from 1-4 groups independently selected from: halo, alkyl, —N(R⁹)₂ or —O-alkyl.

In another embodiment, X is O; Y is O; R¹ is —CH₂—; and R¹⁰ is pyridyl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In an further embodiment, X is O; Y is O; R¹ is —CH₂—; and R¹⁰ is pyridyl, which is substituted with from 1-4 groups independently selected from: halo, alkyl, —N(R⁹)₂ or —O-alkyl.

In one embodiment, X is O; Y is O; R¹ is —CH₂—; and R¹⁰ is pyridyl, which is substituted with an —N(R⁹)₂ group.

In another embodiment, X is O; Y is O; R¹ is —CH₂—; and R¹⁰ is pyridyl, which is substituted with an —NH₂ group.

In another embodiment, X is O; Y is O; R¹ is —CH₂—; and R¹⁰ is:

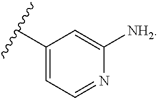

In still another embodiment, X is O; Y is O; R¹ is —CH₂—; and R¹⁰ is quinolinyl, which is substituted with from 1-3 groups independently selected from Cl and F.

In another embodiment, X is O; Y is O; R¹ is —CH₂—; and R¹⁰ is:

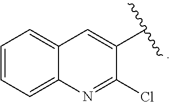

In one embodiment, X is O; Y is O; R¹ is —CH₂—; and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In yet another embodiment, X is O; Y is O; R¹ is —CH₂—; and R¹⁰ is phenyl, which is substituted with one F atom and can be further and optionally substituted with from 1-3 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In one embodiment, X is O; Y is O; R¹ is —CH₂—; and R¹⁰ is phenyl, which is substituted with two F atoms and can be further and optionally substituted with from 1-2 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R¹ is —CH₂—; and R¹⁰ is phenyl, which is substituted with from 1-2 groups independently selected from halo and —NO₂.

In another embodiment, X is O; Y is O; R¹ is —CH₂—; and R¹⁰ is phenyl, which is substituted with from 1-2 groups independently selected from F and —NO₂.

In another embodiment, X is O; Y is O; R¹ is —CH₂—; and R¹⁰ is

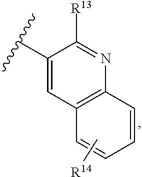

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O; Y is O; R¹ is —CH₂—; and R¹⁰ is

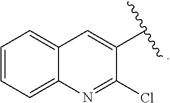

In a further embodiment, X is O; Y is O; R¹ is —CH₂—; and R¹⁰ is:

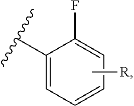

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, X is O; Y is O; R¹ is —CH₂—; and R¹⁰ is

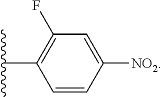

In one embodiment, X is O; Y is O; R¹ is —CH₂—, —CH₂CH₂—, —CH(CH₃)— or

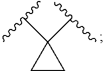

R⁵ is alkyl; and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; R¹ is —CH₂—, —CH₂CH₂—, —CH(CH₃)— or

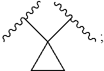

R⁵ is alkyl; and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R^{l} is —CH₂—, —CH₂CH₂—, —CH(CH₃)— or

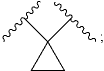

R⁵ is alkyl; and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In one embodiment, X is O; Y is O; R¹ is —CH₂—; R⁵ is alkyl; and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; R¹ is —CH₂—; R⁵ is alkyl; and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R¹ is —CH₂—; R⁵ is alkyl; and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In one embodiment, X is O; Y is O; R¹ is —CH₂—; R⁵ is alkyl; and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R¹ is —CH₂—; R⁵ is alkyl; and R¹⁰ is

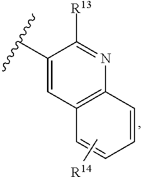

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O; Y is O; R¹ is —CH₂—; R⁵ is alkyl; and R¹⁰ is

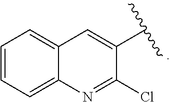

In a further embodiment, X is O; Y is O; R¹ is —CH₂—; R⁵ is alkyl; and R¹⁰ is:

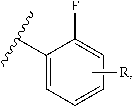

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, X is O; Y is O; R¹ is —CH₂—; R⁵ is alkyl; and R¹⁰ is

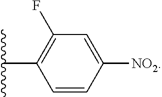

In one embodiment, X is O; Y is O; R¹ is —CH₂—; R⁵ is ethyl; and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; R¹ is —CH₂—; R⁵ is ethyl; and R¹⁰ is

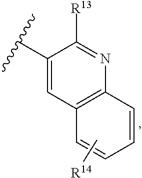

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O; Y is O; R¹ is —CH₂—; R⁵ is ethyl; and R¹⁰ is

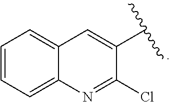

In a further embodiment, X is O; Y is O; R¹ is —CH₂—; R⁵ is ethyl; and R¹⁰ is:

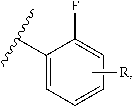

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, X is O; Y is O; R¹ is —CH₂—; R⁵ is ethyl; and R¹⁰ is

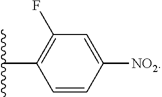

In one embodiment, X is O; Y is O; Z is —CH—; R¹ is —CH₂—, —CH₂CH₂—, —CH(CH₃)— or

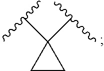

R⁵ is alkyl; and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; Z is —CH—; R¹ is —CH₂—, —CH₂CH₂—, —CH(CH₃)— or

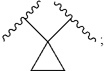

R⁵ is alkyl; and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; Z is —CH—; R¹ is —CH₂—, —CH₂CH₂—, —CH(CH₃)— or

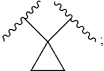

R⁵ is alkyl; and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In one embodiment, X is O; Y is O; Z is —CH—; R¹ is —CH₂—; R⁵ is alkyl; and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; Z is —CH—; R¹ is —CH₂—; R⁵ is alkyl; and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; Z is —CH—; R¹ is —CH₂—; R⁵ is alkyl; and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In one embodiment, X is O; Y is O; Z is —CH—; R¹ is —CH₂—; R⁵ is alkyl; and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; Z is —CH—; R¹ is —CH₂—; R⁵ is alkyl; and R¹⁰ is

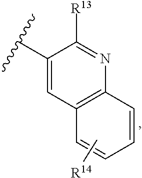

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O; Y is O; Z is —CH—; R¹ is —CH₂—; R⁵ is alkyl; and R¹⁰ is

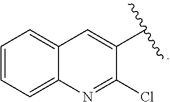

In a further embodiment, X is O; Y is O; Z is —CH—; R¹ is —CH₂—; R⁵ is alkyl; and R¹⁰ is:

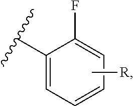

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, X is O; Y is O; Z is —CH—; R¹ is —CH₂—; R⁵ is alkyl; and R¹⁰ is

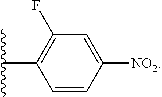

In one embodiment, X is O; Y is O; Z is —CH—; R¹ is —CH₂—; R⁵ is ethyl; and R¹⁰ is aryl or heteroaryl, either of which can be optionally substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In another embodiment, X is O; Y is O; Z is —CH—; R¹ is —CH₂—; R⁵ is ethyl; and R¹⁰ is aryl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; Z is —CH—; R¹ is —CH₂—; R⁵ is ethyl; and R¹⁰ is heteroaryl, which is substituted with from 1-4 groups independently selected from: halo, alkyl, —N(R⁹)₂, —CN, —NO₂, —S(O)₂NH₂, —S(O)₂-haloalkyl, —C(O)NH₂, —C(O)NH-alkyl, —OH, NHS(O)₂-alkyl, —NHS(O)₂-cycloalkyl, —O-alkyl, —C(O)NH-alkylene-cycloalkyl, haloalkyl, —S(O)₂-alkyl, —S-alkyl or —NHS(O)₂-alkyl.

In one embodiment, X is O; Y is O; Z is —CH—; R¹ is —CH₂—; R⁵ is ethyl; and R¹⁰ is phenyl, which can be optionally substituted with from 1-4 groups independently selected from: halo, alkyl, —CN, —NO₂, —N(R⁹)₂, —S(O)₂NH₂, —C(O)NH₂, —S(O)₂-haloalkyl, —C(O)NH-alkyl, —NHS(O)₂-alkyl, —NHS(O)₂-cycloalkyl, —O-alkyl, —C(O)NH-alkylene-cycloalkyl, —OH, haloalkyl, —S(O)₂-alkyl, —S-alkyl or —NHS(O)₂-alkyl.

In another embodiment, X is O; Y is O; Z is —CH—; R¹ is —CH₂—; R⁵ is ethyl; and R¹⁰ is

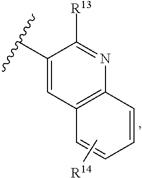

wherein R¹³ is F or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is O; Y is O; Z is —CH—; R¹ is —CH₂—; R⁵ is ethyl; and R¹⁰ is

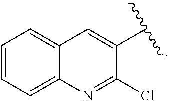

In a further embodiment, X is O; Y is O; Z is —CH—; R¹ is —CH₂—; R⁵ is ethyl; and R¹⁰ is:

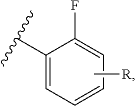

wherein R represents up to 2 optional and additional phenyl substituents, each independently selected from halo, —O-alkyl, alkyl, —CF₃, —CN, —NHSO₂-alkyl, —NO₂, —C(O)NH₂, —C(O)OH, —NH₂, —SO₂-alkyl, —SO₂NH-alkyl, —S-alkyl, —CH₂NH₂, —SO₂NH₂, —NHC(O)-alkyl, —C(O)O-alkyl, —C(O)-heterocycloalkyl and heteroaryl.

In another embodiment, X is O; Y is O; Z is —CH—; R¹ is —CH₂—; R⁵ is ethyl; and R¹⁰ is

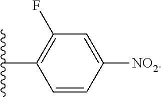

In another embodiment, X is —O—, —OCH₂O—, —NH— or —OCH₂NH—; Z is —C(R³¹)—; R¹ is —[C(R¹²)₂]ᵣ—; and R⁴ and R⁷ are each independently H, halo or hydroxy.

In another embodiment, X is —O—, —OCH₂O—, —NH— or —OCH₂NH—; Z is —C(R³¹)—; R¹ is —[C(R¹²)₂]ᵣ—; and R⁵ is H, alkyl, —O-alkyl, cycloalkyl, halo, haloalkyl, hydroxy, hydroxyalkyl, —NH₂ or —CN.

In still another embodiment, X is —O—, —OCH₂O—, —NH— or —OCH₂NH—; Z is —C(R³¹)—; R¹ is —[C(R¹²)₂]ᵣ—; and R⁴ and R⁵ groups, together with the common carbon atom to which they are attached, join to form a cycloalkyl, heterocycloalkyl, aryl or heteroaryl group.

In another embodiment, X is —O—, —OCH₂O—, —NH— or —OCH₂NH—; Z is —C(R³¹)—; R¹ is —[C(R¹²)₂]ᵣ—; and R⁵ and R⁶ groups, together with the common carbon atom to which they are attached, join to form a cycloalkyl, heterocycloalkyl, aryl or heteroaryl group.

In another embodiment, X is —O—, —OCH₂O—, —NH— or —OCH₂NH—; Z is —C(R³¹)—; R¹ is —[C(R¹²)₂]ᵣ—; and R⁶ is H, alkyl, —O-alkyl, cycloalkyl, halo, haloalkyl, hydroxy, hydroxyalkyl, NH₂ or —CN.

In yet another embodiment, X is —O—, —OCH₂O—, —NH— or —OCH₂NH—; Z is —C(R³¹)—; R¹ is —CH₂—, —CH₂CH₂—, —CH(CH₃)— or

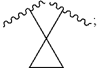

and R¹⁰ is aryl or heteroaryl.

In another embodiment, X is —O—, —OCH₂O—, —NH— or —OCH₂NH—; Z is —C(R³¹)—; R¹ is —CH₂—, —CH₂CH₂—, —CH(CH₃)— or

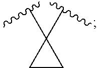

and R¹⁰ is phenyl, naphthyl, pyridyl, quinolinyl or quinoxalinyl.

In one embodiment, X is —O—, —OCH₂O—, —NH— or —OCH₂NH—; Z is —C(R³¹)—; R¹ is —CH₂—, —CH₂CH₂—, —CH(CH₃)— or

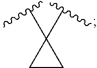

### and R¹⁰ is:

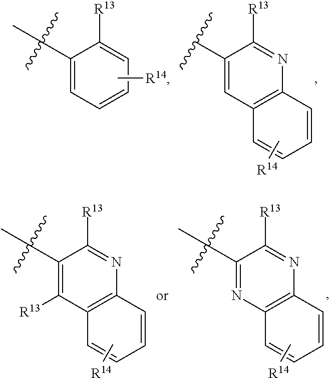

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is —O—, —OCH₂O—, —NH— or —OCH₂NH—; Z is —C(R³¹)—; R¹ is —CH₂—, —CH₂CH₂—, —CH(CH₃)— or

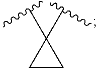

R⁵ is H, alkyl, —O-alkyl, cycloalkyl, halo, haloalkyl, hydroxy, hydroxyalkyl, —NH₂ or —CN; R⁶ is H, alkyl, —O-alkyl, cycloalkyl, halo, haloalkyl, hydroxy, hydroxyalkyl, —NH₂ or —CN; and R¹⁰ is:

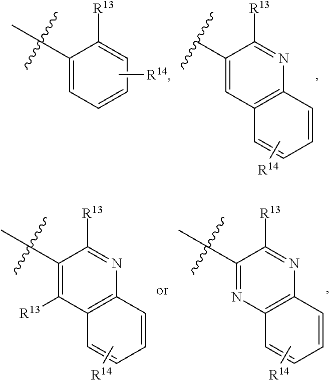

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X is —O—, —OCH₂O—, —NH— or —OCH₂NH—; Z is —C(R³¹)—; R¹ is —CH₂—, —CH₂CH₂—, —CH(CH₃)— or

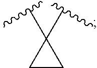

R⁵ is methyl, ethyl or cyclopropyl; R⁶ is H, Cl, F or hydroxy; and R¹⁰ is:

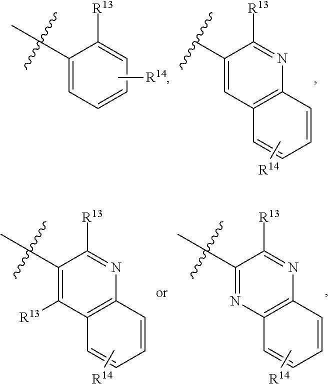

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In one embodiment, X and Y are each O; R¹ is —CH₂—; and R¹⁰ is:

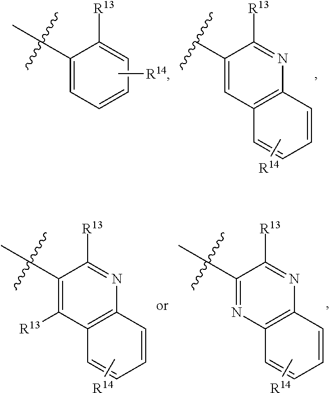

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In another embodiment, X and Y are each O; Z is —CH—; R¹ is —CH₂—; and R¹⁰ is:

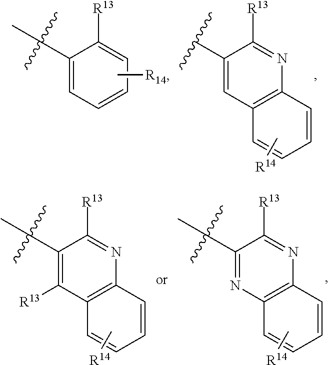

wherein R¹³ is H, F, Br or Cl and R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl.

In one embodiment, X is —O— and Y is ═O or ═N(R⁹)SO₂R¹¹.

In another embodiment, X is —O—; Y is ═O or ═N(R⁹)SO₂R¹¹; R⁹ is H, alkyl, cycloalkyl or heterocycloalkyl; and R¹¹ is alkyl, cycloalkyl, haloalkyl or heterocycloalkyl.

In another embodiment, X is —O—; Y is ═O or ═N(R⁹)SO₂R¹¹; and Z is (C)R³¹.

In still another embodiment, X is —O—; Y is ═O or ═N(R⁹)SO₂R¹¹; and R¹ is —[C(R¹²)₂]₄—.

In another embodiment, X is —O—; Y is ═O or ═N(R⁹)SO₂R¹¹; and R¹ is —CH₂—, —CH₂CH₂—, —CH(CH₃)— or

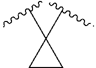

In a further embodiment, X is —O—; Y is ═O or ═N(R⁹)SO₂R¹¹; R¹ is —CH₂—, —CH₂CH₂—, —CH(CH₃)— or

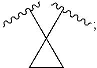

and R⁴ and R⁷ are each independently H, alkyl, halo or hydroxy.

In one embodiment, X is —O—; Y is ═O or ═N(R⁹)SO₂R¹¹; R¹ is —CH₂—, —CH₂CH₂—, —CH(CH₃)— or

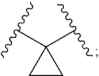

R⁵ is H, alkyl, —O-haloalkyl, —O-alkyl, cycloalkyl, halo, haloalkyl, hydroxy, hydroxyalkyl, —NH₂ or —CN; and R⁶ is H, alkyl, —O-alkyl, —O-haloalkyl, cycloalkyl, halo, haloalkyl, hydroxy, hydroxyalkyl, —NH₂, —NH-alkyl or —CN.

In another embodiment, X is —O—; Y is ═O or ═N(R⁹)SO₂R¹¹; R¹ is —CH₂—, —CH₂CH₂—, —CH(CH₃)— or

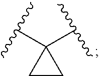

and R⁴ and R⁵, together with the common carbon atom to which they are attached, join to form a -3- to 7-membered cyclic group selected from cycloalkyl, heterocycloalkyl, aryl and heteroaryl.

In another embodiment, X is —O—; Y is ═O or ═N(R⁹)SO₂R¹¹; R¹ is —CH₂—, —CH₂CH₂—, —CH(CH₃)— or

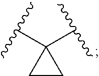

and R⁵ and R⁵, together with the common carbon atom to which they are attached, join to form a -3- to 7-membered cyclic group selected from cycloalkyl, heterocycloalkyl, aryl and heteroaryl.

In still another embodiment, X is —O—; Y is ═O or ═N(R⁹)SO₂R¹¹; R¹ is —CH₂—, —CH₂CH₂—, —CH(CH₃)— or

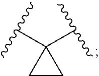

and R⁶ and R⁷, together with the common carbon atom to which they are attached, join to form a -3- to 7-membered cyclic group selected from cycloalkyl, heterocycloalkyl, aryl and heteroaryl.

In another embodiment, X is —O—; Y is ═O or ═N(R⁹)SO₂R¹¹; and R¹⁰ is aryl or heteroaryl.

In another embodiment, X is —O—; Y is ═O or ═N(R⁹)SO₂R¹¹; and R¹⁰ is phenyl, naphthyl, pyridyl, quinolinyl or quinoxalinyl.

In a further embodiment, X is —O—; Y is ═O or ═N(R⁹)SO₂R¹¹; and R¹⁰ is:

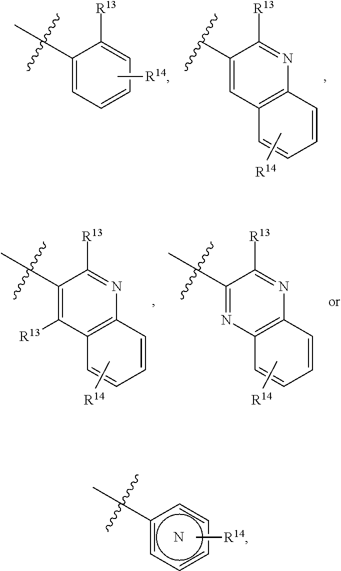

wherein R¹³ is H, F, Br or Cl; R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl; and

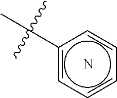

represents a pyridyl group, wherein the ring nitrogen atom can be at any of the five unsubstituted ring atom positions.

In another embodiment, X is —O—; Y is ═O or ═N(R⁹)SO₂R¹¹; R⁵ is H, alkyl, —O-alkyl, cycloalkyl, halo, haloalkyl, hydroxy, hydroxyalkyl, —NH₂ or —CN; R⁶ is H, alkyl, —O-alkyl, cycloalkyl, halo, haloalkyl, hydroxy, hydroxyalkyl, —NH, or —CN; and R¹⁰ is:

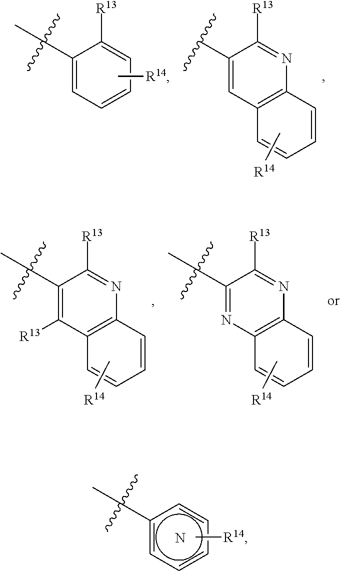

wherein R¹³ is H, F, Br or Cl; R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl; and

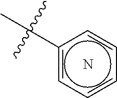

represents a pyridyl group, wherein the ring nitrogen atom can be at any of the five unsubstituted ring atom positions.

In another embodiment, X is —O—; Y is ═O or ═N(R⁹)SO₂R¹¹; R⁵ is methyl, ethyl or cyclopropyl; R⁶ is H, Cl, F or hydroxy; and R¹⁰ is:

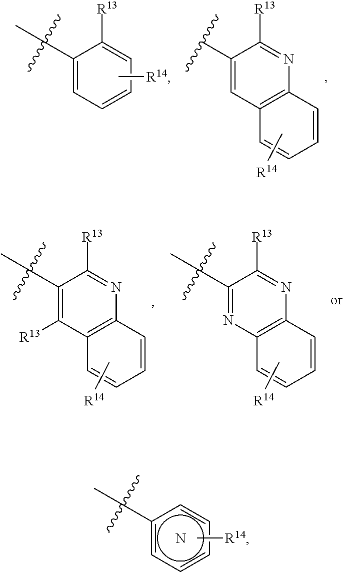

wherein R¹³ is H, F, Br or Cl; R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl; and

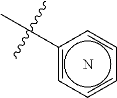

represents a pyridyl group, wherein the ring nitrogen atom can be at any of the five unsubstituted ring atom positions.

In still another embodiment, X is —O—; Y is ═O; R¹ is —CH₂—; R⁵ is methyl, ethyl or cyclopropyl; R⁶ is H, Cl, F or hydroxy; and R¹⁰ is:

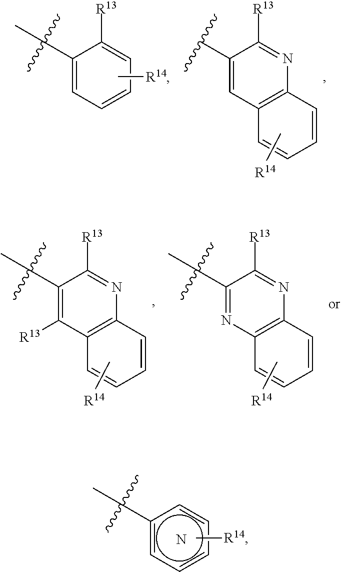

wherein R¹³ is H, F, Br or Cl; R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl; and

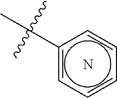

represents a pyridyl group, wherein the ring nitrogen atom can be at any of the five unsubstituted ring atom positions.

In a further embodiment, X is —O—; Y is ═O; Z is —CH—; R¹ is —CH₂—; R⁵ is methyl, ethyl or cyclopropyl; R⁶ is H, Cl, F or hydroxy; and R¹⁰ is:

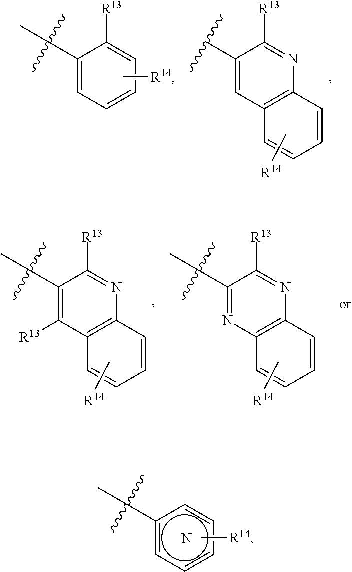

wherein R¹³ is H, F, Br or Cl; R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)0-alkyl, —C(O)-heterocycloalkyl and heteroaryl; and

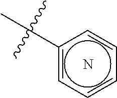

represents a pyridyl group, wherein the ring nitrogen atom can be at any of the five unsubstituted ring atom positions.

In one embodiment, the compound of formula (I) has the formula (Ia):

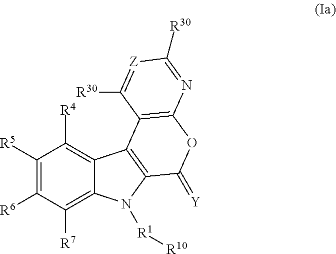

and pharmaceutically acceptable salts, solvates, esters and prodrugs thereof.
wherein:

Y is ═O, ═NH or ═NSO₂R¹¹;

Z is —C(R³¹)—;

R¹ is a bond or an alkylene group;

R⁴ is H or or R⁴ and R⁵, together with the carbon atoms to which they are attached, join to form a 5-membered cyclic group, selected from cycloalkyl, heterocycloalkyl, aryl or heteroaryl;

R⁵ and R⁶ are each independently H, halo, alkyl, —O-alkyl, haloalkyl, —O-haloalkyl, heterocycloalkenyl or cycloalkyl, or R⁵ and R⁶, together with the carbon atoms to which they are attached, join to form a 5-membered cyclic group, selected from cycloalkyl, heterocycloalkyl, aryl or heteroaryl;

R⁷ is H or or R⁶ and R⁷, together with the carbon atoms to which they are attached, join to form a 5-membered cyclic group, selected from cycloalkyl, heterocycloalkyl, aryl or heteroaryl;

R¹⁰ is H, halo, aryl, heterocycloalkenyl or heteroaryl, wherein an aryl or heteroaryl group can be optionally and independently substituted with up to 4 substituents, which are each independently selected from H, alkyl, halo, —NH₂, —OH, —CN, —NO₂, —O-alkyl, —C(O)NH₂, heteroaryl, —SO₂NH₂, —SO₂NH-alkyl, —SO₂-alkyl, phenyl, —NHC(O)OH, —S-alkyl, —NHSO₂-alkyl, —NHSO₂-cycloalkyl, —O-benzyl, —C(O)NH-alkyl, —S-haloalkyl or —S(O)-haloalkyl, such that when R¹ is a bond, R¹⁰ is other than H;

each occurrence of R¹¹ is independently alkyl or cycloalkyl;

each occurrence of R³⁰ is independently, H, alkyl, —O-alkylene-C(O)OH, —O-alkylene-C(O)O-alkyl, or any R³⁰ and R³¹, together with the carbon atoms to which they are attached, join to form a 3- to 7-membered cyclic group, selected from cycloalkyl, heterocycloalkyl, aryl and heteroaryl; and

R³¹ is H or halo.

In one embodiment, a Tetracyclic Indole Derivative is in purified form.

Non-limiting illustrative examples of the Tetracyclic Indole Derivatives are set forth below in Tables 1 and 2 and in the Examples section below herein.

TABLE 1

| No. | STRUCTURE | M + H |
|---|---|---|
| 1 | 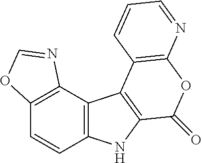 | 278.2 |
| 2 | 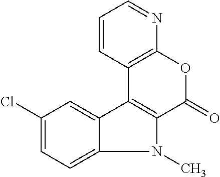 | 285.7 |
| 3 | 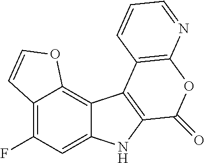 | 295.2 |
| 4 | 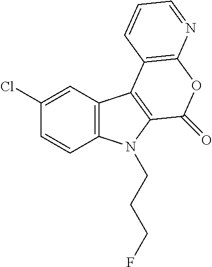 | 331.7 |
| 5 | 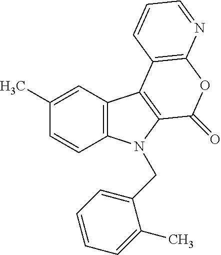 | 355.4 |
| 6 | 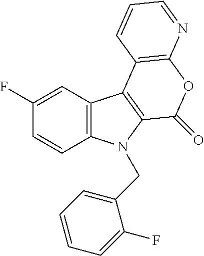 | 363.3 |
| 7 | 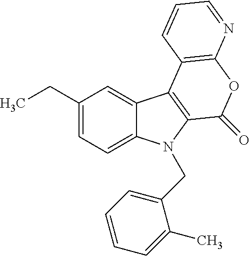 | 369.4 |
| 8 | 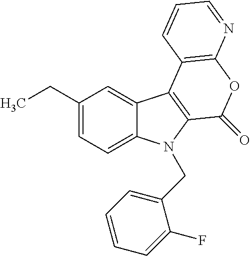 | 373.4 |
| 9 | 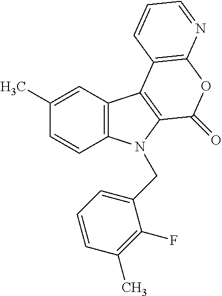 | 373.4 |
| 10 | 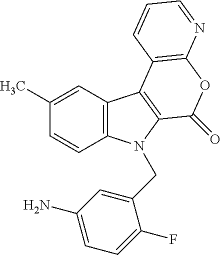 | 374.4 |
| 11 | 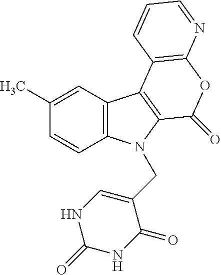 | 375.4 |
| 12 | 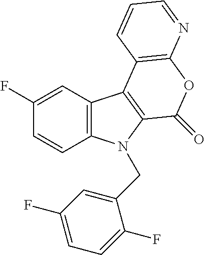 | 381.3 |
| 13 | 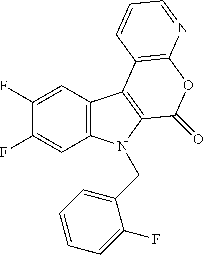 | 381.3 |
| 14 | 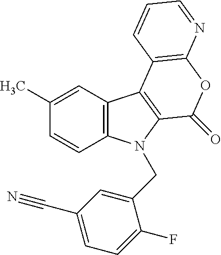 | 384.4 |
| 15 | 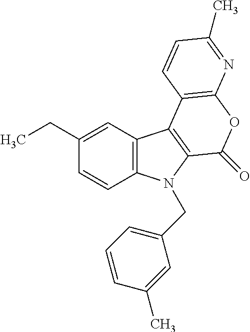 | 385.4 |
| 16 | 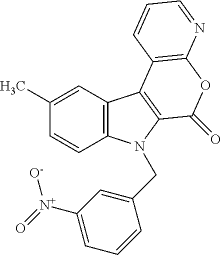 | 386.4 |
| 17 | 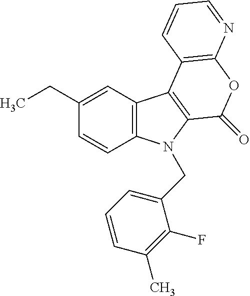 | 387.4 |
| 18 | 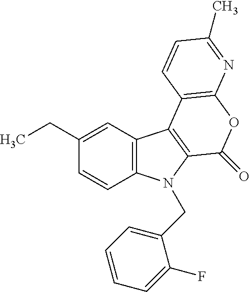 | 387.4 |
| 19 | 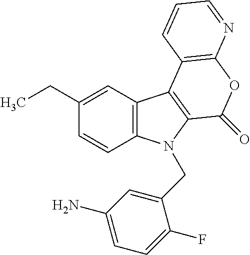 | 388.4 |
| 20 | 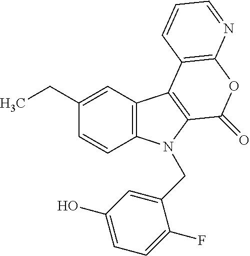 | 389.4 |
| 21 | 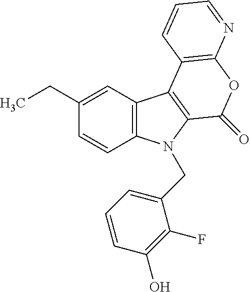 | 389.4 |
| 22 | 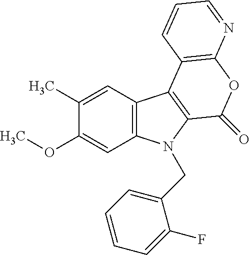 | 389.4 |
| 23 | 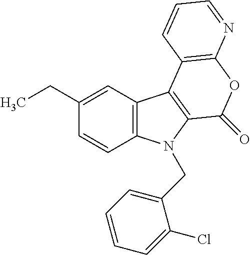 | 389.9 |
| 24 | 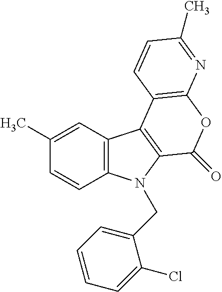 | 389.9 |
| 25 | 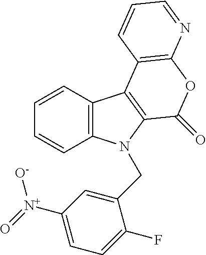 | 390.3 |
| 26 | 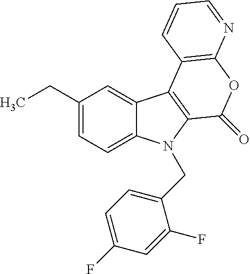 | 391.4 |
| 27 | 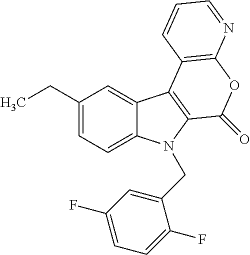 | 391.4 |
| 28 | 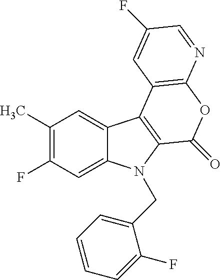 | 395.4 |
| 29 | 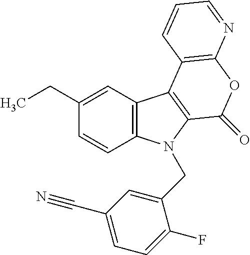 | 398.4 |
| 30 | 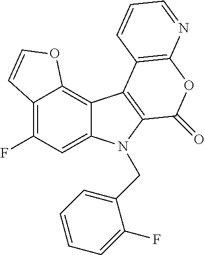 | 403.4 |
| 31 | 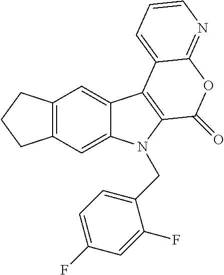 | 403.4 |
| 32 | 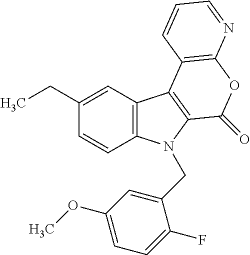 | 403.4 |
| 33 | 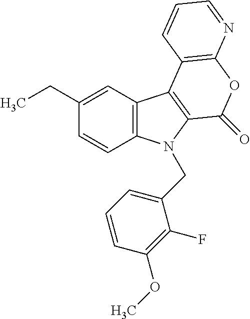 | 403.4 |
| 34 | 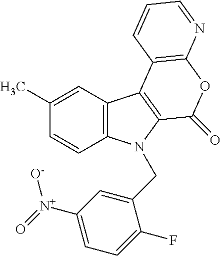 | 404.4 |
| 35 | 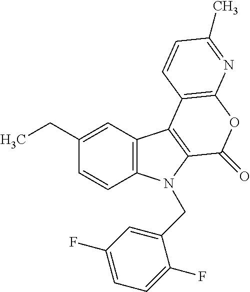 | 405.4 |
| 36 | 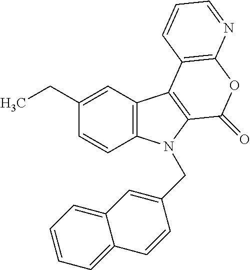 | 405.5 |
| 37 | 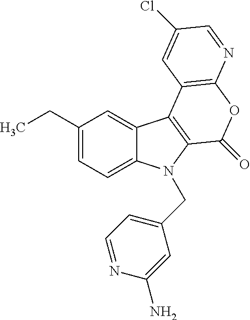 | 405.9 |
| 38 | 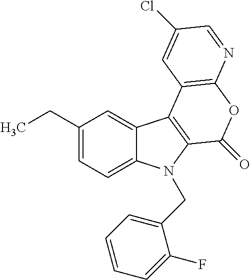 | 407.8 |
| 39 | 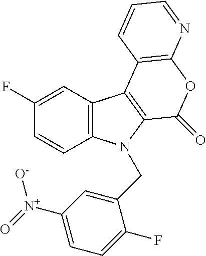 | 408.3 |
| 40 | 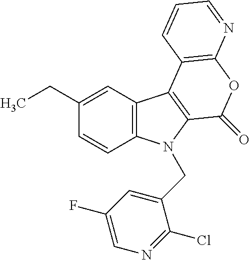 | 408.8 |
| 41 | 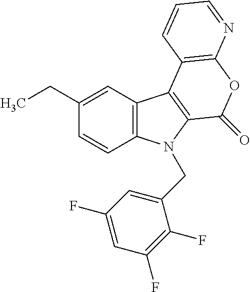 | 409.4 |
| 42 | 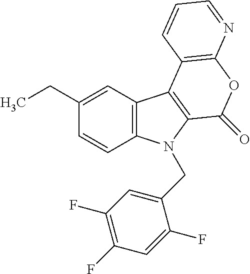 | 409.4 |
| 43 | 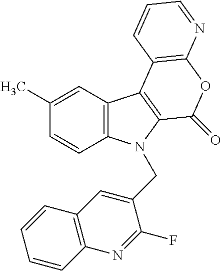 | 410.4 |
| 44 | 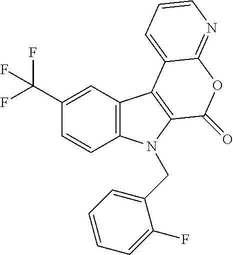 | 413.3 |
| 45 | 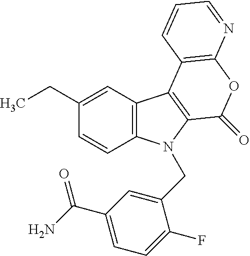 | 416.4 |
| 46 | 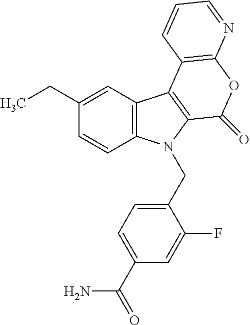 | 416.4 |
| 47 | 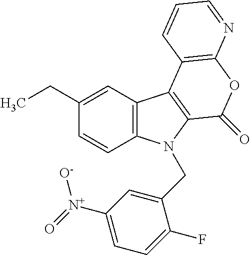 | 418.4 |
| 48 | 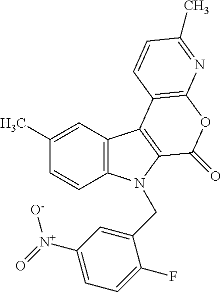 | 418.4 |
| 49 | 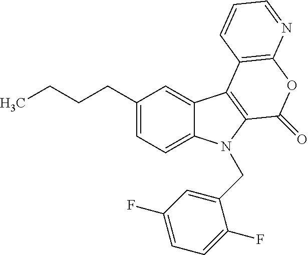 | 419.4 |
| 50 | 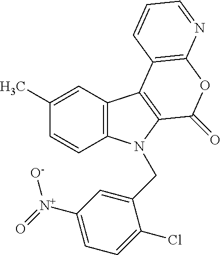 | 420.8 |
| 51 | 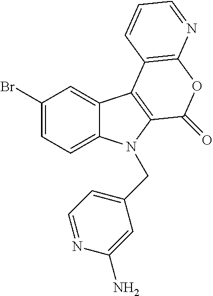 | 422.3 |
| 52 | 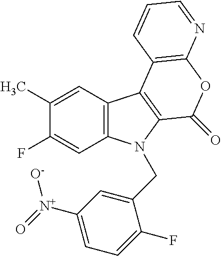 | 422.4 |
| 53 | 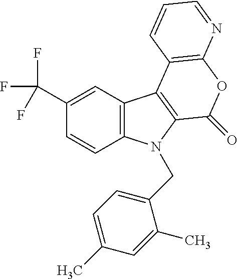 | 423.4 |
| 54 | 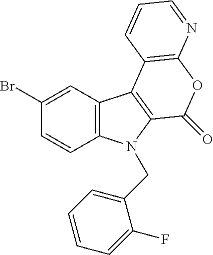 | 424.2 |
| 55 | 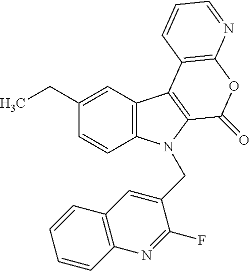 | 424.5 |
| 56 | 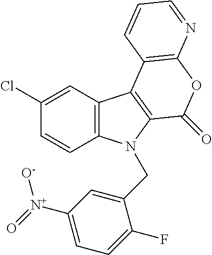 | 424.8 |
| 57 | 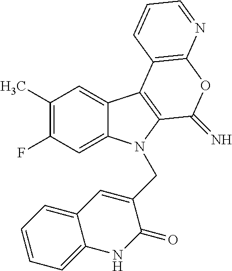 | 425.4 |
| 58 | 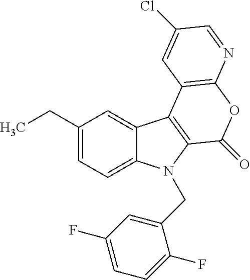 | 425.8 |
| 59 | 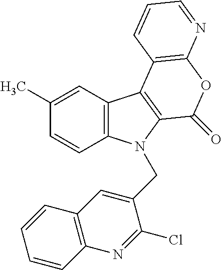 | 426.9 |
| 60 | 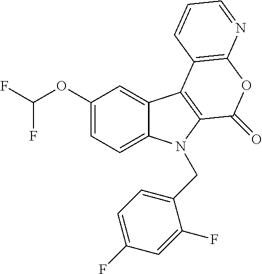 | 429.3 |
| 61 | 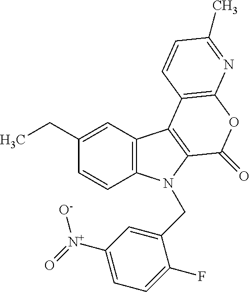 | 432.4 |
| 62 | 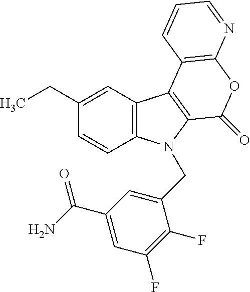 | 434.4 |
| 63 | 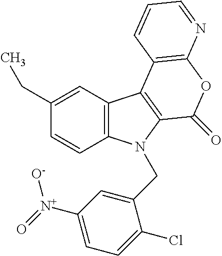 | 434.9 |
| 64 | 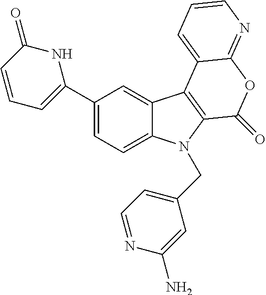 | 436.4 |
| 65 | 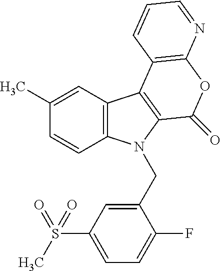 | 437.5 |
| 66 | 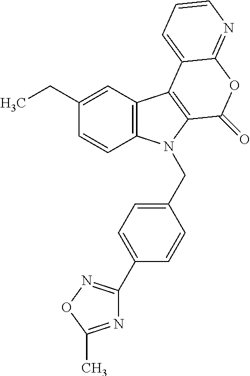 | 437.5 |
| 67 | 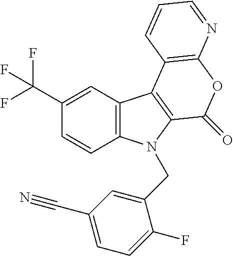 | 438.4 |
| 68 | 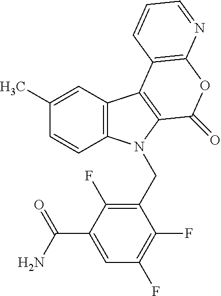 | 438.4 |
| 69 | 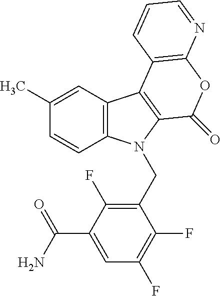 | 438.4 |
| 70 | 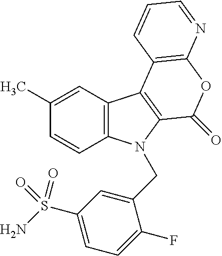 | 438.5 |
| 71 | 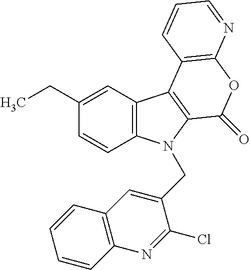 | 440.9 |
| 72 | 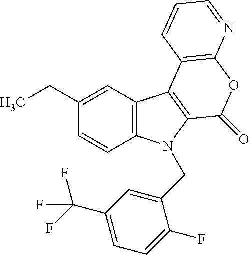 | 441.4 |
| 73 | 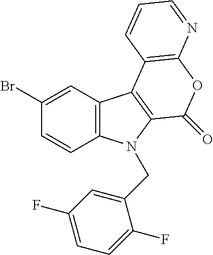 | 442.2 |
| 74 | 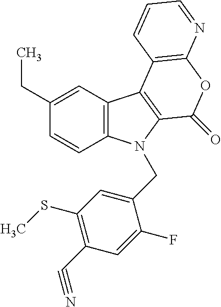 | 444.5 |
| 75 | 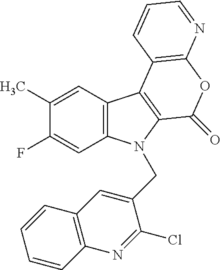 | 444.9 |
| 76 | 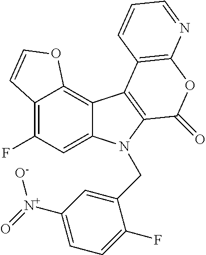 | 448.4 |
| 77 | 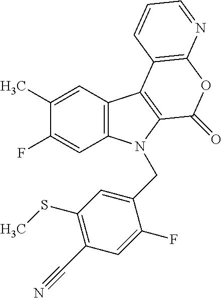 | 448.5 |
| 78 | 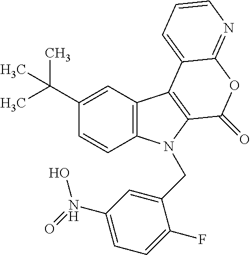 | 448.5 |
| 79 | 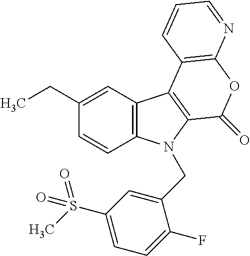 | 451.5 |
| 80 | 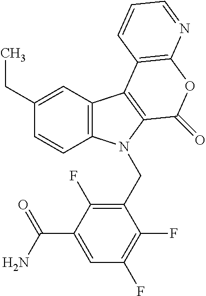 | 452.4 |
| 81 | 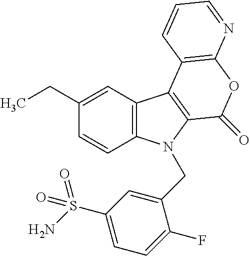 | 452.5 |
| 82 | 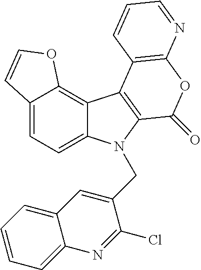 | 452.9 |
| 83 | 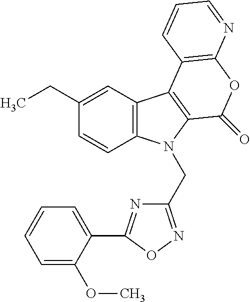 | 453.5 |
| 84 | 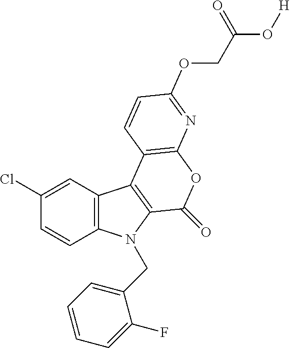 | 453.8 |
| 85 | 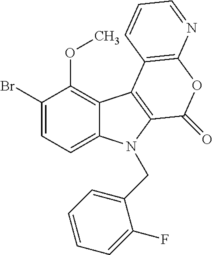 | 454.3 |
| 86 | 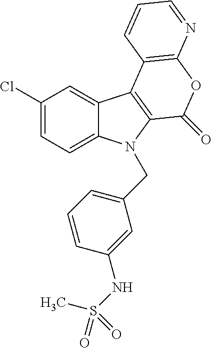 | 454.9 |
| 87 | 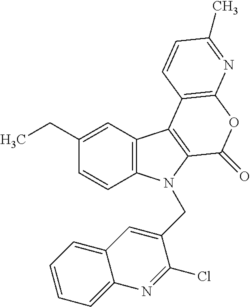 | 454.9 |
| 88 | 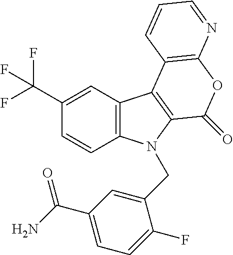 | 456.4 |
| 89 | 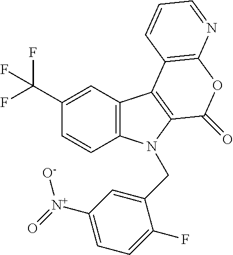 | 458.3 |
| 90 | 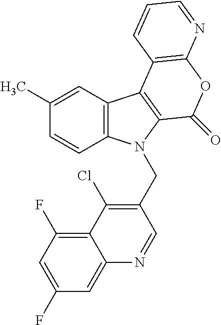 | 462.9 |
| 91 | 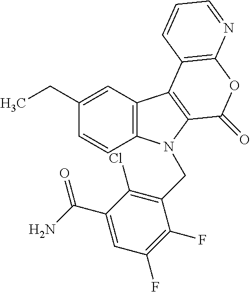 | 468.9 |
| 92 | 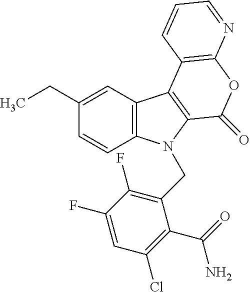 | 468.9 |
| 93 | 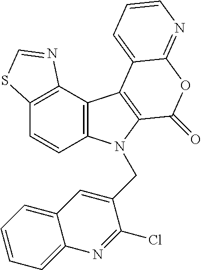 | 469.9 |
| 94 | 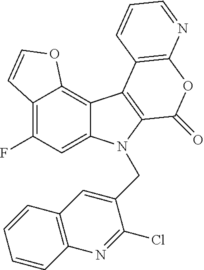 | 470.9 |
| 95 | 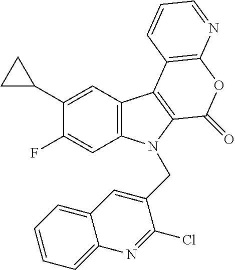 | 470.9 |
| 96 | 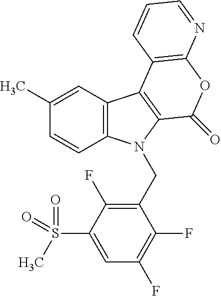 | 473.4 |
| 97 | 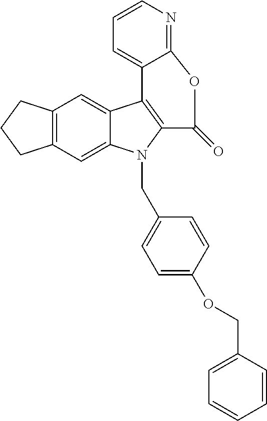 | 473.5 |
| 98 | 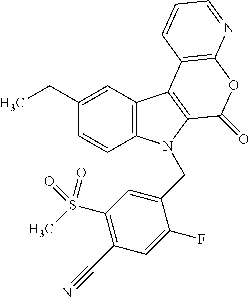 | 476.5 |
| 99 | 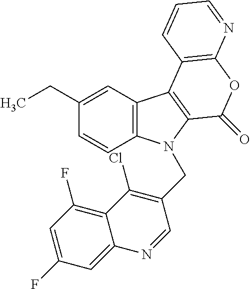 | 476.9 |
| 100 | 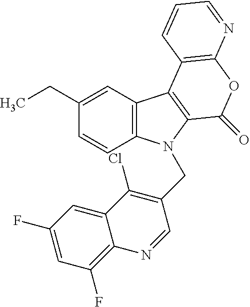 | 476.9 |
| 101 | 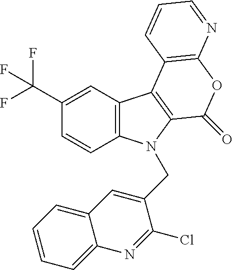 | 480.8 |
| 102 | 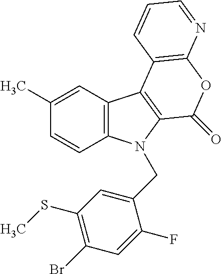 | 484.4 |
| 103 | 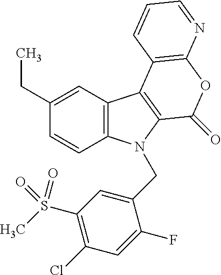 | 485.9 |
| 104 | 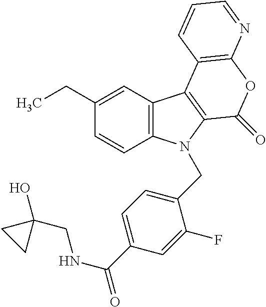 | 486.5 |
| 105 | 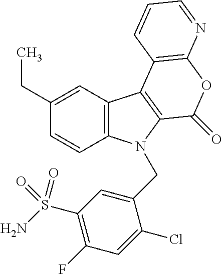 | 486.9 |
| 106 | 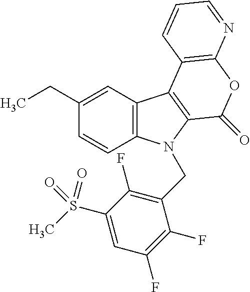 | 487.5 |
| 107 | 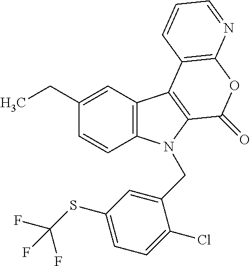 | 489.9 |
| 108 | 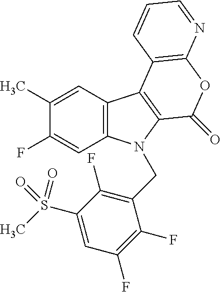 | 491.4 |
| 109 | 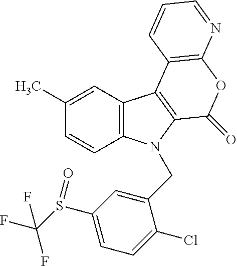 | 491.9 |
| 110 | 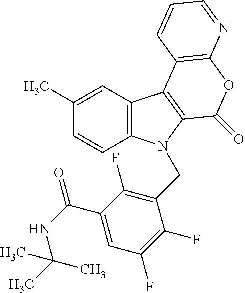 | 494.5 |
| 111 | 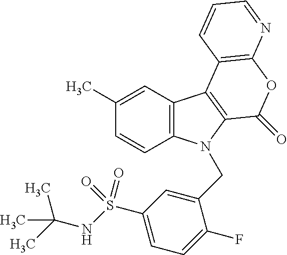 | 494.6 |
| 112 | 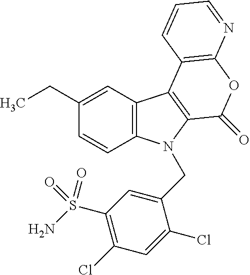 | 503.4 |
| 113 | 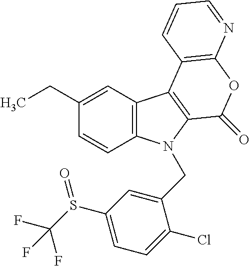 | 505.9 |
| 114 | 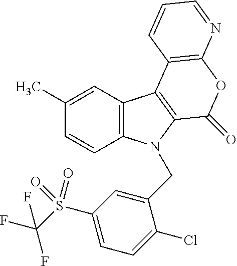 | 507.9 |
| 115 | 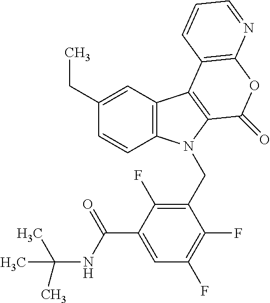 | 508.5 |
| 116 | 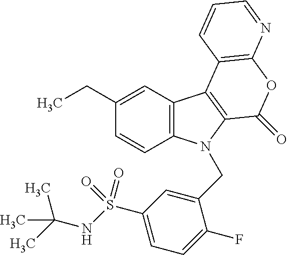 | 508.6 |
| 117 | 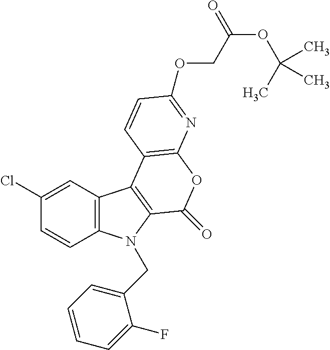 | 509.9 |
| 118 | 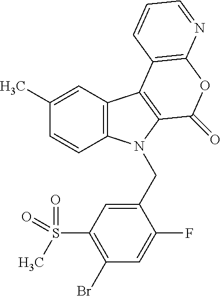 | 516.4 |
| 119 | 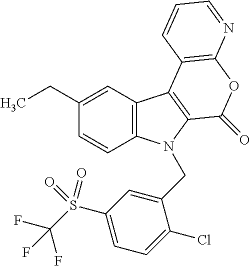 | 521.9 |
| 120 | 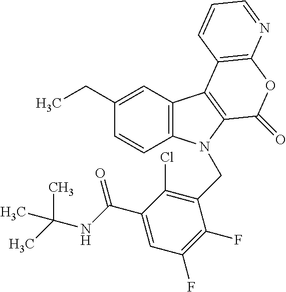 | 525.0 |
| 121 | 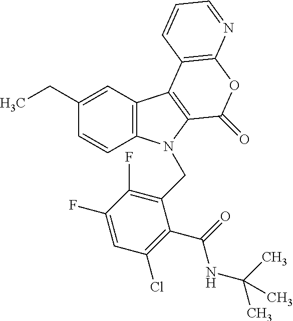 | 525.0 |
| 122 | 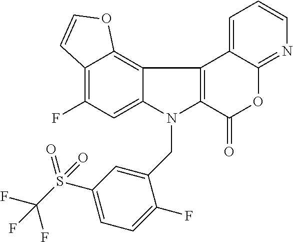 | 535.4 |
| 123 | 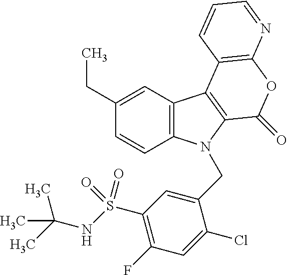 | 543.0 |
| 124 | 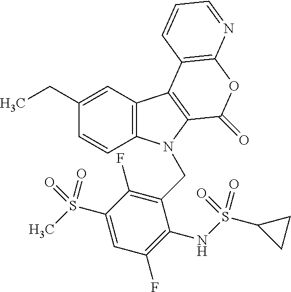 | 588.6 |
| 125 | 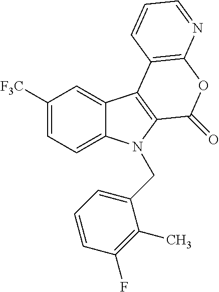 | 427.2 |
| 126 | 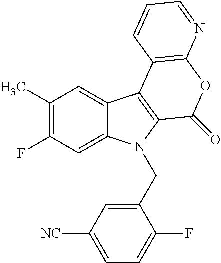 | 402.9 |
| 127 | 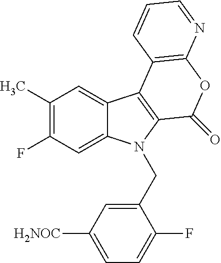 | 420.1 |
| 128 | 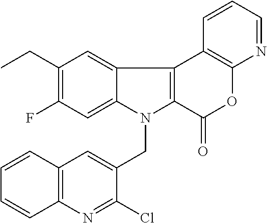 | NA |
| 129 | 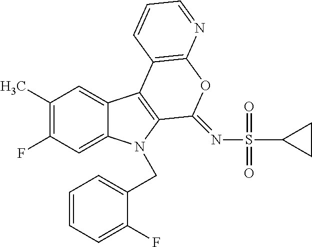 | 480.3 |

TABLE 2

| No. | STRUCTURE | M + H |
|---|---|---|
| 130 | 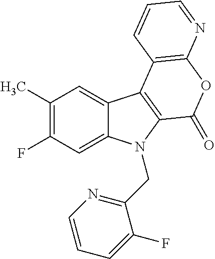 | 378.4 |
| 131 | 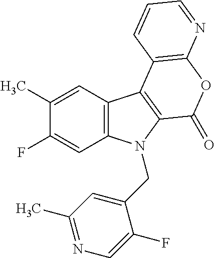 | 393.4 |
| 132 | 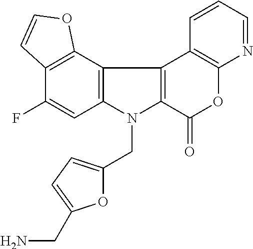 | 404.4 |
| 133 | 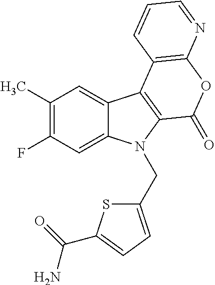 | 408.4 |
| 134 | 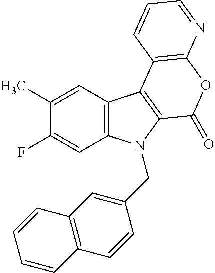 | 409.4 |
| 135 | 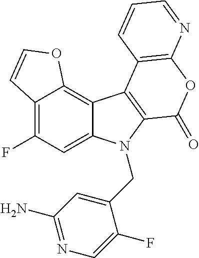 | 419.4 |
| 136 | 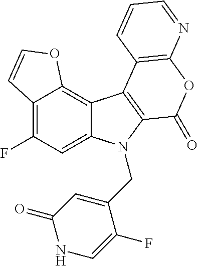 | 420.3 |
| 137 | 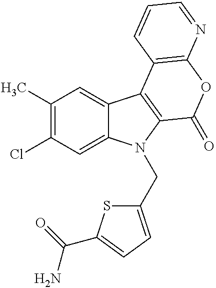 | 424.9 |
| 138 | 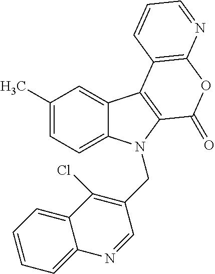 | 426.9 |
| 139 | 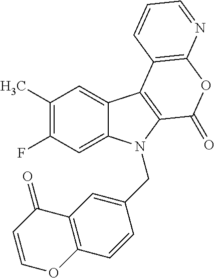 | 427.4 |
| 140 | 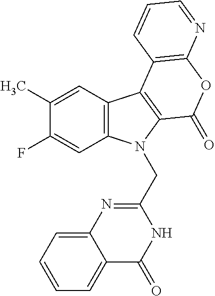 | 427.4 |
| 141 | 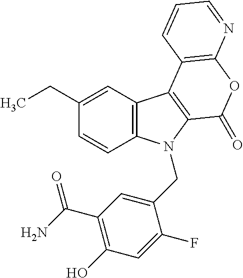 | 432.4 |
| 142 | 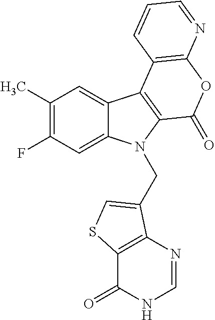 | 433.4 |
| 143 | 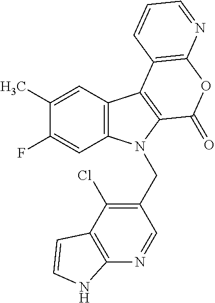 | 433.8 |
| 144 | 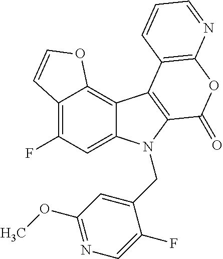 | 434.4 |
| 145 | 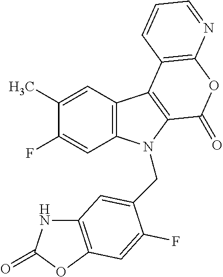 | 434.4 |
| 146 | 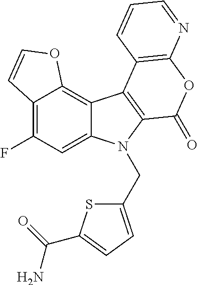 | 434.4 |
| 147 | 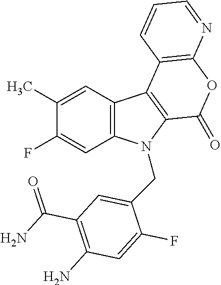 | 435.4 |
| 148 | 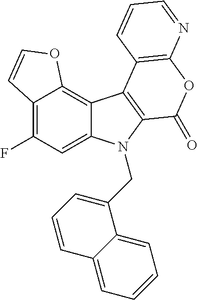 | 435.4 |
| 149 | 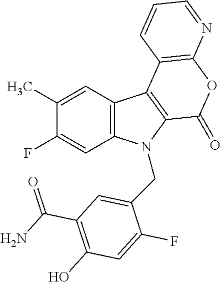 | 436.4 |
| 150 | 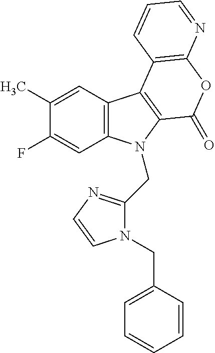 | 439.5 |
| 151 | 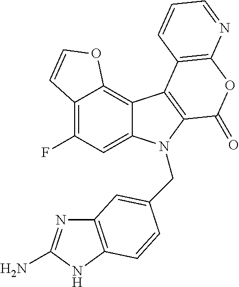 | 440.4 |
| 152 | 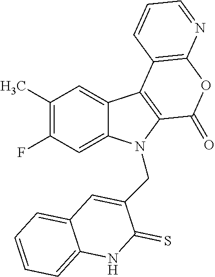 | 442.5 |
| 153 | 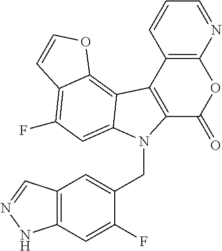 | 443.4 |
| 154 | 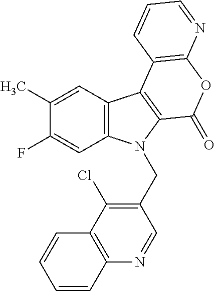 | 444.9 |
| 155 | 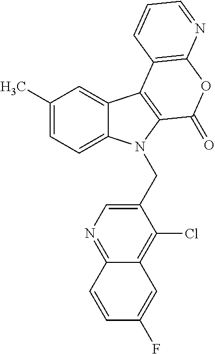 | 444.9 |
| 156 | 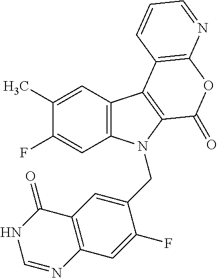 | 445.4 |
| 157 | 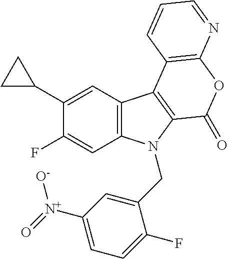 | 448.4 |
| 158 | 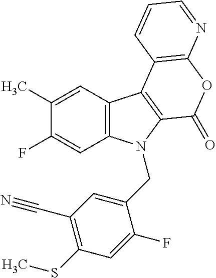 | 448.5 |
| 159 | 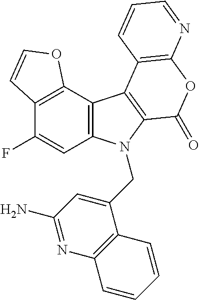 | 451.4 |
| 160 | 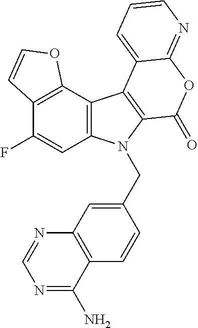 | 452.4 |
| 161 | 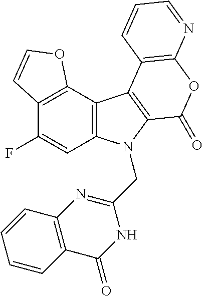 | 453.4 |
| 162 | 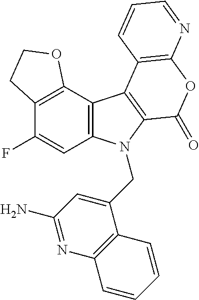 | 453.4 |
| 163 | 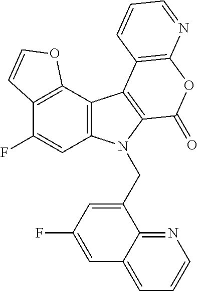 | 454.4 |
| 164 | 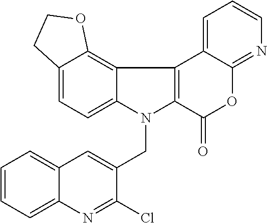 | 454.9 |
| 165 | 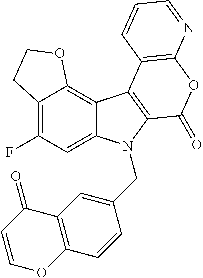 | 455.4 |
| 166 | 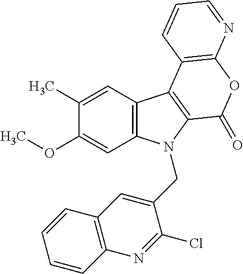 | 456.9 |
| 167 | 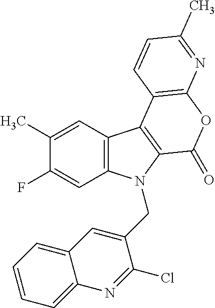 | 458.9 |
| 168 | 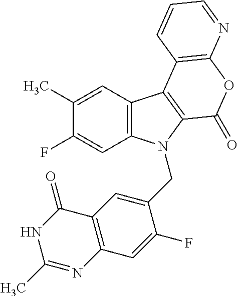 | 459.4 |
| 169 | 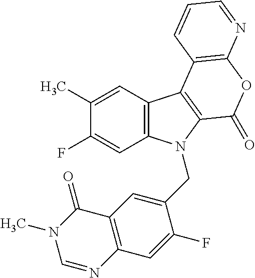 | 459.4 |
| 170 | 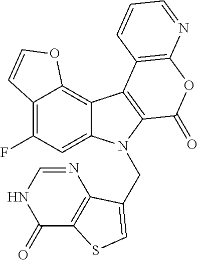 | 459.4 |
| 171 | 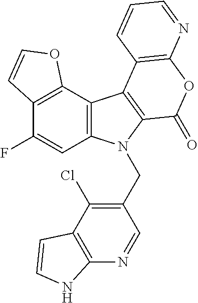 | 459.8 |
| 172 | 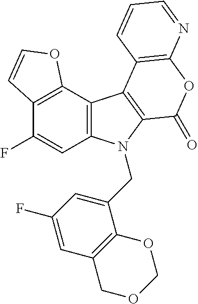 | 461.4 |
| 173 | 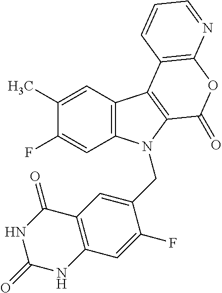 | 461.4 |
| 174 | 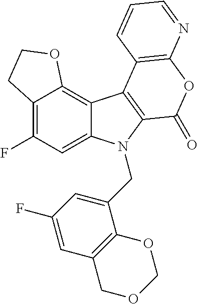 | 463.4 |
| 175 | 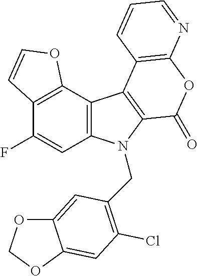 | 463.8 |
| 176 | 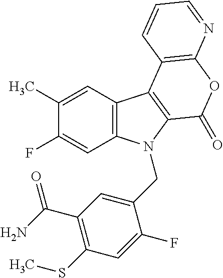 | 466.5 |
| 177 | 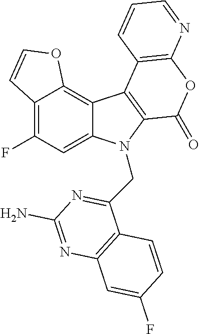 | 470.4 |
| 178 | 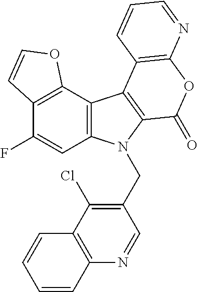 | 470.9 |
| 179 | 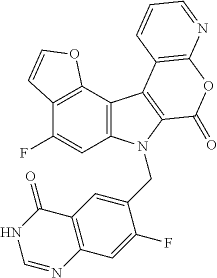 | 471.4 |
| 180 | 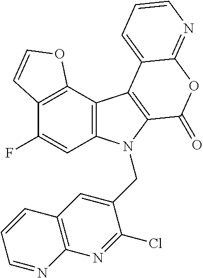 | 471.9 |
| 181 | 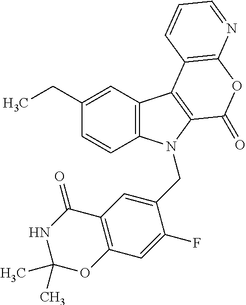 | 472.5 |
| 182 | 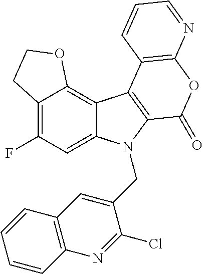 | 472.9 |
| 183 | 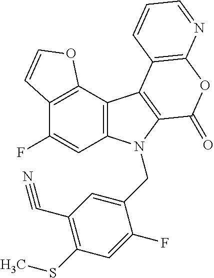 | 474.5 |
| 184 | 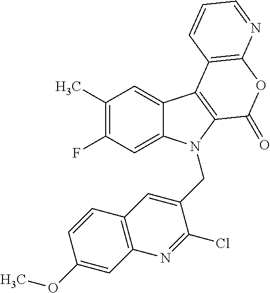 | 474.9 |
| 185 | 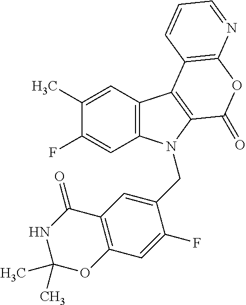 | 476.5 |
| 186 | 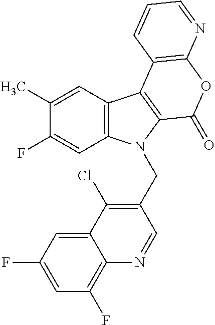 | 480.8 |
| 187 | 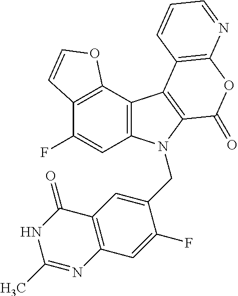 | 485.4 |
| 188 | 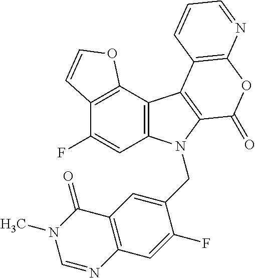 | 485.4 |
| 189 | 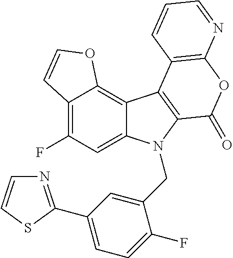 | 486.5 |
| 190 | 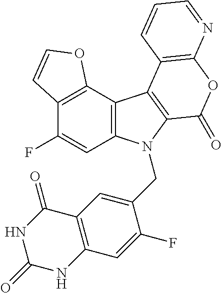 | 487.4 |
| 191 | 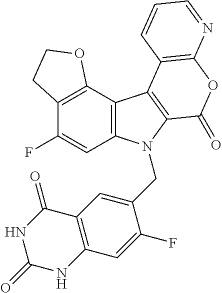 | 489.4 |
| 192 | 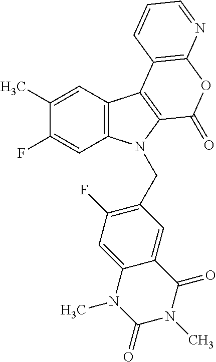 | 489.5 |
| 193 | 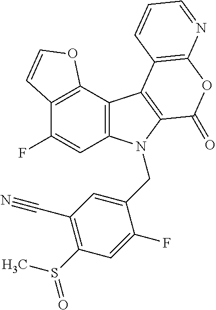 | 490.5 |
| 194 | 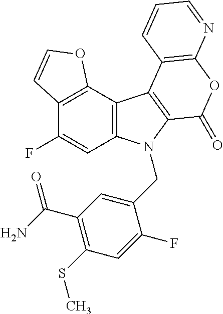 | 492.5 |
| 195 | 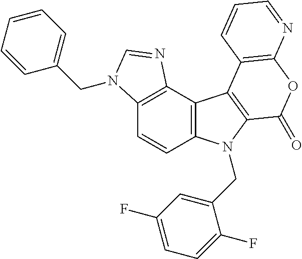 | 493.5 |
| 196 | 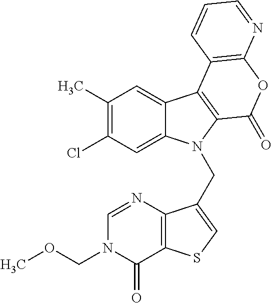 | 493.9 |
| 197 | 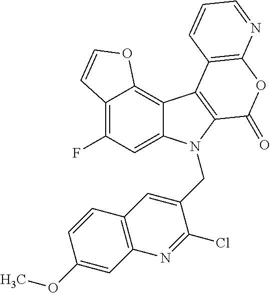 | 500.9 |
| 198 | 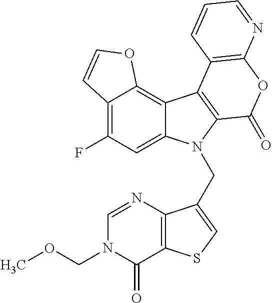 | 503.5 |
| 199 | 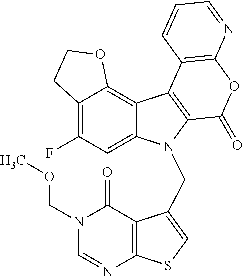 | 505.5 |
| 200 | 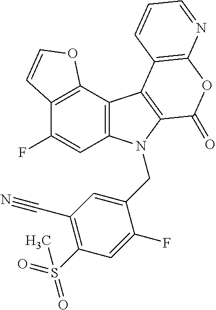 | 506.5 |
| 201 | 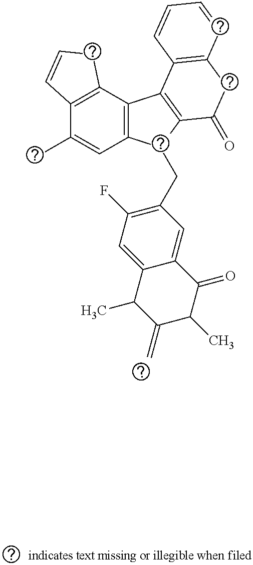 | 515.4 |
| 202 | 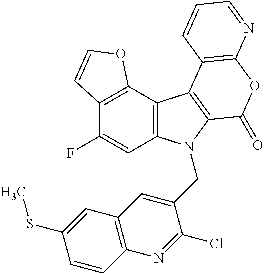 | 517.0 |
| 203 | 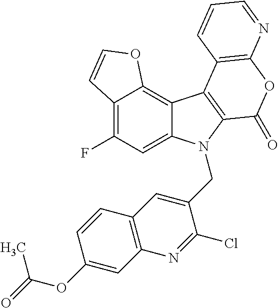 | 528.9 |
| 204 | 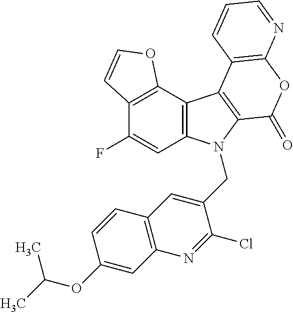 | 528.9 |
| 205 | 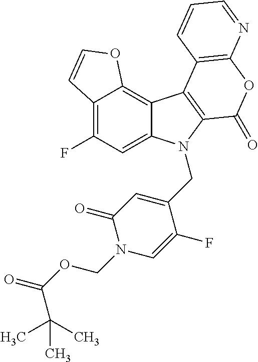 | 534.5 |
| 206 | 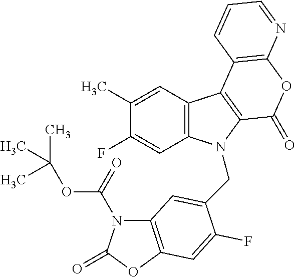 | 534.5 |
| 207 | 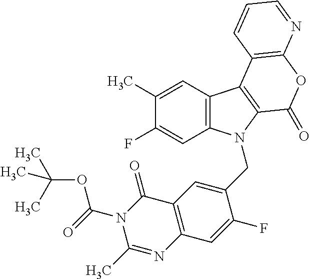 | 559.5 |
| 208 | 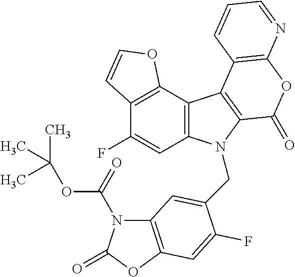 | 560.5 |
| 209 | 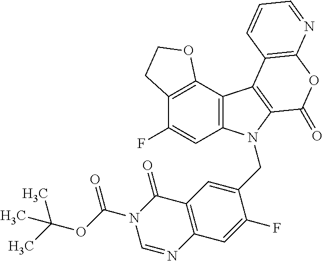 | 573.5 |
| 210 | 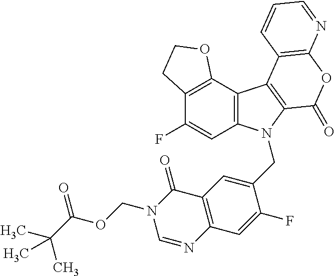 | 585.5 |
| 211 | 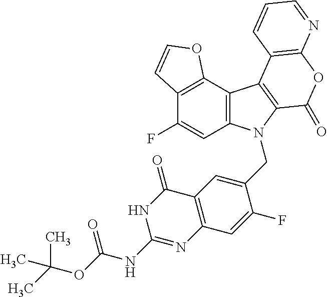 | 586.5 |
| 212 | 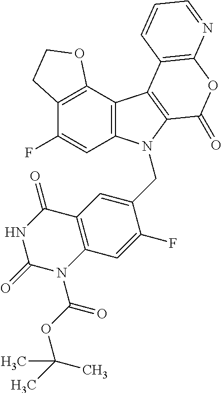 | 589.5 |
| 213 | 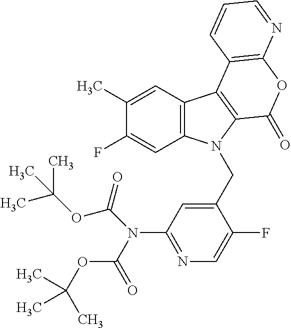 | 593.6 |
| 214 | 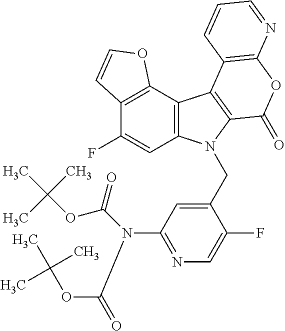 | 619.6 |
| 215 | 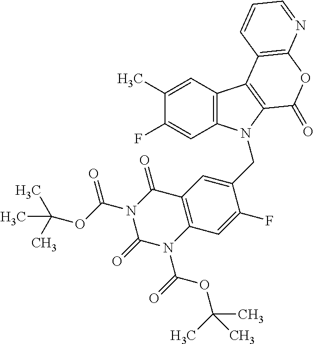 | 661.6 |
| 216 | 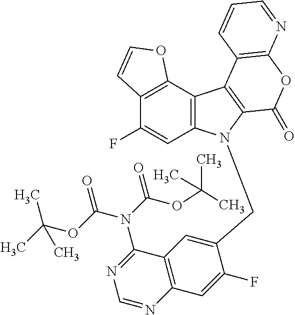 | 670.6 |
| 217 | 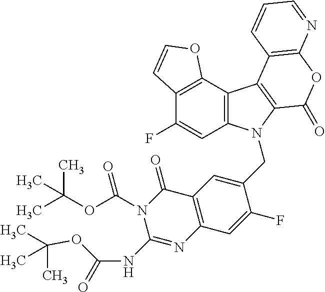 | 686.6 |
| 218 | 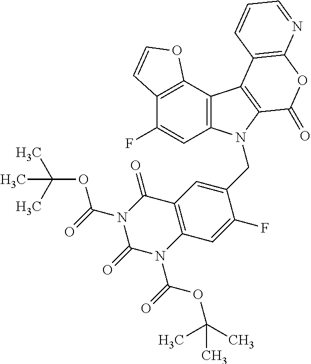 | 687.6 |
| 219 | 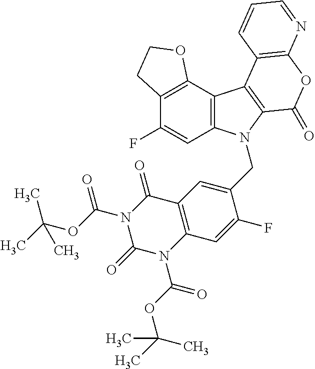 | 689.6 |
| 220 | 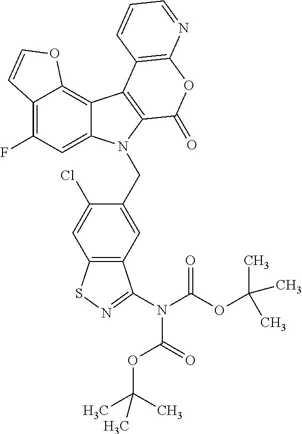 | 692.1 |
| 221 | 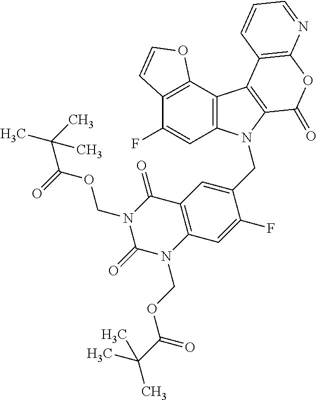 | 715.7 |
| 222 | 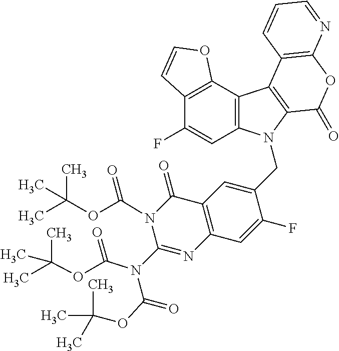 | 786.8 |
| 223 | 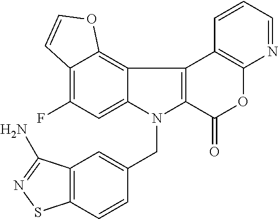 | 457.5 |
| 224 | 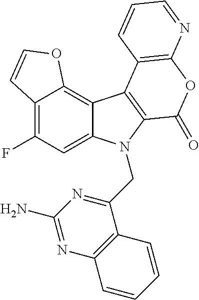 | 452.5 |
| 225 | 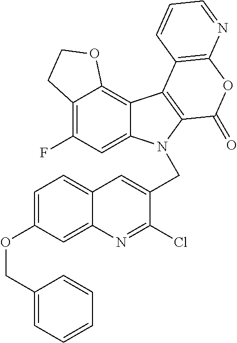 255 | 579 |
| 226 | 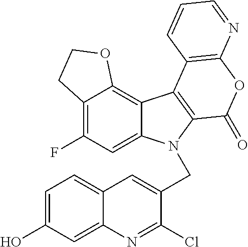 226 | 488.9 |
| 227 | 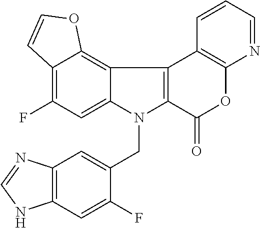 | 443.4 |
| 228 | 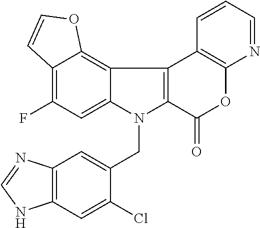 | 459.8 |
| 229 | 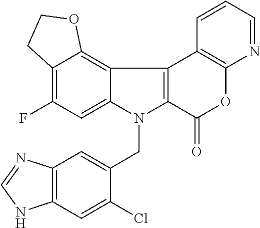 | 461.8 |
| 230 | 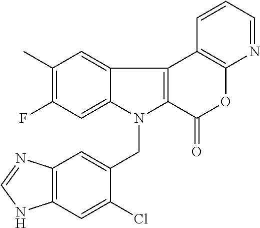 | 433.8 |

and pharmaceutically acceptable salts, solvates, esters and prodrugs thereof.

### Methods for Making the Tetracyclic Indole Derivatives

Methods useful for making the Tetracyclic Indole Derivatives are set forth in the Examples below and generalized in Schemes 1-4. Examples of commonly known methodologies useful for the synthesis of indoles are set forth, for example, in G. R. Humphrey and J. T. Kuethe, Chemical Reviews 106:2875-2911, 2006.

Scheme 1 shows one method for preparing compounds of formula A4, which are useful intermediates for making of the Tetracyclic Indole Derivatives.

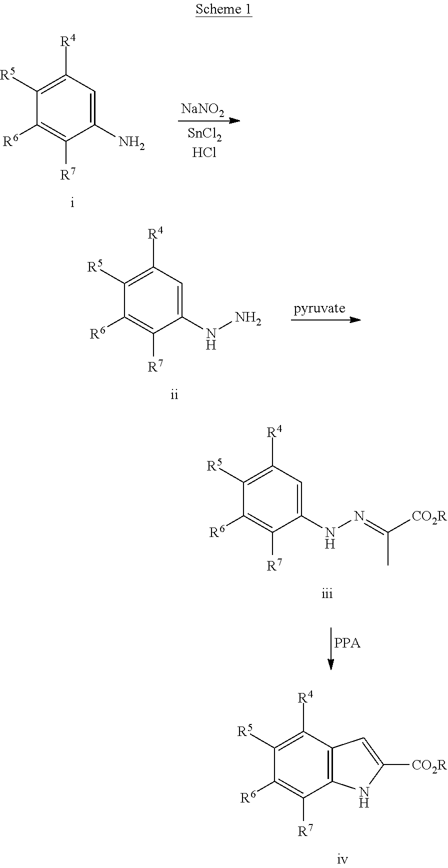

wherein R⁴-R⁷ are defined above for the compounds of formula (I) and R is H, alkyl or aryl.

An aniline compound of formula i can be converted to an indole compound of formula iv using various indole syntheses that are well-known to those skilled in the art of organic synthesis, including but not limited to, a Fischer indole synthesis through intermediates of type and iii, the method set forth in Nazare et al., Angew. Chem., 116:4626-4629 (2004). The compounds of formula iv can be further elaborated to provide the Tetracyclic Indole Derivatives using the method described below in Scheme 4.

Scheme 2 shows methods useful for making compounds viii and x, which are useful intermediates for making of the Tetracyclic Indole Derivatives.

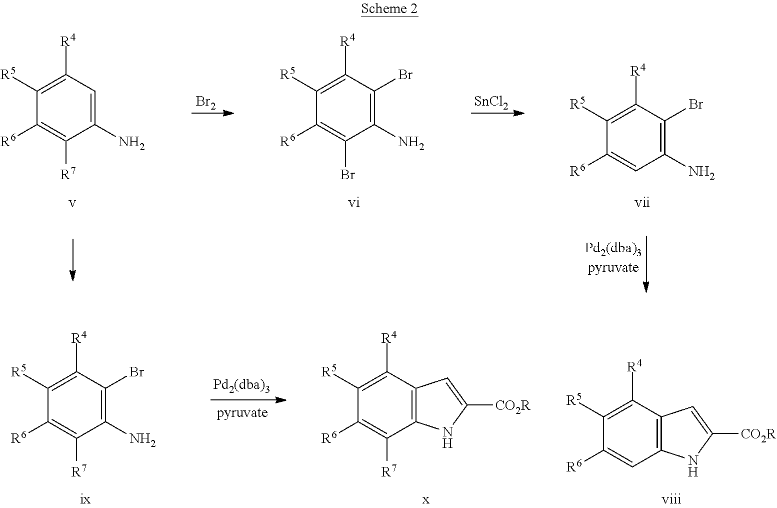

wherein R⁴-R⁷ are defined above for the compounds of formula (I) and R is H, alkyl or aryl.

A benzene derivative of formula v, wherein R⁷ is H, can be di-brominated to provide compound vi. Selective de-bromination provides the corresponding monobromo analog vii, which under palladium catalyzed cyclization conditions provides the desired intermediate viii, wherein R⁷ is H. Alternatively a compound of formula v, wherein R⁷ is other than H, can be monobrominated to provide compound ix. A compound of formula ix can then undergo under palladium catalyzed cyclization conditions provides the desired intermediate x, wherein R⁷ is other than H.

Scheme 3 illustrates methods by which intermediate compounds of formula xi can be further derivatized to provide the Tetracyclic Indole Derivatives, which are intermediates to the title Tetracyclic Indole derivatives.

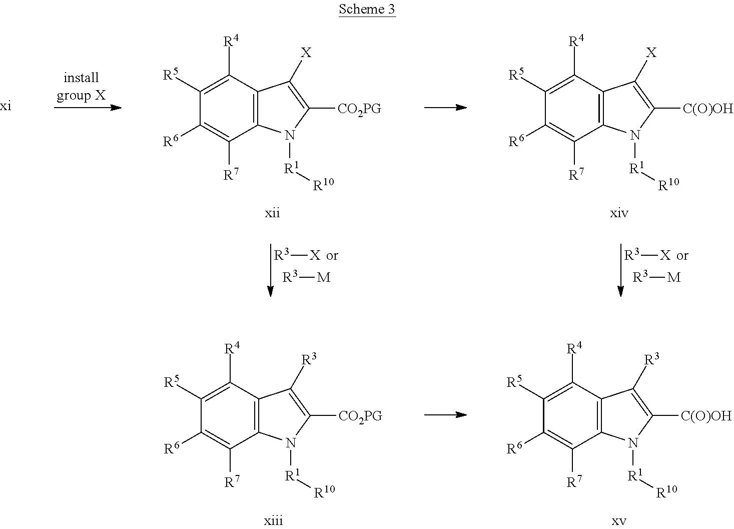

wherein R¹, R³, R⁴-R⁷ and R¹⁰ are defined above for the compounds of formula (I); PG is a carboxy protecting group; and X is halo, —O-triflate, —B(OH)₂, —Si(alkyl)₂OH, —Sn(alkyl)₃, —MgBr, —MgCl, —ZnBr, or —ZnCl; and M is any metal which can participate in an organometallic cross-coupling reaction.

An intermediate compound of formula xi can be converted to a 3-substituted indole of formula xii using methods well-known to one skilled in the art of organic synthesis. A compound of formula xii, wherein X is halo or —O-triflate can then be coupled with an appropriate compound of formula R³-M (wherein M is —B(OH)₂, —Si(alkyl)₂OH, —Sn(alkyl)₃, —MgBr, —MgCl, —ZnBr, —ZnCl, or any metal which can participate in an organometallic cross-coupling reaction) using an organometallic cross-coupling method. Alternatively, a compound of formula xii, wherein X is —B(OH)₂, —Si(alkyl)₂OH, —Sn(alkyl)₃, —MgBr, —MgCl, —ZnBr, —ZnCl, or any metal which can participate in an organometallic cross-coupling reaction, can then be coupled with an appropriate compound of formula R³-M (wherein M is halo or —O-triflate) using an organometallic cross-coupling method. Suitable cross-coupling methods include, but not limited to, a Stille coupling (see Choshi et al., J. Org. Chem., 62:2535-2543 (1997), and Scott et al., J. Am. Chem. Soc., 106:4630 (1984)), a Suzuki coupling (see Miyaura et al., Chem. Rev., 95:2457 (1995)), a Negishi coupling (see Thou et al., J. Am. Chem. Soc., 127:12537-12530 (2003)), a silanoate-based coupling (see Denmark et al., Chem. Eur. J. 12:4954-4963 (2006)) and a Kumada coupling (see Kumada, Pure Appl. Chem., 52:669 (1980) and Fu et al., Angew. Chem. 114:4363 (2002)) to provide a compound of formula F. The carboxy protecting group, PG, can then be removed from the compound of formula xiv and the resulting carboxylic acid can be derivatized using the methods described below in order to make the appropriate R² groups and make the compounds of formula xv, which correspond to the compounds of formula (I), wherein R² is —C(O)OH. Alternatively, a compound of formula xii can first be deprotected and the R² group attached using the above methods to provide a compound of formula xiii. A compound of formula xiii can then be cross-coupled with a compound of R³-X or R³-M as described above to provide make the compounds of formula xv.

Scheme 4 shows a method useful for making the Tetracyclic Indole Derivatives.

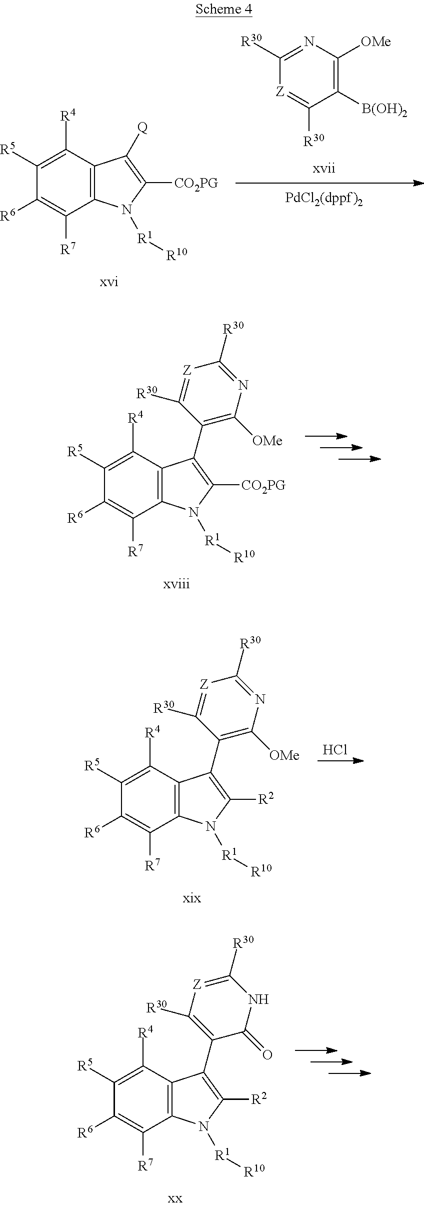

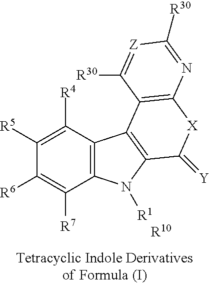

wherein X, Y, Z, R¹, R³, R⁴, R⁵, R⁶, R⁷, R¹⁰ and R³⁰ are as defined above for the Tetracyclic Indole Derivatives and Q is a halo group.

A 3-haloindole compound of formula xvi can be coupled with a boronic acid of formula xvii using a Suzuki coupling reaction to provide the R³-substituted indole compounds of formula xviii. A compound of formula xviii can be further elaborated using methods set forth above to provide the compounds of formula xix. A compound of formula N can be converted to a compound of formula xx using strong acid, such as HCl. A compound of formula xx can than be reacted with a base or dehydrating agent to provide the Tetracyclic Indole Derivatives. The starting material and reagents depicted in Schemes 1-4 are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the relevant art will recognize that the synthesis of Tetracyclic Indole Derivatives may require the need for the protection of certain functional groups (i.e., derivatization for the purpose of chemical compatibility with a particular reaction condition). Suitable protecting groups for the various functional groups of the Tetracyclic Indole Derivatives and methods for their installation and removal may be found in Greene et al., Protective Groups in Organic Synthesis, Wiley-Interscience, New York, (1999).

One skilled in the relevant art will recognize that one route will be optimal depending on the choice of appendage substituents. Additionally, one skilled in the art will recognize that in some cases the order of steps has to be controlled to avoid functional group incompatibilities. One skilled in the art will recognize that a more convergent route (i.e. non-linear or preassembly of certain portions of the molecule) is a more efficient method of assembly of the target compounds. Methods suitable for the preparation of Tetracyclic Indole Derivatives are set forth above in Schemes 1-4.

The starting materials and the intermediates prepared using the methods set forth in Schemes 1-4 may be isolated and purified if desired using conventional techniques, including but not limited to filtration, distillation, crystallization, chromatography and the like. Such materials can be characterized using conventional means, including physical constants and spectral data.

### EXAMPLES

### General Methods

Solvents, reagents, and intermediates that are commercially available were used as received. Reagents and intermediates that are not commercially available were prepared in the manner as described below. ‘H NMR spectra were obtained on a Bruker Avance 500 (500 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 5-7 min—95% CH₃CN, 7 min—stop. The retention time and observed parent ion are given. Flash column chromatography was performed using pre-packed normal phase silica from Biotage, Inc. or bulk silica from Fisher Scientific.

### Example 1

### Preparation of Intermediate Compound 1E

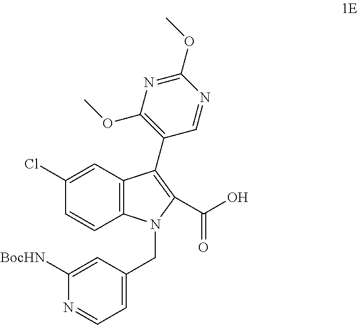

### Step 1:

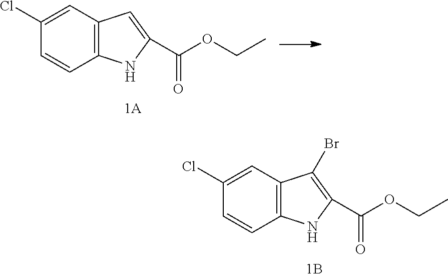

To a solution of ethyl 5-chloroindole-2-carboxylate, 1A (20 g, 89.6 mmol) in THF (200 mL) in a cooled water bath was added N-bromosuccinimide (16.0 g, 89.9 mmol) slowly. The resulting reaction mixture was stirred at room temperature for 18 h before water (700 mL) was added. The mixture was continued to stir at room temperature for 20 min and then filtered. The solids were washed with water (2×100 mL), and dried to afford the crude product 1B (25.8 g, 90% yield). ¹H NMR (500 MHz, CDCl₃) δ 9.06 (s, 1H), 7.66-7.65 (m, 1H), 7.35-7.31 (m, 2H), 4.47 (q, J=7.25 Hz, 2H), 1.46 (t, J=7.09 Hz, 3H).

### Step 2:

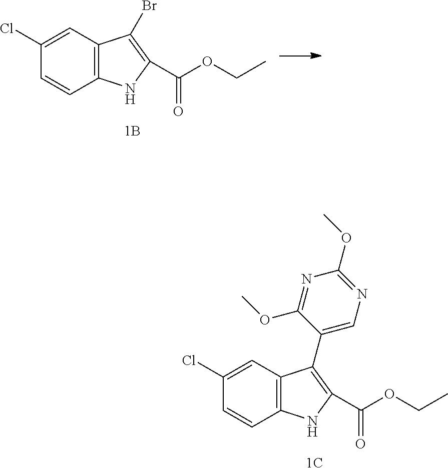

To a mixture of 3-bromo-5-chloro-1H-indole-2-carboxylic acid ethyl ester, 1B (1.00 g, 3.31 mmol), 2,4-dimethoxypyrimidine-5-boronic acid (0.73 g, 3.97 mmol), [1,1′-bis(diphenylphosphino)ferrocene]dichloropalladium(II) with dichloromethane complex (1:1) (0.26 g, 0.32 mmol) in DME (15 mL) was added a solution of sodium carbonate (4.5 mL of 1.5 M, 6.75 mmol) via a syringe. The reaction mixture was stirred at reflux for 6 h before cooled down to room temperature. The mixture was diluted with dichloromethane (50 mL), and was filtered through a pad of celite. The filtrate was concentrated under reduced pressure. The residue was purified by flash chromatography on silica gel (20% ethyl acetate in hexanes) to provide the product 1C as a white solid (0.47 g, 39% yield). M.S. found for C₁₇H₁₆ClN₃O₄: 362.2 (M+H)⁺.

### Step 3:

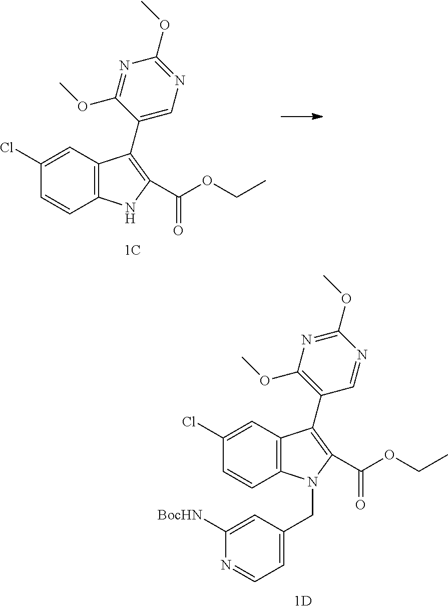

To a solution of 5-chloro-3-(2,4-dimethoxy-pyrimidin-5-yl)-1H-indole-2-carboxylic acid ethyl ester, 1C (620 mg, 1.71 mmol) in DMF was added (4-bromomethyl-pyridin-2-yl)-carbamic acid tert-butyl ester, (490 mg, 1.71 mmol) and cesium carbonate (1100 mg, 3.39 mmol). The resulting suspension was stirred at room temperature for 17 h. The mixture was then diluted with ethyl acetate (80 mL), and washed with water (3×50 mL). The organic layer was dried over sodium sulfate, filtered and concentrated under reduced pressure. The residue was purified by chromatography on silica gel using 30% ethyl acetate in hexanes to deliver the product 1D (705 mg, 73% yield). M.S. found for C₂₈H₃₀ClN₅O₆: 568.3 (M+H)⁺.

### Step 4:

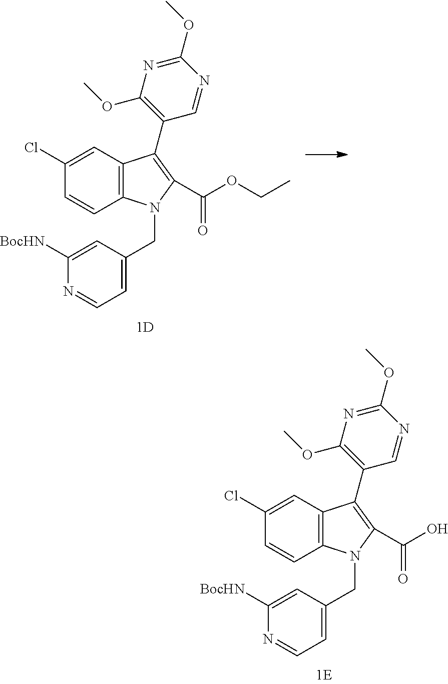

To a solution of 1-(2-tert-butoxycarbonylamino-pyridin-4-ylmethyl)-5-chloro-3-(2,4-dimethoxy-pyrimidin-5-yl)-1H-indole-2-carboxylic acid ethyl ester, 1D (500 mg, 0.88 mmol) in THF (10 mL) was added an aqueous solution of lithium hydroxide (2.0 ml of 1 M, 2.9 mmol). The resulting reaction mixture was stirred at reflux for 16 h. TheReaction was then cooled and concentrated under reduced pressure. The residue was dissolved in methanol (80 mL), neutralized with 1.0 M HCl aqueous solution (2.5 mL, 2.5 mmol) and then concentrated again under reduced pressure. The residue was extracted with dichloromethane (3×30 mL). The combined organic layer was concentrated under reduced pressure, and dried on house vacuum to provide compound 1E (440 mg, 92%). M.S. found for C₂₆H₂₆ClN₅O₆: 540.3 (M+H)⁺.

### Example 2

### Preparation of Intermediate Compound 2E

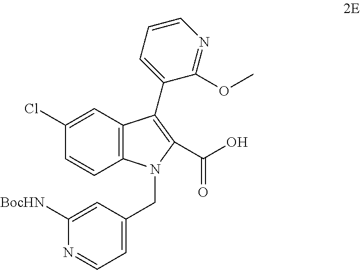

### Step 1:

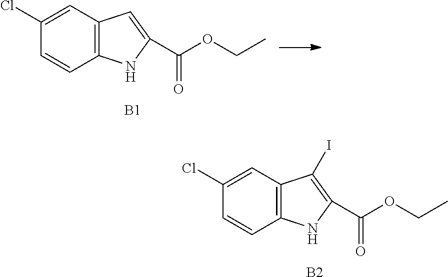

To a solution of 5-chloro-1H-indole-2-carboxylic acid ethyl ester, 2A (5.0 g, 22 mmol) in chloroform (25 mL) at room temperature was added N-iodosuccinimide (5.0 g, 22 mmol). The resulting suspension was stirred at room temperature for 24 h. The mixture was then concentrated under reduced pressure, and the residue dissolved into ethyl acetate (300 mL). The mixture was washed with water (100 mL) and brine respectively. The separated organic layer was dried over sodium sulfate, filtered and concentrated under reduced pressure to give the crude product 2B (7.0 g, 91% yield). M.S. found for C11H9ClINO2: 350.2 (M+H)⁺.

### Step 2:

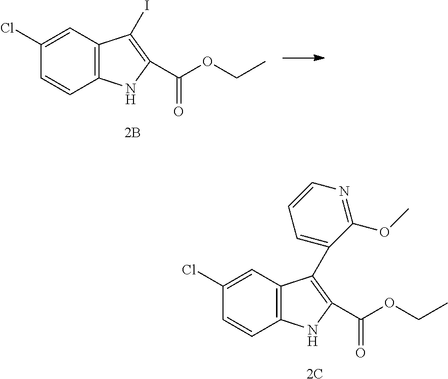

5-Chloro-3-iodo-1H-indole-2-carboxylic acid ethyl ester, 2B (3.0 g, 8.6 mmol) was dissolved into 1,2-dimethoxyethane (40 mL) and PdCl₂(dppf)₂ (0.7 g, 0.86 mmol) was added. The resulting mixture was refluxed at 90° C. for 0.5 h. To the above mixture was added slowly a solution of 2-methoxy-3-pyridine boronic acid (2.9 g, 18.8 mmol) and potassium carbonate (2.4 g, 17.3 mmol) in water (10 mL). The resulting biphasic mixture was vigorously stirred at 90° C. for 1 h before it was cooled to room temperature. The reaction mixture was filtered and concentrated under reduced pressure. The residue was diluted with ethyl acetate (150 mL), and was washed with a solution of sodium sulfite (5 g) in water (50 mL). The aqueous layer was extracted with ethyl acetate (2×100 mL). The combined organic layer was dried over sodium sulfate, filtered and concentrated under reduced pressure. The residue was purified by flash chromatography to provide compound 2C (1.87 g, 66% yield). M.S. found for C17H15ClN2O3: 331.20 (M+H)⁺.

### Step 3:

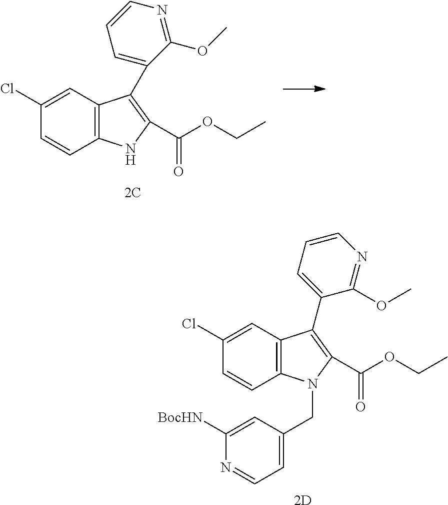

5-Chloro-3-(2-methoxy-pyridin-3-yl)-1H-indole-2-carboxylic acid ethyl ester, 2C (1.0 g, 3.0 mmol) was dissolved in DMF (15 mL) at room temperature. (4-bromomethyl-pyridin-2-yl)-carbamic acid tert-butyl ester (1.0 g, 3.6 mmol) and cesium carbonate (0.9 g, 4.5 mmol) were added sequentially and the resulting suspension stirred at room temperature for 20 h. Ethyl acetate (200 mL) and water (100 mL) were added to the reaction mixture, and the layers were separated. The organic layer was washed with brine, and dried over sodium sulfate, filtered and concentrated under reduced pressure. The crude product was purified by flash chromatography to provide compound 2D (1.49 g, 93% yield). M.S. found for C29H30ClN3O5: 537.27 (M+H)⁺; 437.17 (M-Boc+H)⁺.

### Step 4:

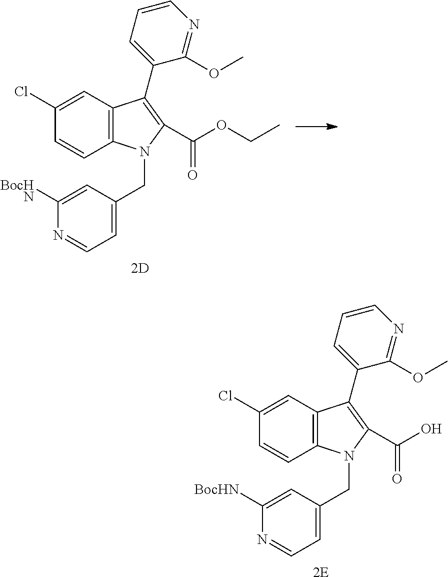

To the solution of 1-(2-tert-butoxycarbonylamino-pyridin-4-ylmethyl)-5-chloro-3-(2-methoxy-pyridin-3-yl)-1H-indole-2-carboxylic acid ethyl ester, 2D (1.5 g, 2.79 mmol) in THF (20 mL) was added the solution of lithium hydroxide (0.3 g, 8.37 mmol) in water (5 mL). The resulting suspension was stirred at 60° C. for 20 h. The mixture was concentrated under reduced pressure. Ethyl acetate (150 mL) and water (100 mL) were added to the residue. The aqueous layer was acidified to pH=1-2 by adding aqueous 1N HCl solution, and was saturated with NaCl salts. The layers were separated, and the aqueous layer was further extracted with ethyl acetate (2×100 mL). The combined organic layer was dried over sodium sulfate, filtered and concentrated under reduced pressure to yield the crude product 2E (100% yield).¹NMR (500 MHz, CDCl3) δ 9.36 (s, 1H), 8.22 & 8.21 (dd, J=1.89 Hz & 5.04 Hz, 1H), 8.07 (s, 1H), 7.81 (d, J=5.68 Hz, 1H), 7.70 & 7.68 (dd, J=1.89 Hz & 7.25 Hz, 1H), 7.45 (d, J=1.89 Hz, 1H), 7.31 & 7.29 (dd, J=1.89 Hz & 8.83 Hz, 1H), 7.23 (d, J=8.83 Hz, 1H), 7.01 (q, J=5.04 Hz & 2.21 Hz, 1H), 6.36 (d, J=5.04 Hz, 1H), 5.85 (s, 2H), 3.80 (s, 3H), 1.46 (s, 9H).

### Example 3

### Preparation of Intermediate Compound 3E

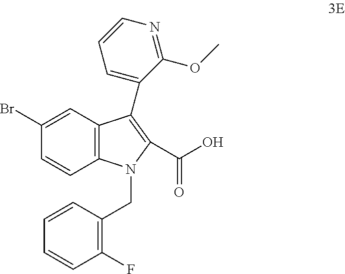

### Step 1:

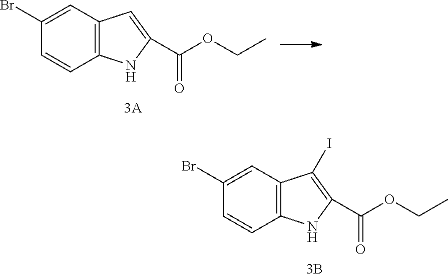

Ethyl 5-bromo 2-indole carboxylate, 3A (4.0 g, 14.9 mmol) was dissolved into acetone (200 mL) at room temperature. To the mixture was added N-iodosuccinimide (3.65 g, 15.4 mmol). The resulting suspension was stirred at room temperature for 3 h. The mixture was concentrated under reduced pressure, and the residue was dissovled into ethyl acetate (150 mL). The mixture was washed with saturated aqueous sodium thiosulfate solution (50 mL). The layers were separated, and the aqueous layer was extracted with ethyl acetate (2×100 mL). The combined organic layer was dried (magnesium sulfate), filtered and concentrated under reduced pressure to give the crude product 3B (100% yield). ¹NMR (400 MHz, d₆-DMSO): δ 12.48 (s, 1H), 7.55 (s, 1H), 7.45-7.44 (m, 2H), 4.39 (q, J=6.59 & 7.32 Hz, 2H), 1.38 (t, J=7.32 Hz, 3H).

### Step 2:

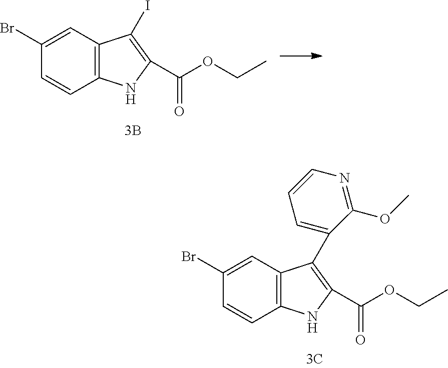

5-Bromo-3-iodo-1H-indole-2-carboxylic acid ethyl ester, 3B (8.66 g, 21.9 mmol) was dissolved into 1,2-dimethoxyethane (400 mL). And PdCl₂(dpp0₂ (1.80 g, 2.20 mmol) was added. The resulting mixture was de-gassed with nitrogen bubbling for 5 min before it was heated to 90° C. and stirred for 15 min. In a second flask, the mixture of 2-methoxy-3-pyridine boronic acid (3.72 g, 24.3 mmol) and potassium carbonate (15.2 g, 110 mmol) in dimethoxyethane (100 mL) and water (100 mL) was de-gassed with nitrogen bubbling for 5 min. The mixture was then transferred in three portions to the first flask. The resulting bi-phasic mixture was vigorously stirred at 90° C. for 3.5 h before it was cooled to room temperature. The reaction was quenched by addition of a solution of sodium sulfite (15 g) in water (200 mL) at room temperature. Ethyl acetate (200 mL) was added, and the layers were separated. The aqueous layer was extracted with ethyl acetate (2×300 mL). The combined organic layer was dried (magnesium sulfate), filtered and concentrated under reduced pressure to give the crude product 3C (100% yield). M.S. calc'd for C17H15BrN2O3: 375.22. Found: 377.00.

### Step 3:

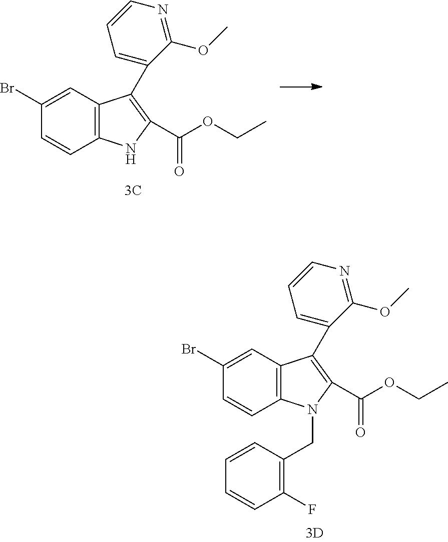

5-Bromo-3-(2-methoxy-pyridin-3-yl)-1H-indole-2-carboxylic acid ethyl ester, 3C (0.66 g, 1.59 mmol) was dissolved into DMF (50 mL) at room temperature. To the mixture were added 2-fluorobenzyl bromide (0.42 g, 2.23 mmol) and cesium carbonate (0.84 g, 2.40 mmol). The resulting suspension was stirred at room temperature for 18 h. Ethyl acetate (200 mL) and water (100 mL) were added to the reaction mixture, and the layers were separated. The aqueous layer was extracted with ethyl acetate (2×100 mL). The combined organic layer was washed with water (2×100 mL). The separated organic layer was dried (magnesium sulfate), filtered and concentrated under reduced pressure to give the crude product. The crude product was purified by flash chromatography to give product 3D (0.32 g, 42% yield). M.S. calc'd for C24H20N2O3BrF: 483.33. Found: 485.3.

### Step 4:

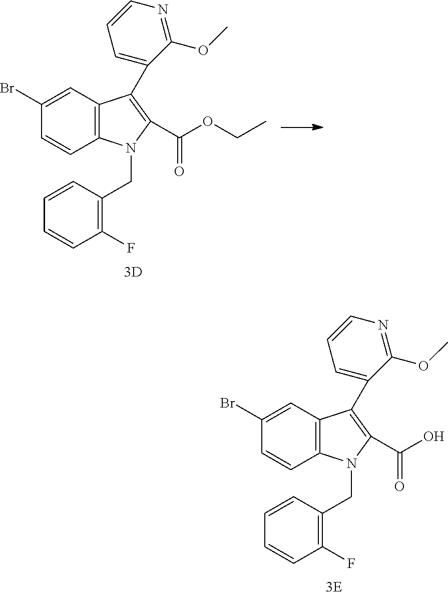

To the solution of 5-bromo-1-(2-fluoro-benzyl)-3-(2-methoxy-pyridin-3-yl)-1H-indole-2-carboxylic acid ethyl ester, 3D (0.32 g, 0.66 mmol) in methanol (5 mL) was added lithium hydroxide monohydrate (110 mg, 2.64 mmol). And water (0.2 mL) was added to improve the solubility. The resulting suspension was stirred at room temperature for 5 min before being placed in microwave reactor for 20 min (120° C., high power). The mixture was concentrated under reduced pressure. Ethyl acetate (50 mL) and water (50 mL) were added to the residue. The aqueous layer was acidified to pH=2 by adding aqueous 1N HCl solution, and was saturated with NaCl salts. The layers were seperated, and the aqueous layer was further extracted with ethyl acetate (2×50 mL). The combined organic layer was dried (magnesium sulfate) and filtered and concentrated under reduced pressure to provide compound 3E (93% yield). M.S. calc'd for C22H16N2O3BrF: 455.28. Found: 456.01 (M+H)⁺.

### Example 4

### Preparation of Intermediate Compound 4E

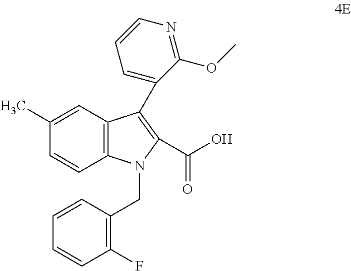

### Step 1:

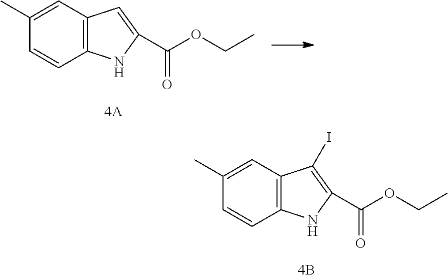

To the solution of ethyl 5-methyl indole carboxylate, 4A (5.0 g, 24.6 mmol) in acetone (200 mL) was added N-iodosuccinimide (3.65 g, 15.4 mmol). The resulting suspension was stirred at room temperature for 4 h. The mixture was concentrated under reduced pressure, and the residue was dissolved into ethyl acetate (200 mL). The mixture was washed with saturated aqueous sodium thiosulfate solution (100 mL). The layers were separated, and the aqueous layer was extracted with ethyl acetate (2×100 mL). The combined organic layer was washed with water (200 mL), and was then dried (magnesium sulfate), filtered and concentrated under reduced pressure to give the crude product 4B (7.62 g, 94% yield).

### Step 2:

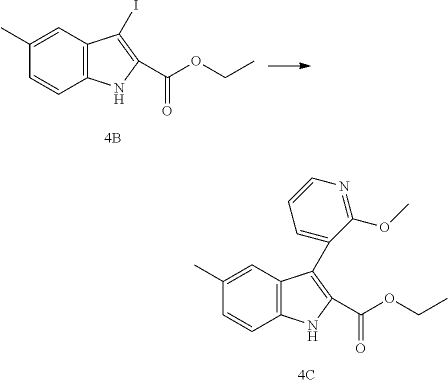

3-Iodo-5-methyl-1H-indole-2-carboxylic acid ethyl ester, 4B (7.62 g, 23.2 mmol) was dissolved into 1,2-dimethoxyethane (100 mL) and PdCl₂(dpp02 (1.89 g, 2.32 mmol) was added. The resulting mixture was de-gassed with nitrogen bubbling for 10 min. In a second flask, the mixture of 2-methoxy-3-pyridine boronic acid (4.26 g, 27.8 mmol) and potassium carbonate (16.0 g, 115.8 mmol) in dimethoxyethane (50 mL) and water (50 mL) was de-gassed with nitrogen bubbling for 5 min. The mixture was then transferred slowly to the first flask. The resulting biphasic mixture was stirred at room temperature for 15 min, and then vigorously stirred at 90° C. for 4 h. The reaction mixture was cooled to room temperature, and was quenched by addition of a solution of sodium sulfite (5 g) in water (100 mL) at room temperature. Ethyl acetate (200 mL) was added, and the layers were seperated. The aqueous layer was extracted with ethyl acetate (2×300 mL). The combined organic layer was filtered through a pad of celite, dried over magnesium sulfate, and concentrated under reduced pressure to give the crude product 4C (4.12 g, 57% yield). M.S. calc'd for C18H18N2O3: 310.35. Found: 311.15 (M+H)⁺.

### Step 3:

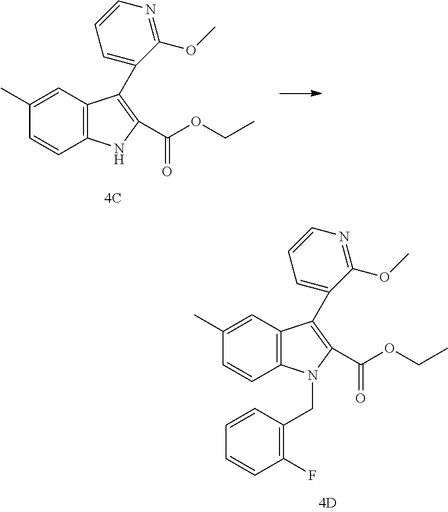

3-(2-Methoxy-pyridin-3-yl)-5-methyl-1H-indole-2-carboxylic acid ethyl ester, 4C (0.70 g, 2.25 mmol) was dissolved into DMF (25 mL) at room temperature. To the mixture were added 2-fluorobenzyl bromide (0.68 g, 3.60 mmol) and cesium carbonate (1.60 g, 4.50 mmol). The resulting suspension was stirred at room temperature for 18 h. 300 mL of THF/ethyl acetate (1:3) and 50 mL of water were added to the reaction mixture, and the layers were separated. The aqueous layer was extracted with 100 mL of THF/ethyl acetate (1:3). The combined organic layer was washed with water (3×100 mL). The separated organic layer was dried over magnesium sulfate, filtered and concentrated under reduced pressure. The crude product obtained was purified by flash chromatography to provide compound 4D(0.75 g, 79% yield). M.S. calc'd for C25H23FN2O3: 418.46. Found: 419.27 (M+H)⁺.

### Step 4:

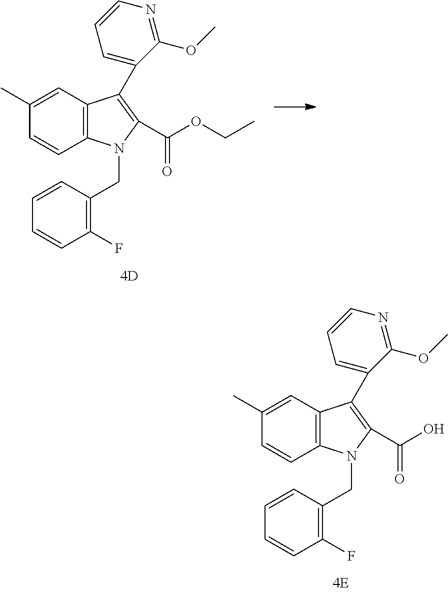

To the solution of 1-(2-fluoro-benzyl)-3-(2-methoxy-pyridin-3-yl)-5-methyl-1H-indole-2-carboxylic acid ethyl ester, 4D (0.75 g, 1.79 mmol) in methanol (20 mL) was added lithium hydroxide monohydrate (220 mg, 5.24 mmol). Water (0.2 mL) was added to improve the solubility. The resulting suspension was stirred at room temperature for 5 min before being placed in microwave reactor for 20 min (120° C., high power). The mixture was concentrated under reduced pressure, and 30 mL of water was added. The aqueous layer was acidified to pH=2 by adding aqueous 1N HCl solution, and the mixture was extracted three times with 100 mL of THF/ethyl acetate (3:1). The combined organic layer was dried over magnesium sulfate, filtered and concentrated under reduced pressure to yield the crude product 4E (0.70 g, 99% yield). M.S. calc'd for C23H19FN2O3: 390.41. Found: 391.2 (M+H)⁺.

### Example 5

### Preparation of Intermediate Compound 5J

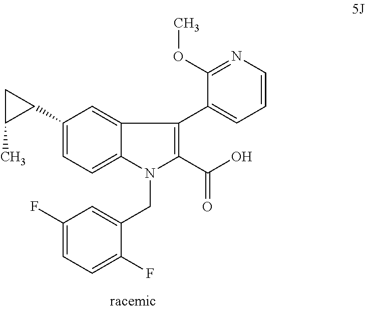

### Step 1:

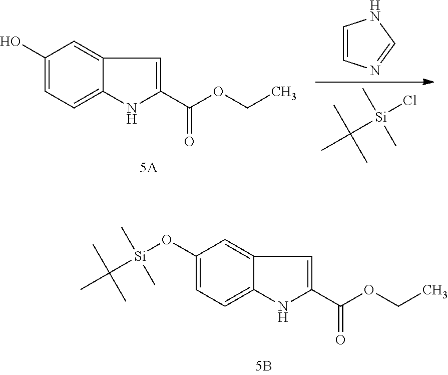

A solution of ethyl 5-hydroxy-1H-indole-2-carboxylate 5A (6.0 g; 29.24 mmol) in 300 mL of dichloromethane was treated with imidazole (4.0 eq, 7.96 g) and tert-butyldimethylsilyl chloride (2.0 eq, 8.82 g). The reaction was stirred at room temp for 3 h. A small sample (1 mL) was taken from reaction mixture, diluted with dichloromethane (10 mL) and washed with water. Evaporation of the solvent and NMR analysis showed all starting material had been consumed. The reaction mixture was diluted with dichloromethane (300 mL) and washed with water (2×100 mL) and brine (100 mL). The organic layer was dried over magnesium sulfate, filtered and concentrated to provide compound 5B (9.20 g; 98%) as a white solid.

### Step 2:

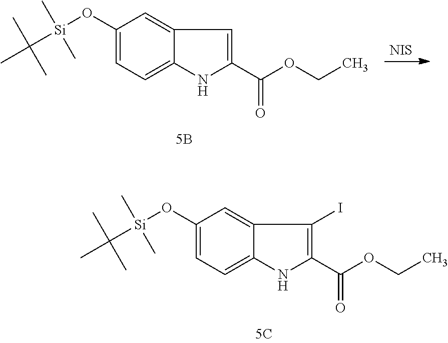

A solution of ethyl 5-tert-butyldimethylsilyloxy-1H-indole-2-carboxylate 5B (9.0 g) in 300 mL of chloroform was ice-cooled and treated with N-iodosuccinimide (1.1 eq, 6.97 g). The mixture was stirred at 0° C. for 10 min and then at room temp for 2 h. NMR analysis of a small aliquot showed complete conversion of starting material. The reaction mixture was diluted with dichloromethane (300 mL) and washed with aq saturated sodium thiosulfate (150 mL), aq saturated sodium bicarbonate (150 mL) and brine (100 mL). The organic layer was dried over magnesium sulfate, filtered and concentrated to provide compound 5C (11.58 g; 92%) as a white solid. M.S. found for C17H24INO3Si: 446.36 (M+H)⁺.

### Step 3:

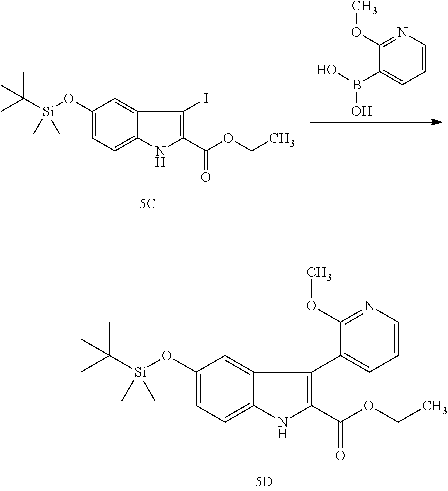

The 2-methoxy-3-pyridine boronic acid (1.05 eq, 3.27 g) was added to a solution of 5C (9.06 g; 20.345 mmol) in 100 mL of 1,2-dimethoxyethane. The mixture was degassed (vaccum/argon flush) and PdCl₂(dppf)₂ (10 mol %, 1.66 g) was added and the resulting orange solution was stirred for 30 min at room temp. A solution of potassium carbonate (4.0 eq, 81 mL of aq 1M solution) was added and the resulting brown solution was stirred at 90° C. for 2 h. The reaction mixture was cooled to room temperature and concentrated. The residue was diluted with ethyl acetate (600 mL) and washed with aq saturated sodium bicarbonate (100 mL) and brine (100 mL). The organic layer was dried over magnesium sulfate, filtered and concentrated. The residue was divided into two equal portions and each was purified by silica gel chromatography (Biotage 75-M column; gradient: 0 to 30% ethyl acetate in hexanes) to provide compound 5D as a white solid (6.76 g; 65%). M.S. found for C23H30N2O4Si: 427.56 (M+H)⁺.

### Step 4:

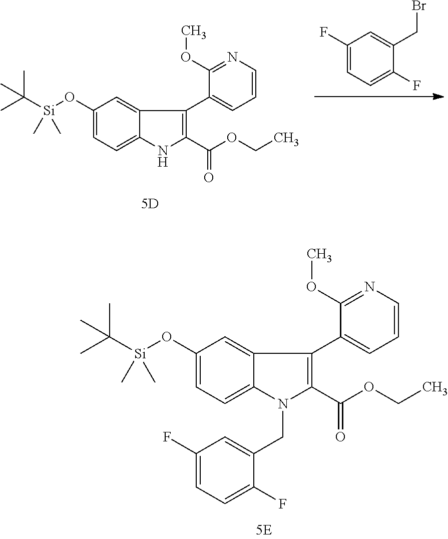

A solution of indole derivative 5D (6.5 g, 15.237 mmol) in 50 mL of dry THF was added to an ice-cooled suspension of sodium hydride (1.3 eq, 792 mg of 60% suspension in mineral oil) in 50 mL of dry THF. The resulting solution was stirred for 10 min followed by addition of 2,5-difluorobenzyl bromide (1.3 eq, 2.54 mL, d 1.613). A catalytic amount of tetrabutylammonium iodide (0.2 eq, 1.12 g) was added to the reaction mixture and stirring was continued for 18 h (temperature from 0 to 25° C.). The reaction was quenched by addition of water (10 mL) and the mixture was diluted with ethyl acetate (500 mL). The organic layer was washed with water (2×100 mL) and brine (80 mL), dried over magnesium sulfate, filtered and concentrated to afford the crude product 5E as a colorless foam contaminated with undesired bis-N,O-difluorobenzyl product. The crude mixture was used for next reaction without further any further purification.

### Step 5:

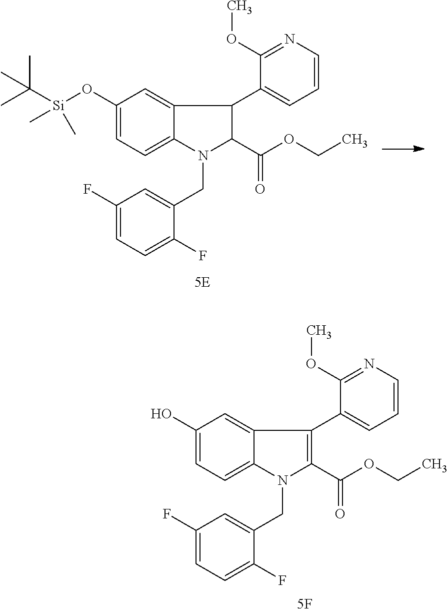

A solution of crude silylether 5E (15.237 mmol; 8.4 g) in 100 mL of THF (NOTE: 5E contains an impurity identified as the bis-N,O-difluorobenzyl compound) was ice-cooled and treated with ca 1.0 eq of TBAF (15 mL of 1.0M solution in THF). The mixture immediately turned yellow-green in color and TLC after 5 min (30% ethyl acetate in hexanes) showed no more starting material left. The mixture was diluted with ethyl acetate (500 mL) and washed with water (100 mL), aq saturated sodium bicarbonate (100 mL) and brine (100 mL). The organic layer was dried over magnesium sulfate, filtered and concentrated. The residue was purified by silica gel chromatography (Biotage 75-M column; gradient: 10 to 50% ethyl acetate in hexanes) to provide compound 5F as a white solid (5.8 g; 88% for two steps).

### Step 6:

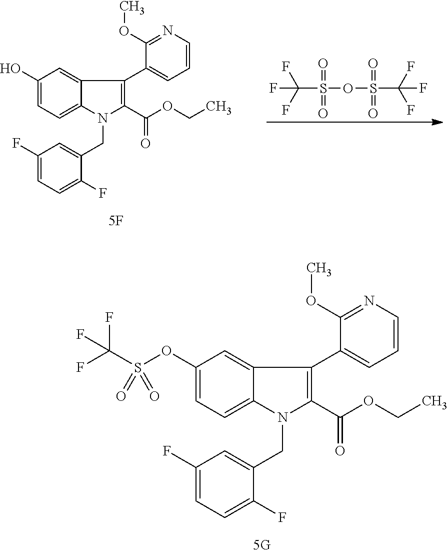

A solution of 1-(2,5-Difluoro-benzyl)-5-hydroxy-3-(2-methoxy-pyridin-3-yl)-1H-indole-2-carboxylic acid ethyl ester 5F (2.0 g; 4.56 mmol) in 20 mL of dry dichloromethane was ice cooled and treated with pyridine (4 mL) and triflic anhydride (2.1 eq, 1.61 mL, d 1.677). The mixture was stirred for 10 min and treated with a catalytic amount of 4-dimethylamino pyridine. The cooling bath was removed and the reaction was stirred for 2 h. TLC (10% ethyl acetate in hexanes) showed no more starting material left and the mixture was diluted with ethyl acetate (200 mL) and washed with water (50 mL) and brine (50 mL). The organic layer was dried over magnesium sulfate, filtered and concentrated. The residue was purified by silica gel chromatography (Biotage 40-M column; gradient: 0 to 20% ethyl acetate in hexanes) to provide compound 5G (2.50 g; 96%) as a colorless oil.

### Step 7:

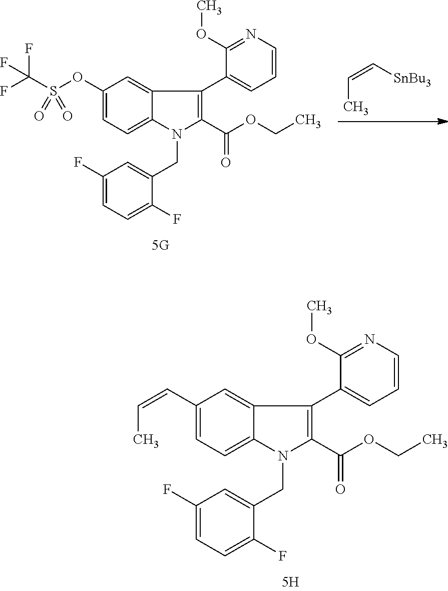

A solution of 1-(2,5-difluoro-benzyl)-3-(2-methoxy-pyridin-3-yl)-5-trifluoromethanesulfonyloxy-1H-indole-2-carboxylic acid ethyl ester 5G (650 mg; 1.13 mmol) in 10 mL of THF was treated with lithium chloride (7.0 eq, 336 mg) and (Z)-1-propenyltributyl stannane (1.5 eq, 0.51 mL, d 1.1). The mixture was degassed (vacuum/nitrogen flush) and tetrakis(triphenylphosphine)palladium was added (10 mol %, 130 mg). The reaction mixture was heated to 70° C. and stirred overnight. TLC (10% ethyl acetate in hexanes) and MS analyses showed complete conversion of starting material. The mixture was diluted with ethyl acetate (80 mL) and washed successively with water (10 mL), 10% aq ammonium hydroxide (10 mL), water (10 mL), and brine (10 mL). The organic layer was dried over magnesium sulfate, filtered and concentrated in vacuo. The residue was purified by silica gel chromatography (Biotage 25-M column; gradient: 80 mL of hexanes then 0 to 25% ethyl acetate in hexanes) to provide compound 5H (400 mg; 77%) as a colorless oil.

### Step 8:

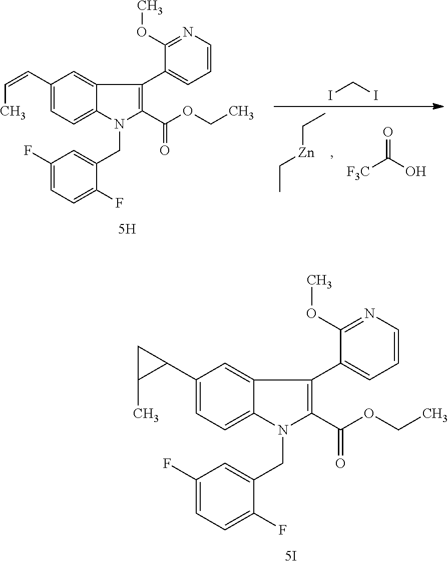

To a vigorously stirred solution of diethylzinc (10.0 eq, 3.9 mL of 1M solution in heptane) in 2 mL of dry dichloromethane at 0° C. (ice-water bath) was added dropwise a solution of trifluoroacetic acid (10.0 eq, 0.299 mL, d 1.480) in 0.5 mL of dichloromethane. The resulting mixture was stirred for 10 min after which a solution of diiodomethane (10.0 eq, 0.31 mL, d 3.325) in 0.5 mL of dichloromethane was added dropwise. The mixture was stirred for 10 min followed by addition of a solution of 1-(2,5-difluoro-benzyl)-3-(2-methoxy-pyridin-3-yl)-5-prop-Z-enyl-1H-indole-2-carboxylic acid ethyl ester 5H (180 mg; 0.389 mmol) in 1 mL of dry dichloromethane. The reaction was stirred at 0° C. and monitored by TLC and MS analyses (NOTE: Rf of starting material and product is the same in different solvent systems). After 4 h the reaction was quenched by addition of aq saturated sodium bicarbonate (10 mL). The mixture was extracted with ethyl acetate (50 mL). The organic layer was washed with aq 1M HCl (10 mL), aq saturated sodium bicarbonate (10 mL), and brine (10 mL). The organic layer was dried over magnesium sulfate, filtered and concentrated. The residue was purified by silica gel chromatography (Biotage 12-S column, gradient: 0 to 20% ethyl acetate in hexanes) to provide compound 51 as a colorless oil.

### Step 9:

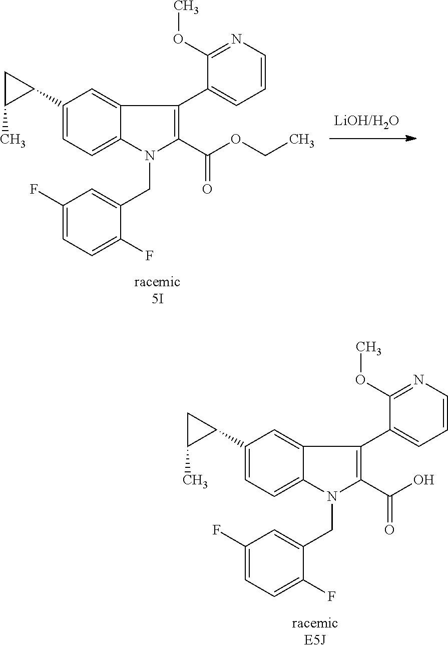

A solution of 1-(2,5-difluoro-benzyl)-3-(2-methoxy-pyridin-3-yl)-5-(2-cis-methyl-cyclopropyl)-1H-indole-2-carboxylic acid ethyl ester 5I (230 mg; 0.482 mmol) in 10 mL of a 5:1:1 THF/water/methanol mixture was treated with lithium hydroxide monohydrate (5.0 eq, 101 mg). The mixture was heated to 50° C. for 5 h. TLC (20% ethyl acetate in hexanes) showed complete consumption of the starting material. The mixture was diluted with aq 1M HCl (40 mL) and the product was taken into dichloromethane (3×25 mL). The combined organic layers were dried over magnesium sulfate, filtered and concentrated to provide compound 5J (205 mg; 95%) as a white solid.

### Example 6

### Preparation of Intermediate Compound 6H

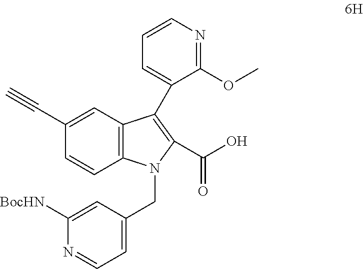

### Step 1:

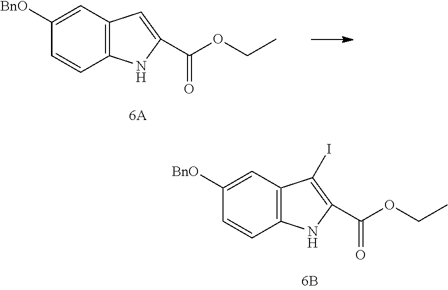

Ethyl 5-benzyloxyindole-2-carboxylate, 6A (5.0 g, 16.9 mmol) was dissolved into acetone (400 mL) at room temperature. To the mixture was added N-iodosuccinimide (4.0 g, 16.9 mmol). The resulting suspension was stirred at room temperature for 3 h. The mixture was concentrated under reduced pressure, and the residue was dissolved into ethyl acetate (300 mL). The mixture was washed with saturated aqueous sodium thiosulfate solution (100 mL). The layers were separated, and the aqueous layer was extracted with ethyl acetate (2×150 mL). The combined organic layer was dried over magnesium sulfate, filtered and concentrated under reduced pressure to give the crude product 6B (100% yield). M.S. found for C18H16INO3: 421.89 (M+H)⁺.

### Step 2:

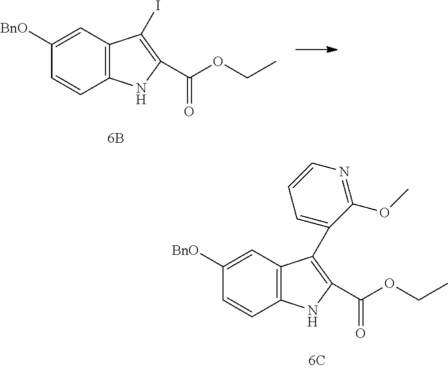

5-Benzyloxy-3-iodo-1H-indole-2-carboxylic acid ethyl ester, 6B (4.0 g, 9.48 mmol) was dissolved into 1,2-dimethoxyethane (90 mL). And PdCl₂(dppf)₂ (775 mg, 0.95 mmol) was added. The resulting mixture was de-gassed with argon bubbling for 5 min before it was heated to 90° C. and stirred for 30 min. In a second flask, the mixture of 2-methoxy-3-pyridine boronic acid (1.95 g, 11.4 mmol) and potassium carbonate (6.6 g, 47.8 mmol) in dimethoxyethane (30 mL) and water (30 mL) was de-gassed with argon bubbling for 5 min. The mixture was then transferred in three portions to the first flask. The resulting bi-phasic mixture was vigorously stirred at 90° C. for 4 h before it was cooled to room temperature. The reaction was quenched by addition of a solution of sodium sulfite (10 g) in water (400 mL) at room temperature. Ethyl acetate (500 mL) was added, and the layers were seperated. The aqueous layer was extracted with ethyl acetate (2×500 mL). The combined organic layer was dried over magnesium sulfate, filtered and concentrated under reduced pressure to give the crude product 6C (3.2 g, 84% yield). M.S. found for C24H22N2O4: 403.2 (M+H)⁺.

### Step 3:

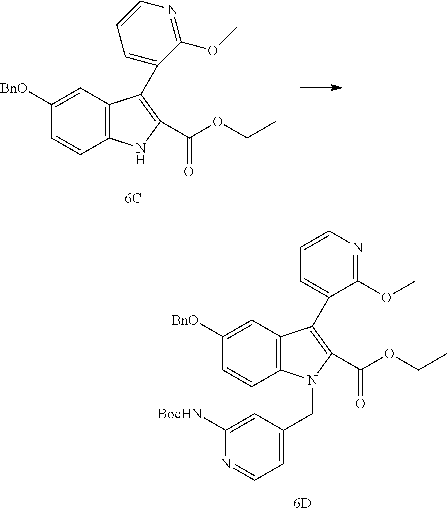

5-Benzyloxy-3-(2-methoxy-pyridin-3-yl)-1H-indole-2-carboxylic acid ethyl ester, 6C (2.0 g, 4.96 mmol) was dissolved into DMF (60 mL) at room temperature. To the mixutre were added (4-bromomethyl-pyridin-2-yl)-carbamic acid tert-butyl ester (1.4 g, 4.88 mmol) and cesium carbonate (3.6 g, 11.0 mmol). The resulting suspension was stirred at room temperature for 18 h. Ethyl acetate (200 mL) and water (150 mL) were, and the layers were seperated. The aqueous layer was extracted with ethyl acetate (2×150 mL). The combined organic layer was dried over magnesium sulfate, filtered and concentrated under reduced pressure to give the crude product 6D (1.95 g, 65% yield). M.S. found for C35H36N4O6: 609.4 (M+H)⁺.

### Step 4:

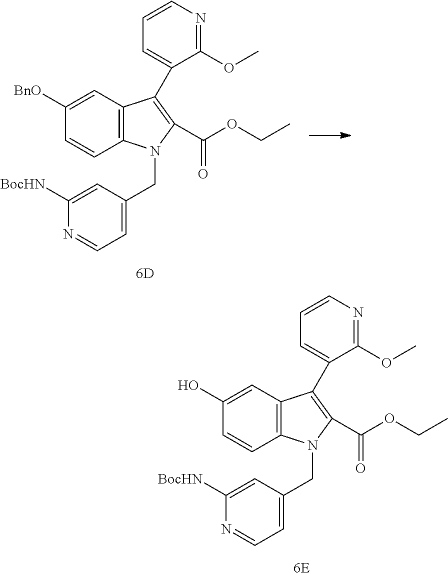

To the solution of 5-benzyloxy-1-(2-tert-butoxycarbonylamino-pyridin-4-ylmethyl)-3-(2-methoxy-pyridin-3-yl)-1H-indole-2-carboxylic acid ethyl ester, 6D (1.90 g, 3.12 mmol) in EtOH was added 10% Pd-C (1.0 g). The flask was vacuumed, and then charged with H₂ gas.

The reaction mixture was stirred at room temperature under H₂ gas for 3 h. The palladium catalyst was filtered off through a pad of celite, and was washed with 100 mL of MeOH/THF (1:1). The filtrate collected was concentrated under reduced pressure to give the crude product 6E (1.54 g, 95% yield). M.S. found for C28H30N4O6: 519.5 (M+H)⁺.

### Step 5:

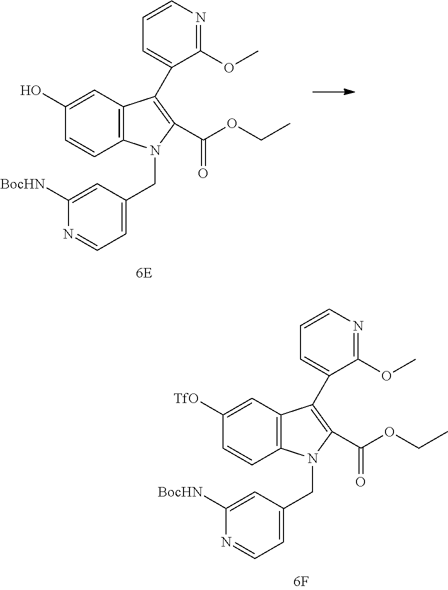

To the mixture of 1-(2-tert-butoxycarbonylamino-pyridin-4-ylmethyl)-5-hydroxy-3-(2-methoxy-pyridin-3-yl)-1H-indole-2-carboxylic acid ethyl ester, 6E (1.54 g, 2.97 mmol) and triethyl amine (1.0 mL, 7.17 mmol) in dichloromethane (50 mL) was added PhN(SO₂CF₃)₂ (1.35 g, 3.78 mmol). The resulting reaction mixture was stirred at 0° C. to room temperature for 18 h. The mixture was then diluted with dichloromethane (100 mL), and was washed with aqueous 1N sodium carbonate solution (2×50 mL). The separated aqueous solution was extracted with dichloromethane (100 mL). The combined organic layer was dried over magnesium sulfate, filtered and concentrated under reduced pressure. The crude product was purified by flash chromatography to yield the product 6F (1.55 g, 80% yield). M.S. found for C29H29F3N4O8S: 651.5 (M+H)⁺.

### Step 6:

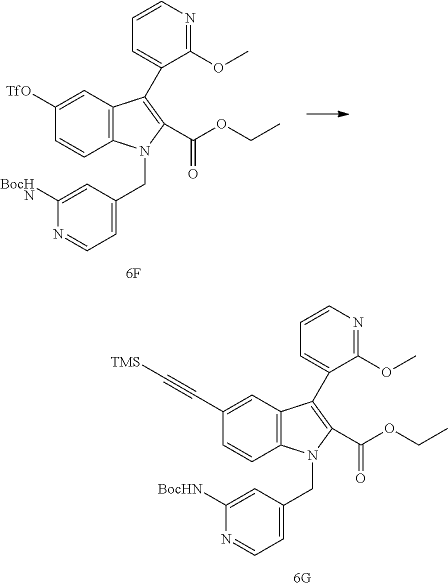

To the solution of 1-(2-tert-butoxycarbonylamino-pyridin-4-ylmethyl)-3-(2-methoxy-pyridin-3-yl)-5-trifluoromethanesulfonyloxy-1H-indole-2-carboxylic acid ethyl ester, 6F (600 mg, 0.92 mmol), TMS acetylene (0.65 mL, 4.69 mmol) and nBu₄N⁺T (409 mg, 1.11 mmol) in DMF (20 mL) were added PdCl₂(PPh₃)₂(65 mg, 0.09 mmol), CuI (53 mg, 0.28 mmol) and triethyl amine (0.65 mL, 4.66 mmol). The resulting reaction mixture was stirred in a sealed tube at 65° C. for 18 h. The mixture was cooled down to room temperature, and was diluted with water (90 mL) and EtOAc (150 mL). The layers were separated, and the aqueous layer was extracted with EtOAc (2×90 mL). The combined organic layer was washed with water (2×50 mL) before it was dried over magnesium sulfate, filtered and concentrated under reduced pressure to give the crude product 6F (514 mg, 93% yield). M.S. found for C33H38N4O5Si: 599.5 (M+H)⁺.

Step 7:

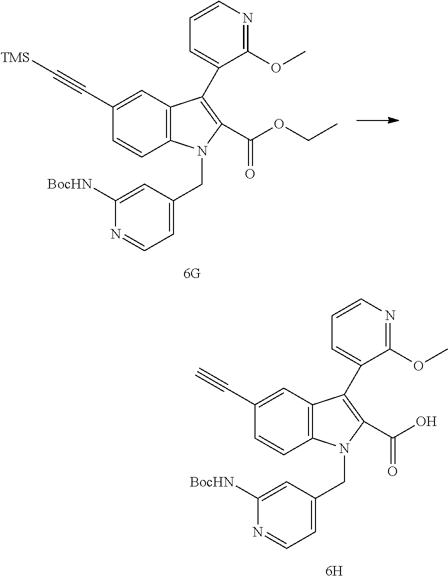

To the solution of 1-(2-tert-butoxycarbonylamino-pyridin-4-ylmethyl)-3-(2-methoxy-pyridin-3-yl)-5-trimethylsilanylethynyl-1H-indole-2-carboxylic acid ethyl ester, 6G (251 mg, 0.42 mmol) in water (3 mL) and THF (3 mL) was added aqueous 1 N lithium hydroxide solution (1.3 mL). The resulting suspension was stirred at 70° C. for 18 h. The mixture was cooled to room temperature, and the aqueous layer was acidified to pH=2 by adding aqueous 1N HCl solution. The mixture was diluted with ethyl acetate (50 mL) and water (30 mL), and the layers were separated. The aqueous layer was extracted twice with 50 mL of THF/ethyl acetate (1:1). The combined organic layer was dried over magnesium sulfate, filtered and concentrated under reduced pressure to yield the crude product 6H (191 mg, 91% yield). M.S. found for C28H26N405: 499.4 (M+H)⁺.

### Example 7

### Preparation of Intermediate Compound 7H

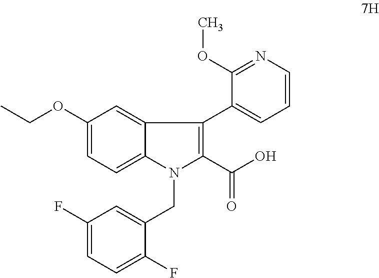

### Step 1:

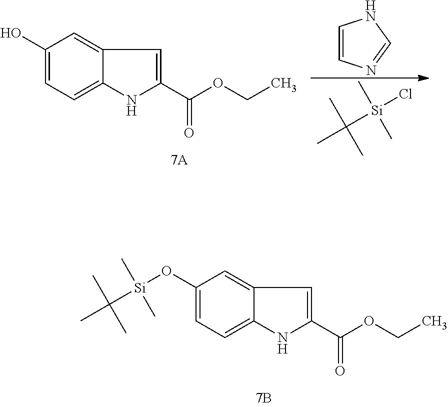

A solution of ethyl 5-hydroxy-1H-indole-2-carboxylate 7A (10.0 g; 48.73 mmol) in 300 mL of dichloromethane was treated with imidazole (4.0 eq, 13.27 g) and tert-butyldimethylsilyl chloride (2.0 eq, 14.69 g). The reaction was stirred at room temp for 3 h. A small sample (1 mL) was taken from reaction mixture, diluted with dichloromethane (10 mL) and washed with water. Evaporation of the solvent and NMR analysis showed all starting material had been consumed. The reaction mixture was diluted with dichloromethane (300 mL) and washed with water (2×200 mL) and brine (200 mL). The organic layer was dried over magnesium sulfate, filtered and concentrated to provide compound 7B (15.75 g) as a white solid.

### Step 2:

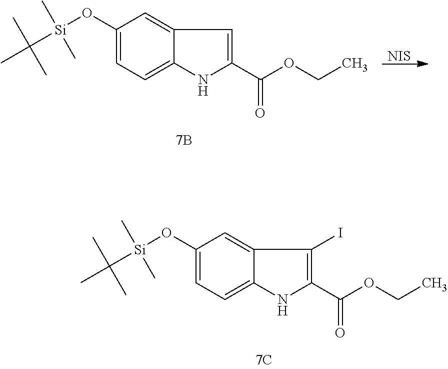

A solution of ethyl 5-tert-butyldimethylsilyloxy-1H-indole-2-carboxylate 7B (15.6 g) in 500 mL of chloroform was ice-cooled and treated with N-iodosuccinimide (1.1 eq, 12.06 g). The mixture was stirred at 0° C. for 10 min and then at room temp for 2 h. NMR analysis of a small aliquot showed complete conversion of starting material. The reaction mixture was diluted with dichloromethane (300 mL) and washed with aq saturated sodium thiosulfate (200 mL), aq saturated sodium bicarbonate (200 mL) and brine (200 mL). The organic layer was dried over magnesium sulfate, filtered and concentrated to provide compound 7C (19.47 g; 90%) as a white solid. M.S. found for C17H24INO3Si: 446.36 (M+H)⁺.

### Step 3:

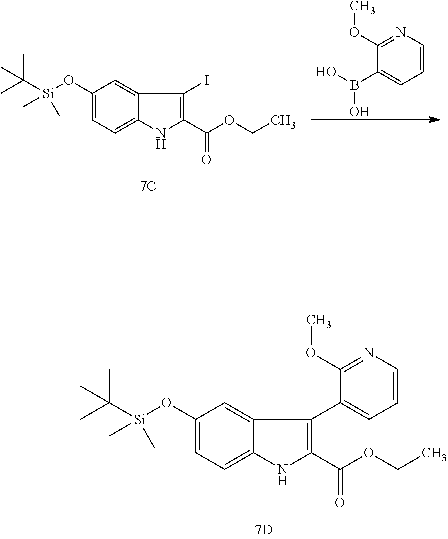

The 2-methoxy-3-pyridine boronic acid (1.05 eq, 6.99 g) was added to a solution of 7C (19.4 g; 43.55 mmol) in 500 mL of 1,2-dimethoxyethane. The mixture was degassed (vaccum/argon flush) and PdCl₂(dppf)₂ (5 mol %, 1.78 g) was added and the resulting orange solution was stirred for 30 min at room temp. A solution of potassium carbonate (4.0 eq, 174 mL of aq 1M solution) was added and the resulting brown solution was stirred at 90° C. for 2 h. The reaction mixture was cooled to room temperature and concentrated. The residue was diluted with ethyl acetate (1 L) and washed with brine (200 mL). The organic layer was dried over magnesium sulfate, filtered and concentrated. The residue was divided into two equal portions and each was purified by silica gel chromatography (Biotage 75-M column; gradient: 0 to 35% ethyl acetate in hexanes) to provide compound 7D as a white solid (14.5 g; 80%). M.S. found for C23H3ON2O4Si: 427.56 (M+H)⁺.

### Step 4:

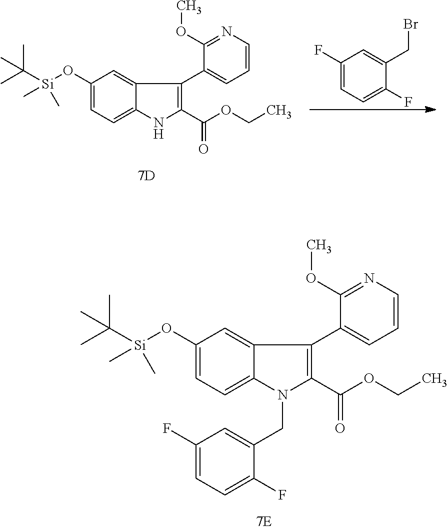

A solution of indole derivative 7D (4.0 g, 9.376 mmol) in 90 mL of dry DMF was ice-cooled and treated with 2,5-difluorobenzyl bromide (1.1 eq, 1.32 mL, d 1.613) and cesium carbonate (3.0 eq, 9.16 g). The mixture turned yellow in color and the ice-water bath was removed. A catalytic amount of tetrabutylammonium iodide (approx 20 mg) was added. The reaction mixture was stirred for 30 min where it became green in color and TLC (20% ethyl acetate in hexanes) showed no more starting materials left. The reaction was quenched by addition of water (10 mL) and the mixture was diluted with ethyl acetate (400 mL). The organic layer was washed with water (3×80 mL) and brine (80 mL), dried over magnesium sulfate, filtered and concentrated to afford the crude product 7E. The crude mixture was used for next reaction without further any further purification.

### Step 5:

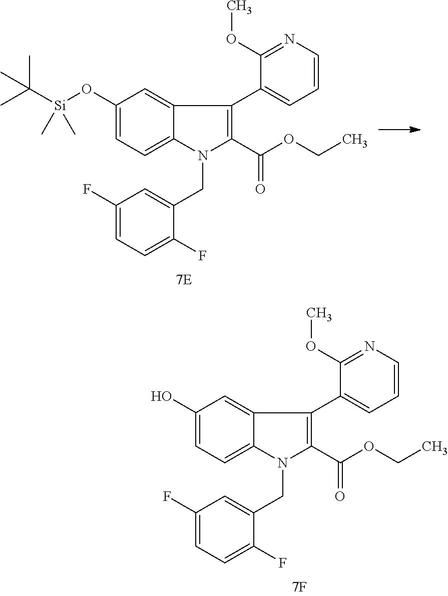

A solution of crude silylether 7E (9.376 mmol) in 100 mL of THF was ice-cooled and treated with ca 1.0 eq of TBAF (9.3 mL of 1.0M solution in THF). The mixture immediately turned yellow-green in color and TLC after 5 min (20% ethyl acetate in hexanes) showed no more starting material left. The mixture was diluted with ethyl acetate (400 mL) and washed with water (100 mL), aq saturated sodium bicarbonate (100 mL) and brine (100 mL). The organic layer was dried over magnesium sulfate, filtered and concentrated. The residue was purified by silica gel chromatography (Biotage 75-M column; gradient: 10 to 50% ethyl acetate in hexanes) to provide compound 7F as a white solid (3.81 g; 94%). ¹H NMR (400 MHz, d₆-DMSO): δ 9.12 (s, 1H), 8.18 & 8.17 (dd, J=1.46 & 5.13 Hz, 1H), 7.74 & 7.72 (dd, J=2.20 & 7.32 Hz, 1H), 7.46 (d, J=9.52 Hz, 1H), 7.31-7.25 (m, 1H), 7.16-7.07 (m, 1H), 6.87 (d, J=8.79 Hz, 1H), 6.67 (s, 1H), 6.40-6.35 (m, 1H), 5.80 (s, 2H), 3.99 (q, J=7.32 Hz, 2H), 3.75 (s, 3H), 0.845 (t, J=7.32 Hz, 3H).

### Step 6:

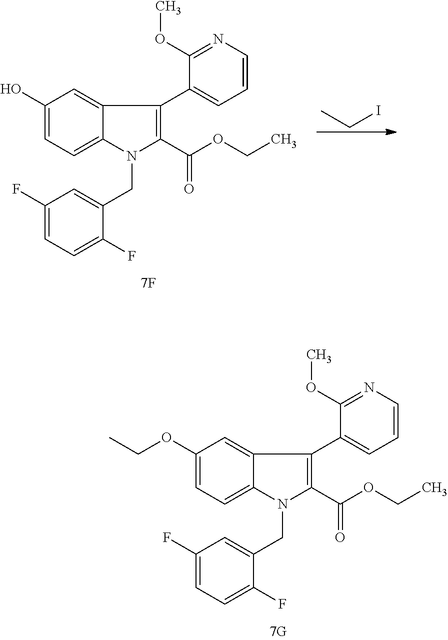

A solution of 1-(2,5-Difluoro-benzyl)-5-hydroxy-3-(2-methoxy-pyridin-3-yl)-1H-indole-2-carboxylic acid ethyl ester 7F (600 mg; 1.368 mmol) in 10 mL of dry DMF was ice cooled and treated with iodoethane (3.0 eq, 0.34 mL, d 1.950) and cesium carbonate (2.5 eq, 1.11 g). The resulting yellow solution was stirred at 50° C. for 30 min at which time TLC (20% ethyl acetate in hexanes) showed no more starting material left and the mixture was diluted with ethyl acetate (100 mL) and washed with water (3×20 mL) and brine (10 mL). The organic layer was dried over magnesium sulfate, filtered and concentrated. The residue was purified by silica gel chromatography (Biotage 25-M column; gradient: 0 to 20% ethyl acetate in hexanes) to provide compound 7G (530 mg; 87%) as a white solid. MS found for C26H24F2N2O4: 467.13 (M+H)⁺.

### Step 7:

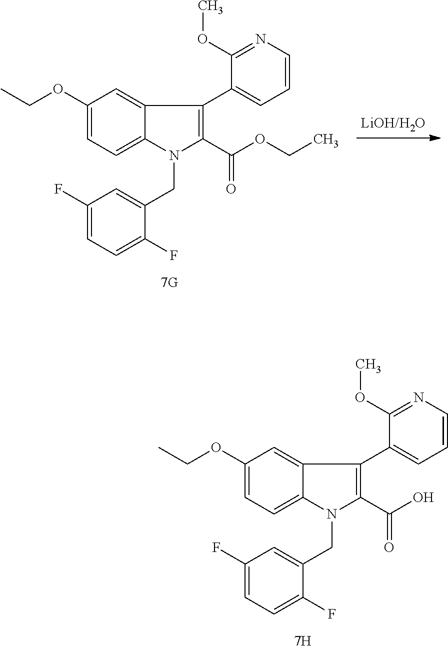

A solution of 7G (530 mg; 1.136 mmol) in 12 mL of a 4:1:1 THF/water/methanol mixture was treated with lithium hydroxide monohydrate (5.0 eq, 238 mg). The mixture was heated to 60° C. for 5 h. TLC (20% ethyl acetate in hexanes) showed complete consumption of the starting material. The mixture was diluted with aq 1M HCl (50 mL) and the product was taken into dichloromethane (3×40 mL). The combined organic layers were dried over magnesium sulfate, filtered and concentrated to provide compound 7H (0.912 mmol; 80%) as a white solid. MS found for C24H20F2N2O4: 439.02 (M+H)⁺.

### Example 8

### Preparation of Intermediate Compound 8F

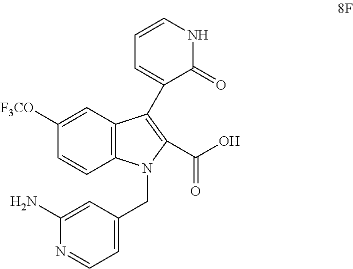

### Step 1:

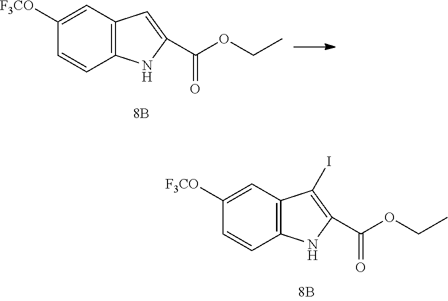

To the solution of ethyl 5-(trifluoromethoxy)-1H-indole-2-carboxylate, 8A (1.95 g, 7.14 mmol) in acetone (40 mL) was added N-iodosuccinimide (1.61 g, 7.14 mmol). The resulting suspension was stirred at room temperature for 3.75 h. The reaction was quenched with aqueous sodium thiosulfate solution (50 mL). The volatiles was evaporated under reduced pressure, and the residue was dissolved into ethyl acetate (500 mL) and water (100 mL). The mixture was washed with aqueous saturated sodium thiosulfate solution (100 mL). The layers were separated, and the aqueous layer was extracted with ethyl acetate (2×100 mL). The combined organic layer was washed with aqueous 1N sodium bicarbonate solution (100 mL) and brine (50 mL). The organic layer was then dried over magnesium sulfate, filtered and concentrated under reduced pressure to give the crude product 8B (2.8 g, 98% yield). ¹H NMR (400 MHz, CDC13) δ 9.28 (s, 1H), 7.44 (s, 1H), 7.40 (d, J=8.79 Hz, 1H), 7.24 (s, 1H), 4.48 (q, J=6.59 Hz & 7.32 Hz, 2H), 1.48 (t, J=7.32 Hz, 3H).

### Step 2:

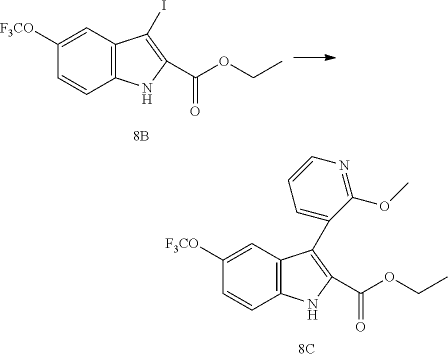

To the solution of 3-iodo-5-trifluoromethoxy-1H-indole-2-carboxylic acid ethyl ester, 8B (2.80 g, 7.02 mmol) in 1,2-dimethoxyethane (90 mL) was added PdCl₂(dppf)₂ (573 mg, 0.70 mmol). The resulting mixture was de-gassed with nitrogen bubbling for 10 min. In a second flask, the mixture of 2-methoxy-3-pyridine boronic acid (1.29 g, 8.42 mmol) and potassium carbonate (4.85 g, 35.1 mmol) in dimethoxyethane (30 mL) and water (30 mL) was de-gassed with nitrogen bubbling for 5 min. The mixture was then transferred slowly to the first flask. The resulting biphasic mixture was vigorously stirred at 90° C. for 4.25 h before it was cooled to room temperature. The reaction was quenched by the addition of a solution of sodium sulfite (5 g) in water (100 mL) at room temperature. Ethyl acetate (100 mL) was added, and the layers were separated. The aqueous layer was extracted with ethyl acetate (2×100 mL). The combined organic layer was dried over magnesium sulfate, filtered and concentrated under reduced pressure to give the crude product 8C (1.44 g, 54% yield). M.S. found for C18H15F3N2O4: 381.04 (M+H)⁺.

### Step 3:

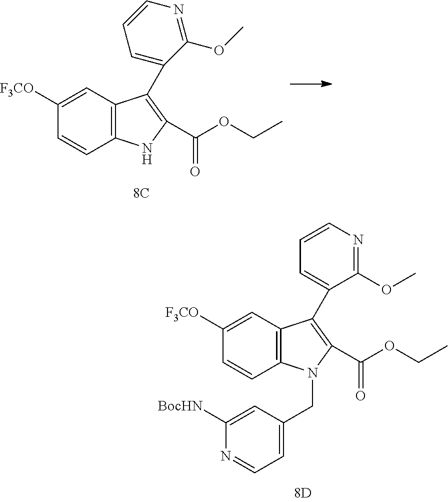

3-(2-Methoxy-pyridin-3-yl)-5-trifluoromethoxy-1H-indole-2-carboxylic acid ethyl ester, 8C (1.0 g, 2.63 mmol) was dissolved into DMF (100 mL) at room temperature. To the mixture were added (4-bromomethyl-pyridin-2-yl)-carbamic acid tert-butyl ester (0.83 g, 2.89 mmol) and cesium carbonate (1.29 g, 3.95 mmol). The resulting suspension was stirred at room temperature for 18 h. The reaction mixture was diluted with ethyl acetate (500 mL), and was washed with water (3×80 mL), aqueous saturate sodium bicarbonate (2×50 mL) and brine respectively. The separated organic layer was dried over magnesium sulfate, filtered and concentrated under reduced pressure. The crude product obtained was purified by flash chromatography to provide compound 8D (1.44 g, 93% yield). M.S. found for C29H29F3N4O6: 587.51 (M+H)⁺.

### Step 4:

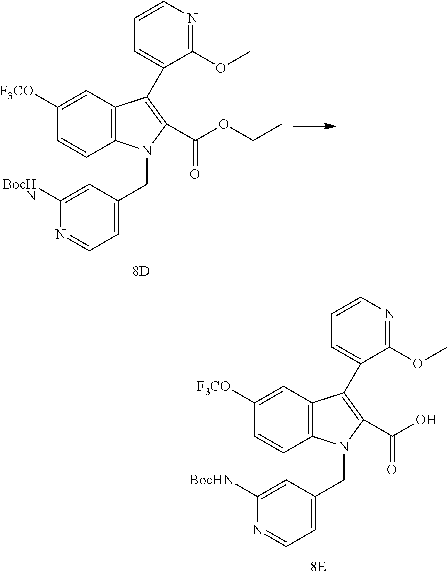

To the solution of 1-(2-tert-butoxycarbonylamino-pyridin-4-ylmethyl)-3-(2-methoxy-pyridin-3-yl)-5-trifluoromethoxy-1H-indole-2-carboxylic acid ethyl ester, 8D (1.42 g, 2.42 mmol) in THF (15 mL) and water (3 mL) was added aqueous 1N lithium hydroxide solution (12.1 mL, 12.10 mmol). The resulting suspension was refluxed at 70° C. for 22 h. The mixture was cooled down to room temperature, and the aqueous layer was acidified to pH=2 with addition of aqueous 1N HCl solution. The mixture was extracted twice with 100 mL of THF/ethyl acetate (1:1). The combined organic layer was dried over magnesium sulfate, filtered and concentrated under reduced pressure to yield the crude product 8E (100% yield). M.S. found for C27H25F3N4O6: 559.21 (M+H)⁺.

### Step 5:

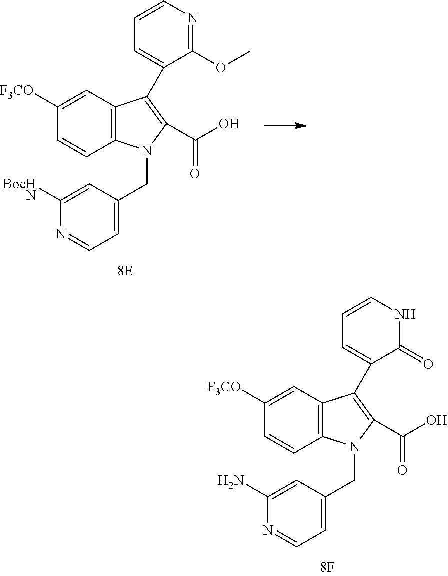

1-(2-tert-Butoxycarbonylamino-pyridin-4-ylmethyl)-3-(2-methoxy-pyridin-3-yl)-5-trifluoromethoxy-1H-indole-2-carboxylic acid, 8E (24 mg, 0.04 mmol) was dissolved into 4N HCl in 1,4-dioxane (2 mL) in a tube. Water (1 drop) was added afterwards. The reaction mixture was stirred at 90° C. in the sealed tube for 3 h. The reaction mixture was cooled down to room temperature before being concentrated under reduced pressure to provide compound 8F (100% yield). M.S. found for C21H15F3N4O4: 445.2 (M+H)⁺.

### Example 9

### Preparation of Compound 125

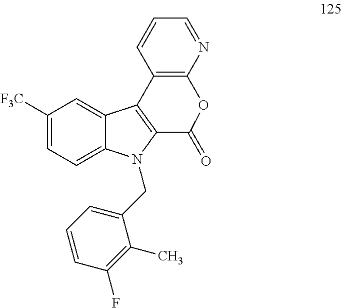

### Step 1:

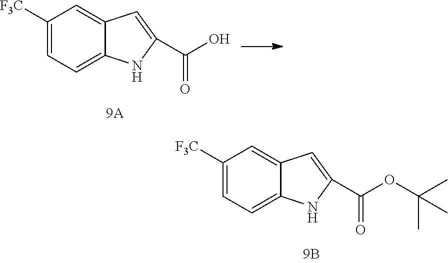

To a solution of the indole 9A (1.6 g, 6.9 mmol) in toluene (5.0 mL) was added N,N-dimethylformamide di-tert butyl acetal (5 mL), and heated to 90° C. for 12 h, cooled to room temperature, another aliquot of N,N-dimethylformamide di-tert butyl acetal (5 mL) was added and the reaction mixture was heated to 90° C. for 12 h, cooled to room temperature, diluted with ethyl acetate (10.0 mL), washed with water (2×10.0 mL), brine, dried over MgSO₄, filtered and concentrated to yield compound 9B (1.2 g, 60%) as a white solid. ‘H NMR (400 MHz, CDCl3); δ 9.17 (s, 1H), 7.97 (s, 1H), 7.51 (s, 2H), 7.21 (s, 1H), 1.63 (s, 9H).

### Step 2:

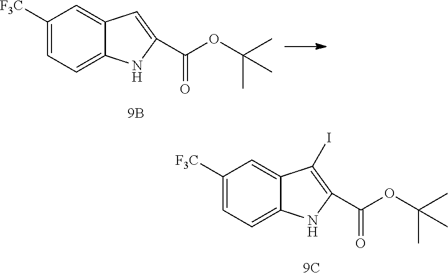

To a solution of 9B (1.2 g, 4.2 mmol) in CHCl₃ (25 mL) was added N-iodosuccinimide (946 mg, 4.2 mmol) and the reaction allowed to stir at room temperature for 12 hours. The reaction mixture concentrated in vacuo, diluted with water and extracted in EtOAc (200 mL).

The combined organic layers were dried (MgSO₄), filtered, and concentrated in vacuo. The brown residue was taken in minimum amount of CH₂Cl₂ and triturated with hexanes. Compound 9C was separated out as a brown solid which was filtered, and dried in vacuo. (1.23 g, 72% yield). ¹H NMR (400 MHz, CDC13); δ 9.34 (s, 1H), 7.87 (s, 1H), 7.57 (d, J=8.06 Hz, 1H), 7.49 (d, J=8.79 Hz, 1H), 1.68 (s, 9H).

### Step 3:

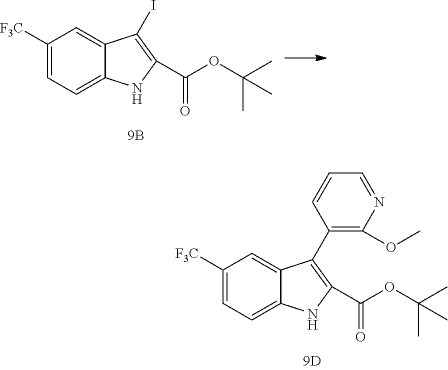

To a solution of compound 9C (1.23 g, 3.0 mmol) in DME (30 mL) under nitrogen atmosphere was added with 2-methoxy-3-pyridyl boronic acid (0.482 g, 3.15 mmol) and Pd (dppf)₂Cl₂ (245 mg, 0.3 mmol) and the resulting reaction was allowed to stir at room temperature under nitrogen for 0.5 hours. The reaction mixture was then treated with a solution of potassium carbonate (1.6 g, 12 mmol) in water (12 mL) and the resulting solution was heated to 90° C. and allowed to stir at this temperature for 1 hour. The reaction mixture was then diluted with EtOAc (200 mL) and the resulting solution was concentrated in vacuo to provide a crude residue which was purified using flash column chromatography (EtOAc/Hexanes, 0 to 30% EtOAc) to provide the product 9D as a solid (820.0 mg). M.S. found for C20H19F3N2O3: 393.2 (M+H)⁺.

### Step 4:

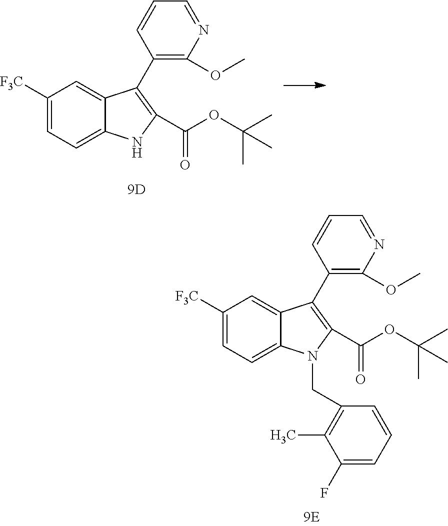

To a solution of indole 9D (10.0 g, 25.4 mmol) in DMF (100 mL) was added cesium carbonate (9.93 g, 30.5 mmol) and 3-fluoro-3-methylbenzyl bromide (3.57 mL, 30.5 mmol) and allowed to stir at room temperature for 12 hours. The reaction mixture was diluted with EtOAc (500 mL), washed with water (3×100 mL) and with brine (2×100 mL). The combined organic layers were dried (MgSO₄), filtered, and concentrated in vacuo and purified using flash column chromatography on silica gel to provide the product 9E as a colorless solid.

### Step 5:

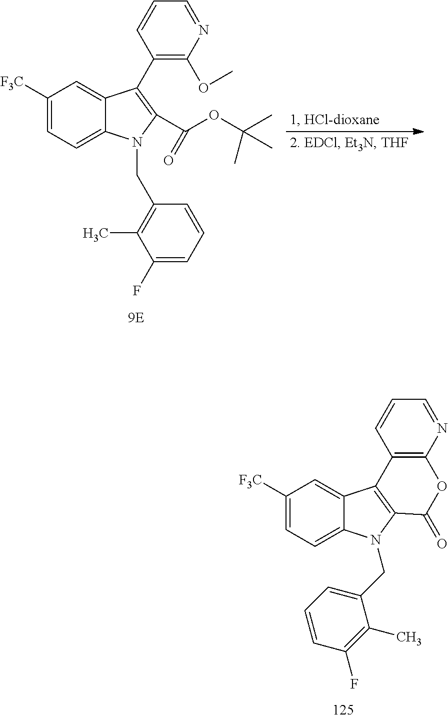

A solution of compound 9E (1.0 g, 1.94 mmol) was dissolved in 4N HCl in dioxane (20 mL) and heated at 80° C. overnight. After cooling the volatiles were removed under reduced pressure to give the crude product, which was used directly in the next step. The residue from the first step was dissolved in anhydrous THF (10.0 mL) and EDCI (3.8 mmol, 746 mg) and

Et₃N (2.55 mL, 19.0 mmol) were added to it. The reaction mixture was stirred at room temperature for 12 hours, washed with 1N HCl and extracted with CH₂Cl₂ (3×20 mL). The combined organic layer was washed with brine and dried over Mg50₄, filtered and concentrated to yield the product 125 (724 mg). M.S. found for C23H14F4N2O2: 427.2 (M+H)⁺.

### Example 10

### Preparation of Compound 44

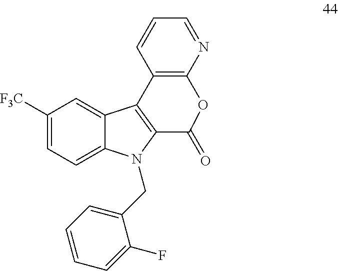

### Step 1:

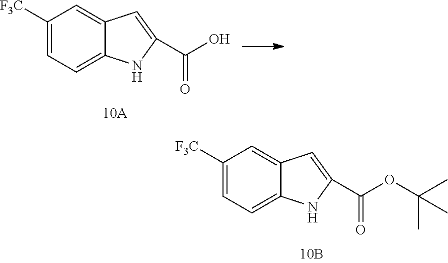

To a solution of the indole 10A (1.6 g, 6.9 mmol) in Toluene (5.0 mL) was added N,N-dimethylformamide di-tert butyl acetal (5 mL), and heated to 90° C. for 12 h, cooled to room temperature, another aliquot of N,N-dimethylformamide di-tert butyl acetal (5 mL) was added and the reaction mixture was heated to 90° C. for 12 h, cooled to room temperature, diluted with ethyl acetate (10.0 mL), washed with water (2×10.0 mL), brine, dried over MgSO₄, filtered and concentrated to yield the product 10B (1.2 g, 60%) as a white solid.

### Step 2:

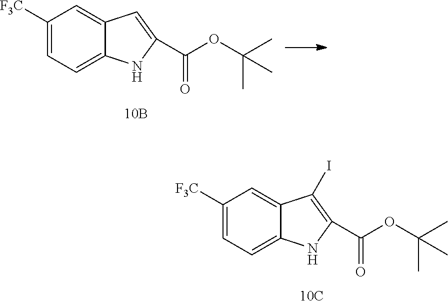

To a solution of compound 10B (1.2 g, 4.2 mmol) in CHCl₃ (25 mL) was added N-iodosuccinimide (946 mg, 4.2 mmol) and the reaction allowed to stir at room temperature for 12 hours. The reaction mixture concentrated in vacuo, diluted with water and extracted in EtOAc (200 mL). The combined organic layers were dried (MgSO₄), filtered, and concentrated in vacuo. The brown residue was taken in minimum amount of CH₂Cl₂ and triturated with hexanes. The product 10C was separated out as a brown solid which was filtered, and dried in vacuo. (1.23 g, 72% yield)

### Step 3:

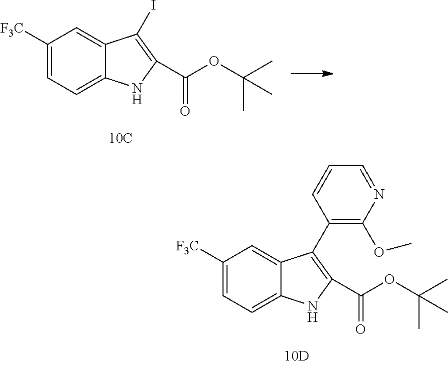

To a solution of compound 10C (1.23 g, 3.0 mmol) in DME (30 mL) under nitrogen atmosphere was added with 2-methoxy-3-pyridyl boronic acid (0.482 g, 3.15 mmol) and Pd (dppf)₂Cl₂ (245 mg, 0.3 mmol) and the resulting reaction was allowed to stir at room temperature under nitrogen for 0.5 hours. The reaction mixture was then treated with a solution of potassium carbonate (1.6 g, 12 mmol) in water (12 mL) and the resulting solution was heated to 90° C. and allowed to stir at this temperature for 1 hour. The reaction mixture was then diluted with EtOAc (200 mL) and the resulting solution was concentrated in vacuo to provide a crude residue which was purified using flash column chromatography (EtOAc/Hexanes, 0 to 30% EtOAc) to provide the product 10D as a solid (820.0 mg).

### Step 4:

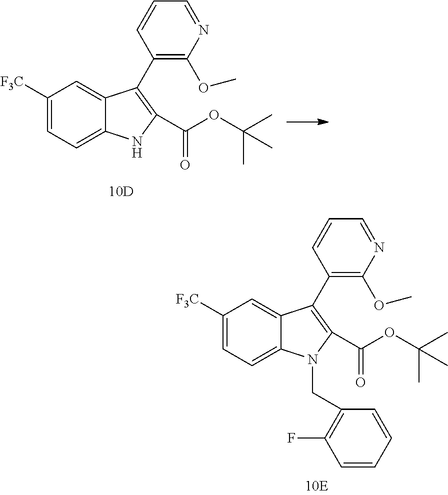

To a solution of indole 10D (10.0 g, 25.4 mmol) in DMF (100 mL) was added cesium carbonate (9.93 g, 30.5 mmol) and 2-fluorobenzyl bromide (3.57 mL, 30.5 mmol) and allowed to stir at room temperature for 12 hours. The reaction mixture was diluted with EtOAc (500 mL), washed with water (3×100 mL) and with brine (2×100 mL). The combined organic layers were dried (MgSO₄), filtered, and concentrated in vacuo and purified using flash column chromatography on silica gel to provide the product 10E as a colorless solid.

### Step 5:

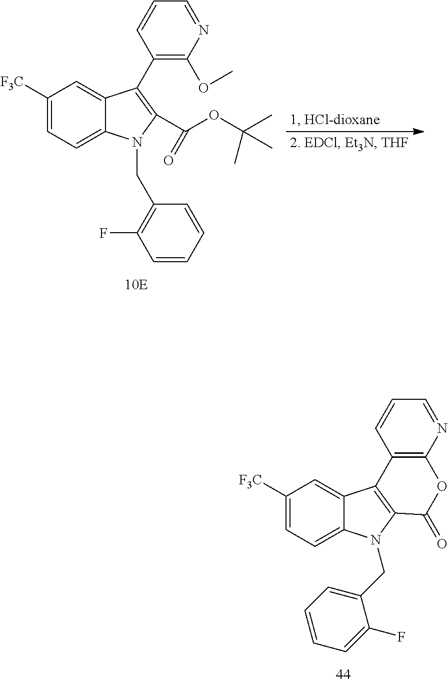

4N HCl in dioxane (20 mL) was added to the Indole 10E (1.30 g)sealed tube and heated to 80° C. (oil bath) overnight. After cooling to room temperature, the solvents were removed under reduced pressure to give a crude product which was dissolved in anhydrous THF (20 mL) and EDCI (1.15 g) followed by Et3N (4.10 mL) were added and the resulting reaction mixture was stirred overnight at room temperature. The reaction mixture was partitioned between diluted aq. HCl (-10%) and CH₂Cl₂. The organic phase was separated, extracted with CH₂Cl₂ two times. The combined organic phases were washed with water, dried (MgSO₄) and concentrated to provide compound 44 as a light brown solid (0.991 g). ¹H NMR (400 MHz, d₆-DMSO) δ 9.13 & 9.11 (dd, J=1.46 & 8.06 Hz, 1H), 8.94 (s, 1H), 8.48 &8.46 (dd, J=1.46 & 5.13 Hz, 1H), 7.99 (d, J=8.79 Hz, 1H), 7.89 (d, J=8.79 Hz, 1H), 7.57 (dd, J=4.39 & 8.06 Hz, 1H), 7.33-7.21 (m, 2H), 7.01 (t, J=7.32 Hz, 1H), 6.77 (t, J=7.32 Hz, 1H), 6.12 (s, 2H). M.S. found for C22H12F4N2O2: 412.93 (M+H)+.

### Example 11

### Preparation of Intermediate Compound 11E

### Step 1:

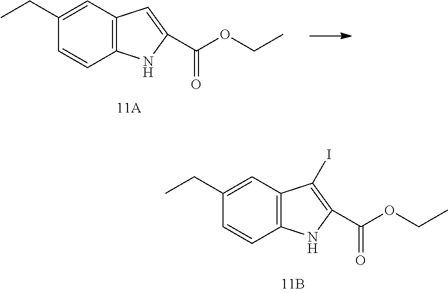

The starting materials 11A (15.0 g, 69.04 mmol) and THF (100 ml) were added to a 1000 ml round-bottomed flask. The resulting solution was cooled with a water bath. To this stirring solution, N-iodosuccinimide (15.30 g, 68.80 mmol) was added slowly. The resulting solution was allowed to stir at room temperature for 5 hours before 700 ml of water was added. The resulting mixture was continued to stir at room temperature for 30 min and then filtered. The cake was washed with water (2×40 ml), dried by air and then on house vacuum to provide compound 11B as an off-white solid (23.0 g, 97%). M.S. found for C₁₃H₁₄INO₂: 344.2 (M+H)⁺.

### Step 2:

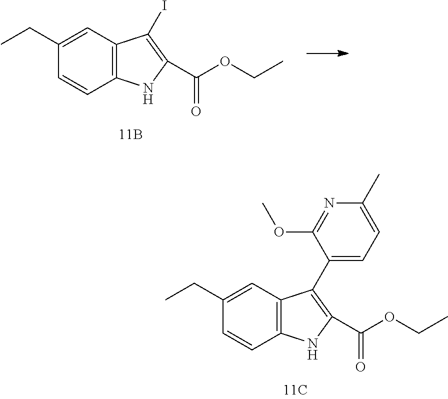

A 200 ml round-bottomed flask was charged with 11B (2.45 g, 7.14 mmol), 6-methyl-2-methoxypyridine-3-boronic acid (0.98 g, 5.87 mmol), [1,1’ bis(diphenylphosphino)ferrocene]dichloropalladium(Il) complex with dichloromethane (1:1) (0.58 g, 0.71 mmol), and DME (50 ml). To the stirring solution, a solution of sodium carbonate (10 ml of 1.5 M, 15.0 mmol) was added via a syringe. The reaction mixture was maintained reflux for 4 hours before cooled to room temperature. After concentration, the residue was taken up with ethyl acetate (200 ml), washed with water (3×100 ml), and dried over sodium sulfate. The solvent was removed by distillation under reduced pressure and the residue was purified by Combiflash chromatography on silica gel using 0-10% ethyl acetate in hexanes as the solvent to provide the product 11C as a white solid (1.51 g, 76%). M.S. found for C₂₀H₂₂N₂O₃: 339.2 (M⇄H)⁺.

### Step 3:

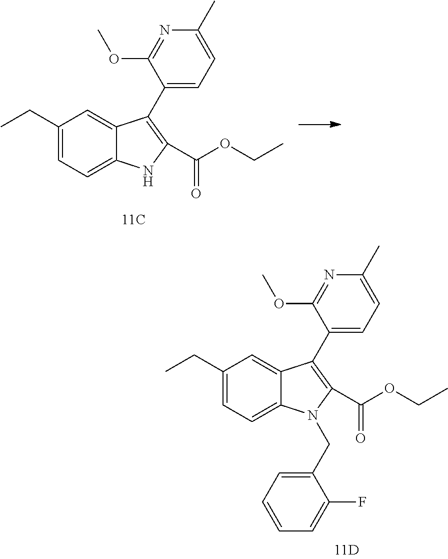

The reaction materials 11C (200 mg, 0.59 mmol), 2-fluorobenzylchloride (170 mg, 1.76 mmol), cesium carbonate (700 mg, 2.16 mmol), and DMF (3 ml) were added to a 100 ml round-bottomed flask. The resulting suspension was stirred at room temperature for 16 hours, diluted with ethyl acetate (100 ml), and washed with water (3×40 ml). The organic solution was dried over sodium sulfate and concentrated. The residue was purified by Combiflash chromatography on silica gel using 0-10% ethyl acetate in hexanes as the eluent to deliver the product 11D as a gel (205 mg, 78%).

### Step 4:

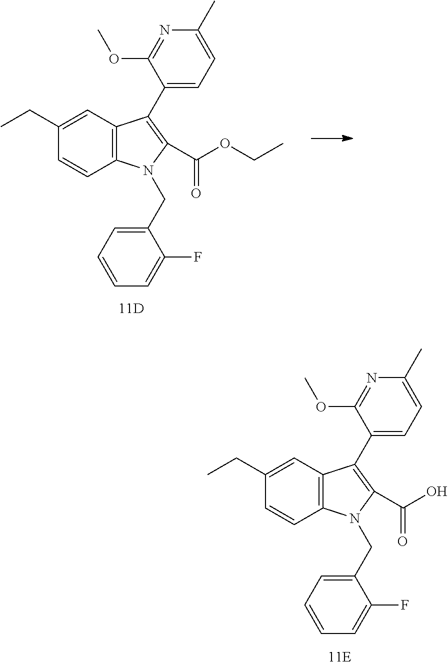

To the stirring mixture of 11D (200 mg, 0.45 mmol) in THF (5 ml) in a 100 ml round-bottomed flask was added with a solution of lithium hydroxide (2.5 ml of 1 M, 2.5 mmol). The resulting solution was maintained at reflux for 4 days before cooled to room temperature. After concentration in vacuo, the residue was dissolved in methanol (5 ml), neutralized with 1.0 M HCl aqueous solution (2.5 ml, 2.5 mmol) and then concentrated again. The residue was extracted with ethyl acetate (3×40 ml). The combined organic solutions were concentrated and dried on house vacuum to provide compound 11E as a white wax (190 mg, ˜100%). M.S. found for C₂₇H₂₅ClFN₂O₃S: 542.3 (M+H)⁺.

### Example 12

### Preparation of Intermediate Compound 12E

### Step 1:

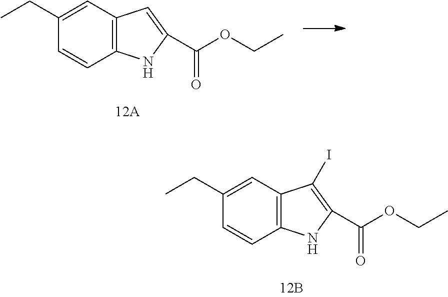

The starting materials 12A (15.0 g, 69.04 mmol) and THF (100 ml) were added to a 1000 ml round-bottomed flask. The resulting solution was cooled with a water bath. To this stirring solution, N-iodosuccinimide (15.30 g, 68.80 mmol) was added slowly. The resulting solution was allowed to stir at room temperature for 5 hours before 700 ml of water was added. The resulting mixture was continued to stir at room temperature for 30 min and then filtered. The cake was washed with water (2×40 ml), dried by air and then on house vacuum to provide compound 12B as an off-white solid (23.0 g, 97%). MS found 344.2 for C₁₃H₁₄INO₂+H⁺.

### Step 2:

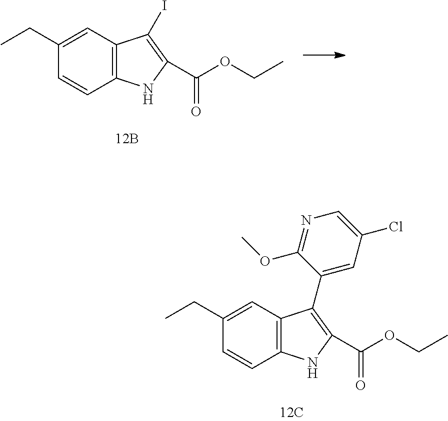

A 250 ml round-bottomed flask was charged with 12B (3.60 g, 10.49 mmol), 5-chloro-2-methoxypyridine-3-boronic acid (2.0 g, 10.67 mmol), [1,1′-bis(diphenylphosphino)ferrocene]dichloropalladium(II) complex with dichloromethane (1:1) (0.87 g, 1.06 mmol), and DME (50 ml). To the stirring solution, a solution of sodium carbonate (10 ml of 1.5 M, 15.0 mmol) was added via a syringe. The reaction mixture was maintained at reflux for 6 hours before cooled to room temperature. After concentration, the residue was taken up with ethyl acetate (200 ml), washed with water (100 ml), and dried over sodium sulfate. The solvent was removed by distillation under reduced pressure and the residue was purified by Combiflash chromatography on silica gel using 0-10% ethyl acetate in hexanes as the solvent to provide the product 12C as a white solid (2.4 g, 64%). M.S. found for C₁₉H₁₉ClN₂O₃: 359.2 (M+H)⁺.

### Step 3:

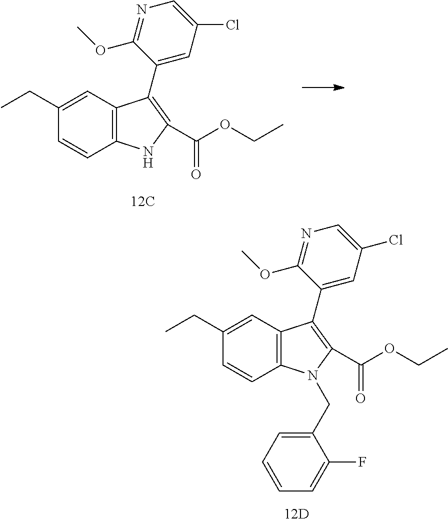

A suspension of 12C (280 mg, 0.78 mmol), 2-fluorobenzylchloride (300 mg, 2.07 mmol), cesium carbonate (400 mg, 1.23 mmol) and DMF (3 ml) was stirred at room temperature for 19 hours, diluted with ethyl acetate (100 ml), and washed with water (3×50 ml). The organic solution was dried over sodium sulfate and concentrated. The residue was purified by Combiflash chromatography on silica gel using 0-5% ethyl acetate in hexanes as the eluent to deliver the product 12D as a gel (318 mg, 87%).

### Step 4:

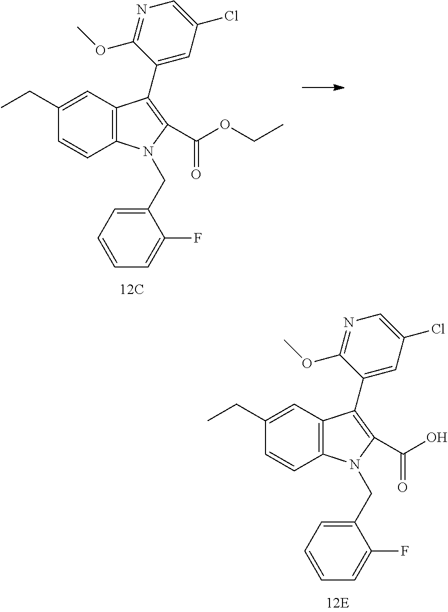

To the stirring mixture of 12D (318 mg, 0.68 mmol) in THF (10 ml) in a 100 ml round-bottomed flask was added with a solution of lithium hydroxide (2.0 ml of 1 M, 2.0 mmol). The resulting solution was maintained at reflux for 5 days before cooled to room temperature. After concentration in vacuo, the residue was dissolved in methanol (5 ml), neutralized with 1.0 M HCl aqueous solution (2.0 ml, 2.0 mmol) and then concentrated again. The residue was extracted with ethyl acetate (3×40 ml). The combined organic solutions were concentrated and dried on house vacuum to provide compound 12E as a white solid (280 mg, 94%). M.S. found for C₂₄H₂₀ClFN₂O₃: 439.2 (M+H)⁺.

### Example 13

### Preparation of Compound 126 and 127

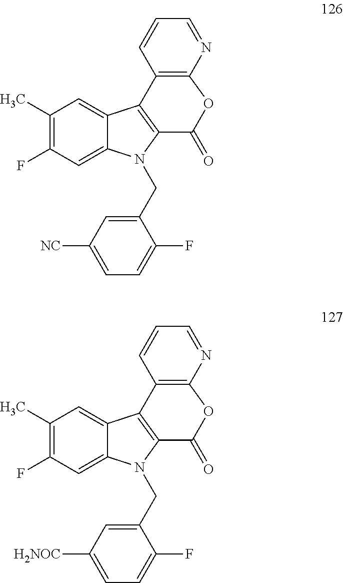

### Step 1:

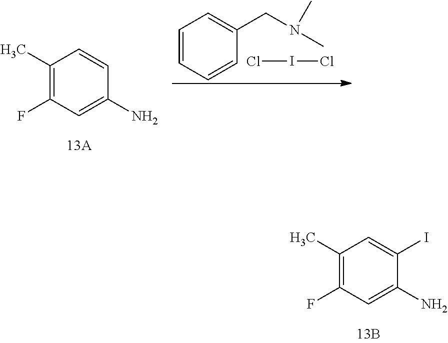

To a solution of 3-Fluoro-4-methyl-phenylamine (13A) (8.0 g, 64 mmol) in dicholoromethane (500 mL) and MeOH (100 mL) was added benzyltrimethylammonium dichloroiodate (23.8 g, 67.4 mmol) and calcium carbonate (12.8 g, 133 mmol). The suspension was stirred at room temperature for 1 h, the solids were removed by filtration and the filtrate was concentrated. The concentrated crude was redissolved in CH₂Cl₂, washed successively with 5% NaHSO₄, saturated NaHCO₃, water, brine and dried over MgSO₄. The organic layer was concentrated and the crude was purified by chromatography over SiO₂ (330 g, flash column) using 0 to 20% ethyl acetate in hexane to give a brown oil 5-fluoro-2-iodo-4-methyl-phenylamine 13B (13.4 g, 87%). ¹H NMR (400 MHz, CDCl₃): δ 2.12 (s, 3H), 4.2 (broad S, 2H), 6.51 (d, J=10.8 Hz, 1H), 7.43 (d, J=8.4 Hz, 1H).

### Step 2:

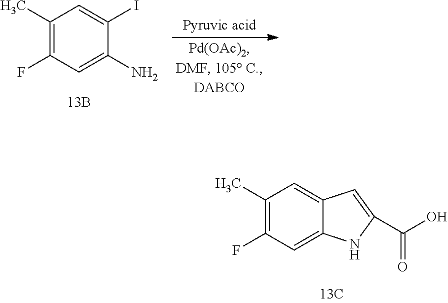

A solution of compound 13B (13.4 g, 53.5 mmol), Pd(OAc)₂ (607 mg, 2.7 mmol), pyruvic acid (14.28 g, 162.0 mmol) and DABCO (18.2 g, 162 mmol) in DMF (120 mL) was degassed and heated to 105° C. for 4 h, cooled to room temperature and partitioned between ethyl acetate and water. The aqueous layer was extracted two more times with ethyl acetate. The organic layer was washed with brine, dried over MgSO₄, concentrated and the brown solid was washed with ethyl acetate/hexanes and filtered to obtain a brownish white solid, 6-Fluoro-5-Methyl-1H-indole-2-carboxylic acid, 13C (8.3 g, 83%) which was used directly in the next step. ¹H NMR (400 MHz, d₆-DMSO): δ 2.0 (broad s, 1H), 2.25 (s, 3H), 7.0 (s, 1H), 7.15 (d, J=11 Hz, 1H), 7.49 (d, J=7.3 Hz, 1H), 11.7(s, 1H).

### Step 3:

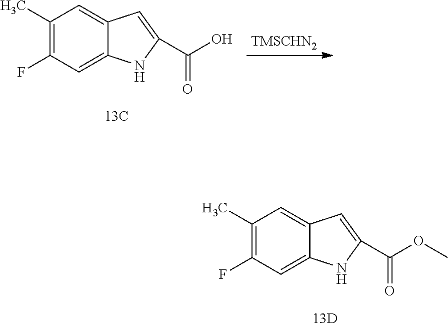

To a cooled solution of 6-Fluoro-5-Methyl-1H-indole-2-carboxylic acid in MeOH/PhMe (13C, 200 mL, 1:1) was added TMSCHN₂ (2.0 M solution in diethylether, 1.05 eq.) dropwise and the reaction was allowed to warm up to room temperature over lh. The reaction mixture was concentrated and purified by triturating with CH₂Cl₂ and hexane and collecting the solids by filtration to provide compound 13D (3.5 g). The concentrated filtrate was purified by chromatography over SiO₂ using 0 to 40% ethyl acetate in hexanes to give an additional amount of compound 13D (1.0 g). Overall yield (60%). ¹H NMR (400 MHz, d₆- DMSO): δ 2.26 (s, 3H), 3.83 (s, 3H), 7.07 (s, 1H), 7.08 (d, J=10.2 Hz, 1H), 7.4 (d, J=8.1 Hz, 1H), 11.9 (s, 1H).

### Step 4:

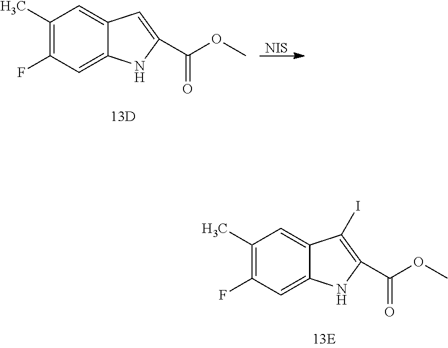

To a solution of 6-Fluoro-5-Methyl-1H-indole-2-carboxylic acid methyl ester 13D (3.53 g, 17.03 mmol) in CHCl₃/THF (100 mL, 5:1) was added N-iodosuccinimide (3.83 g, 17.03 mmol) and the reaction mixture was stirred at room temperature overnight. The reaction mixture was concentrated and redissolved in Ethyl acetate and washed with 1M Na₂S₂O₃, saturated NaHCO₃, water and brine. The organic layer was dried over MgSO₄, filtered, concentrated and the product was triturated using ethyl acetate/hexanes and filtered to provide compound 13E (5.34 g, 94.1%). ¹H NMR (400 MHz, CDCl₃): δ 2.39(s, 3H), 3.97 (s, 3H), 7.07 (d, J=9.5 Hz, 1H), 7.32 (d, J=7.3 Hz, 1H), 9.1 (s, 1H).

### Step 5:

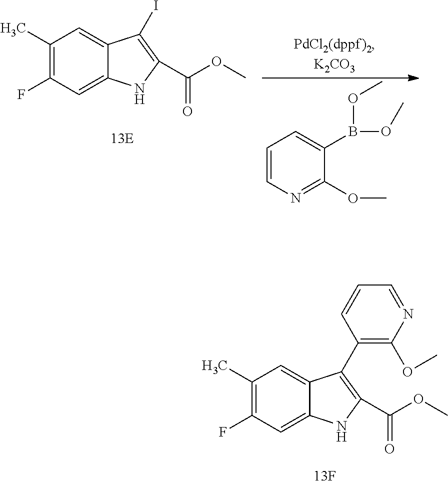

2-methoxy-3-pyridine boronic acid (2.94 g, 19.23 mmol) was added to a solution of 6-Fluoro-3-iodo-5-methyl-1H-indole-2-carboxylic acid methyl ester 13E (5.34 g, 16.03 mmol) in 1, 2 dimethoxyethane (105 mL). The mixture was degassed and PdCl₂(dppf)₂ (1.3 g, 1.60 mmol) was added to the reaction mixture. After the resulting orange solution was stirred at room temperature for 30 min., a solution of K₂CO₃ (8.86 g in 64 mL of H₂O) was added. The resulting brown solution was stirred at 90° C. for 4 h, cooled to room temperature and diluted using ethyl acetate. The organic layer was washed with water, brine and dried over MgSO₄. The concentrated filtrate was purified over SiO₂ using 0 to 30% ethyl acetate in hexanes to afford a white solid 6-Fluoro-3-(2-Methoxy-pyridin-3-yl)-5-methyl-1H-2-carboxylic acid methyl ester 13F (4.14 g, 82%). NMR (400 MHz, d₆-DMSO): δ 2.06 (s, 3H), 3.68 (s, 3H, 3.76 (s, 3H), 7.08 (m, 1H), 7.19 (m, 2H), 7.65 (d, J=10.0 Hz, 1H), 8.20 (m, 1H).

### Step 6:

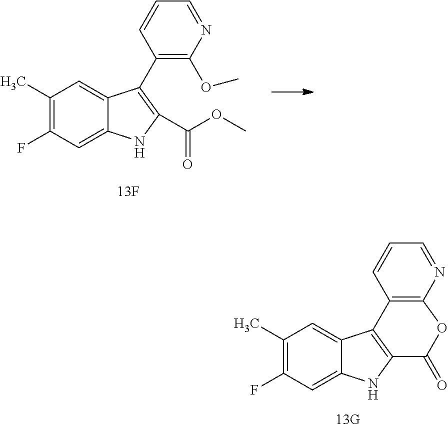

To 6-Fluoro-3-(2-Methoxy-pyridin-3-yl)-5-methyl-1H-2-carboxylic acid methyl ester 13F (4.14 g, 13.17 mmol) was added 4N HCl in dioxane (40 mL) and the reaction mixture was heated at 80° C. for 12 h, cooled, and concentrated to yield 6-Fluoro-3-(2-hydroxy-pyridin-3-yl)-5-methyl-1H-2-carboxylic acid methyl ester. To the crude from last step was added LiOH (1.65 g, 39.51 mmol) in THF/MeOH/H₂O (75 mL, 2:2:1) and the slurry was heated at 65° C. for 12 h, cooled, washed with 1 N HCl and water. The product was filtered, washed with ethyl acetate and dried in vacuo to give 6-Fluoro-3-(2-hydroxy-pyridin-3-yl)-5-methyl-1H-2-carboxylic acid (3.59 g, 95.2% over 2 steps) and used directly in the next step. To the hydroxy acid (3.59 g, 12.54 mmol) from the previous step in DMF (70.0 mL) was added EDCI hydrochloride (4.8 g, 25.08 mmol) and Et₃N (8.73 mL, 62.7 mmol) and the reaction mixture was stirred at room temperature for 12 h. The reaction mixture was diluted with ethyl acetate, the slurry was washed with water and filtered. The ethyl acetate layer was washed with 1N HCl, brine, dried over MgSO₄ and concentrated and the crude was added to the filtrate from the prior step and dried in vacuo to provide compound 13G as a white solid (3.36 g, 75%). ¹H NMR (400 MHz, d₆-DMSO): δ 2.40 (s, 3H), 7.28 (d, J=10 Hz, 1H), 7.54 (m, 1H), 8.40 (m, 2H), 8.87 (d, J=7.2 Hz, 1H).

### Step 7:

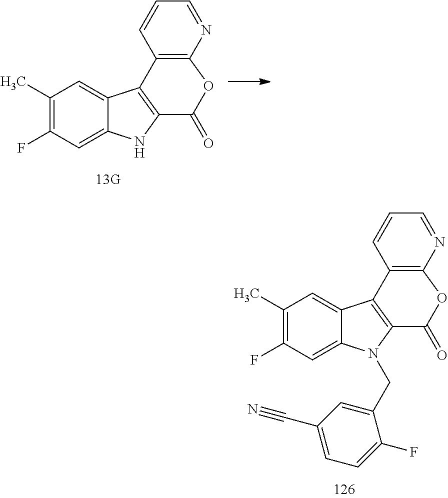

To a solution of compound 13G (167 mg, 0.622 mmol) in DMF (3.0 mL) was added 3-Bromomethyl-4-fluoro-benzonitrile (160.0 mg, 0.747 mmol) and Cs₂CO₃ (243 mg, 0.747 mmol) at room temperature and the reaction mixture was allowed to stir overnight. The reaction mixture was diluted with ethyl acetate, washed with water and brine, dried over MgSO₄, filtered and concentrated. The concentrated crude was purified by chromatography over SiO₂ using 0 to 30% ethyl acetate in hexane to provide compound 126 (200 mg, 80%). M.S. found for C23H13F2N3O2: 402.9 (M+H)⁺.

### Step 8:

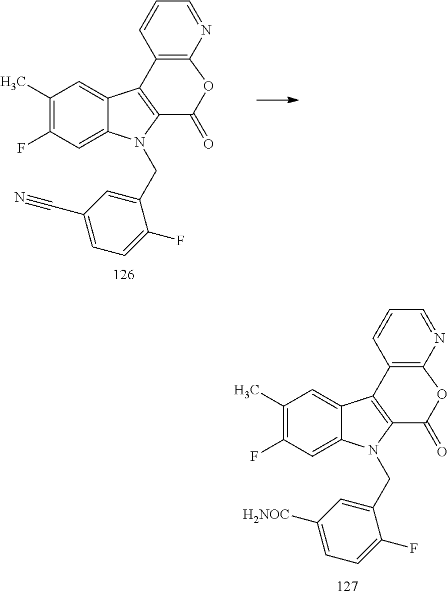

To a solution of 4-Fluoro-3-(9-fluoro-10-methyl-6-oxo-6H-5-oxa-4,7-diaza-benzo[c]fluoren-7-ylmethyl)-benzonitrile 126 (126 mg, 0.313 mmol) in acetic acid (1.0 mL) was added H₂SO₄(4 drops). The reaction mixture was heated at 100° C. for 12 h and concentrated. The solids were washed with water and ethyl acetate and dried under high vacuum to yield a white solid 9-Fluoro-10-methyl-7H-5-oxa-4,7-diaza-benzo[c]fluoren-6-one 127 (124.0 mg, 94%). M.S. found for C23H15F2N303: 420.1 (M+H)⁺.

### Example 14

### Preparation of Compound 128

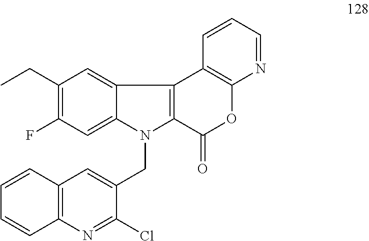

### Step 1:

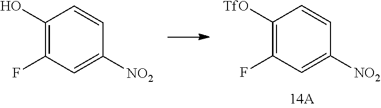

A solution of 2-fluoro-4-nitro-phenol (2.53 g; 16.1 mmol) in 60 mL of dry dichloromethane and 5 mL of dry THF was ice cooled and treated with pyridine (10 mL) and triflic anhydride (1.1 eq, 5.0 g, d 1.677). The mixture was stirred for 10 min and treated with a catalytic amount of 4-dimethylamino pyridine (tip of spatula). The cooling bath was removed and the reaction was stirred for 1 h. TLC (10% ethyl acetate in hexanes) showed no more starting material left and the mixture was diluted with ethyl acetate (300 mL) and washed with aq saturated sodium bicarbonate (80 mL) and brine (80 mL). The organic layer was dried over magnesium sulfate, filtered and concentrated in vacuo. The residue was purified on silica gel (Biotage 40-M column; gradient: 0 to 10% ethyl acetate in hexanes) to provide compound 14A (4.0 g;

### Step 2:

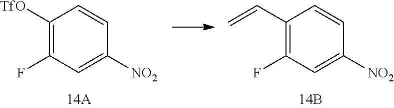

A solution of trifluoro-methanesulfonic acid 2-fluoro-4-nitro-phenyl ester (14A) (13.2 g; 45.64 mmol) in 225 mL of THF was treated with lithium chloride (7.0 eq, 13.5 g) and tributyl(vinyl)tin (2.0 eq, 26.6 mL, d 1.085). The mixture was degassed (vacuum/nitrogen flush) and tetrakis(triphenylphosphine)palladium was added (10 mol %, 5.26 g). The reaction mixture was heated to 80° C. and stirred overnight. TLC (5% ethyl acetate in hexanes) showed complete consumption of starting material. The mixture was diluted with water (100 mL) and extracted with 1:1 ether/ethyl acetate (900 mL). The organic layer was washed with 10% aqueous ammonium hydroxide (100 mL), water (100 mL) and brine (100 mL). The organic layer was dried over magnesium sulfate, filtered and concentrated in vacuo. The residue was adsorbed on silica gel and purified on a Biotage 40-S column (gradient: 0 to 4% ethyl acetate in hexanes) to provide compound 14B (7.6 g; 99%) as a slightly yellow oil which contains some stannane impurities (ca. 1.4 g)

### Step 3:

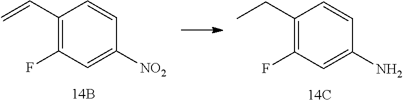

A solution of 2-fluoro-4-nitro-l-vinyl-benzene (14B) (42.65 mmol) in 140 mL of methanol was treated with a catalytic amount of 10% palladium on carbon (aprox 1.0 g). The mixture was hydrogenated at 35 psi for 2 h. TLC (10% ethyl acetate in hexanes) showed complete consumption of starting material. The mixture was diluted with dichloromethane (100 mL) and filtered thru a short path of celite. The solids were washed with dichloromethane (100 mL). The filtrate, which contains the product 14C, was used for next reaction.

### Step 4:

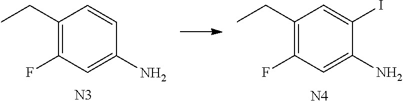

A solution of 4-ethyl-3-fluoro-phenylamine (14C) (the filtrate solution from previous step) was treated with benzyltrimethylammonium dichloroiodate (1.1 eq, 16.3 g) and calcium carbonate (2.0 eq, 8.53 g). The suspension was stirred at room temp for 1 h. TLC (10% ethyl acetate in hexanes) showed complete consumption of starting material. The solids were removed by filtration (whatman #1) and the filtrate was concentrated in rotavap. The residue was partitioned between 800 mL of 1:1 ether/ethyl acetate and aqueous 5% sodium hydrogen sulfate (200 mL). The organic layer was washed with water (200 mL) and brine (200 mL). The organic layer was dried over magnesium sulfate, filtered and concentrated in rotavap. The residue was adsorbed on silica gel and chromatographed on a Biotage 65-M column (gradient: 0 to 10% ether in hexanes) to provide compound 14D (8.5 g; 76%) as a yellow oil which contains some stannane impurities from a previous step.

### Step 5:

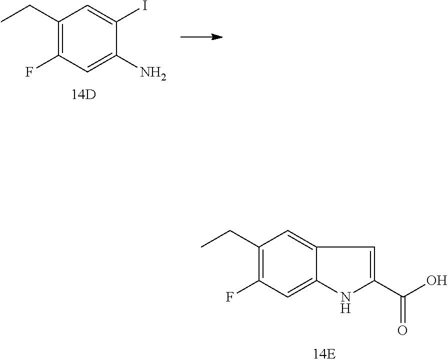

A solution of 4-ethyl-5-fluoro-2-iodo-phenylamine (14D) (7.29 g; 27.50 mmol) in 60 mL of dry DMF was treated with pyruvic acid (3.0 eq, 7.26 g, d 1.267) and DABCO (3.0 eq, 9.24 g). The mixture was degassed (vacuum/nitrogen flush) and palladium(H) acetate (0.05 eq, 308 mg) was added. The resulting solution was heated to 105° C. for 3 h. The volatiles were removed in rotavap (high vacuum pump) and the residue was partitioned between ethyl acetate (200 mL) and water (200 mL). The aqueous layer was back extracted with ethyl acetate (4×100 mL). The combined organic layers were washed with brine, dried over magnesium sulfate, filtered and concentrated in rotavap to give the crude product 14E as a dark brown oil. No further purification was carried out.

### Step 6:

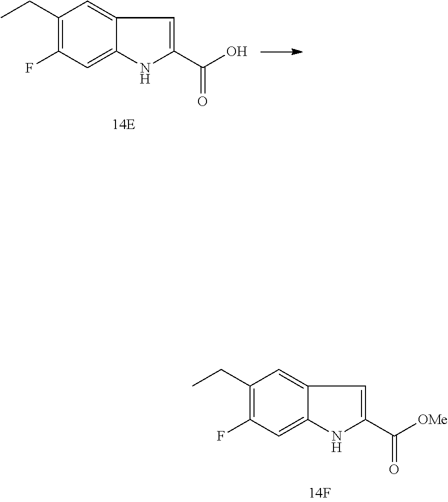

To an ice-cooled solution of 5-ethyl-6-fluoro-1H-indole-2-carboxylic acid (14E) (27.5 mmol) in 300 mL of 2:1 toluene/methanol was slowly added a solution of TMS-diazomethane in ether (2.0 eq, 27.5 mL of 2.0M). After addition was completed the cooling bath was removed and the reaction mixture was stirred for 1 h. The mixture was concentrated in rotavap to give the crude product as a brown solid. The mixture was adsorbed on silica gel and purified on a Biotage 65-M column (gradient: 10 to 50% dichloromethane in hexanes) to provide compound 14F (3.0 g; 50% for two steps) as a white solid.

### Step 7:

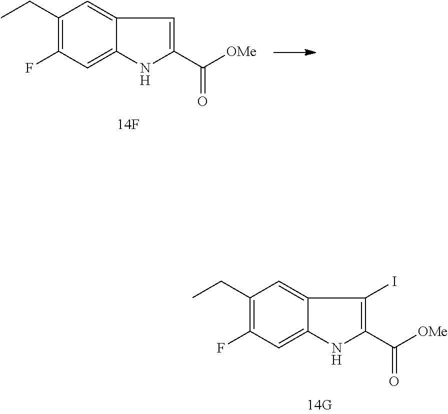

A solution of 5-ethyl-6-fluoro-1H-indole-2-carboxylic acid methyl ester (14F) (2.6 g; 11.75 mmol) in 60 mL of 1:1 THF-chloroform was ice-cooled and treated with N-iodosuccinimide (1.15 eq, 3.04 g). The cooling bath was removed and the mixture was stirred for 2 h. TLC (20% ethyl acetate in hexanes) showed almost complete consumption of starting material. The reaction mixture was diluted with ethyl acetate (300 mL) and washed with aq saturated sodium bicarbonate (2×60 mL) and brine (50 mL). The organic layer was dried over magnesium sulfate, filtered and concentrated in vacuo to give the crude product 14G (4.0 g; 99%) as a slightly yellow solid which was used without further purification.

### Step 8:

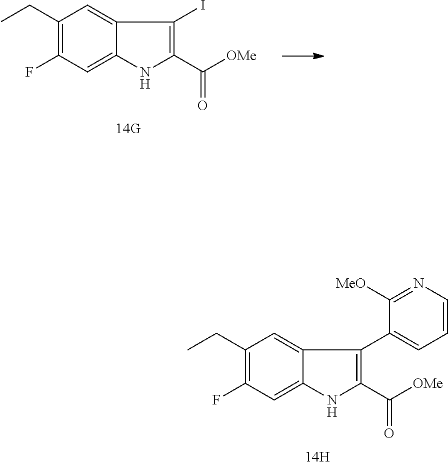

2-Methoxypyridine-3-boronic acid (1.5 eq, 2.69 g) was added to a solution of 5-ethyl-6-fluoro-3-iodo-1H-indole-2-carboxylic acid methyl ester (14G) (11.75 mmol) in 120 mL of 1,2-dimethoxyethane. The mixture was degassed (vaccum/argon flush) and palladium catalyst (10 mol %, 960 mg of PdCl₂(dppf)₂) was added and the resulting orange solution was stirred for 10 min at room temp. A solution of potassium carbonate (4.0 eq, 23.5 mL of aqueous 2M solution) was added and the resulting brown mixture was stirred at 85° C. for 2 h at which point TLC (20% ethyl acetate in hexanes) showed almost complete consumption of starting material. The reaction mixture was cooled to room temp and diluted with ethyl acetate (300 mL), washed with aq saturated sodium bicarbonate (100 mL) and brine (100 mL). The organic layer was dried over magnesium sulfate, filtered and concentrated in rotavap. The crude product was adsorbed on silica gel and purified on a Biotage 65-M column (gradient: 0 to 15% ethyl acetate in 1:1 hexanes-dichloromethane) to provide compound 14H (3.3 g; 86%) as a white solid.

### Step 9:

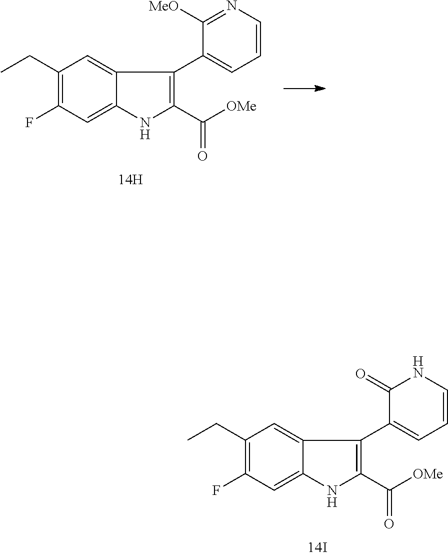

The 5-ethyl-6-fluoro-3-(2-methoxy-pyridin-3-yl)-1H-indole-2-carboxylic acid methyl ester (14H) (3.3 g; 10.05 mmol) was partially dissolved in 10 mL of methanol followed by addition of 40 mL of 4M HCl solution in dioxane. The resulting solution was heated in a sealed tube at 85° C. for 3 h. TLC (40% acetone in 1:1 dichloromethane-hexanes) showed aprox 40% conversion. All the volatiles were removed in vacuo and the residue was re-dissolved in 4M HCl solution in dioxane (40 mL). The mixture was heated in a sealed tube (90° C.) for 3 h. TLC showed some starting material left. All the volatiles were again removed in vacuo and the residue was adsorbed on silica gel. Purification on a Biotage 40-M column (gradient: 20 to 60% acetone in 1:1 dichloromethane-hexanes) to provide compound 141 (2.0 g; 63%) as a slightly yellow solid and recovered starting material 14H (700 mg, 20%).

### Step 10:

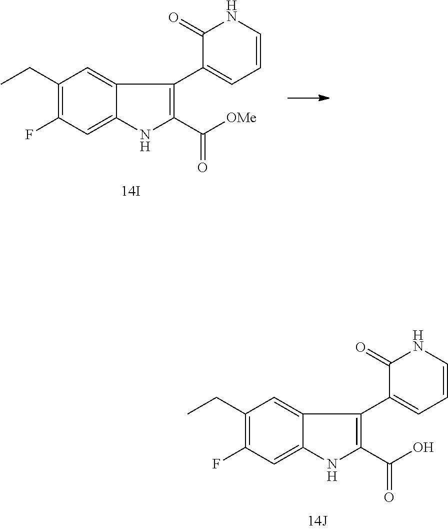

A solution of 5-ethyl-6-fluoro-3-(2-oxo-1,2-dihydro-pyridin-3-yl)-1H-indole-2-carboxylic acid methyl ester (14I) (1.9 g; 6.04 mmol) in 100 mL of 6:1:1 THF/water/methanol was treated with lithium hydroxide monohydrate (2.5 eq, 634 mg). The reaction mixture was stirred at 50° C. and monitored by TLC (50% acetone in 1:1 dichloromethane-hexanes). All the starting material had been consumed after 3 h (the product precipitated in the reaction mixture). The mixture was treated with aqueous 1M HCl (100 mL) and the product 14J (1.80 g; 99%) was recovered by filtration (whatman #1) as a white solid.

### Step 11:

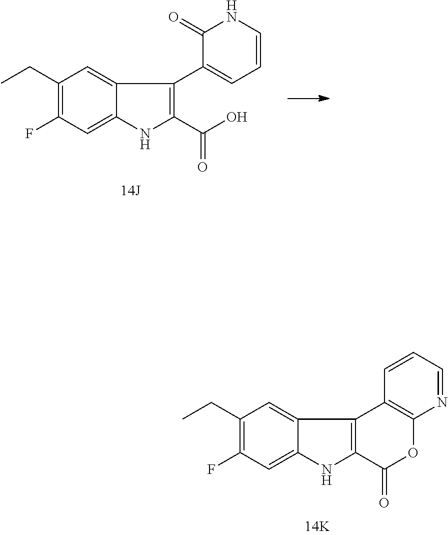

The 5-ethyl-6-fluoro-3-(2-oxo-1,2-dihydro-pyridin-3-yl)-1H-indole-2-carboxylic acid (14J) (500 mg; 1.665 mmol) was suspended in dry DMF (40 mL) and treated with EDCI (2.0 eq, 638 mg) and triethylamine (10.0 eq, 2.33 mL, d 0.72). The mixture was stirred overnight at room temperature. The mixture was concentrated to dryness in vacuo (high vacuum pump).

The residue was treated with methanol (10 mL) to make a homogeneus suspension. The product was recovered by filtration (whatman #1) and washed with methanol (2×5 mL). The product 14K (282 mg; 60%) was thus obtained as a white solid.

### Step 12:

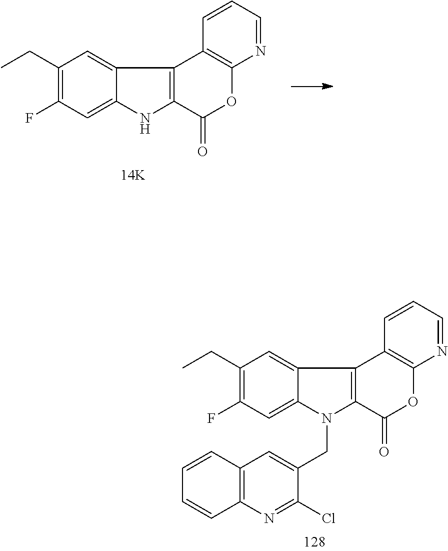

Compound 14K (40 mg, 0.141 mmol) was suspended in 2 mL of dry DMF and treated with 2-chloro-3-chloromethyl-quinoline (1.2 eq, 36 mg) and cesium carbonate (2.0 eq, 92 mg). A catalytic amount of tetrabutylammonium iodide (tip of spatula) was added and the mixture was stirred at room temp. TLC (30% ethyl acetate in hexanes) showed complete consumption of starting material after 1 h. The mixture was diluted with 50 mL of 4:1 dichloromethane-THF and washed with water (10 mL). The organic layer was concentrated in vacuo to provide compound 128 (65 mg, 99%) which was used without further purification.

### Example 15

### Preparation of Compound 129

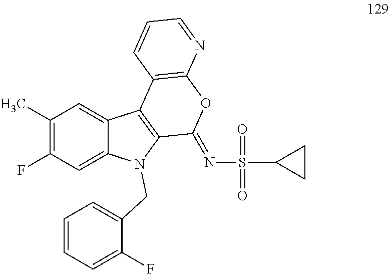

### Step 1:

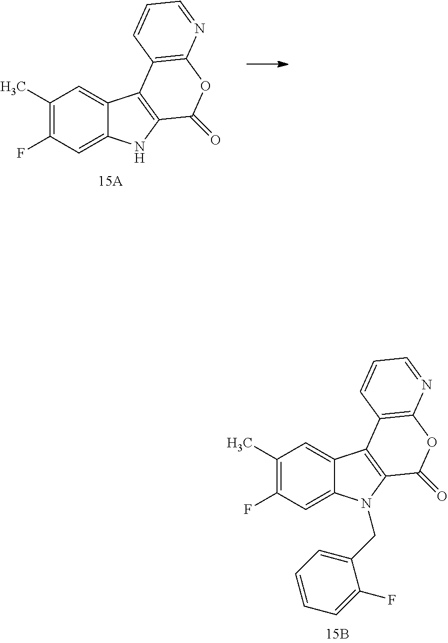

To a solution of 9-fluoro-10-methyl-7H-5-oxa-4,7-diaza-benzo[c]fluoren-6-one 15A (167 mg, 0.622 mmol) in DMF (5.0 mL) was added 3-bromomethyl-4-fluorobenzene (Acros, 1.76 mg, 0.9 mmol) and Cs₂CO₃ (1000 mg, 3 mmol) at room temperature and the reaction mixture was allowed to stir overnight. The reaction mixture was diluted with ethyl acetate, washed with water and brine, dried over MgSO₄, filtered and concentrated. The concentrated crude was purified by chromatography over SiO₂ using 0 to 30% ethyl acetate in hexane provide compound 15B (200 mg, 87%). M.S. found for C22H13F2N2O2: 377 (M+H)⁺.

### Step 2:

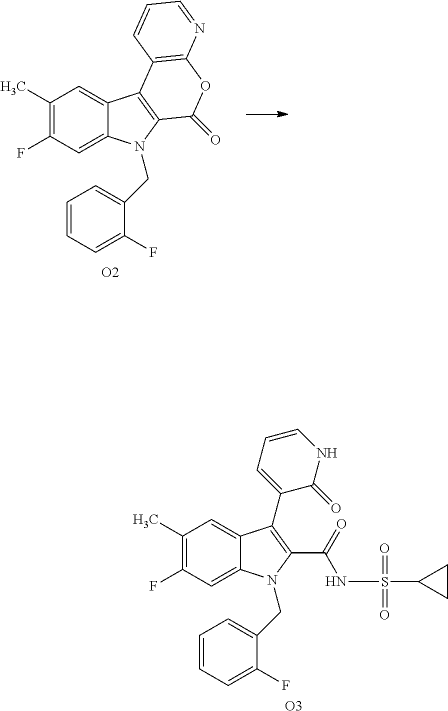

A slurry of compound 15B (34 mg, 0.09 mmol) and cyclopropyl sulfonamide (20.0 mg, 0.165 mmol) in anhydrous DMF (3.0 mL) was treated with NaH (16.0 mg, 0.4 mmol, 60% suspension in mineral oil). The reaction mixture was heated overnight at 40° C. The pH of the cooled reaction mixture was adjusted to pH of 3 with 1N HCl and extracted with ethyl acetate. The ethyl acetate layer was washed with water, brine and filtered through Na₂SO₄. The filtrate was concentrated to provide compound 15C (22 mg, 50% yield) as white solid. MS (CI) M+1=498

### Step 3:

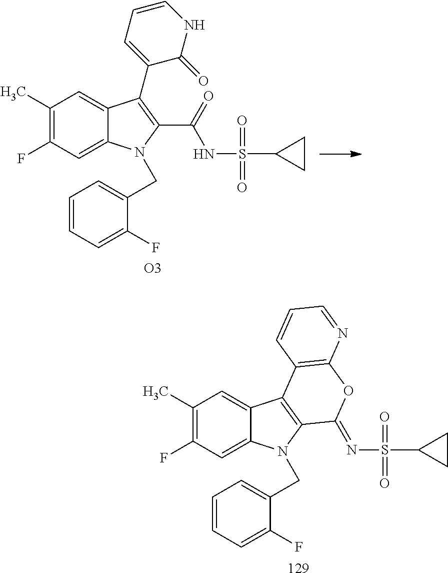

A round bottomed flask was charged with compound 15C (130 mg, 0.26 mmol) and was POCl₃ (neat, 4 mL) was added. The reaction was heated at reflux for 2 h under nitrogen and the disappearance to starting material was confirmed by TLC (Ethyl acetate/ Hexane) . The reaction was cooled and the excess POCl₃ removed in vacuo. The residue obtained was purified using flash chromatography (ethyl acetate/Hexane) to provide compound 129 (3 mg). MS: 480.3 (M+H)⁺; ¹H NMR (500 MHz, CDCl₃): δ 0.99 (m, 2H), 1.30 (m, 2H), 2.50 (s, 3H), 2.85 (m, 1H), 6.07(s, 2H), 6.87 (t, J=7.5 Hz, 1H), 7.00 (t, J=7.5 Hz, 1H), 7.11 (t, J=9.5 Hz, 1H), 7.22 (d, J=10.0 Hz, 1H), 7.22 (d, J=10.0 Hz, 1H), 7.51 (dd, J=4.7 Hz,1H), 8.08 (d, J=7.25 Hz, 1H), 8.49 (dd, J=1.5 Hz, 1H), 8.63 (dd, J=1.8 Hz, 1H).

### Example 16

### Preparation of Intermediate Compound 16L

Step A—Synthesis of Compound 16B

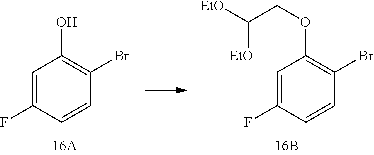

A solution of compound 16A, (228.00 g, 1.19 mmol), Potassium carbonate (247.47 g, 1.79 mol) in DMF (3.00 L) was treated with 2-Bromo-1,1-diethoxyethane (197.54 mL, 1.31 mol) and heated at 135° C. for 7 hours. The reaction mixture was concentrated in vacuo and extracted with EtOAc (3×2 L). The combined organic layers were washed with aqueous NaOH (2M, 4 L). The organic layer was dried (MgSO₄), filtered, concentrated in vacuo to provide compound 16B (362.00 g, 98%) which was used without further purification.

### Step B—Synthesis of Compound 16C

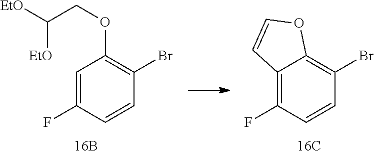

A solution of compound 16B (352.00 g, 1.15 mol) in toluene (2500 mL, 2.3 mol) was treated with polyphosphoric acid (370.00 g, 3.4 mol) and heated at reflux for 5 hours. The reaction mixture was concentrated in vacuo diluted with water (3 L) and the extracted with EtOAc (4 L). The organic layer was washed with aqueous NaOH (2 L), filtered, concentrated in vacuo and purified by distillation at reduced pressure to provide compound 16C (125.00 g, 50.8%). Bp. 80° C. (1 mm/Hg) as a colorless liquid which solidified at room temperature. ¹H NMR (400 MHz,CDCl₃) δ 7.67 (d, 1 H, J=2.2 Hz), 7.39 (dd, 1 H J=5.1 & 3.7 Hz), 6.94 (d, 1H, J=2.2 Hz), 6.86 (t, 1 H, J=8.8 Hz).

### Step C—Synthesis of Compound 16D

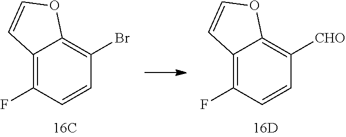

A solution of compound 16C (124.12 g, 577.25 mmol) in ether (2.0 L) was cooled to −78° C. and treated dropwise with a solution of 2.5 M of n-butyllithium in hexane (235.5 mL) and allowed to stir at −78° C. for 15 minutes. To this reaction mixture was added DMF (89.393 mL, 1.15 mol) and allowed to stir at −78° C. for 30 minutes. The reaction mixture was quenched with methanol (23.383 mL, 577.25 mmol) and warmed to room temperature. The reaction mixture was diluted with ether (300 mL) and the organic layer was washed with water (300 mL). The separated organic layer was dried (MgSO₄) filtered, concentrated in vacuo to provide compound 16D (89.00 g, 93.9%).

### Step D—Synthesis of Compound 16E

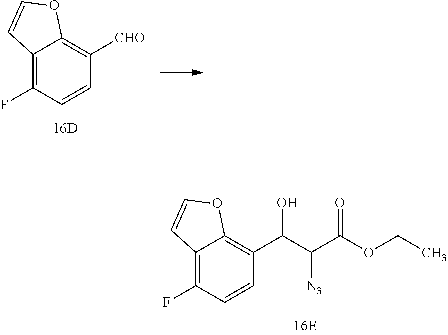

A solution of compound 16D (12.71 g, 77.45 mmol), lithium chloride (6.567 g, 154.9 mmol) and ethyl azidoacetate (20.00 g, 154.9 mmol; added as a 30% solution in CH₂Cl₂), diazabicyclo[5.4.0]undec-7-ene (23.16 mL, 154.9 mmol) and stirred for 2 hours. The completion of the reaction was followed by TLC (EtOAc/Hexanes 1:4). Upon completion, the reaction mixture was diluted with ethyl acetate (1 L) and washed with water and aqueous HCl (400 mL). The combined organic layers were dried (MgSO₄), filtered and concentrated in vacuo and the residue obtained was purified using flash column chromatography SiO₂ (EtOAc/Hexanes) to provide compound 16E (18.3 g, 80.6%) as a colorless oil.

### Step E—Synthesis of Compound 16F

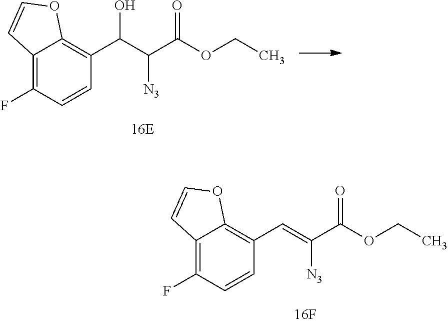

A solution of compound 16E (15.7 g, 53.5 mmol) and methanesulfonyl chloride (8.29 mL, 107 mmol) in methylene chloride (87.7 mL, 1.37 mmol) at −30° C. was treated dropwise with a solution of triethylamine (52.2 mL, 375.0 mmol) in methylene chloride (100 mL). The reaction mixture was allowed to stir at −30° C. for 3 hours, diluted with aqueous saturated sodium bicarbonate and methylene chloride (400 mL). The organic layer was separated and washed with water, aqueous HCl and brine. The organic layer was dried (MgSO₄), filtered, concentrated in vacuo, and purified using flash column chromatography (SiO₂, 10% EtOAc in (1:1) Hexanes/CH₂Cl₂) to provide compound 16F (12.6 g, 85.5%).

### Step F—Synthesis of Compound 16G

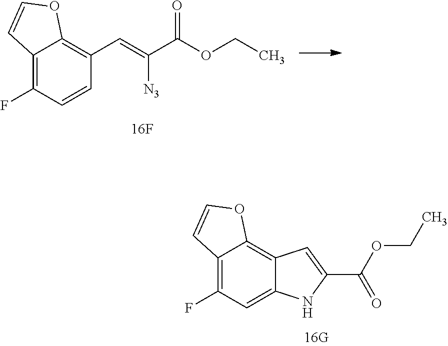

150 mL of xylenes was heated at 165° C. To this boiling solution was added dropwise a solution of compound 16F (11.2 g, 40.7 mmol) in Xylenes (70 mL, 189.4 mmol). The reaction mixture was stirred for additional 20.0 minutes and allowed to cool to room temperature to provide compound 16G as a precipitate (7.00 g, 69.6%), which was filtered, washed with hexanes and dried under vacuum.

### Step G—Synthesis of Compound 16H

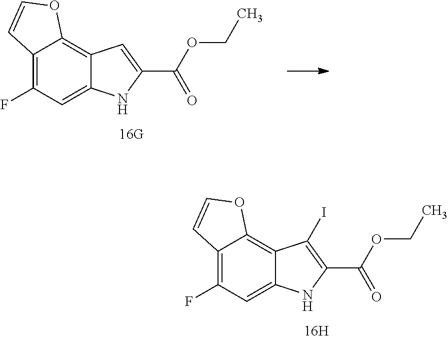

To a solution of compound 16G (15.88 g, 64.23 mmol) in DMF (100 mL) was added N-iodosuccinimide (15.90 g, 70.66 mmol) and allowed to stir at room temperature. for 2 hours.

The reaction mixture was diluted with water (1000 mL) and extracted in EtOAc (1000 mL). The organic layer was washed with water (1000 mL), aqueous sodium thiosulfate (5% aqueous soln. 1 L) and dried (MgSO₄). The organic layer was dried (MgSO₄), filtered, concentrated in vacuo to provide compound 16H (22.30 g, 93.04%) as a solid.

### Step H—Synthesis of Compound 16I

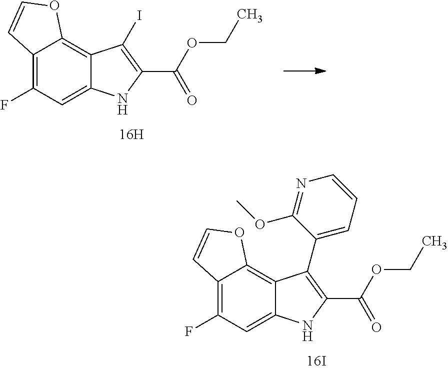

A solution of compound 16H (22.000 g, 58.962 mmol), 2-methoxypyridin-3-ylboronic acid (13.527 g, 88.444 mmol), (PPh₃)₂PdCl₂ (4.13 g, 5.88 mmol) in 1,2-dimethoxyethane (250.0 mL) was degassed for 2 min and allowed to stir at room temperature. for 15 minutes. The orange reaction mixture was treated with a solution of potassium carbonate (30.53 g, 220.9 mmol) in water (250.0 mL) and allowed to stir at 90 ° C. for 3 hours. The yellow reaction turned orange dark with the disappearance of starting material (TLC). The reaction mixture was diluted with EtOAc (1000 mL) and washed with aqueous NaOH (500 mL, 1M), dried (MgSO₄), filtered, concentrated in vacuo, and purified using flash column chromatography SiO₂ (THF/Hexanes 0→60%) to provide compound 16I (16.65 g, 79.7%) as pale brown solid.

### Step I—Synthesis of Compound 16J

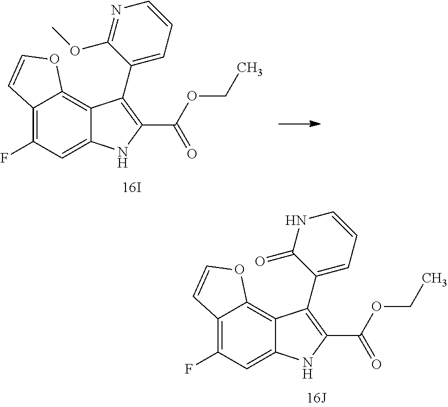

A solution of compound 16I (4.50 g, 12.7 mmol) in methanol (10 mL, 246.9 mmol) was treated with a solution of 4 M HCl in dioxane (100 mL) and heated at 90° C. for 3 hours in a pressure tube. The reaction mixture was concentrated in vacuo and the residue obtained was purified using flash column chromatography (SiO₂, THF/Hexanes 0→100%) to provide compound 16J as a colorless solid.

### Step J—Synthesis of Compound 16K

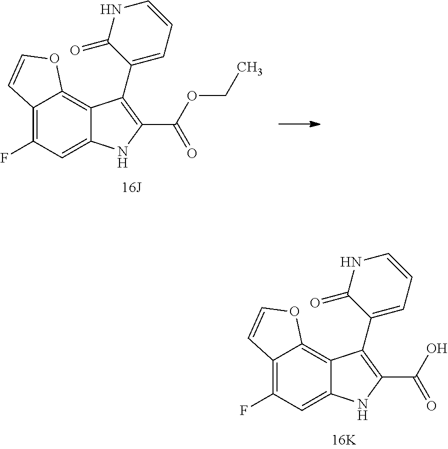

A solution of compound 16J (810.00 mg, 2.38 mmol) in water (25 mL), THF (25 mL) and methanol (25 mL, 780.2 mmol) was treated with lithium hydroxide monohydrate (499.41 mg, 11.901 mmol) and heated at 80° C. for 1 hour. The reaction mixture was then acidified using 1N HCl, filtered and dried in vacuo to provide compound 16K (627.00 mg, 84.4%) as colorless solid.

### Step K—Synthesis of Compound 16L

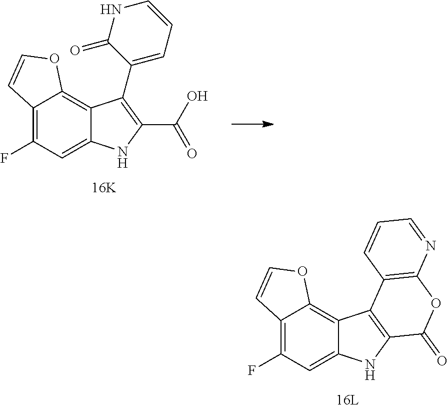

To a suspension of compound 3K (8.00 g, 25.6 mmol) and N-(3-dimethylaminopropyl)-N′-ethylcarbodiimide hydrochloride (9.82 g, 51.2 mmol) in DMF (153.85 mL) was added triethylamine (35.71 mL, 256.2 mmol) and the reaction was stirred overnight at room temperature. The reaction mixture was concentrated in vacuo and the resulting residue was diluted with methanol (100 mL). The resulting precipitate was filtered and dried to provide compound 3L (5.90 g, 78.3%)

### Example 17

### Preparation of Intermediate Compound 17D

### Step A—Synthesis of Compound 17A

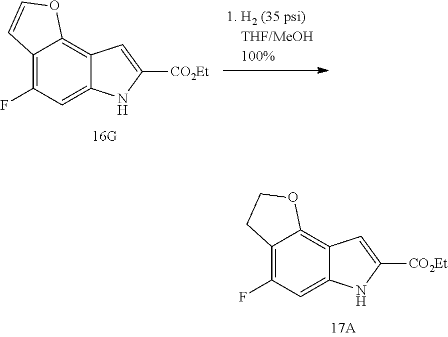

A solution of 16G (5.0 g; 20.22 mmol) in 220 mL of a 2:1 MeOH/THF mixture was treated with a catalytic amount of 10% palladium on carbon (5 mol %, 1.07 g). The mixture was hydrogenated at 35 psi for 18 h. NMR of an aliquot showed complete conversion into product. The mixture was diluted with dichloromethane (300 mL) and the solids were removed by filtration through a short path of celite. The filtrate was concentrated in vacuo to afford the product 17A (5.03 g; 99%) as a white solid.

### Step B—Synthesis of Compound 17B

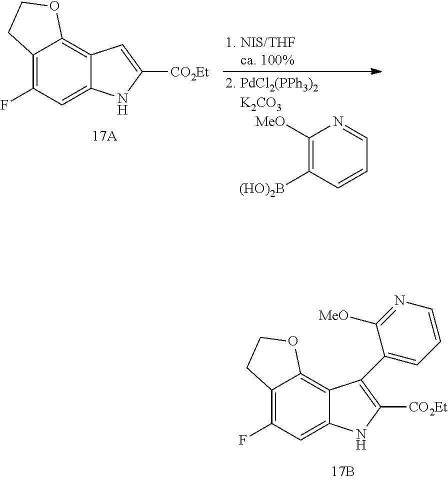

A solution of 17A (7.81 g; 31.34 mmol) in 300 mL of THF was cooled to −78° C. and treated with a solution of N-iodosuccinimide (1.1 eq, 7.75 g in 100 mL of THF). The mixture was stirred for 20 min and TLC (25% THF in hexanes) showed complete consumption of starting material. The reaction was quenched by addition of aqueous saturated sodium bicarbonate soln (100 mL). The mixture was allowed to reach room temperature and the product was dissolved in ethyl acetate (800 mL). The organic layer was washed with aqueous saturated sodium bicarbonate (100 mL) and brine (80 mL). The organic layer was dried over magnesium sulfate, filtered and concentrated in vacuo. The crude product (ca. 100%, 11.75 g) was used directly in the next reaction.

The product above (11.75 g; 40.44 mmol) was dissloved in 400 mL of 1,2-dimethoxyethane was treated with 2-methoxypyridine-3-boronic acid (2.0 eq, 12.3 g) and bis(triphenylphosphine)palladium(II) chloride (0.1 eq, 2.8 g). The mixture was stirred for 10 min followed by addition of aqueous potassium carbonate (4.0 eq, 80.8 mL of 2 M soln). The mixture was stirred at 90° C. and the progress of the reaction was followed by TLC (25% THF in hexanes). The reaction was completed after ˜2 h, the mixture was diluted with ethyl acetate (600 mL) and washed with aqueous saturated sodium bicarbonate (2×200 mL) and brine (200 mL). The organic layer was dried over magnesium sulfate, filtered and concentrated in vacuo to afford the crude product as a brown solid. The crude product was treated with acetonitrile (200 mL) and stirred in an oil bath at 90° C. Acetonitrile was added in portions (50 mL) until the mixture became a homogeneous dark solution (approx. 300 mL). The heating bath was removed and the mixture was allowed to reach room temp. The mixture was then placed in freezer (−20° C.) overnight. The mother liquor was removed (decantation) and the solids were washed with ether (50 mL). The crystallized product 17B was dried under high vacuum (11.66 g, 82%) to give a slightly yellow powder.

### Step C—Synthesis of Compound 17C

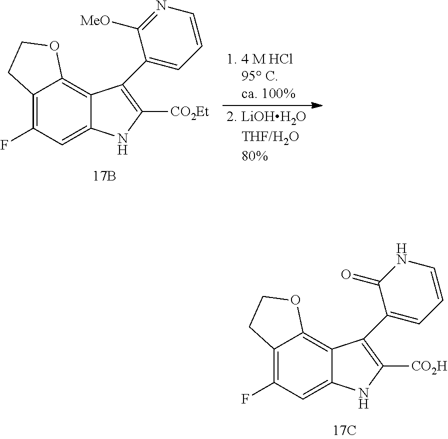

Compound 17B was divided into two batches and treated separately. Each batch was dissolved in 4 M HCl solution in dioxane (100 mL) and methanol (25 mL). The homogeneous solution was heated in a sealed tube (95° C.) until all starting material had been consumed. After 3 h, the mixture was concentrated to dryness in vacuo to give the crude product (ca 100%, 7.97 g) as a slightly yellow solid which was used without further purification.

An aliqiout of the product above (780 mg, 2.278 mmol) was dissolved in 40 mL of 1:1 THF/MeOH and water was added (10 mL). The resulting solution was treated with lithium hydroxide monohydrate (5.0 eq, 478 mg) and heated to 50° C. for 3 h. TLC (50% THF in dichloromethane) showed complete disappearence of the starting material. The mixture was treated with 15 mL of aqueous 1 M HCl and the volatiles were removed in vacuo. The crude product was diluted with aqueous 1 M HCl (20 mL) and the solids recovered by filtration (whatman #1) and washed with ether (30 mL) to give the product 17C (560 mg; 78%) as a slightly yellow solid.

### Step D—Synthesis of Compound 17D

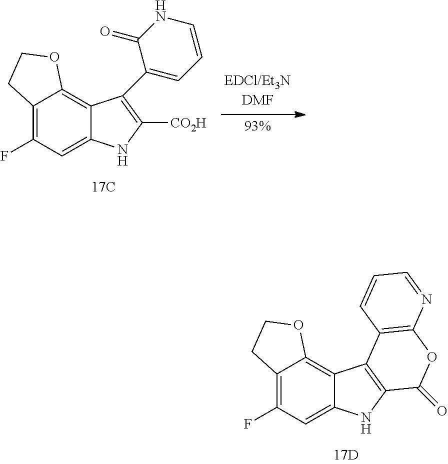

Compound 4C (4.75 g, 15.11 mmol) was suspended in 150 mL of dry DMF and treated with EDCI (2.0 eq, 5.79 g) and triethylamine (10 eq, 21.2 mL, d 0.720). The mixture was stirred overnight at room temp. All the volatiles were removed in vacuo (high vacuum pump) and the residue was treated with methanol (30 mL). The product precipitated as a slightly yellow solid which was recovered by filtration. The product was washed with methanol (10 mL) and hexanes (20 mL) and dried under vacuum to afford 17D (4.2 g; 93%) as a slightly yellow solid.

### Example 18

### Preparation of Compound 223

### Step A—Synthesis of Compound 18B

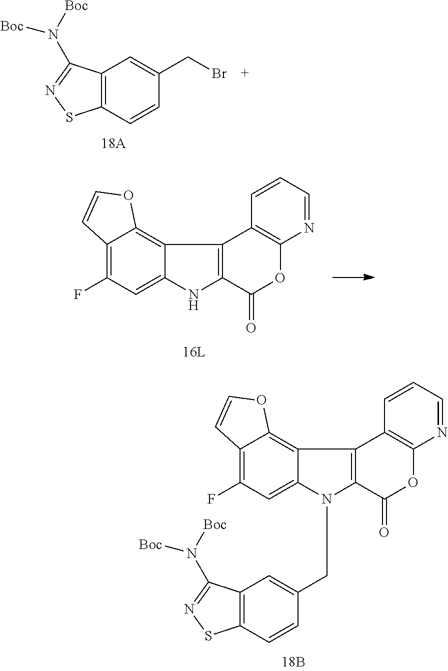

A mixture of lactone 16L (215 mg; 0.733 mmol) and N,N-bis-Boc-5-bromomethyl-benzo[d]isothiazol-3-ylamine 18A (1.2 eq, 390 mg) was suspended in dry DMF (7 mL) and treated with cesium carbonate (2.0 eq, 477 mg). The slurry was stirred overnight. The mixture was treated with water (10 mL) and the product was recovered by filtration (whatman #1). The solids were washed with water (2×5 mL) to give the product 18B (480 mg; 99%) as a white solid which did not require further purification. ¹H-NMR (dmso-d₆; 400 MHz): δ 9.28 (1H, dd, J=1.83, 7.93 Hz), 8.50 (1H, dd, J=1.22, 4.88 Hz), 8.28 (1H, d, J=2.44 Hz), 8.20 (1H, d, J=8.54 Hz), 7.75 (1H, d, J=10.37 Hz), 7.66 (2H, m), 7.36 (1H, s), 7.28 (1H, d, J=1.83 Hz), 6.25 (2H, s), 1.12 (18H, s).

### Step B—Synthesis of Compound 223

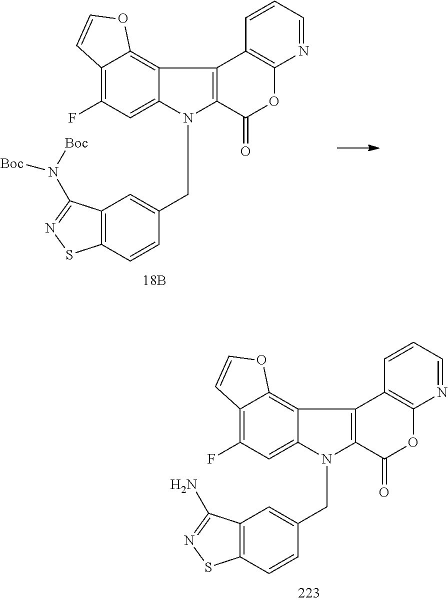

The N,N-bis-Boc protected aminoisothiazole 11A (480 mg; 0.730 mmol) was treated with 4 M HCl in dioxane (15 mL). The resulting slurry was stirred for 3 h at which point no more starting material remained according to TLC (50% ethyl acetate in hexanes). The mixture was concentrated to dryness in vacuo to give the crude product 11B (ca 99%; 333 mg) as a slightly yellow solid which was used without further purification. ¹H-NMR (dmso-d₆; 400 MHz): δ 9.28 (1H, dd, J=1.83, 7.93 Hz), 8.50 (1H, dd, J=1.83, 4.88 Hz), 8.28 (1H, d, J=2.44 Hz), 7.95 (1H, d, J=8.54 Hz), 7.77 (1H, s), 7.70 (2H, broad s), 7.65 (1H, dd, J=4.88, 7.93 Hz), 7.61 (1H, d, J=10.37 Hz), 7.58 (1H, dd, J=1.83, 8.54 Hz), 7.28 (1H, d, J=1.83 Hz), 6.14 (2H, s).

### Example 19

### Preparation of Compound 224

### Step A—Synthesis of Compound 19A

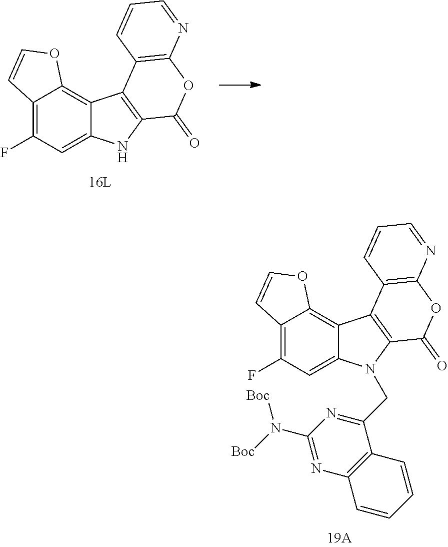

A solution of lactone 16L (244.1 mg, 0.83 mmol) in 10 mL of dry DMF was treated with N,N-bis-Boc-4-bromomethyl-quinazolin-2-ylamine 086951-092-36 (1.1 eq; 400 mg) and cesium carbonate (3.0 eq, 811 mg). The slurry was set to stir overnight. The reaction was quenched with water (5 mL), stirred for 10 minutes and dried under reduced pressure and heat. The residual paste was diluted with ethyl acetate (400 mL) and washed with water (2×30 mL) and brine (2×30 mL). The organic layer was separated, dried over magnesium sulfate, filtered, and concentrated under reduced pressure. Product 19A was afforded as a crude yellow gum and was not purified further (300 mg; 55%).

### Step B—Synthesis of Compound 18B

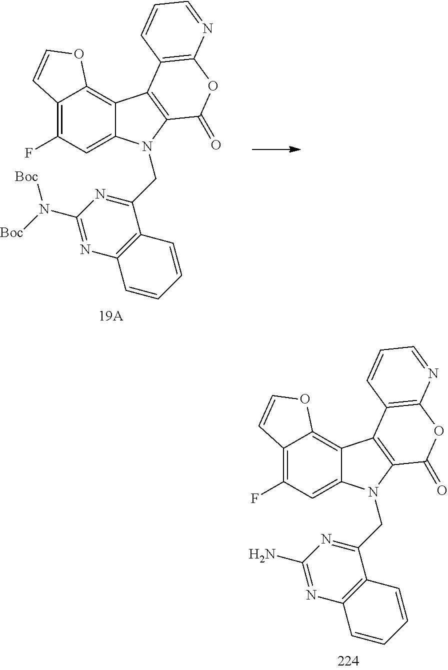

Lactone 19A (380 mg; 0.631 mmol) was diluted with 5 mL methylene dichloride to which 5 mL difluoro acetic acid was added. The reaction was stirred for 3 h. The reaction mixture was dried under reduced pressure and set to dry further under vacuum for 48 h. to afford dry 224.

### Example 20

### Preparation of Compounds 225

### Step A—Synthesis of Compound 225

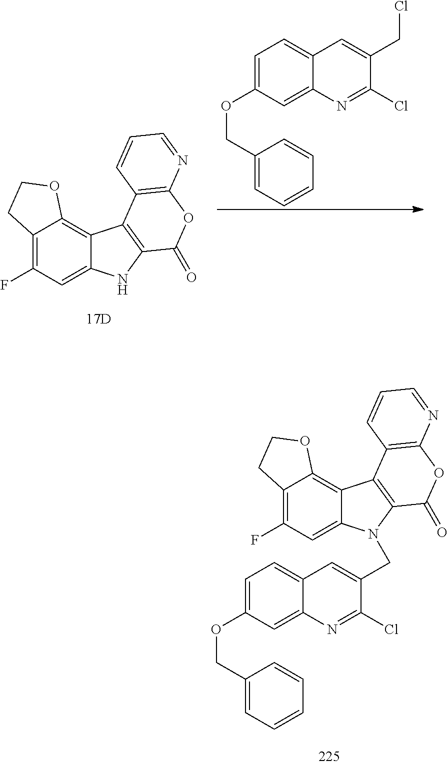

To a solution of compound 4D (0.65 g, 2.2 mmol) in DMF (10 mL) was added cesium carbonate (0.72 g, 2.2 mmol) and compound 19C (0.71 g, 2.2 mmol) and the resulting reaction was allowed to stir at room temperature for 24 hours. The reaction mixture was diluted with EtOAc and washed with water, brine. The combined organic layers were dried (Na₂SO₄), filtered, and concentrated in vacuo, and purified using flash chromatography, to provide compound 21A (0.8 g, 68%).

### Example 21

### Preparation of Compounds 226

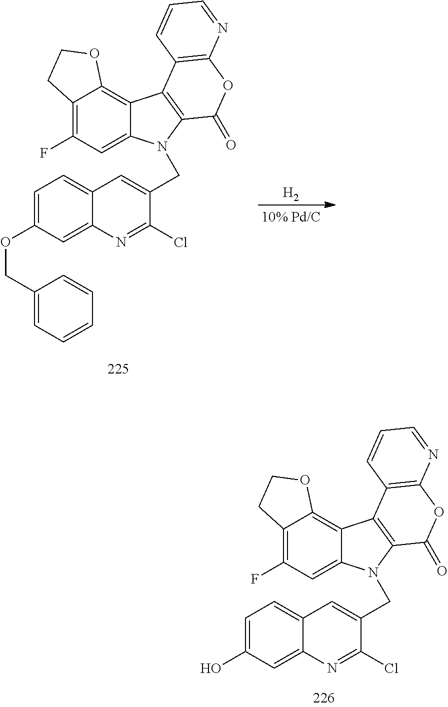

To solution of the compound 225 (0.2 g, 0.35 mmol) in THF was added 10% Pd/C and treated with hydrogen in balloon for 24 hours. Reaction mixture was diluted with Ethyl Acetate and filtered through celite, concentrated in vacuo, purified using flash chromatography, to provide compound 226 (0.12 g, 71%).

### Example 22

### Preparation of Compound 203

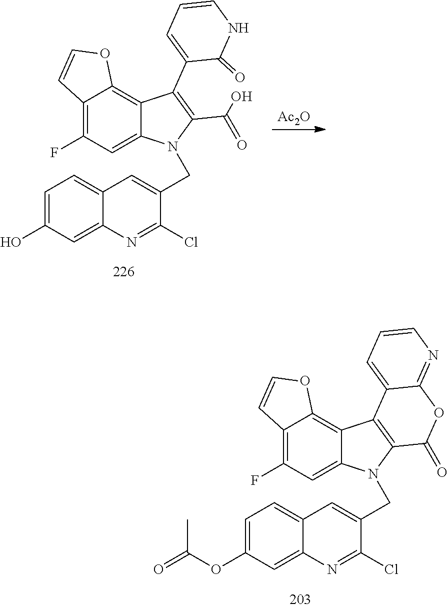

Compound 226 (200 mg 0.4 mmol) was dissolved in 5 mL DMF at room temperature and 1 mL acetic anhydride. 1 mL triethyl amine was added and stirred for 1 hour at 60° C. The reaction mixture was diluted with ethyl acetate and washed with water, brine. The combined organic layers were dried (Na₂SO₄), filtered, and concentrated in vacuo, product was washed with methanol, dried in vacuum for 24 hours to provide compound 203 (77 mg, 37%).

### Example 23

### Preparation of Compound 159

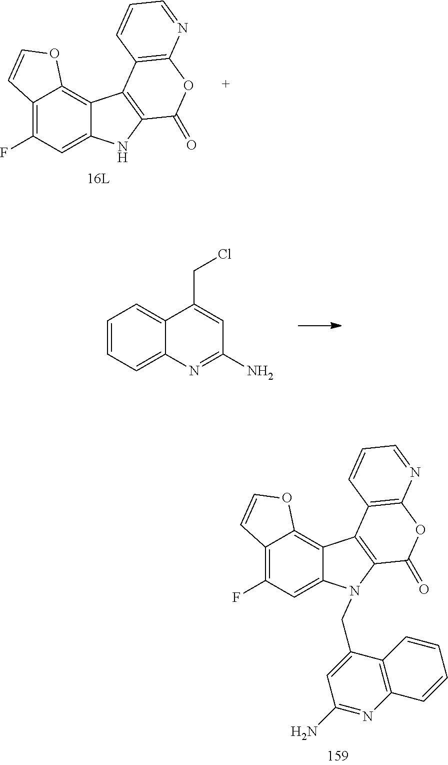

The suspension of 16L (305 mg, 1.04 mmol), aminoquinoline benzylchloride (300 mg, 1.57 mmol), cesium carbonate (1.97 g, 6.04 mmol) and DMF (5 ml) was stirred at room temperature for 20 hours. Water (10 mL) was added to the reaction mixture before filtration. The cake was washed with MeOH (2×1 ml), dried by air and then on house vacuum to afford 159 as a light yellow powder (280 mg, 60%). This crude product is pure enough for the next reaction without further purification.

### Example 24

### Preparation of Compound 153

### Step A—Synthesis of Compound 153

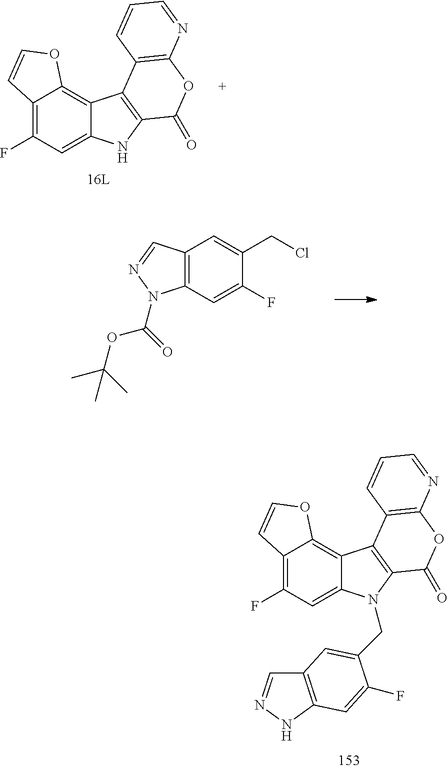

A solution of 16L (900 mg, 3.06 mmol) in DMF (40.00 mL, 516.6 mmol) was treated with tert-butyl 5-(chloromethyl)-6-fluoro-1H-indazole-1-carboxylate (1.09 g, 3.84 mmol) and cesium carbonate (1.50 g, 4.59 mmol) and stirred at rt. overnight. The reaction mixture was concentrated, diluted with CH₂Cl₂ (600 mL), washed with water, dried (MgSO₄), filtered, concentrated in vacuo and purified by chromatography (THF/Hexanes) to yield alkylated product (1.6 g; Yield=96%; The purified solid was dissolved in CH₂Cl₂ (40 mL) and TFA (40 mL) and stirred at rt. for 1 h. The reaction mixture was concentrated and treated with ether and the resulting solid 153 was filtered and dried.

### Example 25

### Preparation of Intermediate Compound 25B

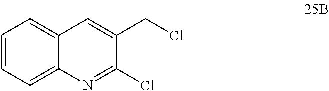

### Step A—Synthesis of Compound 6A

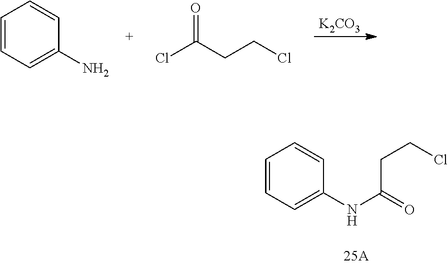

A mixture of aniline (65.04 mL, 713.8 mmol), potassium carbonate (54.4 g, 394 mmol) and water (300 mL) were added to a 2000 mL flask. The resulting reaction was kept at room temperature using a room temperature water bath and stirred with a mechanic stirrer. 3-Chloro-propionyl chloride (75.18 mL, 787.6 mmol) was added dropwise via additional funnel and the resulting suspension was allowed to stir at room temperature for 3 hours. The reaction mixture was filtered and the collected solid was washed sequentially with water (300 mL), aq. HCl (1M, 2×300 mL), and water (300 mL), then dried to provide compound 25A, which was used without purification (114.5 g, 87%).

### Step B—Synthesis of Compound 25B

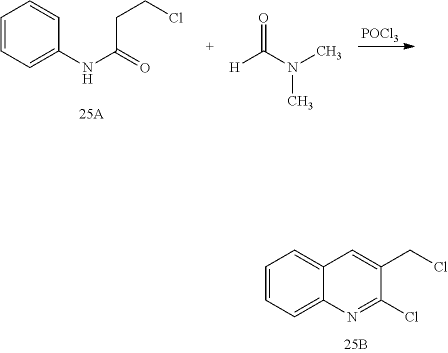

N,N-Dimethylformamide (53.7 mL, 694 mmol) was charged into a three necked flask and cooled to 0° C. and treated with phosphoryl chloride (177.7 mL, 1906 mmol) dropwise. The reaction was stirred at that temperature for 10 min and treated with 3-Chloro-N-phenylpropanamide 25A (50.00 g, 272.3 mmol) and stirred at room temperature. for 30 min. The reaction mixture was heated at 80° C. for 3 h and slowly poured into ice. The solid separating out was filtered and washed extensively with water (2×1000 mL), aq. saturated sodium bicarbonate (500 mL), and taken in EtOAc (1 L), The solution was dried (MgSO₄) filtered concentrated in vacuo and the residue obtained was recrystallized from boiling hexanes to provide compound 25B (20 g).

### Example 26

### Preparation of Intermediate Compounds 26E and 26F

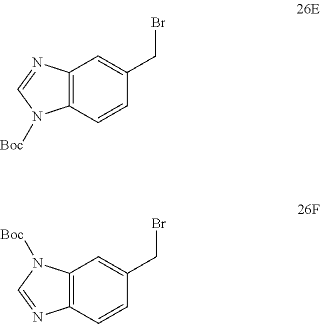

### Step A—Synthesis of Compound 26B

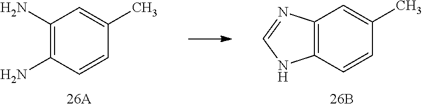

A solution of compound 26A (3 g, 24.5 mmol) in trimethyl orthoformate (15 mL) was treated with 2 drops conc. HCl and heated to 80° C. for 2 hours. The reaction mixture was cooled to room temperature and concentrated in vacuo to provide compound 26B (3.65 g), which was used without further purification. M.S. found for C₈H₈N₂: 133.2 (M+H)⁺.

### Step B—Synthesis of Compounds 26C and 26D

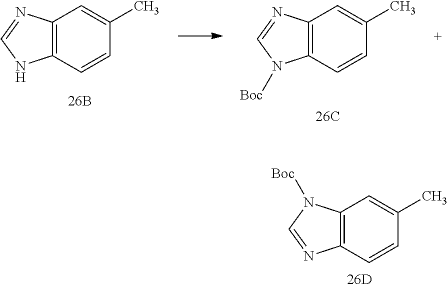

To a solution of compound 26B (24.5 mmol) in CH₃CN (65 mL) was added di-tertbutyl dicarbonate (5.89 g, 27.0 mmol), triethylamine (3.76 mL, 27.0 mmol) and 4-dimethylamino pyridine (300 mg, 2.45 mmol) and the resulting reaction was heated to 80° C. and allowed to stir at this temperature for 1.5 hours. The reaction mixture was cooled to room temperature, concentrated in vacuo, and the residue obtained was purified using flash column chromatography (silica gel, EtOAc/Hexanes 5-20%) to provide a mixture of isomeric compounds 26C and 26D (5.38 g, 94.3% yield over steps A and B).

### Step C—Synthesis of Compounds 26E and 26F

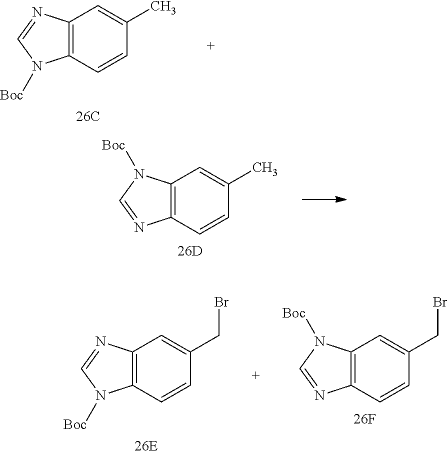

To a solution of compounds 26C and 26D (2 g, 8.61 mmol) in carbon tetrachloride (40 mL) was added N-bromosuccinimide (1.6 g, 9.04 mmol) and dibenzoyl peroxide (41.7 mg, 0.1722 mmol) and the resulting reaction was heated to 90° C. and allowed to stir at this temperature for 12 hours. The reaction was cooled to room temperature, solids were filtered off and the filtrate was washed with water, dried over sodium sulfate and concentrated in vacuo to provide compounds 26E and 26F (2.58 g) which was used without further purification. M.S. found for C₁₃H₁₅BrN₂O₂: 334.7 (M+Na)⁺.

### Example 27

### Preparation of Intermediate Compound 27B

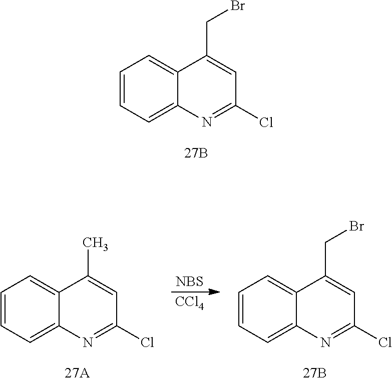

A mixture of compound 27A (1.5 g, 8.44 mmol), NBS (1.8 g, 10.11 mmol) in carbon tetrachloride (50 mL) was heated to reflux, then benzoyl peroxide (0.21 g, 0.866 mmol) was added. The resulting suspension was allowed to stir at reflux for 19 hours, then cooled to room temperature and filtered. The filtrate was washed with saturated sodium carbonate, dried over sodium sulfate and concentrated in vacuo to provide a mixture (1.7 g) which contains about 50% of compound 27B, and was used without further purification.

### Example 28

### Preparation of Intermediate Compound 28G

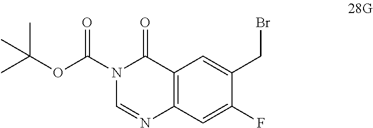

### Step A—Synthesis of Compound 9B

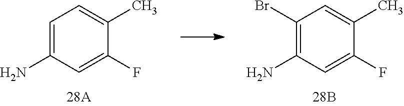

A mixture of compound 28A (6.00 g, 47.9 mmol) and anhydrous potassium carbonate (6.70 g, 48.5 mmol) in anhydrous dichloromethane (130 mL) was cooled to −15° C. in a salt-ice bath and then added dropwise to a solution of bromine (7.70 g, 48.2 mmol) in anhydrous dichloromethane (80 mL). After addition was complete, the reaction was allowed to stir at −15° C. for 1 hour. Ice water (100 mL) was added to the reaction mixture and the aqueous layer was extracted with dichloromethane (2×100 mL). The combined organic layers were dried over MgSO₄ and concentrated in vacuo to provide compound 28B (11.0 g, quant.), which was used without further purification.

### Step B—Synthesis of Compound 28C

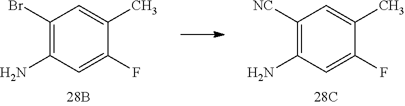

Compound 28B was dissolved in DMF (150 mL) and to this solution was added copper (I) cyanide (11.0 g, 123 mmol). The mixture was heated to 160° C. and allowed to stir at this temperature for 20 hours. After being cooled to room temperature, with water (200 mL), iron (III) chloride (42.0 g, 155 mmol) and concentrated hydrochloric acid (20 mL) were added to the reaction mixture and the resulting reaction was stirred for 45 minutes. The reaction mixture was then basified to pH>10 using commercial ammonium hydroxide solution. The basic solution was then extracted with ethyl acetate (4×400 mL). The combined organic extracts were washed with water, dried over magnesium sulfate, filtered and concentrated in vacuo. The residue obtained was purified using flash chromatography to provide compound 28C (5.82 g, 81%). ¹H NMR (400 MHz, d₆-DMSO): δ 7.34 (d, J=8.4 Hz, 1H), 6.52 (d, J=12.4 Hz, 1H), 6.10 (s, 2H), 2.08 (s, 3H).

### Step C—Synthesis of Compound 28D

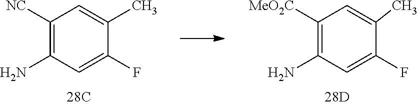

To the solution of 28C (2.0 g, 13.3 mmol) in anhydrous methanol (15 mL) at room temperature was added concentrated sulfuric acid (4.0 mL). The reaction mixture was heated to 70° C. and stirred for four days. After cooled to room temperature, it was poured into with ice water. The mixture was then diluted with ethyl acetate (200 mL) and was made basic (pH>10) with commercial ammonium hydroxide solution. The layers were separated. The aqueous layer was extracted with ethyl acetate (2×100 mL). The combined organic solution was dried over MgSO₄ and concentrated in vacuo to provide the crude product which, was purified using flash chromatography to provide compound 28D (1.0 g, 41%) and some recovered 28C. ¹H NMR (400 MHz, d₆-DMSO): δ 7.61 (d, J=8.8 Hz, 1H), 6.69 (s, 2H), 6.51 (d, J=12.0 Hz, 1H), 3.77 (s, 3H), 2.06 (s, 3H).

### Step D—Synthesis of Compound 28E

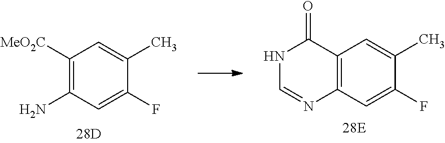

The solution of compound 28D (500 mg, 2.73 mmol) in formamide (6.0 mL) was heated to 150° C. in an oil bath and stirred for 18 hours. After cooled to room temperature, ethyl acetate (100 mL) and water (100 mL) were added and the layers were separated. The organic solution was washed with water (2×60 mL), dried over MgSO₄ and concentrated in vacuo to provide the crude product 28E (0.50 g, quant.) which, was used without further purification. MS found for C₉H₇FN₂O: 179.0 (M+H)⁺.

### Step E—Synthesis of Compound 28F

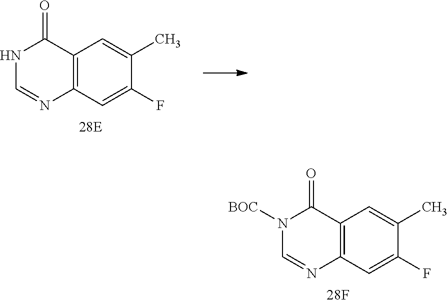

To the solution of 28E (from Step 4) in anhydrous THF (20 mL) at room temperature was added di-tert-butyl dicarbonate (1.84 g, 8.43 mmol), 4-dimethylaminopyridine (350 mg, 2.86 mmol) and triethyl amine (0.40 mL, 2.87 mmol). The reaction mixture was stirred for 18 hours. Ethyl acetate (100 mL) and water (100 mL) were added and the layers were separated. The aqueous layer was extracted with ethyl acetate (2×50 mL). The combined organic solution was dried over MgSO₄ and concentrated in vacuo to provide the crude product which, was purified using flash chromatography to provide compound 28F (285 mg, 36%). MS found for C₁₄H₁₅FN₂O₃: 179.0 (M+H-100)⁺.

### Step F—Synthesis of Compound 28G

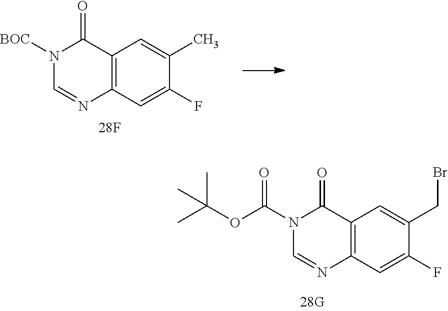

The mixture of 28F (282 mg, 1.01 mmol), NBS (253 mg, 1.42 mmol) and AIBN (58 mg, 0.353 mmol) in anhydrous carbon tetrachloride (60 mL) was heated to 90° C. in an oil bath and stirred for 4 hours. After cooled to room temperature and concentrated in vacuo, the residue was dissolved in ethyl acetate (100 mL) and water (100 mL). The layers were separated. The organic solution was washed with water (100 mL), dried over MgSO₄ and concentrated in vacuo to provide the crude product 28G (453 mg, quant.) which, was used without further purification.

### Example 29

### Preparation of Intermediate Compound 29E

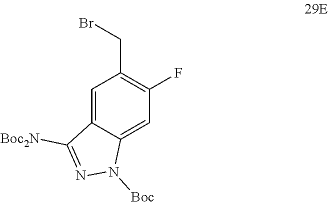

### Step A—Synthesis of Compound 29A

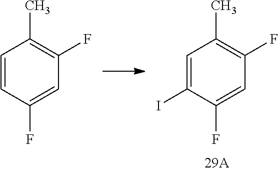

A solution of 2,4-difluorotoluene (4.72 g, 36.8 mmol) in trifluoroacetic acid (12.29 mL, 159.5 mmol) was cooled to 0° C., then N-Iodosuccinimide (9.59 g, 42.6 mmol) was added and the resulting reaction was allowed to stir at room temperature for about 15 hours. The reaction mixture was then concentrated in vacuo and the residue obtained was dissolved in hexanes (100 mL), washed with aquesous sodium thiosulfate (100 mL), brine (100 mL), then dried (MgSO₄), filtered and concentrated in vacuo. The resulting residue was purified using bulb-to-bulb distillation to provide compound 29A (7.2 g, 77%) as a colorless oil.

### Step B—Synthesis of Compound 29B

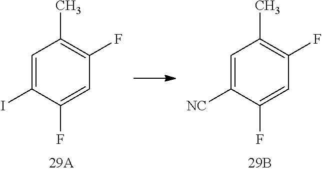

A solution of compound 29A (7.11 g, 28.0 mmol), zinc cyanide (1.97 g, 16.8 mmol) and tetrakis(triphenylphosphine)palladium(0) (3.23 g, 2.80 mmol) in DMF (30 mL) was heated to 90° C. and allowed to stir at this temperature for 1.5 hours. The reaction mixture was concentrated in vacuo and the residue obtained was taken up in water (400 mL) and extracted with ether (400 mL). The organic extract was washed with aqueous ammonium hydroxide solution (1N). The organic layer was dried (MgSO₄) filtered, concentrated in vacuo to provide a residue that was purified using flash column chromatography (SiO₂, EtOAc/Hexanes) to provide a mixture that contained product and triphenylphosphine. This mixture was further purified using sublimation at 1 mm/Hg at 45° C. to provide compound 29B (1.8 g; Yield=42%).

### Step C—Synthesis of Compound 29C

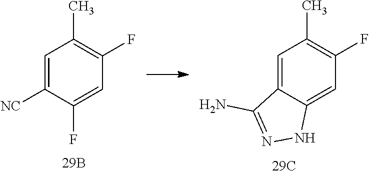

A solution of compound 29B (1.400 g, 9.154 mmol) and hydrazine (0.700 mL, 22.3 mmol) in isopropyl alcohol (50 mL, 653.1 mmol), was heated to reflux and allowed to stir at this temperature for 24 hours. The reaction mixture was cooled to room temperature, concentrated in vacuo and the residue obtained was purified using flash column chromatography (SiO₂, Acetone/Hexanes 0→50%) to provide compound 29C (330 mg, 22%).

### Step D—Synthesis of Compound 10D

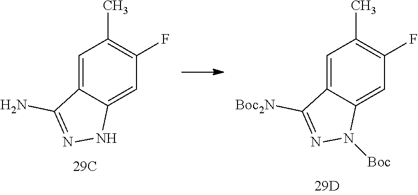

A solution of compound 29C (330.00 mg, 1.998 mmol), di-tert-butyldicarbonate (2.6163 g, 11.98 mmol) and 4-dimethylaminopyridine (48.817 mg, 0.39959 mmol) in acetonitrile (15 mL, 287.2 mmol) was heated to reflux and allowed to stir at this temperature for 2 hours. The reaction mixture was cooled to room temperature, concentrated in vacuo, and the resulting residue was purified using flash column chromatography (SiO₂, EtOAc/(Hexanes 0-20%) to provide compound 29D (640.00 mg, 68%) as a colorless oil.

### Step E—Synthesis of Compound 29E

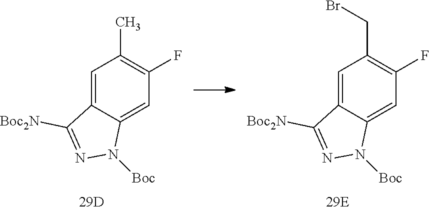

A solution of compound 29D (630.00 mg, 1.3533 mmol), N-bromosuccinimide (337.22 mg, 1.8947 mmol) and benzoyl peroxide (65.563 mg, 0.27067 mmol) in carbon tetrachloride (20 mL) was heated to reflux and allowed to stir at this temperature for 3 hours. The reaction mixture was cooled to room temperature, concentrated in vacuo and the residue obtained was dissolved in EtOAc (300 mL). The resulting solution was washed with aqueous sodium thiosulfate (100 mL), brine (100 mL), dried (MgSO₄), filtered, and concentrated in vacuo. The residue obtained was purified using flash column chromatography (SiO₂, EtOAc/Hexanes) to provide compound 29E as a colorless oil.

### Example 30

### Preparation of Intermediate Compounds 30E and 30F

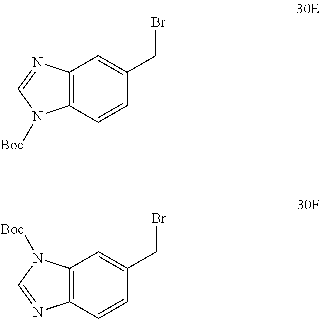

### Step A—Synthesis of Compound 30B

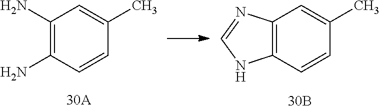

A solution of compound 8A (3 g, 24.5 mmol) in trimethyl orthoformate (15 mL) was treated with 2 drops conc. HCl and heated to 80° C. for 2 hours. The reaction mixture was cooled to room temperature and concentrated in vacuo to provide compound 8B (3.65 g), which was used without further purification. M.S. found for C₈H₈N₂: 133.2 (M+H)⁺.

### Step B—Synthesis of Compounds 30C and 30D

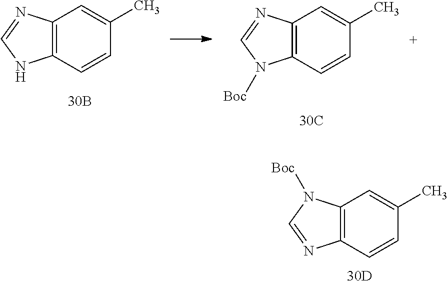

To a solution of compound 30B (24.5 mmol) in CH₃CN (65 mL) was added di-tertbutyl dicarbonate (5.89 g, 27.0 mmol), triethylamine (3.76 mL, 27.0 mmol) and 4-dimethylamino pyridine (300 mg, 2.45 mmol) and the resulting reaction was heated to 80° C. and allowed to stir at this temperature for 1.5 hours. The reaction mixture was cooled to room temperature, concentrated in vacuo, and the residue obtained was purified using flash column chromatography (silica gel, EtOAc/Hexanes 5-20%) to provide a mixture of isomeric compounds 30C and 30D (5.38 g, 94.3% yield over steps A and B).

### Step C—Synthesis of Compounds 30E and 30F

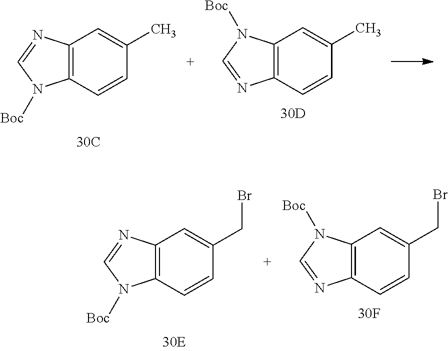

To a solution of compounds 30C and 30D (2 g, 8.61 mmol) in carbon tetrachloride (40 mL) was added N-bromosuccinimide (1.6 g, 9.04 mmol) and dibenzoyl peroxide (41.7 mg, 0.1722 mmol) and the resulting reaction⁻was heated to 90° C. and allowed to stir at this temperature for 12 hours. The reaction was cooled to room temperature, solids were filtered off and the filtrate was washed with water, dried over sodium sulfate and concentrated in vacuo to provide compounds 30E and 30F (2.58 g) which was used without further purification. M.S. found for C₁₃H₁₅BrN₂O₂: 334.7 (M+Na)⁺.

### Example 31

### Preparation of Intermediate Compound 31B

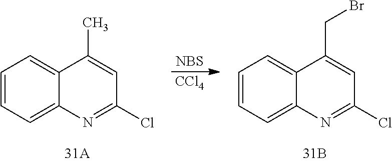

A mixture of compound 31A (1.5 g, 8.44 mmol), NBS (1.8 g, 10.11 mmol) in carbon tetrachloride (50 mL) was heated to reflux, then benzoyl peroxide (0.21 g, 0.866 mmol) was added. The resulting suspension was allowed to stir at reflux for 19 hours, then cooled to room temperature and filtered. The filtrate was washed with saturated sodium carbonate, dried over sodium sulfate and concentrated in vacuo to provide a mixture (1.7 g) which contains about 50% of compound 31B, and was used without further purification.

### Example 32

### Preparation of Intermediate Compound 32D

### Step A—Synthesis of Compound 32B

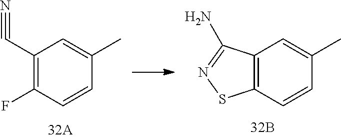

A mixture of 2-fluoro-5-methylbenzonitrile (32A, 2.0 g; 14.799 mmol) and sodium sulfide (1.0 eq, 1.15 g) was dissolved in 150 mL of DMSO and heated at 70° C. overnight. The mixture was placed in an ice-water bath and treated with concentrated aqueous ammonium hydroxide (20 mL) and aqueous sodium hypochlorite (20 mL). The reaction mixture was allowed to warm to room temperature and stirred for 5 h. The mixture was diluted with ethyl acetate (300 mL) and washed with water (2×60 mL) and brine (50 mL). The organic layer was dried over magnesium sulfate, filtered and concentrated in vacuo. The residue was adsorbed on silica gel and purified on a Biotage 40-M silica gel column (gradient: 0 to 30% acetone in hexanes) to give the product 32B (860 mg; 36%) as a white solid. ¹H-NMR (CDCl₃; 400 MHz): δ 7.68 (1H, d, J=8.54 Hz), 7.48 (1H, s), 7.33 (1H, d, J=8.54 Hz), 4.89 (2H, broad s), 2.50 (3H, s).

### Step B—Synthesis of Compound 32C

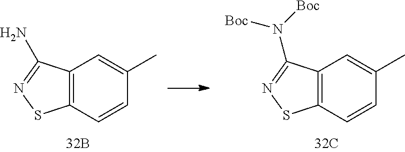

A solution of 5-methylbenzo[d]isothiazol-3-ylamine, (10B, 850 mg; 5.176 mmol) in dry acetonitrile (50 mL) was treated with Boc-anhydride (2.1 eq, 2.37 g) and heated to 50° C. All starting material had been consumed after 2 h and the mixture was concentrated in vacuo to one third of its volume. The residue was dissolved in ethyl acetate (100 mL) and washed with aqueous sodium hydrogen sulfate (20 mL), and brine (20 mL). The organic layer was dried over magnesium sulfate, filtered and concentrated in vacuo. The residue was adsorbed on silica gel and purified on a Biotage 40-M silica gel column (gradient: 0 to 10% ethyl acetate in hexanes) to give the product 10C (1.7 g; 91%) as a white powder. ‘H-NMR (CDCl₃; 400 MHz): δ 7.77 (1H, d, J=8.54 Hz), 7.55 (1H, s), 7.38 (1H, dd, J=1.83, 8.54 Hz), 2.51 (3H, s), 1.36 (18H, s). LR-MS (ESI): caldc for C₁₈H₂₅N₂O₄S [M+H]⁺ 365.15; found 365.23.

### Step C—Synthesis of Compound 32D

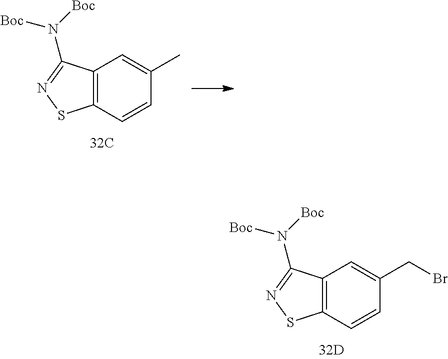

A solution of N,N-bis-Boc-5-methyl-benzo[d]isothiazol-3-ylamine (32D, 500 mg; 1.371 mmol) in 15 mL of carbon tetrachloride was treated N-bromosuccinimide (1.05 eq, 256 mg) and benzoyl peroxide (10 mol %; 33 mg). The solution was degassed (vacuum/argon flush) and then heated to 75° C. for 5 h. The reaction mixture was concentrated to one third of its volume in vacuo and the residue was dissolved in ethyl acetate (50 mL). The solution was washed with aqueous saturated sodium bicarbonate soln (2×10 mL) and brine (10 mL). The organic layer was dried over magnesium sulfate, filtered and concentrated in vacuo. The residue was adsorbed on silica gel and purified on a Biotage 40-S silica gel column (gradient: hexanes then 0 to 10% ethyl acetate in hexanes) to give the product 32D (396 mg; 69%) as a white solid. ¹H-NMR (CDCl₃; 400 MHz): δ 7.87 (1H, d, J=8.54 Hz), 7.78 (1H, s), 7.58 (1H, dd, J=1.83, 8.54 Hz), 4.63 (2H, s), 1.37 (18H, s). LR-MS (ESI): caldc for C₁₈H₂₄BrN₂O₄S [M+H⁺ 445.06; found 445.24.

### Example 33

### Preparation of Intermediate Compound 33D

### Step A—Synthesis of Compound 33B

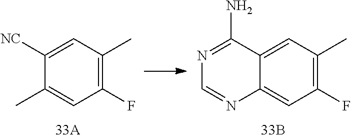

A solution of 33A (0.20 g, 1.33 mmol) in formamide (15 mL) was heated to 150° C. and stirred for 18 h. After cooled to room temperature, ethyl acetate (60 mL) and water (30 mL) were added and the layers were separated. The organic solution was washed with water (3×20 mL), dried (MgSO₄), filtered, and concentrated in vacuo to provide the crude product 33B (0.22 g, 93%). MS found for C₉H₈FN₃: 178.2 (M+H)⁺.

### Step B—Synthesis of Compound 11C

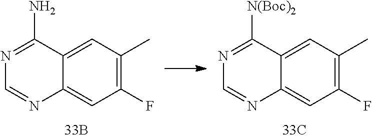

33B was treated with 3.0 equivalent of (Boc)₂O to afford 33C. MS found for C₁₉H₂₄FN₃O₄: 378.4 (M+H)⁺.

### Step C—Synthesis of Compound 33D

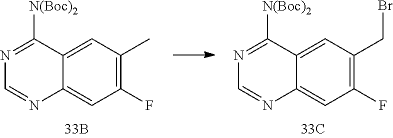

Bromination of 33C understandard N-bromo succinimide conditions afforded 33D. MS found for C₁₉H₂₃BrFN₃O₄: 458.3 (M+H)⁺.

### Example 34

### Preparation of Intermediate Compound 34F

### Step A—Synthesis of Compound 34B

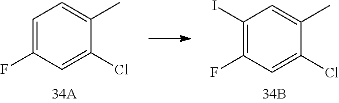

N-iodosuccinimide (1.1 eq; 17.1 g) was added to a solution of 2,4-difluoro toluene (34A, 10.0 g; 69.17 mmol; Alfa Aesar) in trifluoroacetic acid (46 mL). The reaction was set to stir for 12 h. The volatiles were removed under reduced pressure; the remaining slurry was diluted with ether (400 mL) and washed with 5% aq sodium thiosulfate (5×40 mL), water (2×30 mL), and brine (40 mL). The organic layer was collected, dried over magnesium sulfate, filtered, and concentrated under reduced pressure. The reaction was purified via bulb to bulb distillation to afford product 34B as a colorless liquid (17 g; 91%)

### Step B—Synthesis of Compound 34C

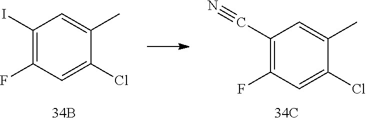

A solution of intermediate 34B (13.0 g; 48.06 mmol) and zinc cyanide (1 eq; 5.644 g) in N,N-dimethlyformamide (50 mL) was treated with tetrakis (triphenylphosphine)palladium(0) (0.1 eq; 5.55 g) and heated at 90° C. for 12 h. The reaction mixture was diluted with ether (600 mL) and ammonium hydroxide (1:1 concentrated ammonium hydroxide: water 200 mL). The organic layer was separated and washed with water (100 mL) and brine (100 mL), dried over magnesium sulfate, filtered, concentrated under reduced pressure, and purified over silica gel first eluting with hexanes, then with 20% ethyl acetate/hexanes. Product 34C (4.48 g; 33%) was afforded as a clear oil.

### Step C—Synthesis of Compound 34D

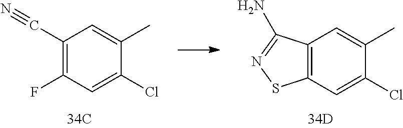

A solution of 34C (2.25 g; 13.27 mmol) and sodium sulfide (1 eq; 1.035 g) was prepared in DMSO (130 mL) and heated at 70° C. overnight. The mixture was placed in an ice water bath and treated with concentrated aqueous ammonium hydroxide (30 mL) and aqueous sodium hypochlorite (30 mL). The reaction mixture was stirred for 5 h (temp from 0 to 25° C.). The mixture was diluted with ethyl acetate (400 mL) and washed with water (2×40 mL) and brine (50 mL). The organic layer was dried over magnesium sulfate, filtered and concentrated in vacuo. The residue was adsorbed on silica gel and purified on an ISCO 330G column (gradient: 0-30% acetone in hexanes), affording product 34D (800 mg; 30.3%) as a white solid.

### Step D—Synthesis of Compound 34E

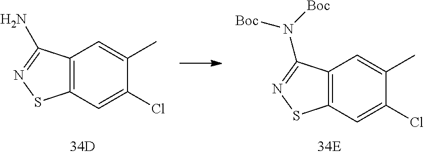

A solution of intermediate 34D (780 mg; 3.93 mmol) in dry acetonitrile (39 mL) was treated with Boc-anhydride (2.2 eq; 1.885 g) and heated to 50° C. All starting material had been consumed after 2 h and the mixture was concentrated in vacuo to one third of its volume. The residue was dissolved in ethyl acetate (100 mL) and washed with aqueous sodium hydrogen sulfate (20 mL) and brine (20 mL). The organic layer was dried over magnesium sulfate, filtered and concentrated in vacuo. The residue was adsorbed on silica gel and purified on a ISCO 80 gram column (gradient: 0 to 10% ethyl acetate in hexanes) to give the product 34E (1.03 g; 66% yield) as a white solid.

### Step E—Synthesis of Compound 34F

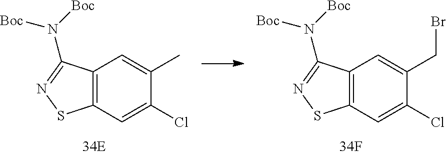

A solution of intermediate 34E (400 mg; 1.003 mmol), N-Bromosuccinimide (1.05 eq; 187.4 mg), and benzoyl peroxide (0.1 eq; 24.3 mg) in dry carbon tetrachloride (10 mL) was prepared and heated at reflux for 12 h. TLC (30% ethyl acetate in hexanes) revealed the reaction had partially progressed. The reaction mixture was concentrated under reduced pressure, diluted with ethyl acetate (100 mL), washed with saturated aqueous sodium bicarbonate (25 mL) and brine (25 mL), dried over magnesium sulfate, filtered, and concentrated under reduced pressure. The residue was then diluted with dichloromethane, adsorbed onto silica gel, and purified on ISCO (25-M Column; 0-40% ethyl acetate in hexanes). The fractions containing product were concentrated under reduced pressure affording intermediate 34F (278 mg; 58%) as a clear yellow oil.

### Example 35

### Preparation of Intermediate Compound 35C

### Step A—Synthesis of Compound 31A

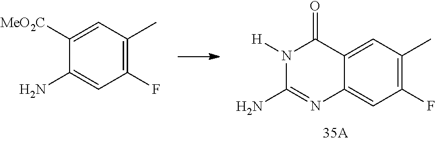

A solid mixture of methyl 2-amino-4-fluoro-5-methylbenzoate (2.66 g, 14.5 mmol), chloroformamidinium hydrochloride (2.6 g, 22.6 mmol) and methyl sulfone (8.5 g, 90.3 mmol) was heated to 150-160° C. in an oil bath with vigorous stirring. It became a clear solution after about 10 min. Heating was continued for a total of 2 h. When cooled to room temperature, it became a solid. The material was taken up with water (200 mL), basified with commercial ammonium hydroxide. After stirred for 1 h, the solid was collected through filtration. It was washed with water (20 mL) and dried under vacuum to give crude product 35A (2.93 g, quant.). MS found for C₉H₈FN₃O: 194.2 (M+H)⁺.

### Step B—Synthesis of Compound 35B

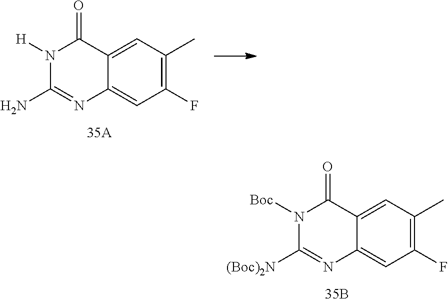

Compound 35B was prepared from 35A according the procedures described, and using 4 equivalents of (Boc)₂O. MS found for C₂₄H₃₂FN₃O₇: 394.3 (M+H-100)⁺.

### Step C—Synthesis of Compound 35C

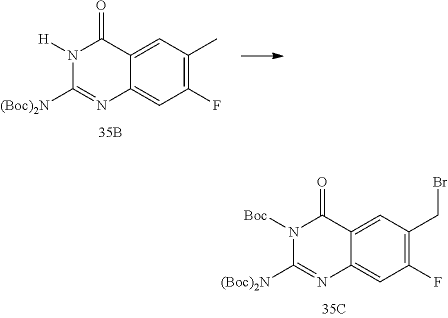

A solution of compound 35B (4.83 g, 9.8 mmol), N-bromosuccinimide (2.70 g, 15.2 mmol) and benzoyl peroxide (600 mg, 2.48 mmol) in carbon tetrachloride (300 mL) was heated to reflux and allowed to stir at this temperature for 18 h. The reaction mixture was cooled to room temperature, concentrated in vacuo and the residue obtained was dissolved in EtOAc (300 mL). The resulting solution was washed with aqueous sodium thiosulfate (100 mL), brine (100 mL), dried (MgSO₄), filtered, and concentrated in vacuo to provide intermediate compound 35C, which was used without further purification. MS found for C₂₄H₃₁BrFN₃O₇: 472.3 (M+H-100)⁺.

### Example 36

### Preparation of Intermediate Compound 36G

### Step A—Synthesis of Compound 36B

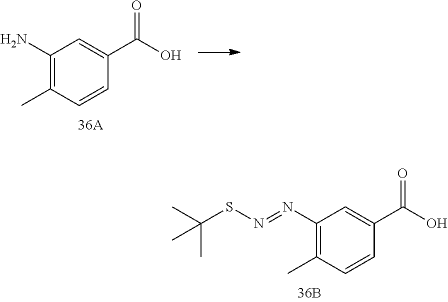

To a stirred solution of aqueous HCl (15 mL of conc HCl in 50 mL of water) was added 3-amino-4-methyl benzoic acid (36 A, 5.0 g; 33.0 mmol). The mixture was cooled in an ice-water bath followed by slow addition of a solution of sodium nitrite (1.1 eq, 2.50 g) in water (12 mL). The mixture was stirred for 30 min at which point the mixture was a homogeneous dark solution. A saturated aqueous solution of sodium acetate was added until pH 6 was attained. Sodium t-butylthiolate (0.5 eq, 1.85 g) was added in one portion. The reaction was stirred for 2 h and the resulting precipitate was collected by filtration (whatman #1), washed with water (20 mL) and dried under vacuum to give the product 36B (2.7 g; 64%) as a tan solid.

### Step B—Synthesis of Compound 36C

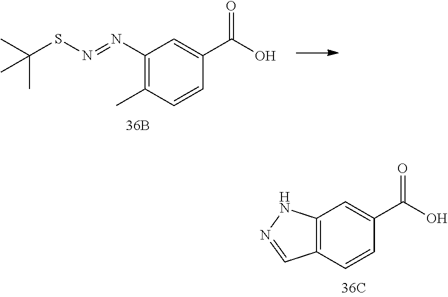

To a stirred solution of potassium tert-butoxide (10.0 eq, 12.0 g) in DMSO (50 mL) was added a solution of t-butyldiazaenyl benzoic acid 36B (2.7 g; 10.70 mmol) in DMSO (30 mL). The mixture was stirred for 6 h and then diluted with ice and acidified with aqueous 1 M HCl until pH 5-6 was attained. The mixture was extracted with ethyl acetate (3×50 mL) and the combined organic layers were washed with water (20 mL) and brine (20 mL). The organic layer was dried over magnesium sulfate, filtered and concentrated in rotavap to give the crude product 36C as a slightly yellow solid which was used without further purification.

### Step C—Synthesis of Compound 36D

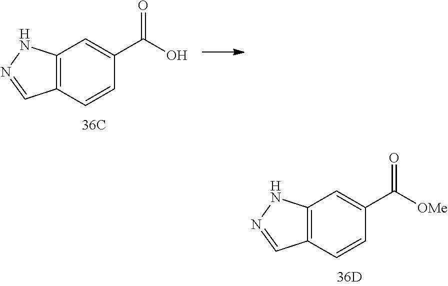

A solution of 1H-indazole-6-carboxylic acid 36C (1.73 g; 10.70 mmol) in toluene (80 mL) and methanol (30 mL) was treated with a solution of TMS-diazomethane (2 M soln in ether) until evolution of gas stopped. The reaction mixture was concentrated in vacuo and the residue was adsorbed on silica gel. The product was purified on a Biotage 40-M silica gel column (gradient: 0 to 20% acetone in hexanes) to give the product 36D (950 mg; 50% for two steps) as a slightly yellow solid. ¹H-NMR (CDCl₃; 400 MHz): δ 8.28 (1H, s), 8.16 (1H, s), 7.86 (1H, d, J=8.54 Hz), 7.81 (1H, d, J=8.54 Hz), 3.98 (3H, s). LR-MS (ESI): caldc for C₉H₉N₂O₂ [M+H]⁺ 177.07; found 177.20.

### Step D—Synthesis of Compound 36E

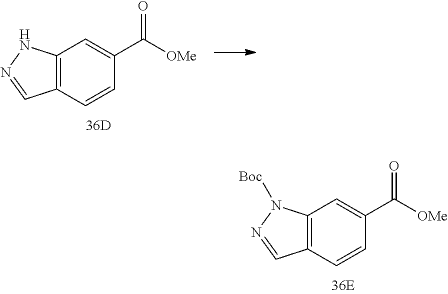

A solution of 1H-indazole-6-carboxylic acid methyl ester 36D (840 mg; 4.76 mmol) in 25 mL of acetonitrile was treated with Boc-anhydride (1.05 eq, 1.09 g) and a catalytic amount of DMAP (tip of spatula). The mixture was stirred at 60° C. for 3 h. The mixture was concentrated to half its volume in rotavap and then diluted with ethyl acetate (100 mL) and washed with aqueous saturated sodium bicarbonate (20 mL) and brine (20 mL). The organic layer was dried over magnesium sulfate, filtered and concentrated in rotavap. The residue was purified on a Biotage 40-M silica gel column (gradient: 0 to 20% ethyl acetate in hexanes) to give the product 36E (1.2 g; 93%) as a colorless oil. ¹H-NMR (CDCl₃; 400 MHz): δ 8.91 (1H, s), 8.22 (1H, s), 7.99 (1H, dd, J=1.22, 8.54 Hz), 7.78 (1H, d, J=8.54 Hz), 3.97 (3H, s), 1.74 (9H, s).

### Step E—Synthesis of Compound 36F

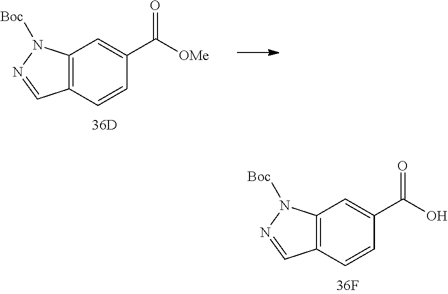

A solution of indazole 36E (460 mg; 1.66 mmol) in 16 mL of dry THF was cooled to −78° C. and treated with lithium triethylborohydride (2.5 eq, 4.15 mL of a 1 M soln in THF). The reaction mixture was stirred at −78° C. and followed by TLC (25% ethyl acetate in hexanes). The reaction was completed in about 1 h and quenched by addition of aqueous saturated sodium hydrogen sulfate (3 mL). The mixture was extracted with ethyl acetate (100 mL) and washed with water (20 mL) and brine (20 mL). The organic layer was dried over magnesium sulfate, filtered and concentrated in rotavap to give the crude product as a colorless oil. The residue was chromatographed on a Biotage 40-S silica gel column (0 to 40% ethyl acetate in hexanes) to give the following: des-Boc starting material (70 mg); alcohol product 36F (160 mg; 40%). ¹H-NMR (CDCl₃; 400 MHz): δ 8.19 (1H, s), 8.13 (1H, s), 7.67 (1H, d, J=7.93 Hz), 7.30 (1H, d, J=7.93 Hz), 5.13 (2H, s), 1.71 (9H, s).

### Step F—Synthesis of Compound 36G

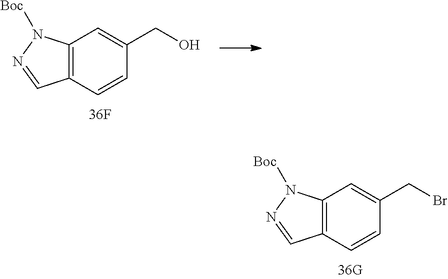

A solution of alcohol 36F (160 mg; 0.644 mmol) in dry chloroform (12 mL) was placed in an ice-water bath and treated with pyridine (4.0 eq, 0.208 mL, d 0.978) and a solution of thionyl bromide (1.2 eq, 0.060 mL, d 2.683) in 1 mL of chloroform. The ice-water bath was removed and the reaction mixture was stirred at room temp for 30 min. TLC (30% ethyl acetate in hexanes) showed about 40% conversion and more thionyl bromide was added (0.2 eq). The mixture was heated to 70° C. for 10 min. Upon cooling the mixture was diluted with ethyl acetate (30 mL) and washed with aqueous saturated sodium bicarbonate (5 mL), aqueous sodium hydrogen sulfate (5 mL) and brine (5 mL). The organic layer was dried over magnesium sulfate, filtered and concentrated in rotavap. The residue was purified on a Biotage 25-S silica gel column (gradient: 0 to 40% ethyl acetate in hexanes) to give the product 36G (76 mg; 38%) as a colorless oil along with unreacted starting material (25 mg; 24%). ¹H-NMR (CDCl₃; 400 MHz): δ 8.23 (1H, s), 8.14 (1H, s), 7.72 (1H, d, J=8.54 Hz), 7.32 (1H, dd, J=1.22, 8.54 Hz), 5.21 (1H, d, J=12.20 Hz), 5.09 (1H, d, J=12.20 Hz), 1.71 (9H, s).

### Example 37

### Preparation of Intermediate Compound 37C

### Step A—Synthesis of Compound 37B

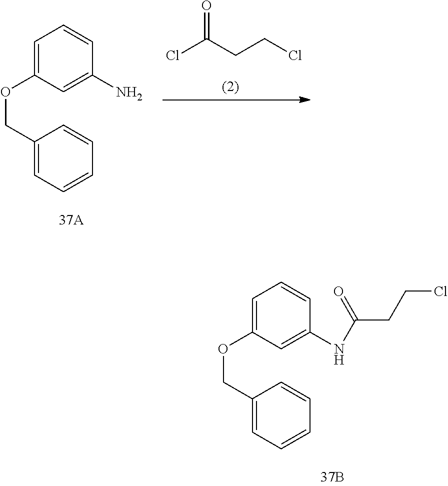

Compound 37A (commercially available) (10.0 g, 50.25 mmol) was dissolved in water at room temperature and to resulting suspension K₂CO₃ (3.8 g, 27.64 mmol) was added. 3-Chloro propionylchloride (7.0 g, 55.28 mmol) was added dropwise for 30 minutes and stirred for 2 hours at RT. The precipitate was filtered and washed with water, 1 N HCl, dried at 50° C. under vacuum overnight to give 7.2 g of the product 37B.

### Step B—Synthesis of Compound 37C

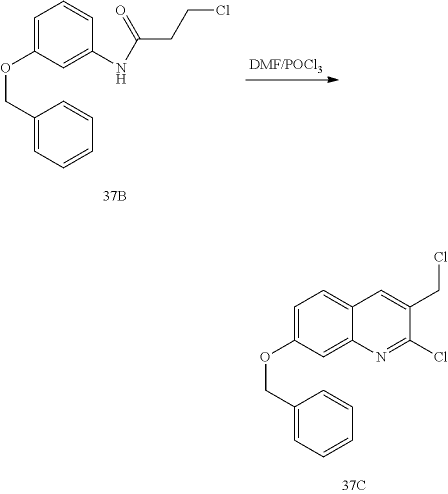

To N,N-Dimethylformamide (3.6 g, 49.66 mmol) at 0° C. was added drop wise POCl₃ (26.6 g, 173.8 mmol) and stirred for 60 minutes, white precipitate was formed. The 7.2 g of the compound 37B was added by portion in reaction mixture and stirred for 24 hours at room temperature. Reaction mixture was diluted with ethyl acetate and slowly added to a beaker with ice, after ice was melted, organic layer was separated and washed with 0.5 N NaOH and water, brine, dried over sodium sulfate, and concentrated in vacuum, purified using flash chromatography, to provide compound 37C (5.5 g, 34% after two steps). M.S. found: 318.04 (M+H)⁺.

### Example 38

### Preparation of Intermediate Compound 38E

### Step A—Synthesis of Compound 38B

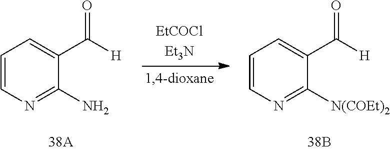

To a solution of 38A (7.2 g, 58.8 mmol) in 1,4-dioxane (39 mL) at 0° C. was added propionyl chloride (37.8 ml, 176.5 mmol) and Et₃N (24.6 mL, 176.5 mmol) with stirring. The reaction mixture was stirred at room temperature for overnight. The solvent was removed under reduced pressure, and the resulting residue was taken up in EtOAc. The organic phase was washed with water, dried over MgSO₄, filtered, and concentrated in vacuo to give a crude residue of 38B.

### Step B—Synthesis of Compound 38C

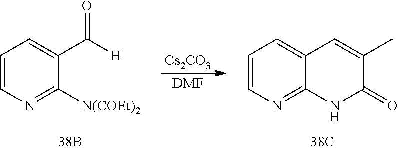

To a suspension of 38B (crude residue from above) in DMF (60 mL) was added cesium carbonate (38 g, 117.6 mmol), and the resulting mixture was heated at 65° C. for overnight. Reaction was cooled to room temperature, and the bulk of DMF was removed under reduced pressure. Water was then added to the crude residue and the mixture was filtered. The filter-cake was washed with water and EtOAc. 5.2 g of 38C was collected as a pale yellow solid.

### Step C—Synthesis of Compound 38D

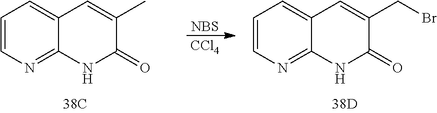

To a suspension of 38C (0.8 g, 5 mmol) in CCl₄ (25 mL) was added NBS (38 g, 117.6 mmol), and benzoyl peroxide (61 mg, 0.25 mmol), and the resulting mixture was then heated at 90° C. for 4 hours. Cooled the reaction to room temperature, and 300 mL of CH₂Cl₂ was added. The mixture was filtered, and filtrate was dried over MgSO₄, filtered, and concentrated in vacuo to give 2 g of crude residue of 38D.

### Step D—Synthesis of Compound 38E

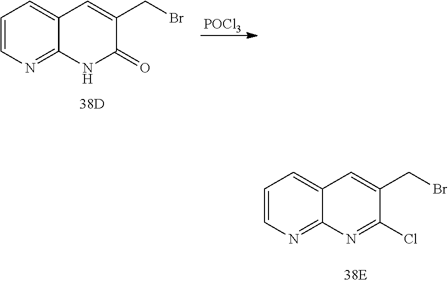

POCl₃ was added to a 100 mL round bottom flask containing crude 38D. The resulting suspension was then heated at 88° C. for 4 hours. Cooled the reaction to room temperature, and then poured into a 1 liter beaker containing ice. The resulting solution was neutralized to ph 8 using 6 N NaOH solution. Solid that precipitated from the solution was collected to give 0.82 g of crude residue which was purified using column chromatography on silica gel (ISCO Combi-Flash Rf; gradient: 5 to 50% ethyl acetate in hexanes) to provide 330 mg of compound 38E.

### Example 39

### Preparation of Intermediate Compound 39D

### Step A—Synthesis of Compound 39B

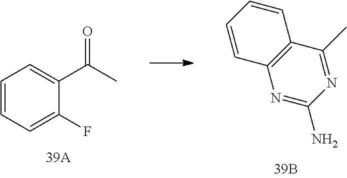

A mixture of ortho-fluoroacetophenone (39A, 3.45 g; 25 mmol) and guanidine carbonate (2 eq; 9.0 g) was prepared in 250 mL of N,N-dimethyl acetamide, set to stir, and heated at 135° C. under nitrogen purge overnight. The solvent was removed under reduced pressure and diluted with ethyl acetate (600 mL). The solution was washed with water (2×100 mL) and brine (40 mL). The organic layer was separated, dried over magnesium sulfate, filtered, and concentrated under reduced pressure. The solid was dissolved in methylene dichloride, loaded on silica gel and dried under reduced pressure. The material was purified on ISCO (80 g column; 0-70% THF in Hexanes). Fractions containing product were collected and concentrated under reduced pressure to afford product 39B as a crème colored solid (880 mg; 22%)

### Step B—Synthesis of Compound 39C

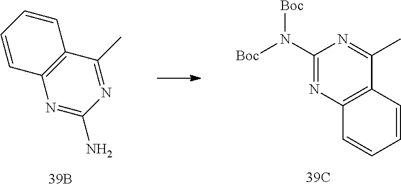

A solution of 4-Methyl-quinazolin-2-ylamine 39B (640 mg; 4.02 mmol) in 10 mL of dry acetonitrile was treated with a solution of Boc-anhydride (2.5 eq; 2.19 g) in 10.0 mL of dry acetonitrile. The resulting solution was treated with DMAP (0.2 eq; 98.2 mg). The mixture was set to stir overnight. TLC (50% THF in hexanes) showed a complete reaction. The mixture was diluted with ethyl acetate (500 mL) and washed with water (3×30 mL), and Brine (40 mL). The organic layer was dried over magnesium sulfate, filtered and concentrated in rotavap. The residue was adsorbed on silica gel and purified on an ISCO column (120 g) (0% to 60% THF in hexanes). The fractions with product were collected and concentrated under reduced pressure to afford product 39C as a light yellow-white solid (1.3 g; 90%).

### Step C—Synthesis of Compound 39D

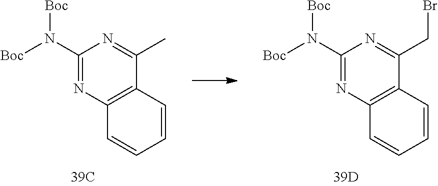

Intermediate 39C (1.11 g; 3.09 mmol), N-Bromosuccinimide (1.05 eq; 577 mg), and benzoyl peroxide (0.1 eq; 75 mg) were combined in round bottom and diluted with dry carbon tetrachloride (31 mL). The reaction was stirred at room temperature for 10 minutes and then heated at reflux overnight. TLC (30% ethyl acetate in hexanes) revealed the reaction has partially progressed. The reaction mixture was concentrated under reduced pressure, diluted with ethyl acetate (300 mL), and washed with sat. aqueous sodium bicarbonate (40 mL) and brine (40 mL), dried over magnesium sulfate, filtered, concentrated under reduced pressure, diluted with methylene dichloride, adsorbed onto silica gel, and purified on ISCO (25-M Column; 0-40% ethyl acetate in hexanes). The fractions containing product were concentrated under reduced pressure and afforded product as a clear oil in a 2:1 mixture of pure product 391) and starting material (Total : 440 mg; 33%).

### Example 40

### Preparation of Intermediate Compound 40C

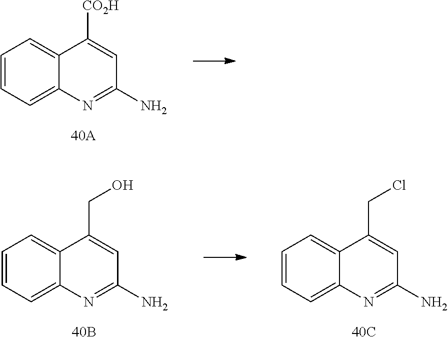

The starting materials 40A (2.0 g, 10.6 mmol), lithium aluminum hydride (2.0 g, 52.7 mmol), and THF (100 ml) were added to a 250 ml round-bottomed flask. The resulting suspension was stirred at room temperature for 18 hours. The reaction was quenched with 10 ml of saturated ammonium chloride solution followed by 200 ml of ethyl acetate. After filtration, the organic layer was washed with brine (2×100 ml), dried over sodium sulfate, and concentrated under vacuum to provide 40B as a yellowish solid (1.05 g, 59%).

A 250 ml round-bottomed flask was charged with 40B (1.05 g, 6.03 mmol) and thionyl chloride (10 ml). The resulting mixture was stirred at 60° C. for 4 hours before cooled to room temperature. After removal of excess of thionyl chloride, the residue was dried under vacuum to afford 40C as an orange solid (1.45 g). This crude material was used without further purification.

### Example 41

### Preparation of Intermediate Compound 41G

### Step A—Synthesis of Compound 41B

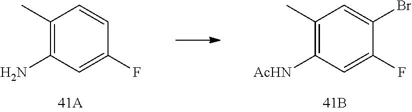

A solution of 5-fluoro-2-methylaniline (41A, 25 g, 200 mmol) in toluene (250 mL) was treated with acetic anhydride (25 mL. 226 mmol) heated at reflux for 1 h. The reaction mixture was cooled when a colorless solid precipitated out which was filtered and washed with a mixture of ether and hexanes. The colorless solid was taken in acetic acid (150 mL) and treated dropwise with a solution of bromine (9.6 mL, 186 mmol) in acetic acid (20 mL) and stirred at rt. for 12 h. The solution was diluted with water and the solid separating out was filtered and washed to yield N-(4-bromo-5-fluoro-2-methylphenyl)acetamide (41B, 40 g) as a colorless solid.

### Step B—Synthesis of Compound 29C

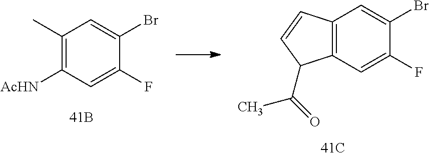

A solution of N-(4-bromo-5-fluoro-2-methylphenyl)acetamide (41B, 10.00 g, 40.64 mmol) in chloroform (100 mL) was treated with acetic anhydride (11.5 mL, 122.0 mmol), potassium acetate (8.00 g, 81.5 mmol), and 18-Crown-6 (540.00 mg, 2.0430 mmol) and then with isoamyl nitrite (12.3 mL, 871 mmol) and heated at 65° C. for 12 h. The reaction mixture was cooled to room temperature and treated with EtOAc (500 mL), washed with water, dried (MgSO₄), filtered, and then concentrated in vacuo. A pale yellow solid of 1-(5-bromo-6-fluoro-1H-indazol-1-yl)ethanone (29C) precipitated out. The initial filtrate was concentrated and the residue was purified by chromatography (SiO₂, EtOAc/Hexanes) to yield more of product 41C.

### Step C—Synthesis of Compound 41D

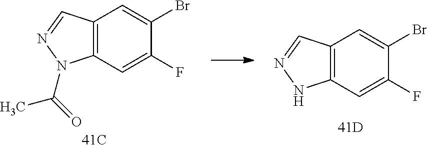

A solution of 1-(5-bromo-6-fluoro-1H-indazol-1-yl)ethanone (41C, 5.0 g, 19.5 mmol) was treated with aq HCl (3M soln., 100 mL) and methanol (20 mL) and heated at 90° C. for 3 h, when the reaction turns homogenous. The reaction mixture was cooled to room temperature and basified with aq. NaOH. A colorless solid precipitated out which was filtered and dried to yield 5-bromo-6-fluoro-1H-indazole (41D)

### Step D—Synthesis of Compound 41E

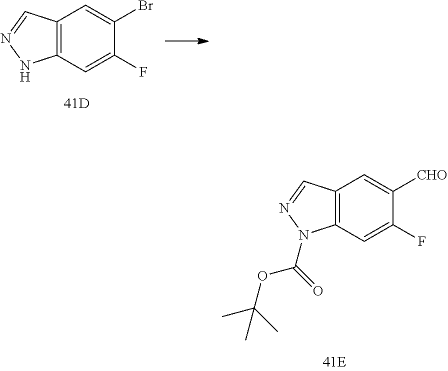

A solution of 5-bromo-6-fluoro-1H-indazole (41D, 3.50 g, 16.28 mmol) in tetrahydrofuran (200.00 mL) was treated with sodium hydride (60% in mineral oil, 1.172 g) at 0° C. and stirred-at rt. for 20 min. The reaction mixture was cooled to −78° C. (dry ice and acetone) and treated with 2.5 M of n-butyl lithium in hexane (8.2 mL, 20.3 mmol) dropwise. The reaction mixture was stirred at that temperature for 20 min and treated with DMF (5.06 mL, 65.11 mmol). The reaction mixture was slowly warmed to room temperature when the viscous solution turn fluidic and stirring was efficient. Analysis of TLC (40% EtOAc/Hexanes) indicated complete conversion of starting material to product. The reaction mixture was acidified with aq. HCl taken up in EtOAc (500 mL) washed with aq. HCl (100 mL), brine (100 mL), dried (MgSO₄), filtered, concentrated in vacuo and used as it is in next step. A solution of product 6-fluoro-1H-indazole-5-carbaldehyde (2.3 g) in THF (100 mL) was treated with di-tert-butyldicarbonate (3.56 g, 16.28 mmol) and DMAP (300 mg) and stirred at room temperature for 3 h. The reaction mixture was concentrated in vacuo and the residue was purified by chromatography (SiO₂, EtOAc/Hexanes gradient 0-40%) to yield [2e] tert-butyl 6-fluoro-5-formyl-1H-indazole-l-carboxylate (41E, 3.5 g; Yield =81%) as a colorless solid.

### Step E—Synthesis of Compound 41F

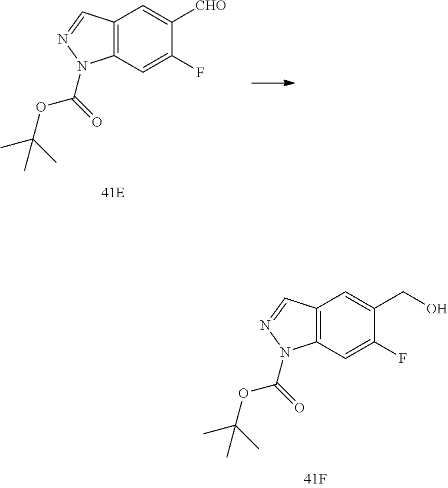

A solution of tert-butyl 6-fluoro-5-formyl-1H-indazole-1-carboxylate (29E, 3.55 g, 13.4 mmol) in methanol (50.00 mL) was treated with NaBH₄ (1.02 g, 26.9 mmol) at 0° C. and stirred for 1 h. The reaction mixture was diluted with water and EtOAc (500 mL). The organic layer was separated and washed with aq. HCl (1M, 200 mL), aq. NaOH (1M, 200 mL) brine (200 mL) dried (MgSO₄), filtered, concentrated in vacuo and residue was purified by chromatography (SiO₂, EtOAc/hexanes) to yield tert-butyl 5-(hydroxymethyl)-6-fluoro-1H-indazole-1-carboxylate (29F, 3.00 g; Yield=83.9%) as a colorless solid.

### Step F—Synthesis of Compound 41G

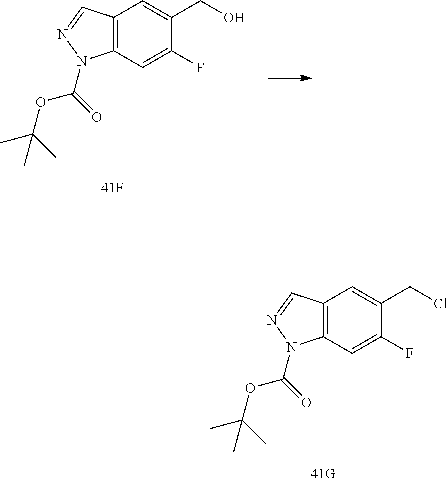

A solution of tert-butyl 5-(hydroxymethyl)-6-fluoro-1H-indazole-l-carboxylate (29F, 3.0 g, 11.27 mmol) in methylene chloride (50.00 mL, 780.0 mmol) at rt. was treated with pyridine (4.56 mL, 56.33 mmol) and methanesulfonyl chloride (1.31 mL) and stirred at rt. for 16 h. The reaction mixture was concentrated in vacuo and the residue was dissolved in EtOAc (300 mL) washed with aq HCl (100 mL), brine (100 mL), dried (MgSO₄), filtered, concentrated in vacuo, and purified by chromatography (SiO₂, EtOAc/Hexanes) to yield tert-butyl 5-(chloromethyl)-6-fluoro-1H-indazole-1-carboxylate (41G, 1.9 g; Yield=59%)

### Example 42

### HCV NS5B Polymerase Inhibition Assay

An in vitro transcribed heteropolymeric RNA known as D-RNA or DCoH has been shown to be an efficient template for HCV NS5B polymerase (S.-E. Behrens et al., EMBO J. 15: 12-22 (1996); WO 96/37619). A chemically synthesized 75-mer version, designated DCoH75, whose sequence matches the 3’-end of D-RNA, and DCoH75ddC, where the 3′-terminal cytidine of DCoH75 is replaced by dideoxycytidine, were used for assaying the NS5B enzyme activity as described in Ferrari et al., 12^{th} International Symposium on HCV and Related Viruses, P-306 (2005). A soluble C-terminal 21-amino acid truncated NS5B enzyme form (NS5BDeltaCT21) was produced and purified from Escherichia coli as C-terminal polyhistidine-tagged fusion protein as described in Ferrari et al., J. Virol. 73:1649-1654 (1999). A typical assay contained 20 mM Hepes pH 7.3, 10 mM MgCl₂, 60 mM NaCl, 100 μg/ml BSA, 20 units/ml RNasin, 7.5 mM DTT, 0.1 μM ATP/GTP/UTP, 0.026 μM CTP, 0.25 mM GAU, 0.03 μM RNA template, 20 μCi/ml [³³P]-CTP, 2% DMSO, and 30 or 150 nM NS5B enzyme. Reactions were incubated at 22° C. for 2 hours, then stopped by adding 150 mM EDTA, washed in DE81 filter plate in 0.5M di-basic sodium phosphate buffer, pH 7.0, and counted using Packard TopCount after the addition of scintillation cocktail. Polynucleotide synthesis was monitored by the incorporation of radiolabeled CTP. The effect of the Tetracyclic Indole Derivatives on the polymerase activity was evaluated by adding various concentrations of a Tetracyclic Indole Derivative, typically in 10 serial 2-fold dilutions, to the assay mixture. The starting concentrations of the indole derivatives ranged from 200 μM to 1 μM. An IC₅₀ value for the inhibitor, defined as the compound concentration that provides 50% inhibition of polymerase activity, was determined by fitting the cpm data to the Hill equation Y=100/(1+10̂((LogIC50-X)*HillSlope)), where X is the logarithm of compound concentration, and Y is the % inhibition. Ferrari et al., 12^{th} International Symposium on HCV and Related Viruses, P-306 (2005) described in detail this assay procedure. It should be noted that such an assay as described is exemplary and not intended to limit the scope of the invention. The skilled practitioner can appreciate that modifications including but not limited to RNA template, primer, nucleotides, NS5B polymerase form, buffer composition, can be made to develop similar assays that yield the same result for the efficacy of the compounds and compositions described in the invention.

NS5B polymerase inhibition data for selected Tetracyclic Indole Derivatives of the present invention was obtained using the above method and calculated IC₅₀ values ranged from about 1 μM to about 14000 μM.

### Example 43

### Cell-based HCV Replicon Assay

To measure cell-based anti-HCV activity of the a Tetracyclic Indole Derivative, replicon cells were seeded at 5000 cells/well in 96-well collagen I-coated Nunc plates in the presence of the Tetracyclic Indole Derivative. Various concentrations of a Tetracyclic Indole Derivative, typically in 10 serial 2-fold dilutions, were added to the assay mixture, the starting concentration of the compound ranging from 250 μM to 1 μM. The final concentration of DMSO was 0.5%, fetal bovine serum was 5%, in the assay media. Cells were harvested on day 3 by the addition of 1× cell lysis buffer (Ambion cat #8721). The replicon RNA level was measured using real time PCR (Taqman assay). The amplicon was located in 5B. The PCR primers were: 5B.2F, ATGGACAGGCGCCCTGA; 5B.2R, TTGATGGGCAGCTTGGTTTC; the probe sequence was FAM-labeled CACGCCATGCGCTGCGG. GAPDH RNA was used as endogenous control and was amplified in the same reaction as NS5B (multiplex PCR) using primers and VIC-labeled probe recommended by the manufacturer (PE Applied Biosystem). The real-time RT-PCR reactions were run on ABI PRISM 7900HT Sequence Detection System using the following program: 48° C. for 30 min, 95° C. for 10 min, 40 cycles of 95° C. for 15 sec, 60° C. for 1 min. The OCT values (CT_{5B}-CT_{GAPDH}) were plotted against the concentration of test compound and fitted to the sigmoid dose-response model using XLfit4 (MDL). EC₅₀ was defined as the concentration of inhibitor necessary to achieve ΔCT=1 over the projected baseline; EC₉₀ the concentration necessary to achieve ΔCT=3.2 over the baseline. Alternatively, to quantitate the absolute amount of replicon RNA, a standard curve was established by including serially diluted T7 transcripts of replicon RNA in the Taqman assay. All Taqman reagents were from PE Applied Biosystems. Such an assay procedure was described in detail in e.g. Malcolm et al., Antimicrobial Agents and Chemotherapy 50: 1013-1020 (2006).

HCV Replicon assay data for selected Tetracyclic Indole Derivatives of the present invention was obtained using the above method and calculated EC₅₀ values ranged from about 1 μM to about 14000 μM.

### Uses of the Tetracyclic Indole Derivatives

The Tetracyclic Indole Derivatives are useful in human and veterinary medicine for treating or preventing a viral infection or a virus-related disorder in a patient. In accordance with the invention, the Tetracyclic Indole Derivatives can be administered to a patient in need of treatment or prevention of a viral infection or a virus-related disorder.

Accordingly, in one embodiment, the invention provides methods for treating a viral infection in a patient comprising administering to the patient an effective amount of at least one Tetracyclic Indole Derivative or a pharmaceutically acceptable salt, solvate, ester or prodrug thereof. In another embodiment, the invention provides methods for treating a virus-related disorder in a patient comprising administering to the patient an effective amount of at least one Tetracyclic Indole Derivative or a pharmaceutically acceptable salt, solvate, ester or prodrug thereof.

### Treatment or Prevention of a Viral Infection

The Tetracyclic Indole Derivatives can be used to treat or prevent a viral infection. In one embodiment, the Tetracyclic Indole Derivatives can be inhibitors of viral replication. In a specific embodiment, the Tetracyclic Indole Derivatives can be inhibitors of HCV replication. Accordingly, the Tetracyclic Indole Derivatives are useful for treating viral diseases and disorders related to the activity of a virus, such as HCV polymerase.

Examples of viral infections that can be treated or prevented using the present methods, include but are not limited to, hepatitis A infection, hepatitis B infection and hepatitis C infection.

In one embodiment, the viral infection is hepatitis C infection.

In one embodiment, the hepatitis C infection is acute hepatitis C. In another embodiment, the hepatitis C infection is chronic hepatitis C.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a, 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al, J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Treatment or Prevention of a Virus-Related Disorder

The Tetracyclic Indole Derivatives can be used to treat or prevent a virus-related disorder. Accordingly, the Tetracyclic Indole Derivatives are useful for treating disorders related to the activity of a virus, such as liver inflammation or cirrhosis. Virus-related disorders include, but are not limited to, RNA-dependent polymerase-related disorders and disorders related to HCV infection.

### Treatment or Prevention of a RNA-Dependent Polymerase-Related Disorder

The Tetracyclic Indole Derivatives are useful for treating or preventing a RNA dependent polymerase (RdRp) related disorder in a patient. Such disorders include viral infections wherein the infective virus contain a RdRp enzyme.

Accordingly, in one embodiment, the present invention provides a method for treating a RNA dependent polymerase-related disorder in a patient, comprising administering to the patient an effective amount of at least one Tetracyclic Indole Derivative or a pharmaceutically acceptable salt, solvate, ester or prodrug thereof.

### Treatment or Prevention of a Disorder Related to HCV Infection

The Tetracyclic Indole Derivatives can also be useful for treating or preventing a disorder related to an HCV infection. Examples of such disorders include, but are not limited to, cirrhosis, portal hypertension, ascites, bone pain, varices, jaundice, hepatic encephalopathy, thyroiditis, porphyria cutanea tarda, cryoglobulinemia, glomerulonephritis, sicca syndrome, thrombocytopenia, lichen planus and diabetes mellitus.

Accordingly, in one embodiment, the invention provides methods for treating an HCV-related disorder in a patient, wherein the method comprises administering to the patient a therapeutically effective amount of at least one Tetracyclic Indole Derivative, or a pharmaceutically acceptable salt, solvate, ester or prodrug thereof.

### Combination Therapy

In another embodiment, the present methods for treating or preventing a viral infection can further comprise the administration of one or more additional therapeutic agents which are not Tetracyclic Indole Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Tetracyclic Indole Derivative, or a pharmaceutically acceptable salt, solvate, ester or prodrug thereof, and (ii) at least one other antiviral agent that is other than a Tetracyclic Indole Derivative, wherein the amounts administered are together effective to treat or prevent a viral infection.

When administering a combination therapy of the invention to a patient, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Tetracyclic Indole Derivative and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

In one embodiment, the at least one Tetracyclic Indole Derivative is administered during at time when the additional antiviral agent(s) exert their prophylactic or therapeutic effect, or vice versa.

In another embodiment, the at least one Tetracyclic Indole Derivative and the additional antiviral agent(s) are administered in doses commonly employed when such agents are used as monotherapy for treating a viral infection.

In another embodiment, the at least one Tetracyclic Indole Derivative and the additional antiviral agent(s) are administered in doses lower than the doses commonly employed when such agents are used as monotherapy for treating a viral infection.

In still another embodiment, the at least one Tetracyclic Indole Derivative and the additional antiviral agent(s) act synergistically and are administered in doses lower than the doses commonly employed when such agents are used as monotherapy for treating a viral infection.

In one embodiment, the at least one Tetracyclic Indole Derivative and the additional antiviral agent(s) are present in the same composition. In one embodiment, this composition is suitable for oral administration. In another embodiment, this composition is suitable for intravenous administration.

Viral infections and virus-related disorders that can be treated or prevented using the combination therapy methods of the present invention include, but are not limited to, those listed above.

In one embodiment, the viral infection is HCV infection.

The at least one Tetracyclic Indole Derivative and the additional antiviral agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of at least one Tetracyclic Indole Derivative and the additional antiviral agent(s) may inhibit the resistance of a viral infection to these agents.

Non-limiting examples of other therapeutic agents useful in the present compositions and methods include an HCV polymerase inhibitor, an interferon, a viral replication inhibitor, an antisense agent, a therapeutic vaccine, a viral protease inhibitor, a virion production inhibitor, an antibody therapy (monoclonal or polyclonal), and any agent useful for treating an RNA-dependent polymerase-related disorder.

In one embodiment, the other antiviral agent is a viral protease inhibitor.

In another embodiment, the other antiviral agent is an HCV protease inhibitor.

In another embodiment, the other antiviral agent is an interferon.

In still another embodiment, the other antiviral agent is a viral replication inhibitor.

In another embodiment, the other antiviral agent is an antisense agent.

In another embodiment, the other antiviral agent is a therapeutic vaccine.

In a further embodiment, the other antiviral agent is an virion production inhibitor.

In another embodiment, the other antiviral agent is antibody therapy.

In another embodiment, the other antiviral agents comprise a protease inhibitor and a polymerase inhibitor.

In still another embodiment, the other antiviral agents comprise a protease inhibitor and an immunosuppressive agent.

In yet another embodiment, the other antiviral agents comprise a polymerase inhibitor and an immunosuppressive agent.

In a further embodiment, the other antiviral agents comprise a protease inhibitor, a polymerase inhibitor and an immunosuppressive agent.

In another embodiment the other agent is ribavirin.

HCV polymerase inhibitors useful in the present methods and compositions include, but are not limited to VP-19744 (Wyeth/ViroPharma), HCV-796 (Wyeth/ViroPharma), NM-283 (Idenix/Novartis), R-1626 (Roche), MK-0608 (Merck), A848837 (Abbott), GSK-71185 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Interferons useful in the present methods and compositions include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™), interferon alpha-2c (Berofor Alpha™, Boehringer Ingelheim, Ingelheim, Germany), interferon alpha fusion polypeptides, or consensus interferon as defined by determination of a consensus sequence of naturally occurring interferon alphas (Infergen™, Amgen, Thousand Oaks, Calif.).

Antibody therapy agents useful in the present methods and compositions include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like). Viral protease inhibitors useful in the present methods and compositions include, but are not limited to, NS3 serine protease inhibitors (including, but are not limited to, those disclosed in U.S. Pat. Nos. 7,012,066, 6,914,122, 6,911,428, 6,846,802, 6,838,475, 6,800,434, 5,017,380, 4,933,443, 4,812,561 and 4,634,697; and U.S. Patent Publication Nos. US20020160962, US20050176648 and US20050249702), HCV protease inhibitors (e.g., SCH503034 (Schering-Plough), VX-950 (Vertex), GS-9132 (Gilead/Achillion), ITMN-191 (InterMune/Roche)), amprenavir, atazanavir, fosemprenavir, indinavir, lopinavir, ritonavir, nelfinavir, saquinavir, tipranavir and TMC114.

Viral replication inhibitors useful in the present methods and compositions include, but are not limited to, NS3 helicase inhibitors, NS5A inhibitors, ribavirin, viramidine, A-831 (Arrow Therapeutics); an antisense agent or a therapeutic vaccine.

In one embodiment, viral replication inhibitors useful in the present methods and compositions include, but are not limited to, NS3 helicase inhibitors or NS5A inhibitors.

Examples of protease inhibitors useful in the present methods include, but are not limited to, an HCV protease inhibitor and a NS-3 serine protease inhibitor.

Examples of HCV protease inhibitors useful in the present methods include, but are not limited to, those disclosed in Landro et al., Biochemistry, 36(31):9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); Bio World Today, 9(217):4 (Nov. 10, 1998); and International Publication Nos. WO 98/14181; WO 98/17679, WO 98/17679, WO 98/22496 and WO 99/07734.

Further examples of protease inhibitors useful in the present methods include, but are not limited to,

Additional examples of other therapeutic agents useful in the present methods include, but are not limited to, Levovirin™ (ICN Pharmaceuticals, Costa Mesa, Calif.), VP 50406™ (Viropharma, Incorporated, Exton, Pa.), ISIS 14803™ (ISIS Pharmaceuticals, Carlsbad, Calif.), Heptazyme™ (Ribozyme Pharmaceuticals, Boulder, Colo.), VX-950™ (Vertex Pharmaceuticals, Cambridge, Mass.), Thymosin™ (SciClone Pharmaceuticals, San Mateo, Calif.), Maxamine™ (Maxim Pharmaceuticals, San Diego, Calif.), NKB-122 (JenKen Bioscience Inc., North Carolina), mycophenolate mofetil (Hoffman-LaRoche, Nutley, N.J.).

The doses and dosage regimen of the other agents used in the combination therapies of the present invention for the treatment or prevention of a viral infection can be determined by the attending clinician, taking into consideration the the approved doses and dosage regimen in the package insert; the age, sex and general health of the patient; and the type and severity of the viral infection or related disease or disorder. When administered in combination, the Tetracyclic Indole Derivative(s) and the other agent(s) for treating diseases or conditions listed above can be administered simultaneously (i.e., in the same composition or in separate compositions one right after the other) or sequentially. This is particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the at least one Tetracyclic Indole Derivative and the additional antiviral agent(s), when administered as combination therapy, can range from about 0.1 to about 2000 mg per day, although variations will necessarily occur depending on the target of the therapy, the patient and the route of administration. In one embodiment, the dosage is from about 10 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when the other therapeutic agent is INTRON-A interferon alpha 2b (commercially available from Schering-Plough Corp.), this agent is administered by subcutaneous injection at 3MIU(12 mcg)/0.5 mL/TIW is for 24 weeks or 48 weeks for first time treatment.

In another embodiment, when the other therapeutic agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when the other therapeutic agent is ROFERON A inteferon alpha 2a (commercially available from Hoffmann-La Roche), this agent is administered by subcutaneous or intramuscular injection at 3MIU(11.1 mcg/mL)/TIW for at least 48 to 52 weeks, or alternatively 6MIU/TIW for 12 weeks followed by 3MIU/TIW for 36 weeks.

In still another embodiment, when the other therapeutic agent is PEGASUS interferon alpha 2a pegylated (commercially available from Hoffmann-La Roche), this agent is administered by subcutaneous injection at 180 mcg/1 mL or 180 mcg/0.5 mL, once a week for at least 24 weeks.

In yet another embodiment, when the other therapeutic agent is INFERGEN interferon alphacon-1 (commercially available from Amgen), this agent is administered by subcutaneous injection at 9mcg/TIW is 24 weeks for first time treatment and up to 15 mcg/TIW for 24 weeks for non-responsive or relapse treatment.

In a further embodiment, when the other therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

### Compositions and Administration

Due to their activity, the Tetracyclic Indole Derivatives are useful in veterinary and human medicine. As described above, the Tetracyclic Indole Derivatives are useful for treating or preventing a viral infection or a virus-related disorder in a patient in need thereof.

When administered to a patient, the IDs can be administered as a component of a composition that comprises a pharmaceutically acceptable carrier or vehicle. The present invention provides pharmaceutical compositions comprising an effective amount of at least one Tetracyclic Indole Derivative and a pharmaceutically acceptable carrier. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

The Tetracyclic Indole Derivatives of the present invention may also be deliverable transdermally. The transdermal compositions can take the form of creams, lotions, aerosols and/or emulsions and can be included in a transdermal patch of the matrix or reservoir type as are conventional in the art for this purpose.

Additionally, the compositions of the present invention may be formulated in sustained release form to provide the rate controlled release of any one or more of the components or active ingredients to optimize the therapeutic effects, i.e. anti-inflammatory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Tetracyclic Indole Derivatives are administered orally.

In another embodiment, the one or more Tetracyclic Indole Derivatives are administered intravenously.

In another embodiment, the one or more Tetracyclic Indole Derivatives are administered topically.

In still another embodiment, the one or more Tetracyclic Indole Derivatives are administered sublingually.

In one embodiment, a pharmaceutical preparation comprising at least one Tetracyclic Indole Derivative is in unit dosage form. In such form, the preparation is subdivided into unit doses containing appropriate quantities of the active component, e.g., an effective amount to achieve the desired purpose.

Compositions can be prepared according to conventional mixing, granulating or coating methods, respectively, and the present compositions can contain, in one embodiment, from about 0.1% to about 99% of the Tetracyclic Indole Derivative(s) by weight or volume. In various embodiments, the the present compositions can contain, in one embodiment, from about 1% to about 70% or from about 5% to about 60% of the Tetracyclic Indole Derivative(s) by weight or volume.

The quantity of Tetracyclic Indole Derivative in a unit dose of preparation may be varied or adjusted from about 0.1 mg to about 2000 mg. In various embodiment, the quantity is from about 1 mg to about 2000 mg, 100 mg to about 200 mg, 500 mg to about 2000 mg, 100 mg to about 1000 mg, and 1 mg to about 500 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Tetracyclic Indole Derivatives will be regulated according to the judgment of the attending clinician considering such factors as age, condition and size of the patient as well as severity of the symptoms being treated. Generally, a total daily dosage of the Tetracyclic Indole Derivatives range from about 0.1 to about 2000 mg per day, although variations will necessarily occur depending on the target of the therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Tetracyclic Indole Derivative or a pharmaceutically acceptable salt, solvate, ester or prodrug thereof; (ii) one or more additional therapeutic agents that are not a Tetracyclic Indole Derivative; and (iii) a pharmaceutically acceptable carrier, wherein the amounts in the composition are together effective to treat a viral infection or a virus-related disorder.

### Kits

In one aspect, the present invention provides a kit comprising a therapeutically effective amount of at least one Tetracyclic Indole Derivative, or a pharmaceutically acceptable salt, solvate, ester or prodrug of said compound and a pharmaceutically acceptable carrier, vehicle or diluent.

In another aspect the present invention provides a kit comprising an amount of at least one Tetracyclic Indole Derivative, or a pharmaceutically acceptable salt, solvate, ester or prodrug of said compound and an amount of at least one additional therapeutic agent listed above, wherein the amounts of the two or more ingredients result in a desired therapeutic effect.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparant to those skilled in the art and are intended to fall within the scope of the appended claims.

A number of references have been cited herein, the entire disclosures of which are incorporated herein by reference.

What is claimed is:

1. A compound having the formula: 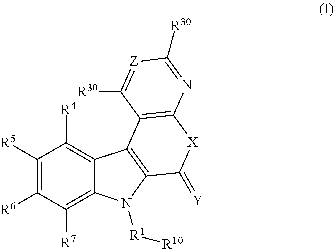
or a pharmaceutically acceptable salt, solvate, ester or prodrug thereof.
wherein:
X is —O—, —S—, —NH—, —N(R⁹)—, —OC(R⁸)₂O— or —OC(R⁸)₂N(R⁹)—;
Y is ═O, ═NH, ═NR⁹, ═NSOR¹¹, ═NSO₂R¹¹ or ═NSO₂N(R¹¹)₂;
Z is —N— or —C(R³¹)—;
R¹ is a bond, —[C(R¹²)₂]ᵣ—, —[C(R¹²)₂]ᵣ—O—[C(R¹²)₂]_{q}—, —[C(R¹²)₂]ᵣ—N(R⁹)—[C(R¹²)₂]_{q}—, —[C(R¹²)₂]_{q}—CH═CH—[C(R¹²)₂]_{q}—, —[C(R¹²)₂]_{q}—C≡C—[C(R¹²)₂]_{q}—, or —[C(R¹²)₂]_{q}—SO₂—[C(R¹²)₂]_{q}—;
R⁴, R⁵, R⁶ and R⁷ are each, independently, H, alkyl, alkenyl, alkynyl, aryl, —[C(R¹²)₂]_{q}-cycloalkyl, —[C(R¹²)₂]_{q}-cycloalkenyl, —C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —C(R¹²)₂]_{q}-hydroxyalkyl, halo, hydroxy, —OR⁹, —CN, —[C(R¹²)₂]_{q}—C(O)R⁸, —[C(R¹²)₂]_{q}—C(O)OR⁹, —[C(R¹²)₂]₁—C(O)N(R⁹)₂, —[C(R¹²)₂]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂R¹¹, —[C(R¹²)₂]_{q}—S(O)ₚR¹¹, —[C(R¹²)₂]_{q}—SO₂N(R⁹)₂—SO₂N(R⁹)C(O)N(R⁹)₂, or R⁴ and R⁵, together with the carbon atoms to which they are attached, join to form a 3- to 7-membered cyclic group, selected from cycloalkyl, heterocycloalkyl, aryl or heteroaryl, or R⁵, and R⁶, together with the carbon atoms to which they are attached, join to form a 3- to 7-membered cyclic group, selected from cycloalkyl, heterocycloalkyl, aryl or heteroaryl, or R⁶ and R⁷, together with the carbon atoms to which they are attached, join to form a 3- to 7-membered cyclic group, selected from cycloalkyl, heterocycloalkyl, aryl or heteroaryl;
each occurrence of R⁸ is independently H, alkyl, alkenyl, alkynyl, —[C(R¹²)₂]_{q}aryl, —[C(R¹²)₂]_{q}-cycloalkyl, —[C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, haloalkyl or hydroxyalkyl;
each occurrence of R⁹ is independently H, alkyl, alkenyl, alkynyl, —[C(R¹²)₂]_{q}aryl, —[C(R¹²)₂]_{q}-cycloalkyl, —C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, haloalkyl or hydroxyalkyl;
R¹⁰ is H, halo, cycloalkyl, cycloalkenyl, heterocycloalkyl, heterocycloalkenyl, aryl, heteroaryl, wherein a cycloalkyl, cycloalkenyl, heterocycloalkyl, heterocycloalkenyl, aryl or heteroaryl group can be optionally and independently substituted with up to 4 substituents, which are each independently selected from H, alkyl, alkenyl, alkynyl, aryl, —[C(R¹²)²]_{q}-cycloalkyl, —[C(R¹²)₂]_{q}-cycloalkenyl, —[C(R¹²)₂]_{q}-heterocycloakyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —O R⁹, —CN, —[C(R¹²)₂]_{q}—C(O)R⁸, —[C(R¹²)₂]_{q}—C(O)OR⁹, —[C(R¹²)₂]_{q}—C(O)N(R⁹)₂, —[C(R¹²)₂]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂R¹¹, —[C(R¹²)₂]_{q}—S(O)ₚR¹¹, —[C(R¹²)₂]_{q}—SO₂N(R⁹)₂ and —SO₂N(R⁹)C(O)N(R⁹)₂, such that when R^{I} is a bond, R¹⁰ is other than H;
each occurrence of R¹¹ is independently alkyl, aryl, cycloalkyl, cycloalkenyl, heterocycloalkyl, heterocycloalkenyl, heteroaryl, haloalkyl, hydroxy or hydroxyalkyl, wherein a cycloalkyl, cycloalkenyl, heterocycloalkyl, heterocycloalkenyl, aryl or heteroaryl group can be optionally and independently substituted with up to 4 substituents, which are each independently selected from -H, alkyl, alkenyl, alkynyl, aryl, —[C(R¹²)₂]_{q}-cycloalkyl, —[C(R¹²)₂]_{q}-cycloalkenyl, —[C(R¹²)₂]_{q}-heterocycloalkyl, —C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹, —CN, —[C(R¹²)₂]_{q}—C(O)R⁸, —[C(R¹²)₂]_{q}—C(O)OR⁹, —[C(R¹²)₂]_{q}—C(O)N(R⁹)₂, —[C(R¹²)₂]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹)₂, —[C(R¹²)₂]_{q}—NHSO₂alkyl, —[C(R¹²)₂]_{q}—NHSO₂cycloalkyl, —[C(R¹²)₂]—NHSO₂aryl, —[C(R¹²)₂]_{q}—SO₂N(R⁹)₂ and —SO₂N(R⁹)C(O)N(R⁹)₂;
each occurrence of R¹² is independently H, halo, —N(R⁹)₂, —OR⁹, alkyl, cycloalkyl, cycloalkenyl, heterocycloalkyl or heterocycloalkenyl, wherein a cycloalkyl, cycloalkenyl, heterocycloalkyl or heterocycloalkenyl group can be optionally and independently substituted with up to 4 substituents, which are each independently selected from alkyl, halo, haloalkyl, hydroxyalkyl, hydroxy, —CN, —C(O)alkyl, —C(O)Oalkyl, —C(O)NHalkyl, —C(O)N(alkyl)₂, —O—-alkyl, —NH₂, —NH(alkyl), —N(alkyl)₂, —NHC(O)alkyl, —NHSO₂alkyl, —SO₂alkyl or —SO₂NH— alkyl, or two germinal R¹² groups, together with the common carbon atom to which they are attached, join to form a 3- to 7-membered cycloalkyl, 3- to 7-membered heterocycloalkyl or C═O group;
each occurrence of R³⁰ is independently, H, alkyl, alkenyl, alkynyl, aryl, —[C(R¹²)₂]_{q}-cycloalkyl, —C(R¹²)₂]_{q}-cycloalkenyl, —[C(R¹²)₂]_{q}-heterocycloalkyl, —[C(R¹²)₂]_{q}-heterocycloalkenyl, —[C(R¹²)₂]_{q}-heteroaryl, —[C(R¹²)₂]_{q}-haloalkyl, —[C(R¹²)₂]_{q}-hydroxyalkyl, halo, hydroxy, —OR⁹, —CN, —[C(R¹²)₂]_{q}—C(O)R⁸, —[C(R¹²)₂]_{q}—C(O)OR⁹, —[C(R¹²)₂]_{q}—C(O)N(R⁹)₂, —[C(R¹²)₂]_{q}—OR⁹, —[C(R¹²)₂]_{q}—N(R⁹)₂, —[C(R¹²)₂]_{q}—NHC(O)R⁸, —[C(R¹²)₂]_{q}—NR⁸C(O)N(R⁹₂, —[C(R¹²)₂]_{q}—NHSO₂R¹¹, —[C(R¹²)₂]_{q}—S(O)ₚR¹¹, —[C(R¹²)₂]_{q}—SO₂N(R⁹)₂—SO₂N(R⁹)C(O)N(R⁹)₂, or any R³⁰ and R³¹, together with the carbon atoms to which they are attached, join to form a 3- to 7-membered cyclic group, selected from cycloalkyl, heterocycloalkyl, aryl and heteroaryl;
R³¹ is H, alkyl, alkenyl, alkynyl, aryl, —[C(R¹²)₂]_{q}-cycloalkyl, —C(R¹²)₂]_{q}-cycloalkenyl, —[C(R¹²)₂]_{q}-heterocycloalkyl, —[C(R¹²)₂]_{q}-heterocycloalkenyl, —C(R¹²)₂]_{q}-heteroaryl, —[C(R¹²)₂]_{q}-haloalkyl, —[C(R¹²)₂]_{q}hydroxyalkyl, halo, hydroxy, —OR⁹ or —CN;
each occurrence of p is independently 0, 1 or 2;
each occurrence of q is independently an integer ranging from 0 to 4; and
each occurrence of r is independently an integer ranging from 1 to 4.

2. The compound of claim 1, wherein X is —O—, —OCH(R⁸)O—, —NH—, or —OCH(R⁸)NH—.

3. The compound of claim 2, wherein Y is αO, ═NH, ═N(R⁹)SOR¹¹, ═N(R⁹)SO₂R¹¹ or ═N(R⁹)SO₂N(R¹¹)₂.

4. The compound of claim 1, wherein X is —O— and Y is ═O or ═N(R⁹)SO₂R¹¹.

5. The compound of claim 4, wherein R¹¹ is alkyl, cycloalkyl, haloalkyl or heterocycloalkyl and R⁹ is H, alkyl, cycloalkyl or heterocycloalkyl.

6. The compound of claim 4, wherein Z is (C)R³¹.

7. The compound of claim 4 wherein R¹ is —[C(R¹²)₂]ᵣ—.

8. The compound of claim 7 wherein R¹ is —CH₂—, —CH₂CH₂—, —CH(CH₃)— or

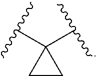

9. The compound of claim 8, wherein R⁴ and R⁷ are each independently H, alkyl, halo or hydroxy.

10. The compound of claim 8 wherein R⁵ is H, alkyl, —O-haloalkyl, —O-alkyl, cycloalkyl, halo, haloalkyl, hydroxy, hydroxyalkyl, -NH₂ or —CN; and R⁶ is H, alkyl, —O-alkyl, —O-haloalkyl, cycloalkyl, halo, haloalkyl, hydroxy, hydroxyalkyl, —NH₂, —NH-alkyl or —CN.

11. The compound of claim 8 wherein R⁴ and R⁵, together with the common carbon atom to which they are attached, join to form a -3- to 7-membered cyclic group selected from cycloalkyl, heterocycloalkyl, aryl and heteroaryl.

12. The compound of claim 8 wherein R⁵ and R⁶, together with the common carbon atom to which they are attached, join to form a cycloalkyl, heterocycloalkyl, aryl or heteroaryl group.

13. The compound of claim 8 wherein R⁶ and R⁷, together with the common carbon atom to which they are attached, join to form a cycloalkyl, heterocycloalkyl, aryl or heteroaryl group.

14. The compound of claim 4, wherein R¹⁰ is aryl or heteroaryl.

15. The compound of claim 14, wherein R¹⁰ is phenyl, naphthyl, pyridyl, quinolinyl or quinoxalinyl.

16. The compound of claim 14, wherein R¹⁰ is: 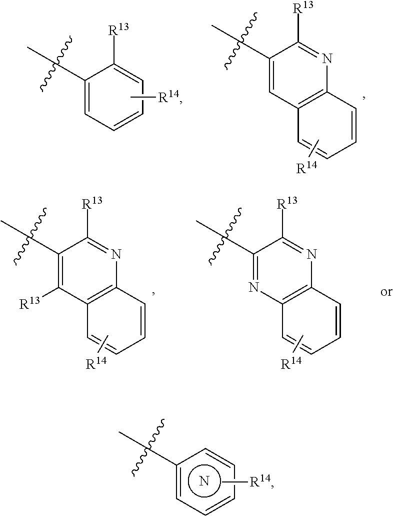
wherein R¹³ is H, F, Br or Cl; R¹⁴ represents up to 4 optional and additional substituents, each independently selected from alkyl, cycloalkyl, CF₃, —CN, halo, —O-alkyl, —NHSO₂-alkyl, —NO₂, —C(O)NH₂, —C(O)NH-alkyl, —C(O)OH, hydroxy, —NH₂, —SO₂alkyl, —SO₂NHalkyl, —S-alkyl, —CH₂NH₂, —CH₂OH, —SO₂NH₂, —NHC(O)-alkyl, —C(O)O-alkyl, —C(O)-heterocycloalkyl and heteroaryl; and 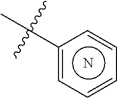
represents a pyridyl group, wherein the ring nitrogen atom can be at any of the five unsubstituted ring atom positions.

17. The compound of claim 16, wherein R⁵ is H, alkyl, —O-alkyl, cycloalkyl, halo, haloalkyl, hydroxy, hydroxyalkyl, —NH₂ or —CN, and R⁶ is H, alkyl, —O-alkyl, cycloalkyl, halo, haloalkyl, hydroxy, hydroxyalkyl, —NH₂ or —CN.

18. The compound of claim 17, wherein R⁵ is methyl, ethyl or cyclopropyl, and R⁶ is H, Cl, For hydroxy.

19. The compound of claim 18, wherein X is —O—; Y is ═O; and R₁ is —CH₂—.

20. The compound of claim 19, wherein Z is —CH—.

21. The compound of claim 1 having the formula: 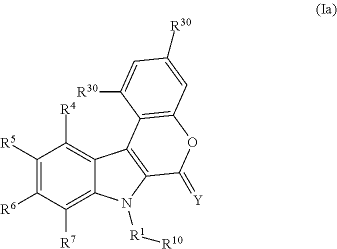
or a pharmaceutically acceptable salt, solvate, ester or prodrug thereof.
wherein:
Y is ═O, ═NH or ═NSO₂R¹¹;
Z is —C(R³¹)—;
R¹ is a bond or an alkylene group;
R⁴ is H or or R⁴ and R⁵, together with the carbon atoms to which they are attached, join to form a 5-membered cyclic group, selected from cycloalkyl, heterocycloalkyl, aryl or heteroaryl;
R⁵ and R⁶ are each independently H, halo, alkyl, —O-alkyl, haloalkyl, —O-haloalkyl, heterocycloalkenyl or cycloalkyl, or R⁵ and R⁶, together with the carbon atoms to which they are attached, join to form a 5-membered cyclic group, selected from cycloalkyl, heterocycloalkyl, aryl or heteroaryl;
R⁷ is H or or R⁶ and R⁷, together with the carbon atoms to which they are attached, join to form a 5-membered cyclic group, selected from cycloalkyl, heterocycloalkyl, aryl or heteroaryl;
R¹⁰ is H, halo, aryl, heterocycloalkenyl or heteroaryl, wherein an aryl or heteroaryl group can be optionally and independently substituted with up to 4 substituents, which are each independently selected from H, alkyl, halo, —NH₂, —OH, —CN, —NO₂, —O-alkyl, —C(O)NH₂, heteroaryl, —SO₂NH₂, —SO₂NH-alkyl, —SO₂-alkyl, phenyl, —NHC(O)OH, —S-alkyl, —NHSO₂-alkyl, alkyl, —NHSO₂-cycloalkyl, —O-benzyl, —C(O)NH-alkyl, —S-haloalkyl or —S(O)-haloalkyl, such that when R¹ is a bond, R¹⁰ is other than H;
each occurrence of R¹¹ is independently alkyl or cycloalkyl;
each occurrence of R³⁰ is independently, H, alkyl, —O-alkylene-C(O)OH, —O-alkylene-C(O)O-alkyl, or any R³⁰ and R³¹, together with the carbon atoms to which they are attached, join to form a 3- to 7-membered cyclic group, selected from cycloalkyl, heterocycloalkyl, aryl and heteroaryl; and
R³¹ is H or halo.

22. Any one of compounds numbered 1-230 in the above specification, or a pharmaceutically acceptable salt, solvate, ester or prodrug thereof.

23. A pharmaceutical composition comprising at least one compound of claim 1 or a pharmaceutically acceptable salt, solvate, ester or prodrug thereof, and at least one pharmaceutically acceptable carrier.

24. A method for treating a viral infection in a patient, the method comprising administering to the patient an effective amount of at least one compound of claim 1 or a pharmaceutically acceptable salt, solvate, ester or prodrug thereof.

25. The method of claim 24, further comprising administering to the patient at least one additional antiviral agent, wherein the additional agent is selected from an HCV polymerase inhibitor, an interferon, a viral replication inhibitor, an antisense agent, a therapeutic vaccine, a viral protease inhibitor, a virion production inhibitor, an antibody therapy (monoclonal or polyclonal), and any agent useful for treating an RNA-dependent polymerase-related disorder.
